(19) United States
(12) Patent Application Publication
(10) Pub. No.: US 2009/0227571 A1
(43) Pub. Date: Sep. 10, 2009

(54) Androgen Receptor Modulator Compounds and Methods

(75) Inventors: Jon C. Loren, San Diego, CA (US); Todd Miller, San Marcos, CA (US); Bijan Pedram, San Diego, CA (US); Charlene V. Rowley, Chula Vista, CA (US); Yixing Shen, Encinitas, CA (US); Cornelis Arjan Van Oeveren, San Diego, CA (US); Lin Zhi, San Diego, CA (US)

Correspondence Address: K&L Gates LLP, 3580 Carmel Mountain Road, Suite 200, San Diego, CA, 92130, US

(73) Assignee: LIGAND PHARMACEUTICALS Incorporated

(21) Appl. No.: 11/921,366

(22) PCT Filed: Jun. 30, 2006
(86) PCT No.: PCT/US2006/026067

§ 371 Date: Feb. 27, 2009

Related U.S. Application Data
(60) Provisional application No. 60/695,949, filed on Jul. 1, 2005.

(51) Int. Cl. A61K 31/54; C12N 5/02; C12Q 1/02; C07C 237/00; A61K 31/16; C07D 209/04; A61K 31/405; C07C 303/00; A61K 31/18; A61K 31/445; C07D 211/00; C07D 215/00; A61K 31/47; A61K 31/4427; C07D 471/00; C07D 401/14; A61K 31/497; C07D 413/00; C07D 417/00; A61K 31/5377

(52) U.S. Cl. 514/228.5; 435/375; 435/29; 564/157; 514/616; 548/490; 514/415; 564/86; 514/603; 514/315; 546/184; 546/157; 514/312; 514/292; 546/80; 544/363; 514/253.07; 544/126; 544/60; 514/232.8

(57) ABSTRACT

Provided herein are compounds that bind to androgen receptors and modulate the activity and/or the amount of androgen receptors and to methods for making and using such compounds. Also provided are compositions including such compounds and methods for making and using such compositions.

### RELATED APPLICATIONS

Priority is claimed herein to U.S. provisional patent application Ser. No. 60/695,949, filed Jul. 1, 2005, entitled “ANDROGEN RECEPTOR MODULATOR COMPOUNDS AND METHODS.” Where permitted, the disclosure of the above-referenced provisional application is incorporated herein by reference in its entirety

### FIELD

Provided herein are compounds that bind to androgen receptors and modulate the activity and/or the amount of androgen receptors and to methods for making and using such compounds. Also provided are compositions including such compounds and methods for making and using such compositions.

### BACKGROUND

Certain intracellular receptors (IRs) have been shown to regulate transcription of certain genes. See e.g., R. M. Evans, Science, 240, 889 (1988). Certain of such IRs are steroid receptors, such as androgen receptors, estrogen receptors, mineralocorticoid receptors, and progesterone receptors. Gene regulation by such receptors typically involves binding of an IR by a ligand.

In certain instances, a ligand binds to an IR, forming a receptor/ligand complex. Such a receptor/ligand complex may then translocate to the nucleus of a cell, where it may bind to the DNA of one or more gene regulatory regions. Once bound to the DNA of a particular gene regulatory region, a receptor/ligand complex may modulate the production of the protein encoded by that particular gene. In certain instances, an androgen receptor/ligand complex regulates expression of certain proteins. In certain instances, an androgen receptor/ligand complex may interact directly with the DNA of a particular gene regulatory region. In certain instances, an androgen receptor/ligand complex may interact with other transcription factors, such as activator protein-1 (AP-1) or nuclear factor κB (NFκB). In certain instances, such interactions result in modulation of transcriptional activation.

### SUMMARY

Compounds for use in compositions and methods for modulating the activity of androgen receptor are provided. In certain embodiments, the compounds provided herein are agonists of androgen receptor. In certain embodiments, the compounds provided herein are antagonists of androgen receptor. In certain embodiments, administration of compounds provided herein results in a decrease in the amount of functional androgen receptor present in cells. In certain embodiments, administration of compounds provided herein results in degradation of androgen receptors.

In certain embodiments, provided herein are compounds having a structure selected from Formula I and Formula Ia:

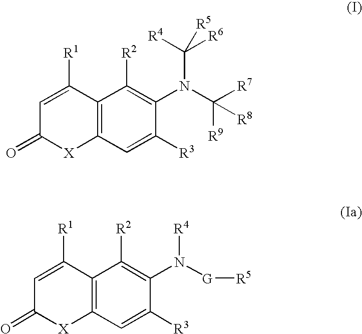

wherein:

R¹, R² and R³ are independently selected from hydrogen, halogen, CN, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R⁴, R⁵, R⁶, R⁷, R⁸ and R⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

m is selected from 0, 1 and 2;

G is selected from —CO—, —CS—, —SO₂— and a bond;

J is selected from optionally substituted aryl, optionally substituted heteroaryl, and optionally substituted non-aromatic heterocycle;

V is selected from O, S and NR^{E};

X is selected from O, S and NR^{A};

provided that at least one of R^{A}, R¹, R², R³, R⁴, R⁵, R⁶, R⁷, R⁸ and R⁹ is QT,

Q is selected from optionally substituted C₂-C₁₂ alkyl, optionally substituted C₂-C₁₂ haloacyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl and optionally substituted C₂-C₁₂ heteroarylheteroalkyl; and

T is selected from NO₂, OH, CN, CO₂R¹, JR^{A}(O)R^{C}R^{D}, R^{C}S(O)₂NR^{C}R^{D}, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, SO₂NR^{C}R^{D}, optionally substituted C₇-C₁₀ haloalkyl and optionally substituted C₇-C₁₀ heteroalkyl,

provided that if R⁴, R⁵, R⁶, R⁷, R⁸, or R⁹ is QT, then T is not S(O)ₘR^{C};

and pharmaceutically acceptable salts and prodrugs thereof.

In certain embodiments of compounds of Formula I and Formula Ia, Q is selected from C₂-C₁₂ alkyl, C₂-C₁₂ haloalkyl, C₂-C₁₂ heteroalkyl, C₂-C₁₂ arylalkyl, C₂-C₁₂ heteroarylalkyl, C₂-C₁₂ arylhaloalkyl, C₂-C₁₂ heteroarylhaloalkyl, C₂-C₁₂ arylheteroalkyl and C₂-C₁₂ heteroarylheteroalkyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, provided herein are compounds having a structure selected from Formula II, Formula Ia and Formula IIb:

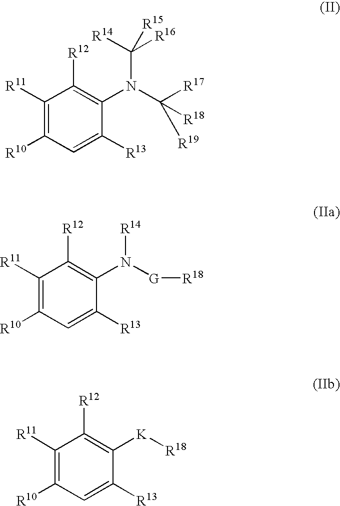

wherein:

R¹⁰ and R¹¹ are independently selected from NO₂, CN, halogen, C₁-C₄ alkyl, C₁-C₄ haloalkyl, C₁-C₄ heteroalkyl and C₁-C₄ heterohaloalkyl;

R¹² and R¹³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R¹⁴, R¹⁵, R¹⁶, R¹⁷, R¹⁸ and R¹⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl, optionally substituted aryl, optionally substituted C₂-C₆ alkenyl and optionally substituted C₂-C₆ heteroalkenyl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

m is selected from 0, 1 and 2;

G is selected from —CO—, —S—, —SO₂— and a bond;

J is selected from optionally substituted aryl, optionally substituted heteroaryl, and optionally substituted non-aromatic heterocycle;

V is selected from O, S and NR^{E};

K is selected from —OP(S)OR^{A}O—, —OP(O)OR^{A}O—, —NR^{B}P(S)OR^{A}O— and —NR^{B}P(O)OR^{A}O—;

provided that at least one of the R^{A}, R¹², R¹³, R¹⁴, R¹⁵, R¹⁶, R¹⁷, R¹⁸ and R¹⁹ is QT,

Q is selected from optionally substituted C₂-C₁₂ alkyl, optionally substituted C₂-C₁₂ haloalkyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₆ alkenyl and optionally substituted C₂-C₆ heteroalkenyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, JS(O)ₘR^{C}R^{D}, JNC(V)NR^{C}R^{D}, JS(O)ₘR^{C}R^{D}, RCS(O)ₘR^{D}, NJC(O)NR^{C}R^{D}, R^{C}J, JR^{C}R^{D}, JC(V)NR^{C}R^{D}, SO₂NR^{C}R^{D}, optionally substituted C₇-C₁₀ haloalkyl and optionally substituted C₇-C₁₀ heteroalkyl;

provided that, if Q is selected from C₂-C₁₂ alkyl, C₂-C₁₂ haloalkyl, C₂-C₁₂ alkoxy, C₂-C₁₂ arylalkyl, C₂-C₁₂ arylhaloalkyl and C₂-C₁₂ heteroarylhaloalkyl, then T is not NO₂, CN, COR^{C}, S(O)₂R^{C}; and if Q is selected from noncyclic C₂-C₈ alkyl and noncyclic C₂-C₈ haloalkyl, then T is not NR^{A}COR^{B}, NR^{A}CO₂R^{B}, or NR^{C}SO₂R^{D}; and pharmaceutically acceptable salts and prodrugs thereof.

In certain embodiments of compounds of Formula II, Formula IIa and Formula IIb, substituents R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, provided herein are compounds having a structure of Formula III:

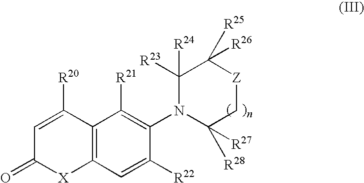

wherein:

R²⁰, R²¹ and R²² are independently selected from hydrogen, halogen, OR^{A}, SR^{A}, NR^{A}R^{B}, optionally substituted C₁-C₄ allyl, optionally substituted C₁-C₄ haloalkyl, optionally substituted C₁-C₄ heteroalkyl, optionally substituted C₁-C₄ heterohaloalkyl and QT;

R²³, R²⁴, R²⁵, R²⁶, R²⁷, R²⁸, R²⁹ and R^{29a} are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl, C₁-C₆ heterohaloalkyl and aryl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

J is selected from optionally substituted aryl, optionally substituted heteroaryl, and optionally substituted non-aromatic heterocycle;

V is selected from O, S and NR^{E};

X is selected from O, S and NR^{A};

Z is selected from O, S, NR²⁹ and CR²⁹R^{29a};

m is selected from 0, 1 and 2;

n is selected from 0, 1, 2 and 3;

provided that at least one of the R^{A}, R²⁰, R²¹, R²², R²³, R²⁴, R²⁵, R²⁶, R²⁷, R²⁸, R²⁹ and R^{29a} is QT;

Q is selected from optionally substituted C₂-C₁₂ alkyl, optionally substituted C₂-C₁₂ haloalkyl, optionally substituted C₂-C₁₂ heteroalkyl, optionally substituted C₂-C₁₂, C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroaryl-heteroalkyl, optionally substituted C₂-C₆ alkenyl and optionally substituted C₂-C₆ heteroalkenyl; and

T is selected from NO₂, CN, CO₂R¹, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, SO₂NR^{C}R^{D}, R^{C}OR^{A}R^{D}, R^{C}S(O)ₘR^{D}R^{E}, NC(O)JNS(O)ₘR^{C}, NR^{C}CR^{D}, VR^{A}(O)J, R^{A}(O)J, R^{A}(O)NR^{C}, C(O)NR^{C}, R^{C}R^{D}, JSO₂R^{D}, VR^{A}(O)NR^{C}, R^{C}S(O)ₘNR^{D}R^{E} and VR^{A}(O)NR^{C}S(O)ₘNR^{C}R^{D}, optionally substituted C₇-C₁₀ haloalkyl and optionally substituted C₇-C₁₀ heteroalkyl;

provided that, if R²³, R²⁴, R²⁵, R²⁶, R²⁷, R²⁸, R²⁹, or R^{29a} is QT, then T is not C₇-C₈ haloalkyl or C₇-C₈ heteroalkyl;

and pharmaceutically acceptable salts and prodrugs thereof.

In certain embodiments of compounds of Formula III, R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, provided herein are compounds having a structure of Formula IV:

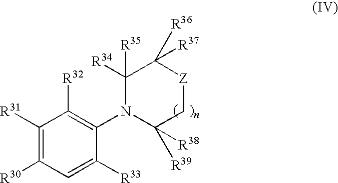

wherein:

R³⁰ and R³¹ are independently selected from NO₂, CN, halogen, C₁-C₄ alkyl, C₁-C₄ haloalkyl, C₁-C₄ heteroalkyl and C₁-C₄ heterohaloalkyl;

R³² and R³³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R³⁴, R³⁵, R³⁶, R³⁷, R³⁸, R³⁹, R^{39a} and R^{39b} are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

V is selected from O, S and NR^{E};

Z is selected from O, S, NR^{39a} and CR^{39a}R^{39b};

m is selected from 0, 1 and 2;

n is selected from 0, 1, 2 and 3;

provided that at least one of the R^{A}, R³², R³³, R³⁴, R³⁵, R³⁶, R³⁷, R³⁸, R³⁹, R^{39a} and R^{39b} is QT,

Q is selected from optionally substituted C₂-C₁₂ alkyl, optionally substituted C₂-C₁₂ haloalkyl, optionally substituted C₂-C₁₂ heteroalkyl, optionally substituted C₂-C₁₂, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₁₂ alkenyl and optionally substituted C₂-C₁₂ heteroalkenylalkenyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, S(O)ₘNR^{C}R^{D}, NR^{C}SO₂R^{D}, SO₂NR^{C}, SO₂NR^{C}R^{D}, C₇-C₁₀ haloalkyl and C₇-C₁₀ heteroalkyl;

provided that if R³⁴, R³⁵, R³⁶, R³⁷, R³⁸, R³⁹, R^{39a}, or R^{39b} is QT, then T is not C₃-C₄ haloalkyl or C₃-C₄ heteroalkyl;

and pharmaceutically acceptable salts and prodrugs thereof.

In certain embodiments of compounds of Formula IV, Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, provided herein are compounds having a structure of Formula V:

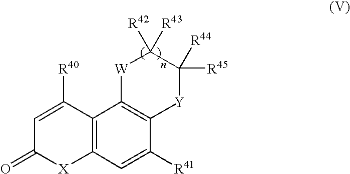

wherein:

R⁴⁰ and R⁴¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R⁴², R⁴³, R⁴⁴, R⁴⁶ and R⁴⁷ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT; or

R⁴² and R⁴⁴ and/or R⁴² and R⁴⁷ and/or R⁴⁴ and R⁴⁶ form a bond;

R⁴⁴ and R⁴⁷ can optionally form a bond when n is 0;

R⁴⁵ is selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl, optionally substituted aryl, R^{C}OR^{D}VJ and QT;

R^{A} and R^{B} are independently selected from hydrogen, C₁-C₆ alkyl, C₁-C₆ haloalkyl, C₁-C₆ heteroalkyl, C₁-C₆ heterohaloalkyl, heteroaryl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl, C₁-C₆ heterohaloalkyl and aryl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

J is selected from optionally substituted aryl, optionally substituted heteroaryl, and optionally substituted non-aromatic heterocycle;

V is selected from O, S and NR^{E};

X is selected from O, S and NR^{A};

Y is selected from O, S, NR⁴⁶ and a bond;

W is selected from O, S, NR⁴⁷ and a bond;

m is selected from 0, 1 and 2;

n is selected from 0, 1, 2 and 3;

provided that at least one of R^{A}, R⁴⁰, R⁴¹, R⁴², R⁴³, R⁴⁴, R⁴⁵, R⁴⁶ and R⁴⁷ is QT,

Q is selected from optionally substituted C₂-C₁₂ alkyl, optionally substituted C₂-C₁₂ haloalkyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₆ alkenyl and optionally substituted C₂-C₆ heteroalkenyl; and

T is a selected from NO₂, CN, OH, C═C, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, SO₂NR^{C}R^{D}, R^{C}NR^{D}, NR^{C}R^{D}, R^{C}R^{D}S(O)ₘJV, VR^{C}S(O)ₘVR^{D}R^{E}, VJ, JR^{C}, R^{C}J, JR^{C}J, JCR^{C}R^{D}JS(O)ₘJ, JR^{C}R^{E}, R^{C}OR^{D}J, JR^{C}JR^{D}R^{E}, JC(O)ₘR^{C}, JR^{C}OR^{D}, optionally substituted C₇-C₁₀ alkyl, optionally substituted C₇-C₁₀ heteroaryl, aryl, optionally substituted C₇-C₁₀ haloalkyl and optionally substituted C₇-C₁₀ heteroalkyl;

and pharmaceutically acceptable salts and prodrugs thereof.

In certain embodiments of compounds of Formula V, substituents R⁴², R⁴³, R⁴⁴, R⁴⁵, R⁴⁶ and R⁴⁷ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl

In certain embodiments, provided herein are compounds having a structure selected from among Formula VI:

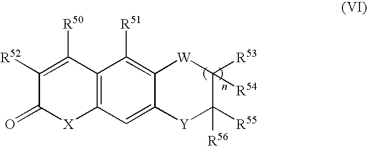

wherein:

R⁵⁰ and R⁵¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R⁵² is selected from hydrogen, F, Cl, Br, CH₃ and CF₃;

R⁵³, R⁵⁴, R⁵⁵, R⁵⁶, R⁵⁷ and R⁵⁸ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R⁵³ and R⁵⁵, R⁵³ and R⁵⁷, or R⁵⁵ and R⁵⁸ can optionally form a bond;

R⁵⁵ and R⁵⁷ can optionally form a bond when n is 0;

R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;

R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;

R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;

V is selected from O, S and NR^{E};

X is selected from O, S and NR^{A};

Y is selected from O, S, NR⁵⁸ and a bond;

W is selected from O, S, NR⁵⁷ and a bond;

m is selected from 0, 1 and 2;

n is selected from 0, 1, 2 and 3;

provided that at least one of the R^{A}, R⁵⁰, R⁵¹, R⁵³, R⁵⁴, R⁵⁵, R⁵⁶, R⁵⁷ and R⁵⁸ is QT

Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, S(O)ₘR^{C}R^{D}, NR^{C}SO₂R^{D}, SO₂NR^{C}, SO₂NR^{C}R^{D}, SO₂NR^{C}, C₇-C₁₀ haloalkyl and C₇-C₁₀ heteroalkyl.

provided that, if W is a bond and Y is NH and n is 3 and each of R⁵¹, R⁵², R⁵⁵ and R⁵⁶ is hydrogen and X is either O or NR^{A} and either the R⁵³ or the R⁵⁴ bound to the carbon that is closest to Y is QT, then T is not NO₂, CN, C₇-C₈ cycloheteroalkyl, NHCOR^{C}, COR^{C}, O₂CR^{C}, CO₂R^{C}, S(O)ₘR^{C}, CONR^{C}R^{D}, NHCO₂R^{C}, OC(O)NR^{C}R^{D}, NR^{C}C(O)NR^{C}R^{D} and NR^{C}SO₂R^{D}; and

if W is a bond, and Y is NH, and n is 3, and each of R⁵¹, R⁵², R⁵⁵, and R⁵⁶ is hydrogen, and X is NR^{A} where R^{A} is QT, and where Q is saturated noncyclic alkyl, then T is not CO₂H;

and pharmaceutically acceptable salts and prodrugs thereof.

In certain embodiments of compounds of Formula VI, T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

Certain compounds have been previously described as receptor modulators or as possible receptor modulators. See e.g., U.S. Pat. Nos. 6,462,038, 5,693,646; 6,380,207; 6,506,766; 5,688,810; 5,696,133; 6,569,896, 6,673,799; 4,636,505; 4,097,578; 3,847,988; U.S. application Ser. No. 10/209,461 (Pub. No. US 2003/0055094); WO 01/27086; WO 02/22585; Zhi, et al. Bioorganic & Medicinal Chemistry Letters 2000, 10, 415-418; Pooley et al., J. Med. Chem. 1998, 41, 3461; Hamann et al. J. Med. Chem. 1998, 41 (4), 623; and Yin et al., Molecular Pharmacology, 2003, 63 (1), 211-223 the entire disclosures of which are incorporated in their entirety.

In certain embodiments, compounds provided herein are selective androgen receptor modulators. In certain embodiments, compounds provided herein are selective androgen receptor agonists. In certain embodiments, compounds provided herein are selective androgen receptor antagonists. In certain embodiments, compounds provided herein are selective androgen receptor partial agonists. In certain embodiments, compounds provided herein are tissue specific selective androgen receptor modulators. In certain embodiments, compounds provided herein are selective androgen receptor binding compounds. In certain embodiments, compounds provided herein are selective androgen receptor reducing compounds. In certain embodiments, compounds provided herein are selective androgen receptor degrading compounds.

In certain embodiments, compounds provided herein are effective for treating one or more androgen receptor mediated disease or condition. In certain embodiments, compounds provided herein are effective for treating one or more diseases or conditions including, but not limited to, increase or maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic myalgia; treatment of acute fatigue syndrome and muscle loss following elective surgery (e.g., post-surgical rehabilitation); accelerating of wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells including the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain embodiments, compounds provided herein are effective for treating one or more of acne, male-pattern baldness, wasting diseases, hirsutism, hypogonadism, osteoporoses, infertility, impotence and cancer.

In certain embodiments, compounds provided herein are effective for treating prostate cancer. In certain embodiments, compounds provided herein are effective for treating androgen dependant prostate cancer. In certain embodiments, compounds provided herein are effective for treating androgen independent prostate cancer. In certain embodiments, compounds provided herein are effective for treating androgen independent androgen receptor dependent prostate cancer.

In certain embodiments, provided herein are methods for modulating an activity of an androgen receptor by contacting an androgen receptor with at least one compound provided herein. In certain such embodiments, the androgen receptor is in a cell.

In certain embodiments, provided herein are methods for decreasing the number of functional androgen receptors present in a cell by contacting an androgen receptor with at least one compound provided herein.

In certain embodiments, provided herein are methods for identifying a compound that is capable of modulating an activity of an androgen receptor and/or decreasing the number of functional androgen receptors by contacting a cell expressing an androgen receptor with a compound provided herein and monitoring, an effect of the compound upon the cell.

In certain embodiments, provided herein are methods for treating a patient by administering to the patient a compound provided herein. In certain embodiments, the methods provided herein are for increase or maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic myalgia; treatment of acute fatigue syndrome and muscle loss following elective surgery (e.g., post-surgical rehabilitation); accelerating of wound healing; accelerating bone fracture repair (such as accelerating the recovery of hip fracture patients); accelerating healing of complicated fractures, e.g. distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells including the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain of such embodiments, the patient has a condition selected from acne, male-pattern baldness, wasting diseases, hirsutism, hypogonadism, osteoporoses, infertility, impotence and cancer.

In certain embodiments, the methods provided herein are for treating a condition including, but not limited to, prostate cancer. In certain such embodiments, the prostate cancer is androgen independent prostate cancer. In certain embodiments, the prostate cancer is androgen independent androgen receptor dependant prostate cancer.

Pharmaceutical compositions formulated for administration by an appropriate route and means including effective concentrations of one or more of the compounds provided herein, or pharmaceutically acceptable derivatives thereof, that deliver amounts effective for the treatment, prevention, or amelioration of one or more symptoms of diseases or disorders that are modulated or otherwise affected by androgen receptor activity, or in which androgen receptor activity is implicated, are also provided. The effective amounts and concentrations are effective for ameliorating any of the symptoms of any of the diseases or disorders.

In certain embodiments, provided herein is a pharmaceutical composition including: i) a physiologically acceptable carrier, diluent and/or excipient; and ii) one or more compounds provided herein.

Articles of manufacture including packaging material, within the packaging material a compound or composition, or pharmaceutically acceptable derivative thereof, which is effective for modulating the activity of androgen receptor, or for treatment, prevention or amelioration of one or more symptoms of androgen receptor mediated diseases or disorders, or diseases or disorders in which androgen receptor activity is implicated, and a label that indicates that the compound or composition, or pharmaceutically acceptable derivative thereof, is used for modulating the activity of androgen receptor, or for treatment, prevention or amelioration of one or more symptoms of androgen receptor mediated diseases or disorders, or diseases or disorders in which androgen receptor activity is implicated, are provided.

### DETAILED DESCRIPTION

### A. Definitions

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that both the foregoing general description and the following detailed description are exemplary and explanatory only and are not restrictive of the subject matter claimed. In this application, the use of the singular includes the plural unless specifically stated otherwise. In this application, the use of “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals and treatises are hereby expressly incorporated by reference in their entirety for any purpose.

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard techniques may be used for chemical syntheses, chemical analyses, pharmaceutical preparation, formulation, and delivery, and treatment of patients. Standard techniques may be used for recombinant DNA, oligonucleotide synthesis and tissue culture and transformation (e.g., electroporation, lipofection). Reactions and purification techniques may be performed e.g., using kits according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “selective binding compound” refers to a compound that selectively binds to any portion of one or more target receptors.

As used herein, the term “selective androgen receptor binding compound” refers to a compound that selectively binds to any portion of a androgen receptor.

As used herein, the term “selective androgen receptor reducing compound” refers to a compound, the presence of which results in a decrease in the number of functional androgen receptors in a cell. In certain embodiments, the presence of a selective androgen receptor reducing compound results in a decrease of at least 5%, 10%, 20%, 30%, 40%, 50%, 60%, 70%, 80%, 90%, 95%, 98%, or 99% of the functional androgen receptors in a cell.

As used herein, the term “functional androgen receptor” refers to an androgen receptor that is capable of performing at least one activity associated with intact or native androgen receptors.

As use herein, the term “selective androgen receptor degrading compound” refers to a compound, the presence of which results in degradation of androgen receptors in a cell. In certain embodiments, the presence of a selective androgen receptor degrading compound results in degradation of at least 5%, 10%, 20%, 30%, 40%, 50%, 60%, 70%, 80%, 90%, 95%, 98%, or 99% of the androgen receptors in a cell.

As used herein, the term “selectively binds” refers to the ability of a selective binding compound to bind to a target receptor with greater affinity than it binds to a non-target receptor. In certain embodiments, specific binding refers to binding to a target with an affinity that is at least 10, 50, 100, 250, 500, 1000 or more times greater than the affinity for a non-target.

As used herein, the term “target receptor” refers to a molecule or a portion of a receptor capable of being bound by a selective binding compound. In certain embodiments, a target receptor is a androgen receptor.

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

As used herein, the term “modulator” refers to a compound that alters an activity of a molecule. For example, a modulator can cause an increase or decrease in the magnitude of a certain activity of a molecule compared to the magnitude of the activity in the absence of the modulator. In certain embodiments, a modulator is an inhibitor, which decreases the magnitude of one or more activities of a molecule. In certain embodiments, an inhibitor completely prevents one or more activities of a molecule. In certain embodiments, a modulator is an activator, which increases the magnitude of at least one activity of a molecule. In certain embodiments the presence of a modulator results in an activity that does not occur in the absence of the modulator.

As used herein, the term “selective modulator” refers to a compound that selectively modulates a target activity.

As used herein, the term “selective androgen receptor modulator” refers to a compound that selectively modulates at least one activity associated with an androgen receptor.

As used herein, the term “selectively modulates” refers to the ability of a selective modulator to modulate a target activity to a greater extent than it modulates a non-target activity. In certain embodiments the target activity is selectively modulated by, for example about 2 fold up to more that about 500 fold, in some embodiments, about 2, 5, 10, 50, 100, 150, 200, 250, 300, 350, 400, 450 or more than 500 fold.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes and amelioration of one or more symptoms associated with a disease or condition.

As used herein, the term “receptor mediated activity” refers to any biological activity that results, either directly or indirectly, from binding of a ligand to a receptor.

As used herein, the term “agonist” refers to a compound, the presence of which results in a biological activity of a receptor that is the same as the biological activity resulting from the presence of a naturally occurring ligand for the receptor.

As used herein, the term “partial agonist” refers to a compound the presence of which results in a biological activity of a receptor that is of the same type as that resulting from the presence of a naturally occurring ligand for the receptor, but of a lower magnitude.

As used herein, the term “antagonist” refers to a compound, the presence of which results in a decrease in the magnitude of a biological activity of a receptor. In certain embodiments, the presence of an antagonist results in complete inhibition of a biological activity of a receptor.

As used herein, the IC₅₀ refers to an amount, concentration or dosage of a particular test compound that achieves a 50% inhibition of a maximal response, such as modulation of androgen receptor activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, C₁-Cₓ includes C₁-C₂, C₁-C₃ . . . C₁-Cₓ.

As used herein, the term “alkyl” alone or in combination refers to a straight, branched, or cyclic chain including at least one carbon atom. An alkyl group can be a “saturated alkyl,” which means that it does not include any alkene or alkyne groups. An alkyl group can be an “unsaturated alkyl,” which means that it includes at least one alkene or alkyne group. In certain embodiments, alkyls are optionally substituted.

In certain embodiments, an alkyl includes 1 to 20 carbon atoms (whenever it appears herein, a numerical range such as “1 to 20” refers to each integer in the given range; e.g., “1 to 20 carbon atoms” means that an alkyl group can include only 1 carbon atom, 2 carbon atoms, 3 carbon atoms, etc., up to and including 20 carbon atoms, although the term “alkyl” also includes instances where no numerical range of carbon atoms is designated). An alkyl can be designated as “C₁-C₄ alkyl” or similar designations. By way of example only, “C₁-C₄ alkyl” indicates an alkyl having one, two, three, or four carbon atoms, i.e., the alkyl is selected from among methyl, ethyl, propyl, iso-propyl, n-butyl, iso-butyl, sec-butyl and t-butyl. Thus C₁-C₄ includes C₁-C₂ and C₁-C₃ alkyl. Alkyls can be substituted or unsubstituted. Alkyls include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, each of which can be optionally substituted.

As used herein, the term “alkenyl” alone or in combination refers to an alkyl group including at least one carbon-carbon double bond. In certain embodiments, alkenyls are optionally substituted.

As used herein, the term “alkynyl” alone or in combination refers to an alkyl group including at least one carbon-carbon triple bond. In certain embodiments, alkynyls are optionally substituted.

As used herein, the term “non-cyclic alkyl” refers to an alkyl that is not cyclic (i.e., a straight or branched chain including at least one carbon atom). Non-cyclic alkyls may be fully saturated or may include non-cyclic alkenes and/or alkynes. Non-cyclic alkyls may be optionally substituted.

As used herein, the term “haloalkyl” alone or in combination refers to an alkyl in which at least one hydrogen atom is replaced with a halogen atom. In certain of the embodiments in which two or more hydrogen atom are replaced with halogen atoms, the halogen atoms are all the same as one another. In certain of such embodiments, the halogen atoms are not all the same as one another. Certain haloalkyls are saturated haloalkyls, which do not include any carbon-carbon double bonds or any carbon-carbon triple bonds. Certain haloalkyls are haloalkenes, which include one or more carbon-carbon double bonds. Certain haloalkyls are haloalkynes, which include one or more carbon-carbon triple bonds. In certain embodiments, haloalkyls are optionally substituted

As used herein, the term “heteroalkyl” alone or in combination refers to a group including an alkyl and one or more heteroatoms. Certain heteroalkyls are saturated heteroalkyls, which do not include any carbon-carbon double bonds or any carbon-carbon triple bonds. Certain heteroalkyls are heteroalkenes, which include at least one carbon-carbon double bond. Certain heteroalkyls are heteroalkynes, which include at least one carbon-carbon triple bond. Certain heteroalkyls are acylalkyls, in which the one or more heteroatoms are within an alkyl chain. Examples of heteroalkyls include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃OCH₂CH₂—, CH₃C(═O)CH₂— and CH₃NHCH₂—. In certain embodiments, heteroalkyls are optionally substituted.

As used herein, the term “heterohaloalkyl” alone or in combination refers to a heteroalkyl in which at least one hydrogen atom is replaced with a halogen atom. In certain embodiments, heterohaloalkyls are optionally substituted.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls) and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

As used herein, the term “heterocycle” refers to a ring wherein at least one atom forming the ring is a carbon atom and at least one atom forming the ring is a heteroatom. Heterocyclic rings may be formed by three, four, five, six, seven, eight, nine, or more than nine atoms. Any number of those atoms may be heteroatoms (i.e., a heterocyclic ring may include one, two, three, four, five, six, seven, eight, nine, or more than nine heteroatoms, provided that at least one atom in the ring is a carbon atom). Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocylic ring will have additional heteroatoms in the ring. Designations such as “4-6 membered heterocycle” refer to the total number of atoms that include the ring (i.e., a four, five, or six membered ring, in which at least one atom is a carbon atom, at least one atom is a heteroatom and the remaining two to four atoms are either carbon atoms or heteroatoms). In heterocycles including two or more heteroatoms, those two or more heteroatoms may be the same or different from one another. Heterocycles may be optionally substituted. Binding to a heterocycle can be at a heteroatom or via a carbon atom. Examples of heterocycles include, but are not limited to the following:

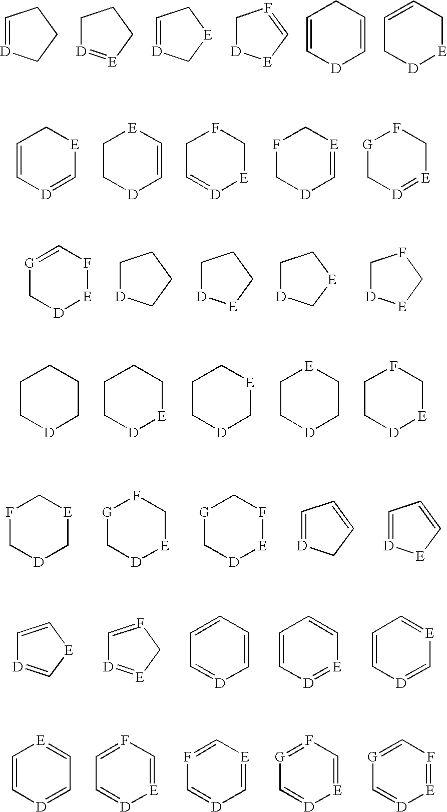

wherein D, E, F and G independently represent a heteroatom. Each of D, E, F and G may be the same or different from one another.

As used herein, the term “heteroatom” refers to an atom other than carbon or hydrogen. Heteroatoms are typically independently selected from among oxygen, sulfur, nitrogen and phosphorus, but are not limited to those atoms. In embodiments in which two or more heteroatoms are present, the two or more heteroatoms can all be the same as one another, or some or all of the two or more heteroatoms can each be different from the others.

As used herein, the term “aromatic” refers to a planar ring having a delocalized n-electron system including 4n+2π electrons, where n is an integer. Aromatic rings may be formed by five, six, seven, eight, nine, or more than nine atoms. Aromatics may be optionally substituted. Examples of aromatic groups include, but are not limited to phenyl, naphthalenyl, phenanthrenyl, anthracenyl, tetralinyl, fluorenyl, indenyl and indanyl. The term “aromatic” includes, for example, benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁-C₆ alkoxy, a C₁-C₆ alkyl, a C₁-C₆ hydroxyalkyl, a C₁-C₆ aminoalkyl, a C₁-C₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups including substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethyl-phenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolylphenyl, 4-triazolylphenyl and 4-(2-oxopyrrolidin-1-yl)phenyl.

As used herein, the term “aryl” refers to an aromatic ring wherein each of the atoms forming the ring is a carbon atom. Aryl rings can be formed by three, four, five, six, seven, eight, nine, or more than nine carbon atoms. Aryl groups can be optionally substituted.

As used herein, the term “heteroaryl” refers to an aromatic ring in which at least one atom forming the aromatic ring is a heteroatom. Heteroaryl rings can be formed by three, four, five, six, seven, eight, nine and more than nine atoms. Heteroaryl groups can be optionally substituted. Examples of heteroaryl groups include, but are not limited to, aromatic C₃-C₈ heterocyclic groups including one oxygen or sulfur atom or up to four nitrogen atoms, or a combination of one oxygen or sulfur atom and up to two nitrogen atoms and their substituted as well as benzo- and pyrido-fused derivatives, for example, connected via one of the ring-forming carbon atoms. In certain embodiments, heteroaryl groups are optionally substituted with one or more substituents, independently selected from halo, hydroxy, amino, cyano, nitro, alkylamido, acyl, C₁-C₆-alkoxy, C₁-C₆-alkyl, C₁-C₆-hydroxyalkyl, C₁-C₆-aminoalkyl, C₁-C₆-alkylamino, alkylsulfenyl, alkylsulfinyl, alkylsulfonyl, sulfamoyl, or trifluoromethyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, phthalimide, quinazoline and quinoxaline. In some embodiments, the substituents are halo, hydroxy, cyano, O—C₁-C₆-alkyl, C₁-C₆-alkyl, hydroxy-C₁-C₆-alkyl and amino-C₁-C₆-alkyl.

As used herein, the term “non-aromatic ring” refers to a ring that does not have a delocalized 4n+2π-electron system.

As used herein, the term “cycloalkyl” refers to a group including a non-aromatic ring wherein each of the atoms forming the ring is a carbon atom. Cycloalkyls can be formed by three, four, five, six, seven, eight, nine, or more than nine carbon atoms. Cycloalkyls can be optionally substituted. In certain embodiments, a cycloalkyl includes one or more unsaturated bonds. Examples of cycloalkyls include, but are not limited to, cyclopropane, cyclobutane, cyclopentane, cyclopentene, cyclopentadiene, cyclohexane, cyclohexene, 1,3-cyclohexadiene, 1,4-cyclohexadiene, cycloheptane and cycloheptene.

As used herein, the term “non-aromatic heterocycle” refers to a non-aromatic ring wherein one or more atoms forming the ring is a heteroatom. Non-aromatic heterocyclic rings can be formed by three, four, five, six, seven, eight, nine, or more than nine atoms. Non-aromatic heterocycles can be optionally substituted. In certain embodiments, non-aromatic heterocycles include one or more carbonyl or thiocarbonyl groups such as, for example, oxo- and thio-including groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine and 1,3-oxathiolane.

As used herein, the term “arylalkyl” alone or in combination, refers to an alkyl substituted with an aryl that may be optionally substituted.

As used herein, the term “heteroarylalkyl” alone or in combination, refers to an alkyl substituted with a heteroaryl that may be optionally substituted.

As used herein, the substituent “R” appearing by itself and without a number designation refers to a substituent selected from alkyl, cycloalkyl, aryl, heteroaryl (bonded through a ring carbon) and non-aromatic heterocycle (bonded through a ring carbon).

As used herein, the term “O-carboxy” refers to a group of formula RC(═O)O—.

As used herein, the term “C-carboxy” refers to a group of formula —C(═O)OR.

As used herein, the term “acetyl” refers to a group of formula —C(═O)CH₃.

As used herein, the term “trihalomethanesulfonyl” refers to a group of formula X₃CS(═O)₂— where X is a halogen.

As used herein, the term “cyano” refers to a group of formula —CN.

As used herein, the term “isocyanato” refers to a group of formula —NCO.

As used herein, the term “thiocyanato” refers to a group of formula —CNS.

As used herein, the term “isothiocyanato” refers to a group of formula —NCS.

As used herein, the term “sulfinyl” refers to a group of formula —S(═O)—R.

As used herein, the term “S-sulfonamido” refers to a group of formula —S(═O)₂NR₂.

As used herein, the term “N-sulfonamido” refers to a group of formula RS(═O)₂NH—.

As used herein, the term “trihalomethanesulfonamido” refers to a group of formula X₃CS(═O)₂NR—.

As used herein, the term “O-carbamyl” refers to a group of formula —OC(═O)—NR₂.

As used herein, the term “N-carbamyl” refers to a group of formula ROC(═O)NH—.

As used herein, the term “O-thiocarbamyl” refers to a group of formula —OC(═S)—NR₂.

As used herein, the term “N-thiocarbamyl” refers to a group of formula ROC(═S)NH—.

As used herein, the term “C-amido” refers to a group of formula —C(═O)—NR₂.

As used herein, the term “N-amido” refers to a group of formula RC(═O)NH—.

As used herein, the term “ester” refers to a chemical moiety with formula —(R)ₙ—COOR′, where R and R′ are independently selected from alkyl, cycloalkyl, aryl, heteroaryl (bonded through a ring carbon) and non-aromatic heterocycle (bonded through a ring carbon), where n is 0 or 1.

As used herein, the term “amide” refers to a chemical moiety with formula —(R)ₙ—C(O)NHR′ or —(R)ₙ—NHC(O)R′, where R and R′ are independently selected from alkyl, cycloalkyl, aryl, heteroaryl (bonded through a ring carbon) and heteroalicyclic (bonded through a ring carbon), where n is 0 or 1. In certain embodiments, an amide can be an amino acid or a peptide.

As used herein, the terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

As used herein, the term “linked to form a ring” refers to instances where two atoms that are bound either to a single atom or to atoms that are themselves ultimately bound, are each bound to a linking group, such that the resulting structure forms a ring. That resulting ring includes the two atoms that are linked to form a ring, the atom (or atoms) that previously linked those atoms and the linker. For example, if A and B below are “linked to form a ring”

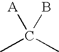

the resulting ring includes A, B, C and a linking group. Unless otherwise indicated, that linking group may be of any length and may be optionally substituted. Referring to the above example, resulting structures include, but are not limited to:

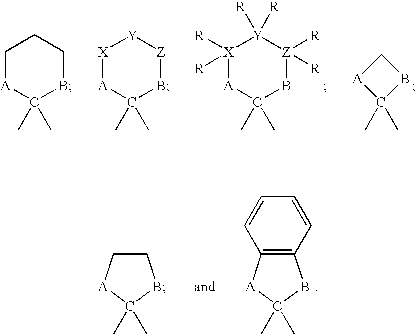

In certain embodiments, the two substituents that together form a ring are not immediately bound to the same atom. For example, if A and B, below, are linked to form a ring:

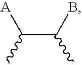

the resulting ring includes A, B, the two atoms that already link A and B and a linking group. Examples of resulting structures include, but are not limited to:

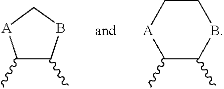

In certain embodiments, the atoms that together form a ring are separated by three or more atoms. For example, if A and B, below, are linked to form a ring:

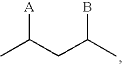

the resulting ring includes A, B, the 3 atoms that already link A and B and a linking group. Examples of resulting structures include, but are not limited to:

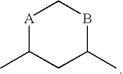

As used herein, the term “together form a bond” refers to the instance in which two substituents to neighboring atoms are null and the bond between the neighboring atoms becomes a double bond. For example, if A and B below “together form a bond”

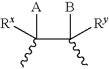

the resulting structure is:

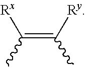

Unless otherwise indicated, the term “optionally substituted,” refers to a group in which none, one, or more than one of the hydrogen atoms has been replaced with one or more group(s) individually and independently selected from: cycloalkyl, aryl, heteroaryl, non-aromatic heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives of amino groups. Such protective derivatives (and protecting groups that can form such protective derivatives) are known to those of skill in the art and can be found in references such as Greene and Wuts, above. In embodiments in which two or more hydrogen atoms have been substituted, the substituent groups can together form a ring.

Throughout the specification, groups and substituents thereof can be chosen by one skilled in the field to provide stable moieties and compounds.

As used herein, the term “carrier” refers to a compound that facilitates the incorporation of another compound into cells or tissues. For example, dimethyl sulfoxide (DMSO) is a commonly used carrier for improving incorporation of certain organic compounds into cells or tissues.

As used herein, the term “pharmaceutical composition” refers to a chemical compound or composition capable of inducing a desired therapeutic effect in a patient. In certain embodiments, a pharmaceutical composition includes an active agent, which is the agent that induces the desired therapeutic effect. In certain embodiments, a pharmaceutical composition includes a prodrug. In certain embodiments, a pharmaceutical composition includes inactive ingredients such as carriers and excipients.

As used herein, the term “therapeutically effective amount” refers to an amount of a pharmaceutical composition sufficient to achieve a desired therapeutic effect.

As used herein, a “prodrug” refers to a compound that is converted from a less active form into a corresponding more active form in vivo. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392 and Silverman, The Organic Chemistry of Drug Design and Drug Action, Academic Press, Inc., (1992); Chapter 8: “Prodrugs and Drug Delivery Systems,” pp 352-401).

As used herein, the term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

As used herein, pharmaceutically acceptable derivatives of a compound include, but are not limited to, salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanol amine and other hydroxyalkylamines, ethylenediamine, N-methyl glucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)-amino-methane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl and heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl and heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

It is to be understood that the compounds provided herein can include chiral centers. Such chiral centers can be of either the (R) or (S) configuration, or can be a mixture thereof. Thus, the compounds provided herein can be enantiomerically pure, or be stereoisomeric or diastereomeric mixtures.

As used herein, the term “substantially pure” means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Thus, substantially pure object species (e.g., compound) is the predominant species present (i.e., on a molar basis it is more abundant than any other individual species in the composition). In certain embodiments, a substantially purified fraction is a composition wherein the object species includes at least about 50 percent (on a molar basis) of all species present. In certain embodiments, a substantially pure composition will include more than about 50%, 60%, 70%, 80%, 85%, 90%, 95%, or 99% of all species present in the composition. In certain embodiments, a substantially pure composition will include more than about 80%, 85%, 90%, 95%, or 99% of all species present in the composition. Methods for purification of the compounds to produce substantially chemically pure compounds are known to those of skill in the art. A substantially chemically pure compound can, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers can be prepared using chiral synthons or chiral reagents, or resolved using conventional techniques, such as reverse phase HPLC. When the compounds described herein include olefinic double bonds or other centers of geometric asymmetry, and unless specified otherwise, it is intended that the compounds include both E and Z geometric isomers. Likewise, all tautomeric forms are also intended to be included.

As used herein, the term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

As used herein, the term “subject” is an animal, typically a mammal, including human.

As used herein, the term “patient” includes human and animal subjects.

As used herein, the term “tissue-selective” refers to the ability of a compound to modulate a biological activity in one tissue to a greater or lesser degree than it modulates a biological activity in another tissue. The biological activities in the different tissues can be the same or they can be different. The biological activities in the different tissues can be mediated by the same type of target receptor. For example, in certain embodiments, a tissue-selective compound can modulate an androgen receptor mediated biological activity in one tissue and fail to modulate, or modulate to a lesser degree, an androgen receptor mediated biological activity in another tissue type.

As used herein, the term “monitoring” refers to observing an effect or absence of any effect. In certain embodiments, one monitors cells after contacting those cells with a compound provided herein. Examples of effects that can be monitored include, but are not limited to, changes in cell phenotype, cell proliferation, androgen receptor activity, or the interaction between an androgen receptor and a natural binding partner.

As used herein, the term “cell phenotype” refers to physical or biological characteristics. Examples of characteristics that constitute phenotype included, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

As used herein, the term “contacting” refers to bringing two or more materials into close enough proximity that they can interact. In certain embodiments, contacting can be accomplished in a vessel such as a test tube, a petri dish, or the like. In certain embodiments, contacting can be performed in the presence of additional materials. In certain embodiments, contacting can be performed in the presence of cells. In certain of such embodiments, one or more of the materials that are being contacted can be inside a cell. Cells can be alive or can be dead. Cells can be intact or can be not intact.

### B. Compounds

Certain compounds that bind to androgen receptors and/or modulate an activity of such receptors play a role in health (e.g., normal growth, development, and/or absence of disease). In certain embodiments, selective androgen receptor modulators, binding compounds, and/or degrading compounds are useful for treating any of a variety of diseases or conditions.

Certain compounds have been previously described as receptor modulators or as possible receptor modulators. See e.g., U.S. Pat. Nos. 6,462,038, 5,693,646; 6,380,207; 6,506,766; 5,688,810; 5,696,133; 6,569,896, 6,673,799; 4,636,505; 4,097,578; 3,847,988; U.S. application Ser. No. 10/209,461 (Pub. No. US 2003/0055094); WO 01/27086; WO 02/22585; Zhi, et al. Bioorganic & Medicinal Chemistry Letters 2000, 10, 415-418; Pooley, et al., J. Med. Chem. 1998, 41, 3461; Hamann, et al. J. Med. Chem. 1998, 41 (4), 623; and Yin, et al., Molecular Pharmacology, 2003, 63 (1), 211-223 the entire disclosures of which are incorporated in their entirety. Exemplary compounds include, but are not limited to: **

1,2,3,6-Tetrahydro-1-methyl-9-(trifluoromethyl)-7H-[1,4]oxazino[3,2-g]quinolin-7-one; 1,2,3,6-Tetrahydro-1,6-dimethyl-9-(trifluoromethyl)-7H-[1,4]oxazino[3,2-g]quinolin-7-one; 1-Ethyl-1,2,3,6-tetrahydro-9-(trifluoromethyl)-7H-[1,4]oxazino[3,2-g]quinolin-7-one; 1-Ethyl-1,2,3,6-tetrahydro-6-methyl-9-(trifluoromethyl)-7H-[1,4]oxazino[3,2-g]-quinolin-7-one; 1,2,3,6-tetrahydro-1-(2,2,2-trifluoroethyl)-9-(trifluoromethyl)-7H-[1,4]oxazino[3,2-g]quinolin-7-one; 8-Fluoro-1,2,3,6-tetrahydro-1-(2,2,2-trifluoroethyl)-9-(trifluoromethyl)-7H-[1,4]oxazino[3,2-g]-quinolin-7-one; 8-Chloro-1,2,3,6-tetrahydro-1-(2,2,2-trifluoroethyl)-9-(trifluoromethyl)-7H-[1,4]oxazino-[3,2-g]-quinolin-7-one; 9-(Difluoromethyl)-1,2,3,6-tetrahydro-1-(2,2,2-trifluoroethyl)-7H-[1,4]oxazino[3,2-g]quinolin-7-one; 1,2,3,6-Tetrahydro-6-methyl-1-(2,2,2-trifluoro-ethyl)-9-(trifluoromethyl)-7H-[1,4]oxazino[3,2-g]-quinolin-7-one; 1,2,3,6-tetrahydro-1-(2,2,2-trifluoroethyl)-9-(trifluoromethyl)-7H-[1,4]oxazino[3,2-g]quinolin-7-thione; 1,2,3,6-Tetrahydro-1-propyl-9-(trifluoromethyl)-7H-[1,4]oxazino[3,2-g]quinolin-7-one; 1,2,3,6-Tetrahydro-1-isobutyl-9-(trifluoromethyl)-7H-[1,4]oxazino[3,2-g]quinolin-7-one; 1,2,3,6-Tetrahydro-1-isobutyl-6-methyl-9-(trifluoromethyl)-7H-[1,4]oxazino[3,2-g]quinolin-7-one; (−)-1,2,3,6-Tetrahydro-3-methyl-1-(2,2,2-trifluoroethyl)-9-(trifluoromethyl)-7H-[1,4]oxazino[3,2-g]-quinolin-7-one; (±)-1,2,3,6-Tetrahydro-3-methyl-1-(2,2,2-trifluoroethyl)-9-(trifluoromethyl)-7H-[1,4]oxazino[3,2-g]-quinolin-7-one; (±)-1,2,3,6-Tetrahydro-1,3-dimethyl-9-(trifluoromethyl)-7H-[1,4]oxazino[3,2-g]quinolin-7-one; (±)-3-Ethyl-1,2,3,6-tetrahydro-1-(2,2,2-trifluoroethyl)-9-(trifluoro-methyl)-7H-[1,4]oxazino[3,2-g]-quinolin-7-one; (±)-3-Ethyl-1,2,3,6-tetrahydro-1-methyl-9-(trifluoromethyl)-7H-[1,4]oxazino[3,2-g]quinolin-7-one, (±)-1,2,3,6-Tetra-hydro-9-(trifluoromethyl)-7H-[1,4]oxazino[3,2-g]quinolin-7-one; 1-Cyclopropylmethyl-1,2,3,6-tetrahydro-9-(trifluoromethyl)-7H-[1,4]oxazino[3,2-g]quinolin-7-one; 1,2,3,6-Tetrahydro-1-(pyridylmethyl)-9-(trifluoromethyl)-7H-[1,4]oxazino[3,2-g]quinolin-7-one; (±)-1,2,3,6-Tetrahydro-2-methyl-1-(2,2,2-trifluoroethyl)-9-(trifluoromethyl)-7H-[1,4]oxazino[3,2-g]-quinolin-7-one; (−)-1,2,3,6-Tetrahydro-2-methyl-1-(2,2,2-trifluoroethyl)-9-(trifluoromethyl)-7H-[1,4]oxazino[3,2-g]-quinolin-7-one; (±)-trans-1,2,3,6-Tetrahydro-2,3-dimethyl-1-(2,2,2-trifluoroethyl)-9-(trifluoromethyl)-7H-[1,4]oxazino-[3,2-g]quinolin-7-one; (±)-cis-1,2,3,6-Tetrahydro-2,3-dimethyl-1-(2,2,2-trifluoroethyl)-9-(trifluoromethyl)-7H-[1,4]oxazino-[3,2-g]quinolin-7-one; (±)-trans-3-Ethyl-1,2,3,6-tetrahydro-2-methyl-1-(2,2,2-trifluoroethyl)-9-(trifluoromethyl)-7H-[1,4]oxazino[3,2-g]quinolin-7-one; (±)-cis-3-Ethyl-1,2,3,6-tetrahydro-2-methyl-1-(2,2,2-trifluoroethyl)-9-(trifluoromethyl)-7H-[1,4]oxazino[3,2-g]quinolin-7-one; (±)-1,2,3,6-Tetrahydro-2-(hydroxymethyl)-1-(2,2,2-trifluoroethyl)-9-(trifluoromethyl)-7H-[1,4]oxazino[3,2-g]quinolin-7-one; (±)-1,2,3,6-Tetrahydro-2-(acetoxymethyl)-1-(2,2,2-trifluoroethyl)-9-(trifluoromethyl)-7H-[1,4]oxazino-[3,2-g]quinolin-7-one; (±)-1,2,3,6-Tetrahydro-2-(methoxymethyl)-1-(2,2,2-trifluoroethyl)-9-(trifluoromethyl)-7H-[1,4]oxazino-[3,2-g]quinolin-7-one; (−)-1,2,3,6-Tetrahydro-2-(methoxymethyl)-1-(2,2,2-trifluoroethyl)-9-(trifluoromethyl)-7H-[1,4]oxazino-[3,2-g]quinolin-7-one; (±)-2-(Ethoxymethyl)-1,2,3,6-tetrahydro-1-(2,2,2-trifluoroethyl)-9-(trifluoromethyl)-7H-[1,4]oxazino-[3,2-g]quinolin-7-one; (±)-1,2,3,6-Tetrahydro-2-(propoxymethyl)-1-(2,2,2-trifluoroethyl)-9-(trifluoromethyl)-7H-[1,4]oxazino-[3,2-g]quinolin-7-one; 1,2-Dihydro-1-(2,2,2-trifluoroethyl)-9-(trifluoromethyl)-3H-[1,4]oxazino[3,2-g]-quinolin-2,7-dione; (±)-1,2,3,6-Tetrahydro-2-hydroxy-2-methyl-1-(2,2,2-trifluoroethyl)-9-(trifluoromethyl)-7H-[1,4]oxazino-[3,2-g]quinolin-7-one; 1,2-Dihydro-3-methyl-1-(2,2,2-trifluoroethyl)-9-(trifluoromethyl)-3H-[1,4]oxazino[3,2-g]-quinolin-2,7-dione; 1,2,3,6-Tetrahydro-1-(2,2,2-trifluoroethyl)-9-(trifluoromethyl)-2-thioxo-7H-[1,4]-oxazino[3,2-g]quinolin-7-one; (±)-1,2,3,6-Tetrahydro-2-methyl-9-(trifluoromethyl)-7H-[1,4]oxazino[3,2-g]quinolin-7-one; 1-Cyclopropylmethyl-1,2,3,6-tetrahydro-2-methyl-9-(trifluoromethyl)-7H-[1,4]oxazino[3,2-g]quinolin-7-one; (±)-2-Ethyl-1,2,3,6-tetrahydro-9-(trifluoromethyl)-7H-[1,4]oxazino[3,2-g]quinolin-7-one; 1-Cyclopropylmethyl-2-ethyl-1,2,3,6-tetrahydro-9-(trifluoromethyl)-7H-[1,4]oxazino[3,2-g]quinolin-7-one; 1,2,3,6-Tetrahydro-1-isopropyl-9-(trifluoromethyl)-7H-[1,4]oxazino[3,2-g]quinolin-7-one; (±)-2-Ethyl-1,2,3,6-tetrahydro-1-(2,2,2-trifluoroethyl)-9-(trifluoromethyl)-7H-[1,4]oxazino[3,2-g]-quinolin-7-one; (±)-1,2-Diethyl-1,2,3,6-tetrahydro-9-(trifluoromethyl)-7H-[1,4]oxazino[3,2-g]quinolin-7-one; (±)-1,2,3,6-Tetrahydro-1-(2,2,2-trifluoroethyl)-2,9-bis(trifluoromethyl)-7H-[1,4]-oxazino[3,2-g]quinolin-7-one; (±)-1,2,3,6-Tetrahydro-1-(2,2,2-trifluoroethyl)-2,9-bis(trifluoromethyl)-7H-[1,4]oxazino[3,2-g]quinolin-7-one; (−)-1,2,3,6-Tetrahydro-1-(2,2,2-trifluoroethyl)-2,9-bis(trifluoromethyl)-7H-[1,4]oxazino[3,2-g]quinolin-7-one; (±)-1-Ethyl-1,2,3,6-tetrahydro-2-methyl-9-(trifluoromethyl)-7H-[1,4]oxazino[3,2-g]-quinolin-7-one; (2R)-(−)-1,2,3,6-Tetrahydro-2-methyl-1-(2,2,2-trifluoroethyl)-9-(trifluoromethyl)-7H-[1,4]oxazino[3,2-g]-quinolin-7-one; (2R)-2-Ethyl-1,2,3,6-tetrahydro-1-(2,2,2-trifluoroethyl)-9-(trifluoromethyl)-7H-[1,4]oxazino[3,2-g]-quinolin-7-one; (2R)-2-Ethyl-1,2,3,6-tetrahydro-1-(2,2,2-trifluoroethyl)-9-(trifluoromethyl)-7H-[1,4]oxazino[3,2-g]-quinolin-7-one; (2R)-1,2,3,6-Tetrahydro-2-isopropyl-1-(2,2,2-trifluoroethyl)-9-(trifluoromethyl)-7H-[1,4]oxazino[3,2-g]-quinolin-7-one; (±)-1,2,3,4,4a,5-Hexahydro-11-(trifluoromethyl)-pyrido[1′,2′:4,5][1,4]oxazino[3,2-g]quinolin-7-one; (R)-2,3,3a,4-Tetrahydro-10-(trifluoromethyl)-pyrrolo[1′,2′:4,5][1,4]oxazino[3,2-g]quinolin-8(7H)-one, 1,3,4,6-Tetrahydro-1,3,3-trimethyl-9-(trifluoromethyl)-pyrazino[3,2-g]quinolin-2,7-dione; 1,2,3,4-Tetrahydro-1,3,3-trimethyl-9-(trifluoromethyl)-pyrazino[3,2-g]quinolin-7(6H)one; 9-(Trifluoromethyl)-1,2,3,6-tetrahydro-7H-[1,4]thiazino[3,2-g]quinolin-7-one; 1-Methyl-9-(trifluoromethyl)-1,2,3,6-tetrahydro-7H-[1,4]thiazino[3,2-g]quinolin-7-one; 1-(2,2,2-Trifluoroethyl)-9-(trifluoromethyl)-1,2,3,6-tetrahydro-7H-[1,4]thiazino[3,2-g]quinolin-7-one; (Z)-5-Butylidene-1,2-dihydro-2,2,4-trimethyl-5H-chromeno[3,4-f]quinoline; (Z)-5-Benzylidene-1,2-dihydro-2,2,4-trimethyl-5H-chromeno[3,4-f]quinoline; (Z)-5-(4-Fluorobenzylidene)-1,2-dihydro-2,2,4-trimethyl-5H-chromeno[3,4-f]quinoline; (Z)-5-(4-Bromobenzylidene)-1,2-dihydro-2,2,4-trimethyl-5H-chromeno[3,4-f]quinoline; (Z)-5-(3-Bromobenzylidene)-1,2-dihydro-2,2,4-trimethyl-5H-chromeno[3,4-f]quinoline; (Z)-5-(3-Chlorobenzylidene)-1,2-dihydro-2,2,4-trimethyl-5H-chromeno[3,4-f]quinoline; (Z)-5-(3-Fluorobenzylidene)-1,2-dihydro-2,2,4-trimethyl-5H-chromeno[3,4-f]-quinoline; (Z)-5-(2-Chlorobenzylidene)-1,2-dihydro-2,2,4-trimethyl-5H-chromeno[3,4-f]quinoline; (Z)-5-(2-Bromobenzylidene)-1,2-dihydro-2,2,4-trimethyl-5H-chromeno-[3,4-f]-quinoline; (Z)-5-(2-Fluorobenzylidene)-1,2-dihydro-2,2,4-trimethyl-5H-chromeno[3,4-f]quinoline; (Z)-5-(2,3-Difluorobenzylidene)-1,2-dihydro-2,2,4-trimethyl-5H-chromeno-[3,4-f]quinoline; (Z)-5-(2,5-Difluorobenzylidene)-1,2-dihydro-2,2,4-trimethyl-5H-chromeno[3,4-f]quinoline; (Z)-9-Fluoro-5-(3-fluorobenzylidene)-1,2-dihydro-2,2,4-trimethyl-5H-chromeno[3,4-f]quinoline; (Z)-9-Fluoro-5-(3-methoxy-benzylidene)-1,2-dihydro-2,2,4-trimethyl-5H-chromeno[3,4-f]quinoline; (Z)-8-Fluoro-5-(3-fluoro-benzylidene)-1,2-dihydro-2,2,4-trimethyl-5H-chromeno[3,4-f]quinoline; (Z)-5-(2,4-Difluorobenzylidene)-1,2-dihydro-2,2,4-trimethyl-5H-chromeno[3,4-f]-quinoline; (Z)-5-(3,4-Difluorobenzylidene)-1,2-dihydro-2,2,4-trimethyl-5H-chromeno[3,4-f]quinoline; (Z)-5-(2,6-Difluorobenzylidene)-1,2-dihydro-2,2,4-trimethyl-5H-chromeno[3,4-f]-quinoline; (Z)-1,2-Dihydro-5-(2-methylbenzylidene)-2,2,4-trimethyl-5H-chromeno[3,4-f]quinoline; (Z)-1,2-Dihydro-5-(2,4,6-trimethylbenzylidene)-2,2,4-trimethyl-5H-chromeno[3,4-f]-quinoline; (Z)-9-Chloro-5-(2,5-difluorobenzylidene)-1,2-dihydro-2,2,4-trimethyl-5H-chromeno[3,4-f]quinoline; (Z)-5-Benzylidene-9-chloro-1,2-dihydro-2,2,4-trimethyl-5H-chromeno[3,4-f]quinoline; (Z)-9-Chloro-1,2-dihydro-2,2,4-trimethyl-5-(2-methylbenzylidene)-5H-chromeno[3,4-f]-quinoline; (Z)-5-Benzylidene-9-chloro-1,2-dihydro-2,2-dimethyl-5H-chromeno[3,4-f]quinoline; (Z)-9-Chloro-5-(2-fluoro-benzylidene)-1,2-dihydro-2,2,4-trimethyl-5H-chromeno[3,4-f]-quinoline; (Z)-9-Chloro-5-(3-fluorobenzylidene)-1,2-dihydro-2,2,4-trimethyl-5H-chromeno[3,4-f]quinoline; (E/Z)-5-Benzylidene-9-fluoro-1,2-dihydro-2,2,4-trimethyl-5H-chromeno[3,4-f]-quinoline; (Z)-5-Benzylidene-8-fluoro-1,2-dihydro-2,2,4-trimethyl-5H-chromeno[3,4-f]-quinoline; (Z)-5-Benzylidene-1,2-dihydro-9-methoxy-2,2,4-trimethyl-5H-chromeno-[3,4-f]-quinoline; (Z)-9-Fluoro-1,2-dihydro-2,2,4-trimethyl-5-(2-methylbenzylidene)-5H-chromeno[3,4-f]quinoline; (Z)-8-Fluoro-1,2-dihydro-2,2,4-trimethyl-5-(2-methyl-benzylidene)-5H-chromeno[3,4-f]quinoline; (Z)-1,2-Dihydro-9-methoxy-2,2,4-trimethyl-5-(2-methylbenzylidene)-5H-chromeno[3,4-f]quinoline; (Z)-5-Benzylidene-9-fluoro-1,2-dihydro-2,2,4,11-tetramethyl-5H-chromeno[3,4-f]quinoline; (Z)-(R/S)-5-(3-Fluorobenzylidene)-1,2,3,4-tetrahydro-2,2,4-trimethyl-5H-chromeno[3,4-f]-3-quinolinone; Z)-(R/S)-5-(Benzylidene)-1,2,3,4-tetrahydro-2,2,4-trimethyl-5H-chromeno[3,4-f]-3-quinolinone; 2,5-dihydro-10-methoxy-2,2,4-trimethyl-5-phenyl-1H-[1]benzopyrano[3,4-f]quinoline; 2,5-dihydro-10-methoxy-2,2,4-trimethyl-5-(2-propenyl)-1H-[1]benzopyrano[3,4-f]quinoline; 10-(difluoromethoxy)-2,5-dihydro-2,2,4-trimethyl-5-(2-propenyl)-1H-[1]benzopyrano[3,4-f]-quinoline; 10-ethoxy-2,5-dihydro-2,2,4-trimethyl-5-phenyl-1H-[1]benzopyrano[3,4-f]quinoline; 5-(3-bromo-5-methylphenyl)-2,5-dihydro-10-methoxy-2,2,4-trimethyl-1H-[1]benzopyrano[3,4-f]-quinoline; 3-(2,5-dihydro-10-methoxy-2,2,4-trimethyl-1H-[1]benzopyrano[3,4-f]-quinolin-5-yl)phenol, acetate(ester); 3-(2,5-dihydro-10-methoxy-2,2,4-trimethyl-1H-[1]benzopyrano[3,4-f]quinolin-5-yl)phenol; 2,5-dihydro-10-methoxy-2,2,4-trimethyl-5-[[3-(methylthio)methoxy]phenyl]-1H-[1]benzopyrano[3,4-f]-quinoline; [3-(2,5-dihydro-10-methoxy-2,2,4-trimethyl-1H-[1]benzopyrano[3,4-f]quinolin-5-yl)phenyl]-dimethyl-carbamate; 5-[3-(2-furanyl)-5-methylphenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-methyl-5-(1-morpholinyl)phenyl]-1H-[1]benzopyrano[3,4-f]-quinoline; 2,5-dihydro-10-methoxy-2,2,4-trimethyl-5-(phenylmethylene)-1H-[1]benzopyrano[3,4-f]quinoline; 5-(3,5-dichlorophenyl)-2,5-dihydro-10-methoxy-2,2,4-trimethyl-1H-[1]benzopyrano-[3,4-f]quinoline; 5-butyl-2,5-dihydro-10-methoxy-2,2,4-trimethyl-1H-[1]benzopyrano-[3,4-f]quinoline; 2,5-dihydro-10-methoxy-2,2,4-trimethyl-5-[3-(trifluoromethyl)-phenyl]-1H-[1]benzopyrano[3,4-f]-quinoline; 2,5-dihydro-10-methoxy-5-(4-methoxy-phenyl)-2,2,4-trimethyl-1H-[1]benzopyrano[3,4-f]quinoline; 5-(3-chlorophenyl)-2,5-dihydro-10-methoxy-2,2,4-trimethyl-1H-[1]benzopyrano[3,4-f]quinoline; 2,5-dihydro-10-methoxy-2,2,4-trimethyl-5-(3-methylphenyl)-1H-[1]benzopyrano[3,4-f]quinoline; (±)-2,5-dihydro-10-methoxy-2,2,4-trimethyl-5-phenyl-1H-[1]benzopyrano[3,4-f]-quinoline; (±)-2,5-dihydro-10-methoxy-2,2,4-trimethyl-5-phenyl-1H-[1]benzopyrano-[3,4-f]quinoline; 5-(3,5-dimethylphenyl)-2,5-dihydro-10-methoxy-2,2,4-trimethyl-1H-[1]benzopyrano[3,4-f]quinoline; 5-(4-chlorophenyl)-2,5-dihydro-10-methoxy-2,2,4-trimethyl-1H-[1]benzopyrano[3,4-f]quinoline; 5-(3,4-dimethylphenyl)-2,5-dihydro-10-methoxy-2,2,4-trimethyl-1H-[1]benzopyrano[3,4-f]quinoline; 5-(4-fluorophenyl)-2,5-dihydro-10-methoxy-2,2,4-trimethyl-1H-[1]benzopyrano[3,4-f]quinoline; 5-[3,5-bis(trifluoromethyl)phenyl]-2,5-dihydro-10-methoxy-2,2,4-trimethyl-1H-[1]benzo-pyrano[3,4-f]-quinoline; (−)-5-(3,5-dichlorophenyl)-2,5-dihydro-10-methoxy-2,2,4-trimethyl-1H-[1]benzopyrano[3,4-f]quinoline; (+)-5-(3,5-dichlorophenyl)-2,5-dihydro-10-methoxy-2,2,4-trimethyl-1H-[1]benzopyrano[3,4-f]quinoline; 5-(3,5-difluoro-phenyl)-2,5-dihydro-10-methoxy-2,2,4-trimethyl-1H-[1]benzopyrano[3,4-f]-quinoline; 2,5-dihydro-10-methoxy-2,2,4,N-tetramethyl-N-phenyl-1H-[1]benzopyrano[3,4-f]quinolin-5-amine; (−)2,5-dihydro-10-methoxy-2,2,4-trimethyl-5-(2-propenyl)-1H-[1]benzopyrano[3,4-f]quinoline; (+)-2,5-dihydro-10-methoxy-2,2,4-trimethyl-5-(2-propenyl)-1H-[1]benzopyrano[3,4-f]quinoline; 2,5-dihydro-10-methoxy-2,2,4-trimethyl-1H-[1]benzopyrano[3,4-f]quinoline; 4-(2,5-dihydro-10-methoxy-2,2,4-trimethyl-1H-[1]benzopyrano[3,4-f]quinolin-5-yl)-N,N-dimethylbenzenamide; 2,5-dihydro-10-methoxy-2,2,4-trimethyl-(5-methoxy-2-thienyl)-1H-[1]benzopyrano[3,4-f]quinoline; 2,5-dihydro-10-methoxy-2,2,4-trimethyl-5-(5-propyl-2-thienyl)-1H-[1]benzopyrano-[3,4-f]quinoline; 2,5-dihydro-10-methoxy-2,2,4-trimethyl-5-[4-(1-morpholinyl)phenyl]-1H-[1]benzopyrano[3,4-f]-quinoline; 1-(2,5-dihydro-10-methoxy-2,2,4-trimethyl-1H-[1]benzopyrano[3,4-f]quinolin-5-yl)-3,3-dimethyl-2-butanone; 2,5-dihydro-10-methoxy-2,2,4-trimethyl-1H-[1]benzopyrano[3,4-f]quinoline-5-carbonitrile; 1-(2,5-dihydro-10-methoxy-2,2,4-trimethyl-1H-[1]benzopyrano[3,4-f]quinolin-5-yl)-2-propanone; methyl-2,5-dihydro-10-methoxy-2,2,4-trimethyl-1H-[1]benzopyrano[3,4-f]quinoline-5-acetate; 2-(2,5-dihydro-10-methoxy-2,2,4-trimethyl-1H-[1]benzopyrano[3,4-f]quinolin-5-yl)-1-phenylethanone; 5-[2-(chloromethyl)-2-propenyl]-2,5-dihydro-10-methoxy-2,2,4-trimethyl-1H-[1]benzopyrano[3,4-f]-quinoline; 2,5-dihydro-10-methoxy-2,2,4-trimethyl-(-methylene-1H-[1]benzopyrano[3,4-f]quinoline-5-propanol, acetate(ester); 2,5-dihydro-10-methoxy-2,2,4-trimethyl-5-(4-methylphenyl)-1H-[1]benzopyrano[3,4-f]quinoline; 5-(3-fluoro-4-methylphenyl)-2,5-dihydro-10-methoxy-2,2,4-trimethyl-1H-[1]benzo-pyrano[3,4-f]-quinoline; 5-(3-bromophenyl)-2,5-dihydro-10-methoxy-2,2,4-trimethyl-1H-[1]benzopyrano[3,4-f]-quinoline; 2,5-dihydro-10-methoxy-2,2,4-trimethyl-5-(phenylmethyl)-1H-[1]benzopyrano[3,4-f]quinoline; 2,5-dihydro-10-methoxy-2,2,4-trimethyl-5-propyl-1H-[1]benzopyrano[3,4-f]quinoline; 5-(4-fluorophenyl)-2,5-dihydro-10-methoxy-2,2,4-trimethyl-1H-[1]benzopyrano[3,4-f]quinoline; 5-(3-fluorophenyl)-2,5-dihydro-10-methoxy-2,2,4-trimethyl-1H-[1]benzopyrano[3,4-f]quinoline; 2,5-dihydro-10-methoxy-2,2,4,5-tetramethyl-1H-[1]benzopyrano[3,4-f]quinoline; 2,5-dihydro-10-methoxy-2,2,4-trimethyl-5-(1-methylethyl)-1H-[1]benzopyrano[3,4-f]-quinoline; 2,5-dihydro-10-methoxy-2,2,4-trimethyl-5-(2-methylpropyl)-1H-[1]benzopyrano[3,4-f]quinoline; 5-ethyl-2,5-dihydro-10-methoxy-2,2,4-trimethyl-1H-[1]benzopyrano[3,4-f]quinoline; 2,5-dihydro-10-methoxy-2,2,4-trimethyl-1H-[1]benzo-pyrano[3,4-f]quinoline-5-carboximidic acid ethyl ester; 2,5-dihydro-10-methoxy-2,2,4-trimethyl-1-methylene-1H-[1]benzopyrano[3,4-f]quinoline-5-propanol; 2,5-dihydro-10-methoxy-2,2,4,N,N-pentamethyl-1H-[1]benzopyrano[3,4-f]quinoline-5-acetamide; 2,5-dihydro-10-methoxy-2,2,4,N,N-pentamethyl-1H-[1]benzopyrano[3,4-f]quinoline-5-ethanamine; N-cyclopropyl-2,5-dihydro-10-methoxy-2,2,4-trimethyl-1H-[1]benzopyrano[3,4-f]quinoline-5-acetamide; 2,5-dihydro-10-methoxy-2,2,4-trimethyl-5-(2-propynyl)-1H-[1]benzopyrano[3,4-f]quinoline; 5-(2,5-dihydro-10-methoxy-2,2,4-trimethyl-1H-[1]benzopyrano[3,4-f]quinolin-5-yl)-2(5H)-furanone; 5-(3-butenyl)-2,5-dihydro-10-methoxy-2,2,4-trimethyl-1H-[1]benzopyrano[3,4-f]quinoline; 2,5-dihydro-10-methoxy-2,2,4-trimethyl-1H-[1]benzopyrano[3,4-f]quinoline-5-propanol; 5-(3,5-dichlorophenyl)-10-ethoxy-2,5-dihydro-2,2,4-trimethyl-1H-[1]benzopyrano[3,4-f]-quinoline; 10-(bromodifluoromethoxy)-2,5-dihydro-2,2,4-trimethyl-5-(2-propenyl)-1H-[1]benzopyrano[3,4-f]-quinoline; [3-(2,5-dihydro-10-methoxy-2,2,4-trimethyl-1H-[1]benzopyrano[3,4-f]quinolin-5-yl)phenyl]-methylcarbonate; 2,5-dihydro-10-methoxy-5-(3-methoxyphenyl)-2,2,4-trimethyl-1H-[1]benzopyrano[3,4-f]quinoline; 2,5-dihydro-10-methoxy-2,2,4-trimethyl-5-[3-(2-propenyloxy)phenyl]-1H-[1]benzopyrano[3,4-f]-quinoline; 2,5-dihydro-10-methoxy-2,2,4-trimethyl-5-[3-(phenylmethoxy)phenyl]-1H-[1]benzopyrano[3,4-f]-quinoline; 5-[3-(cyclopropylmethoxy)phenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-[2-(1-piperidinyl)ethoxy]phenyl]-1H-[1]benzopyrano[3,4-f]-quinoline; 5-(3-hexyloxyphenyl)-2,5-dihydro-10-methoxy-2,2,4-trimethyl-1H-[1]benzopyrano[3,4-f]quinoline; 5-[3-(2,4-dinitrophenoxy)phenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-(2-propynyloxy)phenyl]-1H-[1]benzopyrano[3,4-f]-quinoline; 3-(2,5-dihydro-10-methoxy-2,2,4-trimethyl-1H-[1]benzopyrano[3,4-f]quinolin-5-yl)phenol-4-methyl-benzenesulfonate (ester); 4-(2,5-dihydro-10-methoxy-2,2,4-trimethyl-1H-[1]benzopyrano[3,4-f]quinolin-5-yl)phenolacetate(ester); 4-(2,5-dihydro-10-methoxy-2,2,4-trimethyl-1H-[1]benzopyrano[3,4-f]quinolin-5-yl)phenol; 2,5-dihydro-10-methoxy-2,2,4-trimethyl-5-[[4-(methylthio)methoxy]phenyl]-1H-[1]benzopyrano[3,4-f]-quinoline; [4-(2,5-dihydro-10-methoxy-2,2,4-trimethyl-1H-[1]benzopyrano[3,4-f]quinolin-5-yl)phenyl]-dimethylcarbamate; 2,5-dihydro-10-methoxy-2,2,4-trimethyl-5-[4-(phenylmethoxy)phenyl]-1H-[1]benzopyrano[3,4-f]-quinoline; 2,5-dihydro-10-methoxy-2,2,4-trimethyl-5-[3-(methoxymethoxy)phenyl]-1H-[1]benzopyrano[3,4-f]-quinoline, [(2,5-dihydro-10-methoxy-2,2,4-trimethyl-1H-[1]benzopyrano[3,4-f]quinolin-5-yl)phenyl]1-morpholinecarboxylate; 2,5-dihydro-10-methoxy-2,2,4-trimethyl-5-[3-[(methylsulfinyl)methoxy]phenyl]-1H-[1]benzopyrano-[3,4-f]quinoline; O-[3-(2,5-dihydro-10-methoxy-2,2,4-trimethyl-1H[1]benzopyrano-[3,4-f]quinolin-5-yl)phenyl]ester; 2,5-dihydro-10-methoxy-2,2,4-trimethyl-5-[3-(methylthio)phenyl]-1H-[1]benzopyrano[3,4-f]quinoline; O-[3-(2,5-dihydro-10-methoxy-2,2,4-trimethyl-1H-[1]benzopyrano[3,4-f]quinolin-5-yl)phenyl]-methyl-carbonothioate; [3-(2,5-dihydro-10-methoxy-2,2,4-trimethyl]-1H-[1]benzopyrano-[3,4-f]quinolin-5-yl)phenyl]trifluoro-methanesulfonate; 5-[3-(4,5-dihydro-4,4-dimethyl-2-oxazolyl)phenyl]-2,5-dihydro-10-methoxy-2,2,4-trimethyl-1H-[1]benzopyrano[3,4-f]-quinoline; ethyl 3-(2,5-dihydro-10-methoxy-2,2,4-trimethyl-1H-[1]benzopyrano[3,4-f]-quinolin-5-yl)benzoate; 3-(2,5-dihydro-10-methoxy-2,2,4-trimethyl-1H-[1]benzopyrano[3,4-f]quinolin-5-yl)benzoic acid; 2,5-dihydro-10-methoxy-2,2,4-trimethyl-5-[3-methyl-5-(2-propenyl)phenyl]-1H-[1]benzopyrano[3,4-f]-quinoline; 1-[3-(2,5-dihydro-10-methoxy-2,2,4-trimethyl-1H-[1]benzopyrano[3,4-f]quinolin-5-yl)-5-methylphenyl]-ethanone; 3-(2,5-dihydro-10-methoxy-2,2,4-trimethyl-1H-[1]benzo-pyrano[3,4-f]quinolin-5-yl)-5-trimethylbenzene-methanol; 5-[3-(2-furanyl)phenyl]-2,5-dihydro-10-methoxy-2,2,4-trimethyl-1H-[1]benzopyrano[3,4-f]quinoline; 2,5-dihydro-10-methoxy-2,2,4-trimethyl-5-[3-methyl-5-(1H-pyrrolidin-1-yl)phenyl]-1H-[1]benzo-pyrano-[3,4-f]quinoline; 3-(2,5-dihydro-10-methoxy-2,2,4-trimethyl-1H-[1]benzo-pyrano[3,4-f]quinolin-5-methyl)-5,N-dimethylbenzenamine; 3-(2,5-dihydro-10-methoxy-2,2,4-trimethyl-1H-[1]benzopyrano[3,4-f]quinolin-5-yl)-5-methyl-N-(2-propenyl)benzamide; 3-(2,5-dihydro-10-methoxy-2,2,4-trimethyl-1H-[1]benzopyrano-[3,4-f]quinolin-5-yl)-N-(2-methoxyethyl)-5-methylbenzenamide; 3-(2,5-dihydro-10-methoxy-2,2,4-trimethyl-1H-[1]benzopyrano[3,4-f]quinolin-5-yl)-N-(2-propenyl)-benzenamide; N′-[3-(2,5-dihydro-10-methoxy-2,2,4-trimethyl-1H-[1]benzopyrano[3,4-f]quinolin-5-yl)-5-methylphenyl]-N,N-dimethylurea; N-[3-(2,5-dihydro-10-methoxy-2,2,4-trimethyl-1H-[1]benzopyrano[3,4-f]quinolin-5-yl)phenyl]-benzenemethanamide; 5-[(3,5-dichlorophenyl)methylene]-2,5-dihydro-10-methoxy-2,2,4-trimethyl-1H-[1]-benzopyrano[3,4-f]-quinoline; 5-[(4-chlorophenyl)methylene]-2,5-dihydro-10-methoxy-2,2,4-trimethyl-1H-[1]benzopyrano[3,4-f]-quinoline; 2,5-dihydro-10-methoxy-2,2,4-trimethyl-5-[[3-(trifluoromethyl)phenyl]methylene]-1H-[1]benzopyrano-[3,4-f]-quinoline; 5-[(2,6-difluorophenyl)methylene]-2,5-dihydro-10-methoxy-2,2,4-trimethyl-1H-[1]benzopyrano[3,4-f]-quinoline; 5-[(2-chlorophenyl)methylene]-2,5-dihydro-10-methoxy-2,2,4-trimethyl-1H-[1]benzopyrano[3,4-f]-quinoline; 5-[(2,6-dichlorophenyl)-methylene]-2,5-dihydro-10-methoxy-2,2,4-trimethyl-1H-[1]benzopyrano[3,4-f]-quinoline; 5-[(2-fluorophenyl)methylene]-2,5-dihydro-10-methoxy-2,2,4-trimethyl-1H-[1]benzopyrano[3,4-f]-quinoline; 2,5-dihydro-10-methoxy-2,2,4-trimethyl-5-[(4,5-dihydro-4,4-dimethyl-2-oxazolyl)methylene]-1H-[1]benzopyrano[3,4-f]quinoline; 2,5-dihydro-10-methoxy-2,2,4-trimethyl-5-(2-pyridinylmethylene)-1H-[1]benzopyrano-[3,4-f]quinoline; 2,5-dihydro-10-methoxy-2,2,4-trimethyl-5-(2-thienyl)-1H-[1]benzo-pyrano[3,4-f]quinoline; 2,5-dihydro-9,10-dimethoxy-2,2,4-trimethyl-5-(2-propenyl)-1H-[1]benzopyrano[3,4-f]quinoline; 5-(2-cyclohexen-1-yl)-2,5-dihydro-9,10-dimethoxy-2,2,4-trimethyl-1H-[1]benzopyrano[3,4-f]quinoline; 2,5-dihydro-10-methoxy-5-(3-methyl-3-butenyl)-2,2,4-trimethyl-1H-[1]benzopyrano[3,4-f]quinoline; 2,5-dihydro-10-methoxy-5-(5,5-dimethyl-3-cyclohexenyl)-2,2,4-trimethyl-1H-[1]benzopyrano[3,4-f]-quinoline; rel(5R,2′R)2,5-dihydro-10-methoxy-5-(2-oxo-3-tetrahydropyranyl)-2,2,4-trimethyl-1H-[1]benzopyrano[3,4-f]quinoline; anti(5R,2′S)2,5-dihydro-10-methoxy-5-(2-oxo-3-tetrahydropyranyl)-2,2,4-trimethyl-1H-[1]benzopyrano-[3,4-f]quinoline; 2,5-dihydro-10-methoxy-5-(3-cyclopentenyl)-2,2,4-trimethyl-1H-[1]benzopyrano[3,4-f]quinoline; 2,5-dihydro-10-methoxy-5-(3-cyclohexenyl)-2,2,4-trimethyl-1H-[1]benzo-pyrano[3,4-f]quinoline; 2,5-dihydro-10-methoxy-5-(3-butenyl)-2,2,4-trimethyl-1H-[1]benzopyrano[3,4-f]quinoline; 2,5-dihydro-10-methoxy-5-(1-ethenyl-1-cyclohexyl)-2,2,4-trimethyl-1H-[1]benzopyrano[3,4-f]quinoline; 2,5-dihydro-10-methoxy-5-(4,4-dimethyl-3-cyclohexenyl)-2,2,4-trimethyl-1H-[1]benzopyrano[3,4-f]-quinoline; 2,5-dihydro-10-methoxy-5-(1-methylene-2-cyclohexyl)-2,2,4-trimethyl-1H-[1]benzo-pyrano[3,4-f]-quinoline; 2,5-dihydro-10-methoxy-5-(1-oxo-2-cyclohexyl)-2,2,4-trimethyl-1H-[1]benzopyrano[3,4-f]quinoline; 2,5-dihydro-10-methoxy-5-(3-cyclooctenyl)-2,2,4-trimethyl-1H-[1]benzopyrano[3,4-f]quinoline; 2,5-dihydro-10-methoxy-5-(3-cycloheptenyl)-2,2,4-trimethyl-1H-[1]benzopyrano[3,4-f]quinoline; 2,5-dihydro-10-methoxy-5-(1-cyclohexenylmethyl)-2,2,4-trimethyl-1H-[1]benzo-pyrano[3,4-f]quinoline; 2,5-dihydro-10-methoxy-5-(3,3-dimethyl-6-cyclohexenyl)-2,2,4-trimethyl-1H-[1]benzopyrano[3,4-f]-quinoline; 2,5-dihydro-10-methoxy-5-(2-bromo-3-propenyl)-2,2,4-trimethyl-1H-[1]benzopyrano[3,4-f]quinoline; rel(5R,3′R)2,5-dihydro-10-methoxy-5-(1-hydroxymethyl-3-cyclohexenyl)-2,2,4-trimethyl-1H-[1]benzopyrano[3,4-f]quinoline; rel(5R,3′S)2,5-dihydro-10-methoxy-5-(1-hydroxy-methyl-3-cyclohexenyl)-2,2,4-trimethyl-1H-[1]benzopyrano[3,4-f]quinoline; 2,5-dihydro-10-methoxy-5-(3-hydroxymethyl-3-cyclohexenyl)-2,2,4-trimethyl-1H-[1]benzopyrano[3,4-f]-quinoline; 2,5-dihydro-10-methoxy-5-(3-indolyl)-2,2,4-trimethyl-1H-[1]benzopyrano[3,4-f]quinoline; rel(5S,3′S)2,5-dihydro-10-methoxy-5-(1-methyl-3-cyclohexenyl)-2,2,4-trimethyl-1H-[1]benzopyrano-[3,4-f]quinoline; rel(5R,3′S)2,5-dihydro-10-methoxy-5-(1-methyl-3-cyclohexenyl)-2,2,4-trimethyl-1H-[1]benzopyrano[3,4-f]-quinoline; (−)(5S,3′S)2,5-dihydro-10-methoxy-5-(1-methyl-3-cyclohexenyl)-2,2,4-trimethyl-1H-[1]benzopyrano[3,4-f]quinoline; (−)(5S,3′R)2,5-dihydro-10-methoxy-5-(1-hydroxymethyl-3-cyclohexenyl)-2,2,4-trimethyl-1H-[1]benzopyrano[3,4-f]quinoline; (+)(5R,3′S)2,5-dihydro-10-methoxy-5-(1-hydroxy-methyl-3-cyclohexenyl)-2,2,4-trimethyl-1H-[1]benzopyrano[3,4-f]quinoline; (−)-(5S,3′R)2,5-dihydro-10-methoxy-5-(1-methyl-3-cyclohexenyl)-2,2,4-trimethyl-1H-[1]benzopyrano-[3,4-f]-quinoline; (+)-(5R,3′S)2,5-dihydro-10-methoxy-5-(1-methyl-3-cyclohexenyl)-2,2,4-trimethyl-1H-[1]benzopyrano-[3,4-f]quinoline; 2,5-dihydro-10-methoxy-5-(1-chloromethyl-3-cyclohexenyl)-2,2,4-trimethyl-1H-[1]benzopyrano[3,4-f]-quinoline; rel(5R,3′R)2,5-dihydro-10-methoxy-5-(1-methoxymethyl-3-cyclohexenyl)-2,2,4-trimethyl-1H-[1]benzopyrano[3,4-f]quinoline; rel(5R,3′R)2,5-dihydro-10-methoxy-5-(1-methylthiomethyl-3-cyclohexenyl)-2,2,4-trimethyl-1H-[1]benzopyrano-[3,4-f]quinoline; rel(5R,3′S)2,5-dihydro-10-methoxy-5-(1-acetoxymethyl-3-cyclo-hexenyl)-2,2,4-trimethyl-1H-[1]benzopyrano[3,4-f]quinoline; rel(5R,3′R)2,5-dihydro-10-methoxy-5-(1-acetoxymethyl-3-cyclohexenyl)-2,2,4-trimethyl-1H[1]benzopyrano-[3,4-f]quinoline; rel(5R,3′R)2,5-dihydro-10-methoxy-5-(1-methoxymethyl-3-cyclohexenyl)-2,2,4-trimethyl-1H-[1]benzopyrano[3,4-f]quinoline; rel(5R,3′R)2,5-dihydro-10-methoxy-5-(1-(N,N-dimethylamino)methyl-3-cyclohexenyl)-2,2,4-trimethyl-1H-[1]benzopyrano[3,4-f]-quinoline; rel(5R,3′S)2,5-dihydro-10-methoxy-5-(1-methylthio-methyl-3-cyclohexenyl)-2,2,4-trimethyl-1H-[1]benzopyrano-[3,4-f]quinoline; rel(5R,3′R)2,5-dihydro-10-methoxy-5-(1-(N-morpholine)methyl-3-cyclohexenyl)-2,2,4-trimethyl-1H-[1]benzopyrano[3,4-f]quinoline; rel(5R,3′R)2,5-dihydro-10-methoxy-5-(1-(N-methyl-N-methylsulfonylamino)methyl-3-cyclohexenyl)-2,2,4-trimethyl-1H-[1]benzopyrano[3,4-f]-quinoline; rel(5R,3′S)2,5-dihydro-10-methoxy-S-(1-N,N dimethylamino)methyl-3-cyclohexenyl)-2,2,4-trimethyl-1H-[1]benzopyrano-[3,4-f]quinoline; rel(5R,3′R)2,5-dihydro-10-methoxy-5-(1-(N-methylamino)methyl-3-cyclohexenyl)-2,2,4-trimethyl-1H-[1]benzopyrano[3,4-f]-quinoline; 2,5-dihydro-10-methoxy-5-(2-methyl-3-propenyl)-2,2,4-trimethyl-1H-[1]benzopyrano[3,4-f]-quinoline; 2,5-dihydro-10-methoxy-5-(1,3-butadien-2-yl)-2,2,4-trimethyl-1H-[1]benzopyrano-[3,4-f]quinoline; 2,5-dihydro-10-methoxy-5-(2-carbomethoxy-3-propenyl)-2,2,4-trimethyl-1H-[1]benzopyrano[3,4-f]-quinoline; 2,5-dihydro-10-methoxy-5-(1,2-dihydroxy-3-propyl)-2,2,4-trimethyl-1H-[1]benzopyrano[3,4-f]-quinoline; 2,5-dihydro-10-methoxy-5-(1,2-epoxy-3-propenyl)-2,2,4-trimethyl-1H-[1]benzo pyrano[3,4-f]-quinoline; 2,5-dihydro-10-methoxy-5-(1-(N-phthalimido)-3-propyl)-2,2,4-trimethyl-1H-[1]benzopyrano[3,4-f]-quinoline; 2,5-dihydro-10-methoxy-5-(1-amino-3-propyl)-2,2,4-trimethyl-1H-[1]benzopyrano[3,4-f]quinoline; 2,5-dihydro-10-methoxy-5-(1(hydrazino-carbonylamino)-3-propyl)-2,2,4 trimethyl-1H-[1]benzopyrano[3,4-f]quinoline; (E)-2,5-dihydro-10-methoxy-5-(2-carbomethoxy-1-ethenyl)-2,2,4-trimethyl-1H-[1]benzo-pyrano[3,4-f]-quinoline; (Z)-2,5-dihydro-10-methoxy-5-(1-propenyl)-2,2,4-trimethyl-1H-[1]benzopyrano[3,4-f]quinoline; (E)-2,5-dihydro-10-methoxy-5-(3-hydroxy-1-propenyl)-2,2,4-trimethyl-1H-[1]benzopyrano[3,4-f]-quinoline; (E)-2,5-dihydro-10-methoxy-5-(3-(N,N-dimethylaminocarbonyloxy)-1-propenyl)-2,2,4-trimethyl-1H-[1]benzopyrano[3,4-f]quinoline; (E)-2,5-dihydro-10-methoxy-5-(3-methoxymethoxy-1-propenyl)-2,2,4-trimethyl-1H-[1]benzopyrano-[3,4-f]quinoline; 2,5-dihydro-10-methoxy-5-(3-hydroxy-3-propenyl)-2,2,4-trimethyl-1H-[1]benzopyrano[3,4-f]quinoline; methyl 2-(2,5-dihydro-10-methoxy-2,2,4-trimethyl-1H-[1]benzopyrano[3,4-f]quinolin-5-yl)acetyl hydroxamate; 2-(2,5-dihydro-10-methoxy-2,2,4-trimethyl-1H-[1]benzopyrano[3,4-f]quinolin-5-yl)acetaldehyde; 2,5-dihydro-10-methoxy-5-(2-cyclo-hexylidenylethyl)-2,2,4-trimethyl-1H-[1]benzopyrano[3,4-f]-quinoline; 2,5-dihydro-10-methoxy-5-(2-cyclopentylidenylethyl)-2,2,4-trimethyl-1H-[1]benzopyrano[3,4-f]-quinoline; 2,5-dihydro-10-methoxy-5-(2-cycloheptylidenylethyl)-2,2,4-trimethyl-1H-[1]benzopyrano[3,4-f]-quinoline; 2,5-dihydro-10-methoxy-5-(3-methyl-2-butenyl)-2,2,4-trimethyl-1H-[1]benzopyrano[3,4-f]quinoline; trans-2,5-dihydro-10-methoxy-5-(2-butenyl)-2,2,4-trimethyl-1H-[1]benzopyrano[3,4-f]quinoline; trans-2,5-dihydro-10-methoxy-5-(2-penten-1-yl)-2,2,4-trimethyl-1H-[1]benzopyrano[3,4-f]quinoline; 2,5-dihydro-10-methoxy-5-(1,1-difluoro-1-propen-3-yl)-2,2,4-trimethyl-1H-[1]benzo-pyrano[3,4-f]-quinoline; (E)-methyl 2-(2,5-dihydro-10-methoxy-2,2,4-trimethyl-1H-[1]benzopyrano[3,4-f]quinoline; (E)-2,5-dihydro-10-methoxy-5-(4-hydroxy-2-buten-1-yl)-2,2,4-trimethyl-1H-[1]benzopyrano[3,4-f]-quinoline; (E)-2,5-dihydro-10-methoxy-5-(4-(N,N-dimethylaminocarbonyloxy)-2-buten-1-yl)-2,2,4-trimethyl-1H [1]benzopyrano-[3,4-f]quinoline; (E)-2,5-dihydro-10-methoxy-5-(4-(N-methylaminocarbonyloxy)-2-buten-1-yl)-2,2,4-trimethyl-1H-[1]benzopyrano[3,4-f]quinoline; (E)-2,5-dihydro-10-methoxy-5-(2-butenyl)-2,2,4-trimethyl-1H-[1]benzopyrano[3,4-f]quinoline; 2,5-dihydro-10-methoxy-5-(2-hydroxyethyl)-2,2,4-trimethyl-1H-[1]benzopyrano[3,4-f]-quinoline; 2,5-dihydro-10-methoxy-5-(2-(N-benzylcarbonyloxy)ethyl)-2,2,4-trimethyl-1H-[1]benzopyrano[3,4-f]-quinoline; 2,5-dihydro-10-methoxy-5-(2-(N-morpholino-carbonyloxy)ethyl)-2,2,4-trimethyl-1H-[1]benzopyrano-[3,4-f]quinoline; 2,5-dihydro-10-methoxy-5-(2-(N-(2-methoxyethyl)aminocarbonyloxy)ethyl)-2,2,4-trimethyl-1H-[1]benzopyrano[3,4-f]quinoline; 2,5-dihydro-10-methoxy-5-(2-(N-methylamino-carbonyloxyoxy)ethyl)-2,2,4-trimethyl-1H-[1]benzopyrano[3,4-f]quinoline; 2,5-dihydro-10-methoxy-5-(2-(N,N-dimethylaminocarbonyloxy)ethyl)-2,2,4-trimethyl-1H-[1]benzopyrano[3,4-f]quinoline, 2,5-dihydro-10-methoxy-5-(2-methoxymethoxyethyl)-2,2,4-trimethyl-1H-[1]benzopyrano[3,4-f]quinoline; 2,5-dihydro-10-methoxy-5-(2,2-dimethylethoxycarbonylamino)methyl)-2,2,4-trimethyl-1H-[1]benzopyrano[3,4-f]-quinoline; 2,5-dihydro-10-methoxy-5-(aminomethyl)-2,2,4-trimethyl-1H-[1]benzo-pyrano[3,4-f]-quinoline; 2,5-dihydro-10-methoxy-5-(ethoxycarbonylamino)methyl)-2,2,4-trimethyl-1H-[1]benzopyrano[3,4-f]-quinoline; 2,5-dihydro-10-methoxy-5-(carboethoxy)-2,2,4-trimethyl-1H-[1]benzopyrano[3,4-f]-quinoline; 2,5-dihydro-10-methoxy-5-(cyclopentyl)-2,2,4-trimethyl-1H-[1]benzopyrano[3,4-f]quinoline; 2,5-dihydro-10-methoxy-5-(1-methylpropa-1,2-dienyl)-2,2,4-trimethyl-1H-[1]benzo-pyrano[3,4-f]-quinoline; 2,5-dihydro-10-methoxy-5-(3,4,5-trifluorophenyl)-2,2,4-trimethyl-1H-[1]benzopyrano[3,4-f]quinoline; 2,5-dihydro-10-methoxy-5-(cyclohexyl)-2,2,4-trimethyl-1H-[1]benzopyrano[3,4-f]-quinoline; 2,5-dihydro-10-methoxy-5-(2-pyridyl)-2,2,4-trimethyl-1H-[1]benzopyrano[3,4-f]-quinoline; 2,5-dihydro-10-methoxy-5-(3-pyridyl)-2,2,4-trimethyl-1H-[1]benzopyrano[3,4-f]-quinoline; 2,5-dihydro-10-methoxy-5-(4-pyridyl)-2,2,4-trimethyl-1H-[1]benzopyrano[3,4-f]quinoline; 9-10-methylenedioxy-5-phenyl-2,2,4-trimethyl-1H-2,5-dihydro-[1]benzopyrano[3,4-f]-quinoline; 9-chloro-10-methoxy-5-phenyl-2,2,4-trimethyl-2,5-dihydro-1H-[1]benzopyrano[3,4-f]quinoline; 5-(3-propenyl)-9-chloro-10-difluoromethoxy-2,2,4-trimethyl-2,5-dihydro-1H-[1]benzopyrano[3,4-f]-quinoline; 9-chloro-10-difluoro-methoxy-5-phenyl-2,2,4-trimethyl-2,5-dihydro-1H-[1]benzopyrano[3,4-f]quinoline; 8-fluoro-10-methoxy-5-phenyl-2,2,4-trimethyl-2,5-dihydro-1H-[1]benzopyrano[3,4-f]-quinoline; 5-(3-propenyl)-8-fluoro-10-methoxy-2,2,4-trimethyl-2,5-dihydro-1H-[1]benzopyrano[3,4-f]quinoline; (10-methoxy-9-fluoro-5-(3-propenyl)-2,2,4-trimethyl-1H-2,5-dihydro-[1]benzopyrano[3,4-f]quinoline; 10-methoxy-9-hydroxy-5-(3-propenyl)-2,2,4-trimethyl-1H-2,5-dihydro-[1]benzopyrano[3,4-f]quinoline; (±)2,5-dihydro-9-hydroxy-10-methoxy-2,2,4-trimethyl-5-(3-cyclohexenyl)-1H-[1]benzo-pyrano[3,4-f]-quinoline; (±)2,5-dihydro-9-hydroxy-10-methoxy-2,2,4-trimethyl-5-(1-methylcyclohexen-3-yl)-1H-[1]benzo-pyrano[3,4-f]quinoline; (−)(5S,3′S)-9-hydroxy-5-[1-methyl-3-cyclohexenyl]-10-methoxy-2,2,4-trimethyl-2,5-dihydro-1H-[1]benzo-pyrano[3,4-f]quinoline; (+)(5R,3′R)-9-hydroxy-5-[1-methyl-3-cyclohexenyl]-10-methoxy-2,2,4-trimethyl-2,5-dihydro-1H-[1]benzopyrano[3,4-f]quinoline; (+)(5R,3′S)-9-hydroxy-5-[1-methyl-3-cyclohexenyl]-10-methoxy-2,2,4-trimethyl-2,5-dihydro-1H-[1]benzopyrano[3,4-f]quinoline; (−)(5S,3′R)-9-hydroxy-5-[1-methyl-3-cyclohexenyl]-10-methoxy-2,2,4-trimethyl-2,5-dihydro-1H-[1]benzopyrano[3,4-f]quinoline; rel-(5S,3′R)-9-hydroxy-5-[1-hydroxymethyl-3-cyclohexenyl]-10-methoxy-2,2,4-trimethyl-2,5-dihydro-1H-[1]benzopyrano[3,4-f]quinoline; (±)(5S,3′R)-2,5-dihydro-9-hydroxy-10-methoxy-2,2,4-trimethyl-5-(1-methylcyclohexen-3-yl)-1H-[1]benzopyrano[3,4-f]-quinoline; rel-(5S,3′R)-9-hydroxy-5-[1-methoxymethyl-3-cyclohexenyl]-10-methoxy-2,2,4-trimethyl-2,5-dihydro-1H-[1]benzopyrano[3,4-f]quinoline; 2,5-dihydro-9-hydroxy-10-methoxy-5-propyl-2,2,4-trimethyl-1H-[1]benzopyrano[3,4-f]quinoline; (−)(5S,3′S)-2,5-dihydro-9-hydroxy-10-methoxy-2,2,4-trimethyl-5-(3-cycloheptenyl)-1H-[1]benzo-pyrano[3,4-f]quinoline; (−)(5S,3′R)-2,5-dihydro-9-hydroxy-10-methoxy-2,2,4-trimethyl-5-(3-cycloheptenyl)-1H-[1]benzopyrano[3,4-f]quinoline; 2,5-dihydro-9-hydroxy-10-methoxy-2,2,4-trimethyl-5-phenyl-1H-[1]benzopyrano[3,4-f]quinoline; 2,5-dihydro-9-hydroxy-10-methoxy-2,2,4-trimethyl-5-(3,5-difluorophenyl)-1H-[1]benzopyrano[3,4-f]-quinoline; 2,5-dihydro-9-hydroxy-10-methoxy-2,2,4-trimethyl-5-(3,4,5-trifluorophenyl)-1H-[1]benzopyrano[3,4-f]-quinoline; 5-butyl-2,5-dihydro-9-hydroxy-10-methoxy-2,2,4-trimethyl-1H-[1]benzopyrano[3,4-f]quinoline; (−)(5S,3′S)-2,5-dihydro-9-hydroxy-10-methoxy-2,2,4-trimethyl-5-(3-cyclopentenyl)-1H-[1]benzopyrano[3,4-f]quinoline; (−)(5S,3′R)-2,5-dihydro-9-hydroxy-10-methoxy-2,2,4-trimethyl-5-(3-cyclopentenyl)-1H-[1]benzopyrano[3,4-f]quinoline; 2,5-dihydro-9-hydroxy-10-methoxy-2,2,4-trimethyl-5-(3,4-difluorophenyl)-1H-[1]benzopyrano[3,4-f]-quinoline; 2,5-dihydro-9-hydroxy-10-methoxy-2,2,4-trimethyl-5-(4-fluorophenyl)-1H-[1]benzopyrano[3,4-f]-quinoline; 2,5-dihydro-9-hydroxy-10-methoxy-2,2,4-trimethyl-5-(3-trifluoromethylphenyl)-1H-[1]benzopyrano-[3,4-f]quinoline; 2,5-dihydro-9-hydroxy-10-methoxy-2,2,4-trimethyl-5-(3-5-bistrifluoromethylphenyl)-1H-[1]benzo-pyrano-[3,4-f]quinoline; 2,5-dihydro-9-hydroxy-10-methoxy-2,2,4-trimethyl-5-(3-trifluoro-methyl-4-chlorophenyl)-1H-[1]benzo-pyrano[3,4-f]quinoline; 2,5-dihydro-9-hydroxy-10-methoxy-2,2,4-trimethyl-5-(2-methylpropyl)-1H-[1]benzopyrano[3,4-f]-quinoline; 2,5-dihydro-9-hydroxy-10-methoxy-2,2,4-trimethyl-5-(3-fluoro-4-chlorophenyl)-1H-[1]benzopyrano-[3,4-f]quinoline; 2,5-dihydro-9-hydroxy-10-methoxy-2,2,4-trimethyl-5-(3-butenyl)-1H-[1]benzopyrano[3,4-f]quinoline; 2,5-dihydro-9-hydroxy-10-methoxy-5-(phenylmethyl)-2,2,4-trimethyl-1H-[1]benzopyrano[3,4-f]-quinoline; (−)(5S,3′R) 2,5-dihydro-9-hydroxy-10-methoxy-2,2,4-trimethyl-5-[1-ethyl-3-cyclohexenyl]-1H-[1]benzopyrano[3,4-f]quinoline; (−)(S)-5-cyclopentyl-2,5-dihydro-9-hydroxy-10-methoxy-2,2,4-trimethyl-1-[1]benzopyrano[3,4-f]-quinoline; (+)(R)-5-cyclopentyl-2,5-dihydro-9-hydroxy-10-methoxy-2,2,4-trimethyl-1H-[1]benzopyrano[3,4-f]-quinoline; 2,5-dihydro-9-hydroxy-10-methoxy-5-(3-propynyl)-2,2,4-trimethyl-1H-[1]benzo-pyrano[3,4-f]-quinoline; 2,5-dihydro-9-hydroxy-10-methoxy-2,2,4-trimethyl-5-(2-propyl)-1H-[1]benzopyrano[3,4-f]-quinoline; 2,5-dihydro-9-hydroxy-10-methoxy-2,2,4-trimethyl-5-(5-methoxy-2-thienyl)-1H-[1]benzopyrano[3,4-f]-quinoline; (±)2,5-dihydro-9-hydroxy-10-methoxy-2,2,4-trimethyl-5-(2,3,4,5,6-pentafluorophenyl)-1H-[1]benzo-pyrano[3,4-f]quinoline; (±)2,5-dihydro-9-hydroxy-10-methoxy-2,2,4-trimethyl-5(S)-(3(S)-1-hydroxymethylcyclopenten-3-yl)-1H-[1]benzopyrano[3,4-f]quinoline; (±)2,5-dihydro-9-hydroxy-10-methoxy-2,2,4-trimethyl-5(S)-(3(S)-1-methylcarboxylatecyclopenten-3-yl-1H-[1]benzopyrano[3,4-f]quinoline; (−)(5S,3′S) 2,5-dihydro-9-hydroxy-10-methoxy-2,2,4-trimethyl-5-(3-cyclohexenyl)-1H-[1]benzopyrano[3,4-f]quinoline; (−)(5S,3′R) 2,5-dihydro-9-hydroxy-10-methoxy-2,2,4-trimethyl-5-(3-cyclohexenyl)-1H-[1]benzopyrano[3,4-f]quinoline; 2,5-dihydro-9-hydroxy-10-methoxy-2,2,4-trimethyl-5-(2-thienyl)-1H-[1]benzopyrano[3,4-f]quinoline; (+)2,5-dihydro-9-hydroxy-10-methoxy-2,2,4-trimethyl-5-(2-methylphenyl)-1H-[1]benzopyrano[3,4-f]-quinoline; 2,5-dihydro-9-hydroxy-10-methoxy-2,2,4-trimethyl-5-(2-acetoxymethyl-3propenyl)-1H-[1]benzo-pyrano-[3,4-f]quinoline; (+)(5R,3′S)2,5-dihydro-9-hydroxy-10-methoxy-2,2,4-trimethyl-5-[1-ethyl-3-cyclohexenyl]-1H-[1]benzopyrano[3,4-f]quinoline; 2,5-dihydro-9-hydroxy-10-methoxy-2,2,4-trimethyl-5-cyclohexyl-1H-[1]benzopyrano[3,4-f]quinoline; 2,5,5-trihydro-9-hydroxy-10-methoxy-2,2,4-triethyl-1H-[1]benzo-pyrano[3,4-f]quinoline; 2,5-dihydro-9-hydroxy-10-methoxy-2,2,4-trimethyl-5-(2-hydroxymethyl-3-propenyl)-1H-[1]benzopyrano[3,4-f]quinoline; methyl 2-[2,5-dihydro-9-hydroxy-10-methoxy-2,2,4-trimethyl-1H-[1]benzopyrano[3,4-f]-5-quinolinyl]-acetate; (Z)-2,5-dihydro-9-hydroxy-10-methoxy-2,2,4-trimethyl-5-(2-butenyl)-1H-[1]benzo-pyrano[3,4-f]-quinoline; 2,5-dihydro-9-hydroxy-10-methoxy-2,2,4-trimethyl-5-(3-methyl-2-butenyl)-1H-[1]benzopyrano[3,4-f]-quinoline; (+)(5S,3′S) 2,5-dihydro-9-hydroxy-10-methoxy-2,2,4-trimethyl-5-(3-cyclohexenyl)-1H-[1]benzopyrano[3,4-f]-quinoline; (+)(5R,3′R) 2,5-dihydro-9-hydroxy-10-methoxy-2,2,4-trimethyl-5-(3-cyclohexenyl)-1H-[1]benzopyrano[3,4-f]-quinoline; (+)(5R,3′S)-2,5(R)-dihydro-9-hydroxy-10-methoxy-2,2,4-trimethyl-5-(3-cyclopentenyl)-1H-[1]benzopyrano[3,4-f]-quinoline; (+)(5R,3′R)-2,5(R)-dihydro-9-hydroxy-10-methoxy-2,2,4-trimethyl-5-(3-cyclopentenyl)-1H-[1]benzopyrano[3,4-f]quinoline; rel-(5S)-9-hydroxy-5-[(3R)-(1-methoxycarbonyl)cyclohexen-3-yl]-10-methoxy-2,2,4-trimethyl-1H-[1]benzo-pyrano[3,4-f]quinoline; 2,5-dihydro-9-hydroxy-10-methoxy-2,2,4-trimethyl-5-(2-methyl-3-propenyl)-1H-[1]benzopyrano[3,4-f]-quinoline; 9,10-Dimethoxy-5-(3-propenyl)-2,2,4-trimethyl-1H-2,5-dihydro-[1]benzopyrano[3,4-f]quinoline; 9,10-Dimethoxy-5-[3-cyclohexenyl]-methoxy-2,2,4-trimethyl-2,5-dihydro-1H-[1]benzo-pyrano[3,4-f]-quinoline; 10-methoxy-9-ethoxy-5-(3-propenyl)-2,2,4-trimethyl-1H-2,5-dihydro-[1]benzopyrano[3,4-f]quinoline; 10-methoxy-9-(3-propenyloxy)-5-(3-propenyl)-2,2,4-trimethyl-1H-2,5-dihydro-[1]benzopyrano[3,4-f]-quinoline; 10-methoxy-9-(3-propynyloxy)-5-(3-propenyl)-2,2,4-trimethyl-1H-2,5-dihydro-[1]benzopyrano[3,4-f]-quinoline; 2,5-dihydro-9-acetoxy-10-methoxy-2,2,4-trimethyl-5-(2-propenyl)-1H-[1]benzopyrano[3,4-f]quinoline; 2,5-dihydro-9-(4-N,N-dimethylamino-4-oxo-butanoyloxy)-10-methoxy-2,2,4-trimethyl-5-(2-propenyl)-1H-[1]benzopyrano[3,4-f]-quinoline; 7-bromo-5-[3-cyclohexenyl]-10-methoxy-2,2,4-trimethyl-2,5-dihydro-1H-[1]benzopyrano[3,4-f]-quinoline; 10-methoxy-7-bromo-5-(3-propenyl)-2,2,4-trimethyl-1H-2,5-dihydro-[1]benzopyrano[3,4-f]quinoline; 7-bromo-5-[1-methyl-3-cyclohexenyl]-10-methoxy-2,2,4-trimethyl-2,5-dihydro-1H-[1]benzopyrano[3,4-f]quinoline; 10-methoxy-9-bromo-5-(3-propenyl)-2,2,4-trimethyl-1H-2,5-dihydro-[1]benzopyrano-[3,4-f]quinoline; 7,9-Dibromo-10-methoxy-5-(3-propenyl)-2,2,4-trimethyl-1H-2,5-dihydro-[1]benzopyrano[3,4-f]-quinoline; 7,9-Dibromo-5-[cyclohexen-3-yl]-10-methoxy-2,2,4-trimethyl-2,5-dihydro-1H-[1]benzopyrano[3,4-f]-quinoline; 7,9-Dibromo-5-[1-methyl-3-cyclohexenyl]-10-methoxy-2,2,4-trimethyl-2,5-dihydro-1H-[1]benzopyrano[3,4-f]-quinoline; 10-methoxy-7-(2-ethenyl)-5-(3-propenyl)-2,2,4-trimethyl-1H-2,5-dihydro-[1]benzopyrano[3,4-f]-quinoline; 10-methoxy-7-methyl-5-(3-propenyl)-2,2,4-trimethyl-1H-2,5-dihydro-[1]benzopyrano[3,4-f]quinoline; 10-methoxy-7-acetyl-5-(3-propenyl)-2,2,4-trimethyl-1H-2,5-dihydro-[1]benzopyrano[3,4-f]-quinoline; (±)2,5-dihydro-9-methyl-10-methoxy-2,2,4-trimethyl-5-(1-methylcyclohexen-3-yl)-1H-[1]benzopyrano-[3,4-f]quinoline; 10-methoxy-7-methyl-9-methyl-5-(3-propenyl)-2,2,4-trimethyl-1H-2,5-dihydro-[1]benzopyrano[3,4-f]-quinoline; 2,5-dihydro-10-methoxy-5-(3-(N-methyl-N-(carbomethoxymethyl)aminocarbanyloxy)-phenyl)-2,2,4-trimethyl-1H-[1]benzopyrano[3,4-f]quinoline; 2,5-dihydro-10-methoxy-5-(3-(N-methyl-N—(N-methylcarbonyl)aminocarbonyloxy)phenyl)-2,2,4-trimethyl-1H-[1]benzopyrano[3,4-f]quinoline; 2,5-dihydro-10-methoxy)-5-(3-(N-methylamino-carbonyloxy)phenyl)-2,2,4-trimethyl-1H-[1]benzopyrano[3,4-f]quinoline; 2,5-dihydro-10-methoxy-5-(3-(2-hydroxyethyl)phenyl)-2,2,4-trimethyl-1H-[1]benzopyrano[3,4-f]-quinoline; 2,5-dihydro-10-methoxy-5-(3-(2-methanesulfonyloxyethyl)phenyl)-2,2,4-trimethyl-1H-[1]benzopyrano[3,4-f]quinoline; 2,5-dihydro-10-methoxy-5-(3-(2-methylhioethyl)phenyl)-2,2,4-trimethyl-1H-[1]benzopyrano[3,4-f]-quinoline; 2,5-dihydro-10-methoxy-5-(3-(2-(N,N-dimethylaminocarbonyloxy)ethyl)phenyl)-2,2,4-trimethyl-1H-[1]benzopyrano[3,4-f]quinoline; 2,5-dihydro-10-methoxy-5-(3-(2-(N,N-dimethylamino)ethyl)phenyl)-2,2,4-trimethyl-1H-[1]benzo-pyrano[3,4-f]quinoline; 2,5-dihydro-10-methoxy-5-cyclopropyl-2,2,4-trimethyl-1H-[1]benzopyrano[3,4-f]-quinoline; 2,5-dihydro-10-methoxy-5-ethenyl-2,2,4-trimethyl-1H-[1]benzopyrano-[3,4-f]quinoline; trans-2,5-dihydro-10-methoxy-5-(2-phenylethenyl)-2,2,4-trimethyl-1H-[1]benzopyrano[3,4-f]-quinoline; 2,5-dihydro-10-methoxy-5-(2-phenylethynyl)-2,2,4-trimethyl-1H-[1]benzopyrano[3,4-f]-quinoline; cis-2,5-dihydro-10-methoxy-5-(2-phenylethenyl)-2,2,4-trimethyl-1H-[1]benzopyrano[3,4-f]quinoline; 2,5-dihydro-10-methoxy-5-(2-methylpropenyl)-2,2,4-trimethyl-1H-[1]benzopyrano[3,4-f]quinoline; trans-2,5-dihydro-10-methoxy-5-(1-cyclohexenyl)-2,2,4-trimethyl-1H-[1]benzo-pyrano[3,4-f]quinoline; 2,5-dihydro-5-(3-propenyl)-10-methylthio-2,2,4-trimethyl-1H-[1]benzopyrano[3,4-f]quinoline; (±)-2,5-dihydro-9-(4-acetamidobutanoyloxy)-10-methoxy-2,2,4-trimethyl-5-allyl-1H-[1]benzo-pyrano[3,4-f]quinoline; 10-(difluoro-methoxy)-2,5-dihydro-5-phenyl-2,2,4-trimethyl-1H-[1]benzopyrano[3,4-f]quinoline; 10-(bromodifluoromethoxy)-2,5-dihydro-5-phenyl-2,2,4-trimethyl-1H-[1]benzo-pyrano[3,4-f]quinoline; 2,5-dihydro-9-hydroxy-10-methoxy-2,2,4-trimethyl-5-((2-fluorophenyl)methyl)-1H-[1]benzopyrano[3,4-f]-quinoline; 10-methoxy-5-(5-methyl-isoxazol-3-yl)methylidene-2,5-dihydro-5-phenyl-2,2,4-trimethyl-1H-[1]benzopyrano-[3,4-f]quinoline; 10-methoxy-5-(3-methylisoxazol-5-yl)methylidene-2,5-dihydro-5-phenyl-2,2,4-trimethyl-1H-[1]benzopyrano[3,4-f]quinoline; 10-methoxy-5-(4,5-dimethyl-1,3-oxazol-2-yl)-methylidene-2,5-dihydro-5-phenyl-2,2,4-trimethyl-1H-[1]benzopyrano[3,4-f]quinoline; 10-methoxy-5-(6-chloropyridin-2-methylidene-2,5-dihydro-5-phenyl-2,2,4-trimethyl-1H-[1]benzo-yrano[3,4-f]quinoline; 10-methoxy-5-(pyridin-2-yl)methylidene-2,5-dihydro-5-phenyl-2,2,4-trimethyl-1H-[1-f]quinoline; 10-methoxy-5-(but-3-enylidene)-2,5-dihydro-5-phenyl-2,2,4-trimethyl-1H-[1-f]-quinoline; 10-methoxy-5-(1-methylpropylidene)-2,5-dihydro-5-phenyl-2,2,4-trimethyl-1H-[1-f]quinoline; 10-methoxy-5-(1-butylidene)-2,5-dihydro-5-phenyl-2,2,4-trimethyl-1H-[1-f]quinoline; Z-5-(benzylidenyl)-9-hydroxy-10-methoxy-2,2,4-trimethyl-1H-2,5-dihydro-[1]benzopyrano[3,4-f]-quinoline; Z-5-(2,5-difluorobenzylidenyl)-9-hydroxy-10-methoxy-2,2,4-trimethyl-1H-2,5-dihydro-[1]benzo-pyrano[3,4-f]quinoline; Z-9-hydroxy-10-methoxy-5-(2-picolinylidenyl)-2,2,4-trimethyl-2,5-dihydro-1H-[1]benzopyrano[3,4-f]-quinoline; 9-hydroxy-10-methoxy-5-(3,5-difluorophenyl)-methylidene-2,5-dihydro-5-phenyl-2,2,4-trimethyl-1H-[1]benzopyrano[3,4-f]quinoline; 9-hydroxy-10-methoxy-5-(3,4-difluorophenyl)methylidene-2,5-dihydro-5-phenyl-2,2,4-trimethyl-1H-[1]benzopyrano[3,4-f]quinoline; (Z)-9-hydroxy-10-methoxy-5-((4-fluorophenyl)methylene)-2,2,4-trimethyl-1H-2,5-dihydro-[1]benzo-pyrano[3,4-f]-quinoline; (Z)-9-hydroxy-10-methoxy-5-([2,3-difluorophenyl]methylene)-2,2,4-trimethyl-1H-2,5-dihydro-[1]benzopyrano[3,4-f]quinoline; Z-5-(3-fluorobenzylidenyl)-10-methoxy-9-hydroxy-2,2,4-trimethyl-2,5-dihydro-1H-[1]benzopyrano[3,4-f]-quinoline; 9-hydroxy-10-methoxy-5-ethyl-2,2,4-trimethyl-2,5-dihydro-1H-[1]benzopyrano[3,4-f]quinoline; (±)-2,5-dihydro-9-cyanomethoxy-10-methoxy-2,2,4-trimethyl-5-allyl-1H-[1]benzopyrano[3,4-f]-quinoline; 2,5-dihydro-9-(4-N,N-diethyl-amino-4-oxo-butanoyloxy) 10-methoxy-2,2,4-trimethyl-5-(2-propenyl)-1H-[1]benzo-pyrano[3,4-f]quinoline; 2,5-dihydro-9-(4-N-piperidino-4-oxo-butanoyloxy)-10-methoxy-2,2,4-trimethyl-5(2-propenyl)-1H-[1]benzopyrano[3,4-f]quinoline; 2,5-dihydro-9-(4-N-morpholino4-oxo-butanoyloxy)-10-methoxy-2,2,4-trimethyl-5-(2-propenyl)-1H-[1]benzopyrano[3,4-f]quinoline; 2,5-dihydro-9-(4-N,N-dimethylamino-4-oxo-butanoyloxy)-10-methoxy-2,2,4-trimethyl-5-(3,4,5-trifluorophenyl)-1H-[1]benzopyrano[3,4-f]quinoline; 2,5-dihydro-9-hydroxy-10-methoxy-2,2,4-trimethyl-5-(3-5-difluorophenylmethyl)-1H-[1]benzopyrano-[3,4-f]quinoline; 2,5-dihydro-9-hydroxy-10-methoxy-2,2,4-trimethyl-5-cyclopentyl-1H-[1]benzopyrano[3,4-f]quinoline; 2,5-dihydro-9-hydroxy-10-methoxy-2,2,4-trimethyl-5-((2-fluorophenyl)methyl)-1H-[1]benzopyrano[3,4-f]-quinoline; 2,5-dihydro-9-hydroxymethyl-10-methoxy-2,2,4-trimethyl-5-allyl-1H-[1]benzopyrano[3,4-f]quinoline; 2,5-dihydro-9-hydroxy-10-methoxy-2,2,4-trimethyl-5-(1-pentenyl)-1H-[1]benzopyrano[3,4-f]quinoline; 2,5-dihydro-9-methylcarboxylate-10-methoxy-2,2,4-trimethyl-5-allyl-1H-[1]benzo-pyrano[3,4-f]-quinoline; 2,5-dihydro-9-hydroxy-10-methoxy-2,2,4-trimethyl-5-allenyl-1H-[1]benzopyrano[3,4-f]quinoline; (−)(5S,3′S)-2,5-dihydro-10-methoxy-2,2,4-trimethyl-5-(cyclopenten-3-yl)-1H-[1]benzopyrano[3,4-f]-quinoline; (−)(5S,3′S)-2,5-dihydro-10-methoxy-2,2,4-trimethyl-5-(cyclohexen-3-yl)-1H-[1]benzopyrano[3,4-f]-quinoline; (−)(5S,3′R)-2,5-dihydro-10-methoxy-2,2,4-trimethyl-5-(cyclohexen-3-yl)-1H-[1]benzopyrano[3,4-f]-quinoline; (−)(5S,3′R)-2,5-dihydro-10-methoxy-2,2,4-trimethyl-5-(cyclopenten-3-yl)-1H-[1]benzopyrano[3,4-f]-quinoline; 2,5-dihydro-9-hydroxy-10-methoxy-2,2,4-trimethyl-5-(3(Z)-pentenyl)-1H-[1]benzopyrano[3,4-f]-quinoline; 2,5-dihydro-9-hydroxy-10-methoxy-2,2,4-trimethyl-5-(3-acetoxyphenyl)-1H-[1]benzo-pyrano[3,4-f]-quinoline; 10-difluoromethoxy-5-[[3-(methylthio)methoxy]phenyl]-2,2,4-trimethyl-1H-2,5-dihydro-[1]benzo-pyrano[3,4-f]quinoline; 2,5-dihydro-9-hydroxy-10-methoxy-2,2,4-trimethyl-5-(3-hydroxyphenyl)-1H-[1]benzopyrano[3,4-f]-quinoline; 2,5-dihydro-9-methylthiomethoxy-10-methoxy-2,2,4-trimethyl-5(3(methylthio)-methoxyphenyl)-1H-[1]benzopyrano[3,4-f]quinoline; 2,5-dihydro-9-hydroxy-10-methoxy-2,2,4-trimethyl-5-(3(methylthiomethoxy)phenyl)-1H-[1]benzopyrano[3,4-f]-quinoline; 2,5-dihydro-9-N,N-dimethylcarbamoyloxy-10-methoxy-2,2,4-trimethyl-5-([2-N,N-dimethylcarbamoyl-oxy]phenyl)-1H-[1]benzopyrano[3,4-f]quinoline; 9-hydroxy-10-methoxy-5-(phenylmethylene)-2,2,4-trimethyl-1H-2,5-dihydro-[1]benzopyrano[3,4-f]-quinoline; 9-hydroxy-10-methoxy-5-([3-fluorophenyl]-methylene)-2,2,4-trimethyl-1H-2,5-dihydro-[1]benzo-pyrano[3,4-f]quinoline; rel-(5S)-9-hydroxy-5-[(3S)-(1-methoxycarbonyl)cyclohexen-3-yl]-10-methoxy-2,2,4-trimethyl-2,5-dihydro-1H-[1]benzopyrano[3,4-f]quinoline; 2,5-dihydro-9-hydroxy-10-methoxy-2,2,4-trimethyl-5-(3,5-dichlorophenyl)-1H-[1]benzopyrano[3,4-f]-quinoline; 2,5-dihydro-9-(4-N,N-dimethylamino-4-oxo-butanoyloxy)-10-methoxy-2,2,4-trimethyl-5-(1-methylethyl)-1H-[1]benzopyrano[3,4-f]quinoline; 2,5-dihydro-9-(4-N,N-dimethylamino-4-oxo-butanoyloxy)-10-methoxy-5-(phenylmethyl)-2,2,4-trimethyl-1H-[1]benzopyrano[3,4-f]quinoline; 2,5-dihydro-9-(4-N,N-dimethylamino-4-oxo-butanoyloxy)-10-methoxy-2,2,4-trimethyl-5-(2-thienyl)-1H-[1]benzopyrano[3,4-f]-quinoline; 2,5-dihydro-9-(4-N,N-dimethylaminobutanoyloxy)-10-methoxy-2,2,4-trimethyl-5-(2-propenyl)-1H-[1]benzopyrano[3,4-f]quinoline; 9-(2-ethoxy-2-oxo-ethylaminocarbonyl)-oxy-10-methoxy-5-(3-propenyl)-2,2,4-trimethyl-1H-2,5-dihydro-[1]benzopyrano[3,4-f]quinoline; (±)2,5-dihydro-9-(3-acetamido-propanoyloxy)-10-methoxy-2,2,4-trimethyl-5-allyl-1H-[1]benzopyrano-[3,4-f]quinoline; 9-hydroxy-10-methoxy-5-(phenylmethylene)-2,2,4-trimethyl-1H-2,5-dihydro-[1]benzopyrano[3,4-f]-quinoline; 9-(dimethylaminothiocarbonyl)-oxy-10-methoxy-5-(3-propenyl)-2,2,4-trimethyl-1H-2,5-dihydro-[1]benzopyrano[3,4-f]quinoline; (±)2,5-dihydro-9-(N-carbamoyl-2-aminoacetoxy)-10-methoxy-2,2,4-trimethyl-5-allyl-1H-[1]benzo-pyrano[3,4-f]quinoline; (±)2,5-dihydro-9-(4-ethoxy-4-oxo-butoxy)-10-methoxy-2,2,4-trimethyl-5-allyl-1H-[1]benzopyrano[3,4-f]-quinoline; (±)2,5-dihydro-9-(4-oxo-pentanoyloxy)-10-methoxy-2,2,4-trimethyl-5-allyl-1H-[1]benzopyrano[3,4-f]-quinoline; 2,5-dihydro-9-methylthiomethoxy-10-methoxy-2,2,4-trimethyl-5-allyl-1H-[1]benzo-pyrano[3,4-f]-quinoline; 2,5-dihydro-9-(4-N,N-diethylamino-4-oxo-pentanoyloxy)-10-methoxy-2,2,4-trimethyl-5-(2-propenyl)-1H-[1]benzopyrano[3,4-f]quinoline; 2,5-dihydro-9-(4-N,N-dimethylamino-4-oxo-pentanoyloxy)-10-methoxy-2,2,4-trimethyl-5-(2-propenyl)-1H-[1]benzopyrano[3,4-f]quinoline; 2,5-dihydro-9-(4-N-piperidino-4-oxo-pentanoyloxy)-10-methoxy-2,2,4-trimethyl-5-(2-propenyl)-1H-[1]benzopyrano[3,4-f]quinoline; 2,5-dihydro-9-(4-N-morpholino-4-oxo-pentanoyloxy)-10-methoxy-2,2,4-trimethyl-5-(2-propenyl)-1H-[1]benzopyrano[3,4-f]quinoline; (−)2,5-dihydro-9-(4-N,N-dimethylamino-4-oxo-butanoyloxy)-10-methoxy-2,2,4-trimethyl-5(S)-(3(S)-1-cyclopenten-3-yl)-1H-[1]benzopyrano[3,4-f]quinoline; 10-methoxy-9-(allylaminocarbonyl)oxy-5-(3-propenyl)-2,2,4-trimethyl-1H-2,5-dihydro-[1]benzo-pyrano[3,4-f]quinoline; 10-methoxy-9-(cyclohexylaminocarbonyl)-oxy-5-(3-propenyl)-2,2,4-trimethyl-1H-2,5-dihydro-[1]benzopyrano[3,4-f]-quinoline; 2,5-dihydro-9-hydroxy-10-methoxy-2,2,4-trimethyl-5-(3-thienyl)-1H-[1]benzopyrano[3,4-f]quinoline; 2,5-dihydro-9-hydroxy-10-methoxy-2,2,4-trimethyl-5-(4-(fluorophenyl)methyl)-1H-[1]benzo-pyrano[3,4-f]-quinoline; 10-benzyloxy-5-(2-propenyl)-2,5-dihydro-2,2,4-trimethyl-1H-[1]benzopyrano[3,4-f]quinoline; rel-(5S,3′R)-9-hydroxy-10-methoxy-5-{1-hydroxymethyl-cyclohexyl}-2,2,4-trimethyl-2,5-dihydro-1H-[1]benxopyrano[3,4-f]quinoline; 2,5-dihydro-9-hydroxy-10-methoxy-2,2,4-trimethyl-5-(1-thiazol-2-yl)-1H-[1]benzopyrano[3,4-f]-quinoline; 1,2,3,4-Tetrahydro-2,2,4-trimethyl-6-phenylquinoline; 1,2-Dihydro-2,2,4-trimethyl-6-(1,2,3-thiadiazol-5-yl)quinoline; 1,2-Dihydro-2,2,4-trimethyl-6-(1,3-oxazol-5-yl)quinoline; 6-(4,5-Dichloroimidazol-1-yl)-1,2-dihydro-2,2,4-trimethylquinoline; 6-(4-Bromo-1-methylpyrazol-3-yl)-1,2-dihydro-2,2,4-trimethylquinoline; 1,2-Dihydro-2,2,4-trimethyl-6-(3-pyridyl)quinoline; 6-(4-Fluorophenyl)-1,2-dihydro-2,2,4-trimethylquinoline; 1,2-Dihydro-6-(3-trifluoromethylphenyl)-2,2,4-trimethylquinoline; 1,2-Dihydro-2,2,4-trimethyl-6-(4-nitrophenyl)quinoline; 6-(2,3-Dichlorophenyl)-1,2-dihydro-2,2,4-trimethylquinoline; 1,2-Dihydro-6-(2-hydroxycarbonyl-4-nitrophenyl)-2,2,4-trimethylquinoline; 6-(3,4-Dichlorophenyl)-1,2-dihydro-2,2,4-trimethylquinoline; 4-Ethyl-1,2-dihydro-2,2-dimethyl-6-phenylquinoline; 1,2-Dihydro-2,2-dimethyl-6-phenyl-4-propylquinoline; 6-(2-Chlorophenyl)-1,2-dihydro-2,2,4-trimethylquinoline; 1,2-Dihydro-2,2,4-trimethylindeno[1,2-g]quinoline; 1,2-Dihydro-2,2,4-trimethylindeno[2,1-f]quinoline; 8-Bromo-1,2-dihydro-2,2,4-trimethylindeno[1,2-g]quinoline; 1,2-Dihydro-2,2,4-trimethylbenzo[b]furano[3,2,g]quinoline; 1,2-Dihydro-2,2,4-trimethylbenzo[b]furano-[2,3-f]quinoline; 6-Fluoro-1,2-dihydro-2,2,4-trimethylindeno[2,1-f]quinoline; 9-Fluoro-1,2-dihydro-2,2,4-trimethylindeno[1,2-g]quinoline; 1,2-Dihydro-9-hydroxylmethyl-2,2,4-trimethylindeno[1,2-g]quinoline; 8-Chloro-1,2-dihydro-2,2,4-trimethylindeno[1,2-g]quinoline; 8-Fluoro-1,2-dihydro-2,2,4-trimethylindeno[1,2-g]quinoline; 8-Acetyl-1,2-dihydro-2,2,4-trimethylindeno[1,2-g]quinoline; 6-Fluoro-1,2-dihydro-2,2,4-trimethylindeno[1,2-g]quinoline; 7-Bromo-1,2-dihydro-2,2,4-trimethylindeno[2,1-f]-quinoline; 1,2-Dihydro-2,2,4-trimethyl-7-nitroindeno[2,1-f]quinoline; 1,2-Dihydro-2,2,4-trimethyl-8-nitroindeno[1,2-g]quinoline; 6,9-Difluoro-1,2-dihydro-2,2,4-trimethylindeno[1,2-g]quinoline; 7-Fluoro-1,2-dihydro-2,2,4-trimethyl-11-(thiomethyl)indeno[2,1-f]quinoline; 5,8-Difluoro-1,2-dihydro-10-hydroxy-2,2,4-trimethylindeno[1,2-g]quinoline; 7,9-Difluoro-1,2-dihydro-10-hydroxy-2,2,4-trimethylindeno[1,2-g]quinoline; 7,10-Difluoro-1,2-dihydro-2,2,4-trimethyl-5-oxoindeno[3,2-f]quinoline; 7,9-Difluoro-1,2-dihydro-2,2,4-trimethyl-10-oxoindeno[1,2-g]quinoline; 8-Fluoro-1,2-dihydro-10-hydroxy-2,2,4-trimethylindeno[1,2-g]quinoline; 8-Fluoro-1,2-dihydro-2,2,4-trimethyl-10-oxoindeno[1,2-g]quinoline; 7-Fluoro-1,2-dihydro-2,2,4-trimethyl-8-nitroindeno[1,2-g]quinoline; 5-Chloro-1,2-dihydro-10-hydroxy-2,2,4-trimethylindeno[1,2-g]quinoline; 6-Fluoro-1,2-dihydro-2,2,4-trimethyl-10-oxoindeno[1,2-g]quinoline; 6-Fluoro-1,2-dihydro-10-hydroxy-2,2,4-trimethylindeno[1,2-g]quinoline; 5,8-Difluoro-1,2-dihydro-2,2,4-trimethyl-10-(trifluoroacetoxy)indeno[1,2-g]quinoline; 6-(3,5-Difluorophenyl)-1,2,3,4-tetrahydro-2,2,4-trimethylquinoline; 1,2-Dihydro-2,2,4-trimethylindolo[3,2-g]quinoline; 5-Ethyl-1,2-dihydro-2,2,4-trimethylindolo[2,3-f]quinoline; 6-(3-Chlorophenyl)-1,2-dihydro-2,2,4-trimethylquinoline; 6-(3,5-Difluorophenyl)-1,2-dihydro-2,2,4-trimethylquinoline; 6-(3-Fluorophenyl)-1,2-dihydro-2,2,4-trimethylquinoline; 1,2-Dihydro-2,2,4-trimethyl-6-(4-pyridyl)quinoline; 6-(3-Cyanophenyl)-1,2-dihydro-2,2,4-trimethylquinoline; 6-(3,5-Dichlorophenyl)-1,2-dihydro-2,2,4-trimethylquinoline; 6-(2,3-Difluorophenyl)-1,2-dihydro-2,2,4-trimethylquinoline; 1,2-Dihydro-2,2,4-trimethyl-6-(pentafluoro-phenyl)quinoline; 1,2-Dihydro-2,2,4-trimethyl-6-[4-(trifluoroacetyl)phenyl]quinoline; 1,2-Dihydro-2,2,4-trimethyl-6-(1,3-pyrimid-5-yl)quinoline; 6-(3-Cyanophenyl)-1,2,3,4-tetrahydro-2,2,4-trimethylquinoline; 5,8-Difluoro-1,2-dihydro-2,2,4-trimethyl-indeno[1,2-g]quinoline; 7,10-Difluoro-1,2-dihydro-2,2,4-trimethylindeno[2,1-f]-quinoline; 8-Cyano-1,2-dihydro-2,2,4-trimethylindeno[3,2-e]quinoline; 6-(3-Cyano-5-fluorophenyl)-1,2-dihydro-2,2,4-trimethylquinoline; 6-(3-Cyano-4-fluorophenyl)-1,2-dihydro-2,2,4-trimethylquinoline; 6-(3-Cyano-6-fluorophenyl)-1,2-dihydro-2,2,4-trimethylquinoline; 6-[5-fluoro-3-(trifluoromethyl)phenyl]-1,2-dihydro-2,2,4-trimethyl-quinoline; 6-(3-chloro-2-methylphenyl)-1,2-dihydro-2,2,4-trimethylquinoline; 1,2-Dihydro-2,2,4-trimethyl-6-(3-nitrophenyl)quinoline; 6-(3-Acetylphenyl)-1,2-dihydro-2,2,4-trimethylquinoline; 6-(3-cyano-2-methylphenyl)-1,2-dihydro-2,2,4-trimethyl-quinoline; 1,2-Dihydro-2,2,4-trimethyl-6-(3-methylphenyl)quinoline; 6-(5-Fluoro-3-nitrophenyl)-1,2-dihydro-2,2,4-trimethylquinoline; 1,2-Dihydro-6-(3-methoxyphenyl)-2,2,4-trimethylquinoline; 6-(5-Cyano-3-pyridyl)-1,2-dihydro-2,2,4-trimethylquinoline; 1,2-Dihydro-2,2,4-trimethyl-6-(2-methyl-3-nitrophenyl)quinoline; 6-(2-Amino-3,5-difluorophenyl)-1,2-dihydro-2,2,4-trimethylquinoline; 6-(3-Bromo-2-chloro-5-fluoro-phenyl)-1,2-dihydro-2,2,4-trimethylquinoline; 6-(3-Cyano-5-fluorophenyl)-1,2-dihydro-2,2,4-trimethyl-3-quinolone; 6-(3-Fluoro-2-methylphenyl)-1,2-dihydro-2,2,4-trimethylquinoline; 1,2-Dihydro-2,2,4-trimethyl-6-(3-methylthiophenyl)quinoline; 6-(5-Chloro-2-thienyl)-1,2-dihydro-2,2,4-trimethylquinoline; 1,2-Dihydro-2,2,4-trimethyl-6-(3-methyl-2-thienyl)quinoline; 8-Fluoro-1,2-dihydro-2,2,4-trimethyl-6-(3-nitrophenyl)quinoline; 1,2-Dihydro-6-(3-nitrophenyl)-2,2,4,8-tetramethylquinoline; 6-(5-Bromo-3-pyridyl)-1,2-dihydro-2,2,4-trimethylquinoline; 6-(3-Bromo-2-pyridyl)-1,2-dihydro-2,2,4-trimethylquinoline; 6-(3-Bromo-2-thienyl)-1,2-dihydro-2,2,4-trimethylquinoline; 1,2-Dihydro-6-(2,3,5,6-tetrafluoro-4-pyridyl)-2,2,4-trimethyl-quinoline; 5,8-Difluoro-1,2-dihydro-6-(3-nitrophenyl)-2,2,4-trimethylquinoline; 2,4-Diethyl-8-fluoro-1,2-dihydro-2-methyl-6-(3-nitrophenyl)quinoline; 6-(3-Bromophenyl)-1,2-dihydro-2,2,4-trimethylquinoline; 1,2-Dihydro-2,2,4-trimethyl-6-(5-nitro-2-thienyl)quinoline; 1,2-Dihydro-6-(2,4,5-trifluorophenyl)-2,2,4-trimethyl-quinoline; 6-(3-Bromo-5-fluorophenyl)-1,2-dihydro-2,2,4-trimethylquinoline; 6-(5-Carboxaldehyde-3-thienyl)-1,2-dihydro-2,2,4-trimethylquinoline; 1,2-Dihydro-2,2,4,7-tetramethyl-6-(3-nitrophenyl)quinoline; 6-(5-Fluoro-2-methoxy-3-nitrophenyl)-1,2-dihydro-2,2,4-trimethylquinoline; 6-(3-Chloro-2-methoxyphenyl)-1,2-dihydro-2,2,4-trimethylquinoline; 1,2-Dihydro-2,2,4-trimethyl-6-(2,3,4-trifluorophenyl)quinoline; 6-(3-Bromo-2-methylphenyl)-1,2-dihydro-2,2,4-trimethylquinoline; 7-Chloro-1,2-dihydro-2,2,4-trimethyl-6-(3-nitrophenyl)quinoline; 5-Chloro-1,2-dihydro-2,2,4-trimethyl-6-(3-nitrophenyl)quinoline; 8-Chloro-1,2-dihydro-2,2,4-trimethyl-6-(3-nitrophenyl)quinoline; 8-Ethyl-1,2-dihydro-2,2,4-trimethyl-6-(3-nitrophenyl)quinoline; 9-Chloro-1,2-dihydro-2,2-dimethyl-5-coumarino[3,4-f]-quinoline; 1,2-Dihydro-9-methoxy-2,2,4-trimethyl-5-coumarino[3,4-f]quinoline; 9-Fluoro-1,2-dihydro-2,2,4,11-tetramethyl-5-coumarino[3,4-f]quinoline; 1,2-Dihydro-2,2,4,9-tetramethyl-5-coumarino-[3,4-f]quinoline; 7-Chloro-1,2-dihydro-2,2,4-trimethyl-5-coumarino[3,4-f]quinoline; (R/S)-9-Chloro-1,2-dihydro-5-methoxy-2,2,4-trimethyl-5H-chromeno[3,4-f]quinoline; (R/S)-9-Fluoro-1,2-dihydro-2,2,4-trimethyl-5H-chromeno[3,4-f]quinoline; 6-(5-Cyano-2-thienyl)-1,2-dihydro-2,2,4-trimethylquinoline; 6-(5-Cyano-3-thienyl)-1,2-dihydro-2,2,4-trimethylquinoline; 6-(3-Formylphenyl)-1,2-dihydro-2,2,4-trimethylquinoline; 1,2-Dihydro-2,2,4-trimethyl-6-[3-(methylsulfonyl)phenyl]quinoline; (R/S)-6-(3-Cyano-5-fluorophenyl)-1,2,3,4-Tetrahydro-2,2,4-trimethylquinoline; (R/S)-9-Chloro-1,2-dihydro-2,2,4-trimethyl-5-phenyl-5H-chromeno[3,4-f]quinoline; 2,5-dihydro-10-methoxy-2,2,4-trimethyl-5-(2-propenyl)-1H-[1]benzopyrano[3,4-f]-quinoline; 10-(difluoromethoxy)-2,5-dihydro-2,2,4-trimethyl-5-(2-propenyl)-1H-[1]benzopyrano[3,4-f]-quinoline; 10-ethoxy-2,5-dihydro-2,2,4-trimethyl-5-phenyl-1H-[1]benzopyrano[3,4-f]quinoline; 5-(3-bromo-5-methylphenyl)-2,5-dihydro-10-methoxy-2,2,4-trimethyl-1H-[1]benzopyrano[3,4-f]-quinoline; 3-(2,5-dihydro-10-methoxy-2,2,4-trimethyl-1H-[1]benzopyrano[3,4-f]quinolin-5-yl)phenol, acetate(ester); 3-(2,5-dihydro-10-methoxy-2,2,4-trimethyl-1H-[1]benzopyrano[3,4-f]quinolin-5-yl)phenol; 2,5-dihydro-10-methoxy-2,2,4-trimethyl-5-[[3-(methylthio)methoxy]phenyl]-1H-[1]benzopyrano-[3,4-f]quinoline; [3-(2,5-dihydro-10-methoxy-2,2,4-trimethyl-1H-[1]benzopyrano[3,4-f]quinolin-5-yl)phenyl]-dimethylcarbamate; 5-[3-(2-furanyl)-5-methylphenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-methyl-5-(1-morpholinyl)-phenyl]-1H-[1]benzo-pyrano[3,4-f]quinoline; 2,5-dihydro-10-methoxy-2,2,4-trimethyl-5-(phenylmethylene)-1H-[1]benzopyrano[3,4-f]quinoline; 5-(3,5-dichlorophenyl)-2,5-dihydro-10-methoxy-2,2,4-trimethyl-1H-[1]benzopyrano[3,4-f]quinoline; 5-butyl-2,5-dihydro-10-methoxy-2,2,4-trimethyl-1H-[1]benzopyrano[3,4-f]quinoline; 2,5-dihydro-10-methoxy-2,2,4-trimethyl-5-[3-(trifluoromethyl)phenyl]-1H-[1]benzopyrano[3,4-f]-quinoline; 2,5-dihydro-10-methoxy-5-(4-methoxyphenyl)-2,2,4-trimethyl-1H-[1]benzo-pyrano[3,4-f]-quinoline; 5-(3-chlorophenyl)-2,5-dihydro-10-methoxy-2,2,4-trimethyl-1H-[1]benzopyrano[3,4-f]-quinoline; 2,5-dihydro-10-methoxy-2,2,4-trimethyl-5-(3-methylphenyl)-1H-[1]benzopyrano[3,4-f]-quinoline; (±)-2,5-dihydro-10-methoxy-2,2,4-trimethyl-5-phenyl-1H-[1]benzopyrano[3,4-f]quinoline; (±)-2,5-dihydro-10-methoxy-2,2,4-trimethyl-5-phenyl-1H-[1]benzopyrano[3,4-f]quinoline; 5-(3,5-dimethylphenyl)-2,5-dihydro-10-methoxy-2,2,4-trimethyl-1H-[1]benzopyrano[3,4-f]-quinoline; 5-(4-chlorophenyl)-2,5-dihydro-10-methoxy-2,2,4-trimethyl-1H-[1]benzopyrano[3,4-f]-quinoline; 5-(3,4-dimethylphenyl)-2,5-dihydro-10-methoxy-2,2,4-trimethyl-1H-[1]benzopyrano[3,4-f]-quinoline; 5-(4-fluorophenyl)-2,5-dihydro-10-methoxy-2,2,4-trimethyl-1H-[1]benzopyrano[3,4-f]quinoline; 5-[3,5-bis(trifluoromethyl)phenyl]-2,5-dihydro-10-methoxy-2,2,4-trimethyl-1H-[1]benzo-pyrano[3,4-f]quinoline; (−)-5-(3,5-dichlorophenyl)-2,5-dihydro-10-methoxy-2,2,4-trimethyl-1H-[1]benzopyrano[3,4-f]-quinoline; (+)-5-(3,5-dichlorophenyl)-2,5-dihydro-10-methoxy-2,2,4-trimethyl-1H-[1]benzopyrano[3,4-f]-quinoline; 5-(3,5-difluorophenyl)-2,5-dihydro-10-methoxy-2,2,4-trimethyl-1H-[1]benzopyrano[3,4-f]-quinoline; 2,5-dihydro-10-methoxy-2,2,4,N-tetramethyl-N-phenyl-1H-[1]benzopyrano[3,4-f]quinolin-5-amine; (−)2,5-dihydro-10-methoxy-2,2,4-trimethyl-5-(2-propenyl)-1H-[1]benzopyrano[3,4-f]quinoline; (+)-2,5-dihydro-10-methoxy-2,2,4-trimethyl-5-(2-propenyl)-1H-[1]benzopyrano[3,4-f]-quinoline; 2,5-dihydro-10-methoxy-2,2,4-trimethyl-1H-[1]benzopyrano[3,4-f]quinoline; 4-(2,5-dihydro-10-methoxy-2,2,4-trimethyl-1H-[1]benzopyrano[3,4-f]quinolin-5-yl)-N,N-dimethylbenzenamide; 2,5-dihydro-10-methoxy-2,2,4-trimethyl-(5-methoxy-2-thienyl)-1H-[1]benzopyrano[3,4-f]-quinoline; 2,5-dihydro-10-methoxy-2,2,4-trimethyl-5-(5-propyl-2-thienyl)-1H-[1]benzopyrano[3,4-f]quinoline; 2,5-dihydro-10-methoxy-2,2,4-trimethyl-5-[4-(1-morpholinyl)phenyl]-1H-[1]benzopyrano[3,4-f]-quinoline; 1-(2,5-dihydro-10-methoxy-2,2,4-trimethyl-1H-[1]benzopyrano[3,4-f]quinolin-5-yl)-3,3-dimethyl-2-butanone; 2,5-dihydro-10-methoxy-2,2,4-trimethyl-1H-[1]benzopyrano-[3,4-f]quinoline-5-carbonitrile, 1-(2,5-dihydro-10-methoxy-2,2,4-trimethyl-1H-[1]benzopyrano[3,4-f]quinolin-5-yl)-2-propanone, methyl-2,5-dihydro-10-methoxy-2,2,4-trimethyl-1H-[1]benzopyrano[3,4-f]quinoline-5-acetate; 2-(2,5-dihydro-10-methoxy-2,2,4-trimethyl-1H-[1]benzopyrano[3,4-f]quinolin-5-yl)-1-phenylethanone; 5-[2-(chloromethyl)-2-propenyl]-2,5-dihydro-10-methoxy-2,2,4-trimethyl-1H-[1]benzopyrano[3,4-f]-quinoline; 2,5-dihydro-10-methoxy-2,2,4-trimethyl-(-methylene-1H-[1]benzopyrano[3,4-f]quinoline-5-propanol, acetate(ester); 2,5-dihydro-10-methoxy-2,2,4-trimethyl-5-(4-methylphenyl)-1H-[1]benzopyrano[3,4-f]quinoline; 5-(3-fluoro-4-methylphenyl)-2,5-dihydro-10-methoxy-2,2,4-trimethyl-1H-[1]benzo-pyrano[3,4-f]-quinoline; 5-(3-bromophenyl)-2,5-dihydro-10-methoxy-2,2,4-trimethyl-1H-[1]benzopyrano[3,4-f]quinoline; 2,5-dihydro-10-methoxy-2,2,4-trimethyl-5-(phenylmethyl)-1H-[1]benzopyrano[3,4-f]quinoline; 2,5-dihydro-10-methoxy-2,2,4-trimethyl-5-propyl-1H-[1]benzopyrano[3,4-f]quinoline; 5-(4-fluorophenyl)-2,5-dihydro-10-methoxy-2,2,4-trimethyl-1H-[1]benzopyrano[3,4-f]quinoline; 5-(3-fluorophenyl)-2,5-dihydro-10-methoxy-2,2,4-trimethyl-1H-[1]benzopyrano[3,4-f]quinoline; 2,5-dihydro-10-methoxy-2,2,4,5-tetramethyl-1H-[1]benzopyrano[3,4-f]quinoline; 2,5-dihydro-10-methoxy-2,2,4-trimethyl-5-(1-methylethyl)-1H-[1]benzopyrano[3,4-f]-quinoline; 2,5-dihydro-10-methoxy-2,2,4-trimethyl-5-(2-methylpropyl)-1H-[1]benzopyrano[3,4-f]quinoline; 5-ethyl-2,5-dihydro-10-methoxy-2,2,4-trimethyl-1H-[1]benzopyrano[3,4-f]quinoline; 2,5-dihydro-10-methoxy-2,2,4-trimethyl-1H-[1]benzopyrano[3,4-f]quinoline-5-carboximidic acid ethyl ester; 2,5-dihydro-10-methoxy-2,2,4-trimethyl-(-methylene-1H-[1]benzopyrano[3,4-f]quinoline-5-propanol; 2,5-dihydro-10-methoxy-2,2,4,N,N-pentamethyl-1H-[1]benzopyrano[3,4-f]quinoline-5-acetamide; 2,5-dihydro-10-methoxy-2,2,4,N,N-pentamethyl-1H-[1]benzopyrano[3,4-f]quinoline-5-ethanamine; N-cyclopropyl-2,5-dihydro-10-methoxy-2,2,4-trimethyl-1H-[1]benzopyrano[3,4-f]quinoline-5-acetamide; 2,5-dihydro-10-methoxy-2,2,4-trimethyl-5-(2-propynyl)-1H-[1]benzopyrano[3,4-f]quinoline; 5-(2,5-dihydro-10-methoxy-2,2,4-trimethyl-1H-[1]benzopyrano[3,4-f]quinolin-5-yl)-2(5H)-furanone; 5-(3-butenyl)-2,5-dihydro-10-methoxy-2,2,4-trimethyl-1H-[1]benzopyrano[3,4-f]quinoline; 2,5-dihydro-10-methoxy-2,2,4-trimethyl-1H-[1]benzopyrano[3,4-f]quinoline-5 propanol; 5-(3,5-dichlorophenyl)-10-ethoxy-2,5-dihydro-2,2,4-trimethyl-1H-[1]benzopyrano[3,4-f]quinoline, 10-(bromodifluoromethoxy)-2,5-dihydro-2,2,4-trimethyl-5-(2-propenyl)-1H-[1]benzopyrano[3,4-f]-quinoline; [3-(2,5-dihydro-10-methoxy-2,2,4-trimethyl-1H-[1]benzopyrano[3,4-f]quinolin-5-yl)phenyl]-methylcarbonate; 2,5-dihydro-10-methoxy-5-(3-methoxyphenyl)-2,2,4-trimethyl-1H-[1]benzopyrano[3,4-f]quinoline; 2,5-dihydro-10-methoxy-2,2,4-trimethyl-5-[3-(2-propenyloxy)phenyl]-1H-[1]benzopyrano[3,4-f]-quinoline; 2,5-dihydro-10-methoxy-2,2,4-trimethyl-5-[3-(phenylmethoxy)phenyl]-1H-[1]benzopyrano[3,4-f]-quinoline; 5-[3-(cyclopropylmethoxy)phenyl]-2,5-dihydro-10-methoxy-2,2,4-trimethyl-1H-[1]benzopyrano-[3,4-f]quinoline; 2,5-dihydro-10-methoxy-2,2,4-trimethyl-5-[3-[2-(1-piperidinyl)ethoxy]phenyl]-1H-[1]benzopyrano-[3,4-f]quinoline; 5-(3-hexyloxyphenyl)-2,5-dihydro-10-methoxy-2,2,4-trimethyl-1H-[1]benzopyrano[3,4-f]quinoline; 5-[3-(2,4-dinitrophenoxy)phenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-(2-propynyloxy)phenyl]-1H-[1]benzopyrano[3,4-f]-quinoline; 3-(2,5-dihydro-10-methoxy-2,2,4-trimethyl-1H-[1]benzopyrano[3,4-f]quinolin-5-yl)phenol-4-methylbenzenesulfonate (ester); 4-(2,5-dihydro-10-methoxy-2,2,4-trimethyl-1H-[1]benzopyrano[3,4-f]quinolin-5-yl)phenolacetate(ester); 4-(2,5-dihydro-10-methoxy-2,2,4-trimethyl-1H-[1]benzopyrano[3,4-f]quinolin-5-yl)phenol; 2,5-dihydro-10-methoxy-2,2,4-trimethyl-5-[[4-(methylthio)methoxy]phenyl]-1H-[1]benzo-pyrano[3,4-f]quinoline; [4-(2,5-dihydro-10-methoxy-2,2,4-trimethyl-1H-[1]benzo-pyrano[3,4-f]quinolin-5-yl)phenyl]-dimethylcarbamate; 2,5-dihydro-10-methoxy-2,2,4-trimethyl-5-[4-(phenylmethoxy)phenyl]-1H-[1]benzopyrano[3,4-f]-quinoline; 2,5-dihydro-10-methoxy-2,2,4-trimethyl-5-[3-(methoxymethoxy)phenyl]-1H-[1]benzo-pyrano[3,4-f]-quinoline; [(2,5-dihydro-10-methoxy-2,2,4-trimethyl-1H-[1]benzopyrano-[3,4-f]quinolin-5-yl)phenyl]1-morpholinecarboxylate; 2,5-dihydro-10-methoxy-2,2,4-trimethyl-5-[3-[(methylsulfinyl)methoxy]phenyl]-1H-[1]benzopyrano[3,4-f]quinoline; O-[3-(2,5-dihydro-10-methoxy-2,2,4-trimethyl-1H-[1]benzopyrano[3,4-f]quinolin-5-yl)phenyl]ester; 2,5-dihydro-10-methoxy-2,2,4-trimethyl-5-[3-(methylthio)phenyl]-1H-[1]benzopyrano[3,4-f]-quinoline; O-[3-(2,5-dihydro-10-methoxy-2,2,4-trimethyl-1H-[1]benzopyrano[3,4-f]quinolin-5-yl)phenyl]-methylcarbonothioate; [3-(2,5-dihydro-10-methoxy-2,2,4-trimethyl]-1H-[1]benzopyrano[3,4-f]quinolin-5-yl)phenyl]-trifluoro-methanesulfonate; 5-[3-(4,5-dihydro-4,4-dimethyl-2-oxazolyl)phenyl]-2,5-dihydro-10-methoxy-2,2,4-trimethyl-1H-[1]benzopyrano[3,4-f]-quinoline; ethyl 3-(2,5-dihydro-10-methoxy-2,2,4-trimethyl-1H-[1]benzopyrano[3,4-f]quinolin-5-yl)benzoate; 3-(2,5-dihydro-10-methoxy-2,2,4-trimethyl-1H-[1]benzopyrano[3,4-f]quinolin-5-yl)benzoic acid; 2,5-dihydro-10-methoxy-2,2,4-trimethyl-5-[3-methyl-5-(2-propenyl)phenyl]-1H-[1]benzopyrano-[3,4-f]quinoline; 1-[3-(2,5-dihydro-10-methoxy-2,2,4-trimethyl-1H-[1]benzopyrano[3,4-f]quinolin-5-yl)-5-methylphenyl]ethanone; 3-(2,5-dihydro-10-methoxy-2,2,4-trimethyl-1H-[1]benzopyrano[3,4-f]quinolin-5-yl)-5-trimethylbenzene-methanol; 5-[3-(2-furanyl)phenyl]-2,5-dihydro-10-methoxy-2,2,4-trimethyl-1H-[1]benzopyrano[3,4-f]-quinoline; 2,5-dihydro-10-methoxy-2,2,4-trimethyl-5-[3-methyl-5-(1H-pyrrolidin-1-yl)phenyl]-1H-[1]benzopyrano[3,4-f]quinoline; 3-(2,5-dihydro-10-methoxy-2,2,4-trimethyl-1H-[1]benzopyrano[3,4-f]quinolin-5-methyl)-5N-dimethylbenzenamine; 3-(2,5-dihydro-10-methoxy-2,2,4-trimethyl-1H-[1]benzopyrano[3,4-f]quinolin-5-yl)-5-methyl-N-(2-propenyl)benzamide; 3-(2,5-dihydro-10-methoxy-2,2,4-trimethyl-1H-[1]benzopyrano[3,4-f]quinolin-5-yl)-N-(2-methoxyethyl)-5-methyl-benzenamide; 3-(2,5-dihydro-10-methoxy-2,2,4-trimethyl-1H-[1]benzopyrano[3,4-f]quinolin-5-yl)-N-(2-propenyl)benzenamide; N′-[3-(2,5-dihydro-10-methoxy-2,2,4-trimethyl-1H-[1]benzopyrano[3,4-f]quinolin-5-yl)-5-methylphenyl]-N,N-dimethylurea; N-[3-(2,5-dihydro-10-methoxy-2,2,4-trimethyl-1H-[1]benzopyrano[3,4-f]quinolin-5-yl)phenyl]-benzenemethanamide; 5-[(3,5-dichlorophenyl)methylene]-2,5-dihydro-10-methoxy-2,2,4-trimethyl-1H-[1]benzopyrano[3,4-f]-quinoline; 5-[(4-chlorophenyl)-methylene]-2,5-dihydro-10-methoxy-2,2,4-trimethyl-1H-[1]benzopyrano[3,4-f]-quinoline; 2,5-dihydro-10-methoxy-2,2,4-trimethyl-5-[[3-(trifluoromethyl)phenyl]-methylene]-1H-[1]benzo-pyrano[3,4-f]quinoline; 5-[(2,6-difluorophenyl)methylene]-2,5-dihydro-10-methoxy-2,2,4-trimethyl-1H-[1]benzopyrano-[3,4-f]quinoline; 5-[(2-chlorophenyl)methylene]-2,5-dihydro-10-methoxy-2,2,4-trimethyl-1H-[1]benzopyrano-[3,4-f]-quinoline; 5-[(2,6-dichlorophenyl)methylene]-2,5-dihydro-10-methoxy-2,2,4-trimethyl-1H-[1]benzopyrano-[3,4-f]quinoline; 5-[(2-fluorophenyl)methylene]-2,5-dihydro-10-methoxy-2,2,4-trimethyl-1H-[1]benzopyrano[3,4-f]-quinoline; 2,5-dihydro-10-methoxy-2,2,4-trimethyl-5-[(4,5-dihydro-4,4-dimethyl-2-oxazolyl)methylene]-1H-[1]benzopyrano[3,4-f]quinoline; 2,5-dihydro-10-methoxy-2,2,4-trimethyl-5-(2-pyridinylmethylene)-1H-[1]benzopyrano[3,4-f]-quinoline; 2,5-dihydro-10-methoxy-2,2,4-trimethyl-5-(2-thienyl)-1H-[1]benzopyrano[3,4-f]quinoline; 2,5-dihydro-9,10-dimethoxy-2,2,4-trimethyl-5-(2-propenyl)-1H-[1]benzopyrano[3,4-f]quinoline; 5-(2-cyclohexen-1-yl)-2,5-dihydro-9,10-dimethoxy-2,2,4-trimethyl-1H-[1]benzo-pyrano[3,4-f]-quinoline; 2,5-dihydro-10-methoxy-5-(3-methyl-3-butenyl)-2,2,4-trimethyl-1H-[1]benzopyrano[3,4-f]-quinoline; 2,5-dihydro-10-methoxy-5-(5,5-dimethyl-3-cyclohexenyl)-2,2,4-trimethyl-1H-[1]benzopyrano[3,4-f]-quinoline; rel(5R,2′R)2,5-dihydro-10-methoxy-5-(2-oxo-3-tetrahydropyranyl)-2,2,4-trimethyl-1H-[1]benzopyrano[3,4-f]quinoline; anti(5R,2′S)2,5-dihydro-10-methoxy-5-(2-oxo-3-tetrahydropyranyl)-2,2,4-trimethyl-1H-[1]benzopyrano[3,4-f]quinoline; 2,5-dihydro-10-methoxy-5-(3-cyclopentenyl)-2,2,4-trimethyl-1H-[1]benzopyrano[3,4-f]-quinoline; 2,5-dihydro-10-methoxy-5-(3-cyclohexenyl)-2,2,4-trimethyl-1H-[1]benzopyrano[3,4-f]-quinoline; 2,5-dihydro-10-methoxy-5-(3-butenyl)-2,2,4-trimethyl-1H-[1]benzo-pyrano[3,4-f]quinoline; 2,5-dihydro-10-methoxy-5-(1-ethenyl-1-cyclohexyl)-2,2,4-trimethyl-1H-[1]benzopyrano[3,4-f]-quinoline; 2,5-dihydro-10-methoxy-5-(4,4-dimethyl-3-cyclohexenyl)-2,2,4-trimethyl-1H-[1]benzopyrano[3,4-f]-quinoline; 2,5-dihydro-10-methoxy-5-(1-methylene-2-cyclohexyl)-2,2,4-trimethyl-1H-[1]benzo-pyrano[3,4-f]-quinoline; 2,5-dihydro-10-methoxy-5-(1-oxo-2-cyclohexyl)-2,2,4-trimethyl-1H-[1]benzopyrano[3,4-f]-quinoline; 2,5-dihydro-10-methoxy-5-(3-cyclooctenyl)-2,2,4-trimethyl-1H-[1]benzopyrano[3,4-f]quinoline; 2,5-dihydro-10-methoxy-5-(3-cycloheptenyl)-2,2,4-trimethyl-1H-[1]benzopyrano[3,4-f]quinoline; 2,5-dihydro-10-methoxy-5-(1-cyclohexenylmethyl)-2,2,4-trimethyl-1H-[1]benzo-pyrano[3,4-f]-quinoline; 2,5-dihydro-10-methoxy-5-(3,3-dimethyl-6-cyclohexenyl)-2,2,4-trimethyl-1H-[1]benzopyrano[3,4-f]-quinoline; 2,5-dihydro-10-methoxy-5-(2-bromo-3-propenyl)-2,2,4-trimethyl-1H-[1]benzopyrano[3,4-f]-quinoline; rel(5R,3′R)2,5-dihydro-10-methoxy-5-(1-hydroxymethyl-3-cyclohexenyl)-2,2,4-trimethyl-1H-[1]benzopyrano[3,4-f]quinoline; rel(5R,3′S)2,5-dihydro-10-methoxy-5-(1-hydroxymethyl-3-cyclohexenyl)-2,2,4-trimethyl-1H-[1]benzopyrano[3,4-f]quinoline; 2,5-dihydro-10-methoxy-5-(3-hydroxymethyl-3-cyclohexenyl)-2,2,4-trimethyl-1H-[1]benzopyrano-[3,4-f]quinoline; 2,5-dihydro-10-methoxy-5-(3-indolyl)-2,2,4-trimethyl-1H-[1]benzopyrano[3,4-f]quinoline; rel(5S,3′S)2,5-dihydro-10-methoxy-5-(1-methyl-3-cyclohexenyl)-2,2,4-trimethyl-1H-[1]benzo-pyrano[3,4-f]quinoline; rel(5R,3′S)2,5-dihydro-10-methoxy-5-(1-methyl-3-cyclohexenyl)-2,2,4-trimethyl-1H-[1]benzo-pyrano[3,4-f]quinoline; (−)(5S,3′S)2,5-dihydro-10-methoxy-5-(1-methyl-3-cyclohexenyl)-2,2,4-trimethyl-1H-[1]benzopyrano[3,4-f]quinoline; (−)(5S,3′R)2,5-dihydro-10-methoxy-5-(1-hydroxymethyl-3-cyclohexenyl)-2,2,4-trimethyl-1H-[1]benzopyrano[3,4-f]quinoline; (+)(5R,3′S)2,5-dihydro-10-methoxy-5-(1-hydroxymethyl-3-cyclohexenyl)-2,2,4-trimethyl-1H-[1]benzopyrano[3,4-f]quinoline; (−)-(5S,3′R)2,5-dihydro-10-methoxy-5-(1-methyl-3-cyclohexenyl)-2,2,4-trimethyl-1H-[1]benzopyrano[3,4-f]quinoline; (+)-(5R,3′S)2,5-dihydro-10-methoxy-5-(1-methyl-3-cyclohexenyl)-2,2,4-trimethyl-1H-[1]benzopyrano[3,4-f]quinoline; 2,5-dihydro-10-methoxy-5-(1-chloromethyl-3-cyclohexenyl)-2,2,4-trimethyl-1H-[1]benzo-pyrano[3,4-f]quinoline; rel(5R,3′R)2,5-dihydro-10-methoxy-5-(1-methoxymethyl-3-cyclohexenyl)-2,2,4-trimethyl-1H-[1]benzopyrano[3,4-f]quinoline; rel(5R,3′R)2,5-dihydro-10-methoxy-5-(1-methylthiomethyl-3-cyclohexenyl)-2,2,4-trimethyl-1H-[1]benzopyrano[3,4-f]quinoline; rel(5R,3′S)2,5-dihydro-10-methoxy-5-(1-acetoxymethyl-3-cyclohexenyl)-2,2,4-trimethyl-1H-[1]benzopyrano[3,4-f]quinoline; rel(5R,3′R)2,5-dihydro-10-methoxy-5-(1-acetoxymethyl-3-cyclohexenyl)-2,2,4-trimethyl-1H-[1]benzopyrano[3,4-f]quinoline; rel(5R,3′R)2,5-dihydro-10-methoxy-5-(1-methoxymethyl-3-cyclohexenyl)-2,2,4-trimethyl-1H-[1]benzopyrano[3,4-f]-quinoline; rel(5R,3′R)2,5-dihydro-10-methoxy-5-(1-(N,N-dimethylamino)methyl-3-cyclohexenyl)-2,2,4-trimethyl-1H-[1]benzopyrano[3,4-f]quinoline; rel(5R,3′S)2,5-dihydro-10-methoxy-5-(1-methylthiomethyl-3-cyclohexenyl)-2,2,4-trimethyl-1H-[1]benzopyrano-[3,4-f]quinoline; rel(5R,3′R)2,5-dihydro-10-methoxy-5-(1-(N-morpholine)methyl-3-cyclohexenyl)-2,2,4-trimethyl-1H-[1]benzopyrano[3,4-f]quinoline; rel(5R,3′R)2,5-dihydro-10-methoxy-5-(1-(N-methyl-N-methylsulfonylamino)methyl-3-cyclohexenyl)-2,2,4-trimethyl-1H-[1]benzopyrano[3,4-f]quinoline; rel(5R,3′S)2,5-dihydro-10-methoxy-S-(1-(N,N dimethylamino)methyl-3-cyclohexenyl)-2,2,4-trimethyl-1H-[1]benzopyrano[3,4-f]quinoline; rel(5R,3′R)2,5-dihydro-10-methoxy-5-(1-(N-methyl-amino)methyl-3-cyclohexenyl)-2,2,4-trimethyl-1H-[1]benzopyrano[3,4-f]quinoline; 2,5-dihydro-10-methoxy-5-(2-methyl-3-propenyl)-2,2,4-trimethyl-1H-[1]benzo-pyrano[3,4-f]-quinoline; 2,5-dihydro-10-methoxy-5-(1,3-butadien-2-yl)-2,2,4-trimethyl-1H-[1]benzopyrano[3,4-f]-quinoline; 2,5-dihydro-10-methoxy-5-(2-carbomethoxy-3-propenyl)-2,2,4-trimethyl-1H-[1]benzopyrano[3,4-f]-quinoline; 2,5-dihydro-10-methoxy-5-(1,2-dihydroxy-3-propyl)-2,2,4-trimethyl-1H-[1]benzopyrano[3,4-f]-quinoline; 2,5-dihydro-10-methoxy-5-(1,2-epoxy-3-propenyl)-2,2,4-trimethyl-1H-[1]benzopyrano[3,4-f]-quinoline; 2,5-dihydro-10-methoxy-5-(1-(N-phthalimido)-3-propyl)-2,2,4-trimethyl-1H-[1]benzopyrano[3,4-f]-quinoline; 2,5-dihydro-10-methoxy-5-(1-amino-3-propyl)-2,2,4-trimethyl-1H-[1]benzopyrano[3,4-f]quinoline; 2,5-dihydro-10-methoxy-5-(1-(hydrazinocarbonylamino)-3-propyl)-2,2,4-trimethyl-1H-[1]benzo-pyrano[3,4-f]quinoline; (E)-2,5-dihydro-10-methoxy-5-(2-carbomethoxy-1-ethenyl)-2,2,4-trimethyl-1H-[1]benzopyrano-[3,4-f]quinoline; (Z)-2,5-dihydro-10-methoxy-5-(1-propenyl)-2,2,4-trimethyl-1H-[1]benzopyrano[3,4-f]quinoline; (E)-2,5-dihydro-10-methoxy-5-(3-hydroxy-1-propenyl)-2,2,4-trimethyl-1H-[1]benzopyrano[3,4-f]-quinoline; (E)-2,5-dihydro-10-methoxy-5-(3-(N,N-dimethylaminocarbonyloxy)-1-propenyl)-2,2,4-trimethyl-1H-[1]benzopyrano[3,4-f]quinoline; (E)-2,5-dihydro-10-methoxy-5-(3-methoxymethoxy-1-propenyl)-2,2,4-trimethyl-1H-[1]benzo-pyrano-[3,4-f]-quinoline; 2,5-dihydro-10-methoxy-5-(3-hydroxy-3-propenyl)-2,2,4-trimethyl-1H-[1]benzopyrano[3,4-f]-quinoline; Methyl-2-(2,5-dihydro-10-methoxy-2,2,4-trimethyl-1H-[1]benzopyrano[3,4-f]quinolin-5-yl)acetyl hydroxamate; 2-(2,5-dihydro-10-methoxy-2,2,4-trimethyl-1H-[1]benzopyrano[3,4-f]quinolin-5-yl)acetaldehyde; 2,5-dihydro-10-methoxy-5-(2-cyclohexylidenylethyl)-2,2,4-trimethyl-1H-[1]benzopyrano[3,4-f]-quinoline; 2,5-dihydro-10-methoxy-5-(2-cyclopentylidenylethyl)-2,2,4-trimethyl-1H-[1]benzopyrano[3,4-f]-quinoline; 2,5-dihydro-10-methoxy-5-(2-cycloheptylidenylethyl)-2,2,4-trimethyl-1H-[1]benzopyrano[3,4-f]-quinoline; 2,5-dihydro-10-methoxy-5-(3-methyl-2-butenyl)-2,2,4-trimethyl-1H-[1]benzopyrano[3,4-f]quinoline; trans-2,5-dihydro-10-methoxy-5-(2-butenyl)-2,2,4-trimethyl-1H-[1]benzopyrano[3,4-f]quinoline; trans-2,5-dihydro-10-methoxy-5-(2-penten-1-yl)-2,2,4-trimethyl-1H-[1]benzopyrano[3,4-f]-quinoline; 2,5-dihydro-10-methoxy-5-(1,1-difluoro-1-propen-3-yl)-2,2,4-trimethyl-1H-[1]benzopyrano[3,4-f]-quinoline; (E)-methyl-2-(2,5-dihydro-10-methoxy-2,2,4-trimethyl-1H-[1]benzopyrano[3,4-f]quinoline; (E)-2,5-dihydro-10-methoxy-5-(4-hydroxy-2-buten-1-yl)-2,2,4-trimethyl-1H-[1]benzopyrano[3,4-f]-quinoline; (E)-2,5-dihydro-10-methoxy-5-(4-(N,N-dimethylaminocarbonyloxy)-2-buten-1-yl)-2,2,4-trimethyl-1H-[1]benzopyrano-[3,4-f]quinoline; (E)-2,5-dihydro-10-methoxy-5-(4-(N-methylaminocarbonyloxy)-2-buten-1-yl)-2,2,4-trimethyl-1H-[1]benzopyrano[3,4-f]-quinoline; (E)-2,5-dihydro-10-methoxy-5-(2-butenyl)-2,2,4-trimethyl-1H-[1]benzopyrano[3,4-f]quinoline; 2,5-dihydro-10-methoxy-5-(2-hydroxyethyl)-2,2,4-trimethyl-1H-[1]benzopyrano[3,4-f]quinoline; 2,5-dihydro-10-methoxy-5-(2-(N-benzylcarbonyloxy)ethyl)-2,2,4-trimethyl-1H-[1]benzopyrano-[3,4-f]quinoline; 2,5-dihydro-10-methoxy-5-(2-(N-morpholino-carbonyloxy)ethyl)-2,2,4-trimethyl-1H-[1]benzo-pyrano[3,4-f]quinoline; 2,5-dihydro-10-methoxy-5-(2-(N-(2-methoxyethyl)aminocarbonyloxy)ethyl)-2,2,4-trimethyl-1H-[1]benzopyrano[3,4-f]quinoline; 2,5-dihydro-10-methoxy-5-(2-(N-methylamino-carbonyloxyoxy)ethyl)-2,2,4-trimethyl-1H-[1]benzo-pyrano[3,4-f]quinoline; 2,5-dihydro-10-methoxy-5-(2-(N,N-dimethylaminocarbonyloxy)ethyl)-2,2,4-trimethyl-1H-[1]benzo-pyrano[3,4-f]-quinoline; 2,5-dihydro-10-methoxy-5-(2-methoxymethoxyethyl)-2,2,4-trimethyl-1H-[1]benzopyrano[3,4-f]-quinoline; 2,5-dihydro-10-methoxy-5-(2,2-dimethylethoxycarbonylamino)methyl)-2,2,4-trimethyl-1H-[1]benzopyrano[3,4-f]-quinoline; 2,5-dihydro-10-methoxy-5-(aminomethyl)-2,2,4-trimethyl-1H-[1]benzopyrano[3,4-f]quinoline; 2,5-dihydro-10-methoxy-5-(ethoxycarbonylamino)-methyl)-2,2,4-trimethyl-1H-[1]benzopyrano[3,4-f]-quinoline; 2,5-dihydro-10-methoxy-5-(carboethoxy)-2,2,4-trimethyl-1H-[1]benzopyrano[3,4-f]-quinoline; 2,5-dihydro-10-methoxy-5-(cyclopentyl)-2,2,4-trimethyl-1H-[1]benzopyrano[3,4-f]-quinoline; 2,5-dihydro-10-methoxy-5-(1-methylpropa-1,2-dienyl)-2,2,4-trimethyl-1H-[1]benzopyrano[3,4-f]-quinoline; 2,5-dihydro-10-methoxy-5-(3,4,5-trifluorophenyl)-2,2,4-trimethyl-1H-[1]benzopyrano[3,4-f]-quinoline; 2,5-dihydro-10-methoxy-5-(cyclohexyl)-2,2,4-trimethyl-1H-[1]benzopyrano[3,4-f]quinoline; 2,5-dihydro-10-methoxy-5-(2-pyridyl)-2,2,4-trimethyl-1H-[1]benzopyrano[3,4-f]quinoline; 2,5-dihydro-10-methoxy-5-(3-pyridyl)-2,2,4-trimethyl-1H-[1]benzopyrano[3,4-f]quinoline; 2,5-dihydro-10-methoxy-5-(4-pyridyl)-2,2,4-trimethyl-1H-[1]benzopyrano[3,4-f]quinoline; 9-10-methylenedioxy-5-phenyl-2,2,4-trimethyl-1H-2,5-dihydro-[1]benzopyrano[3,4-f]-quinoline; 9-chloro-10-methoxy-5-phenyl-2,2,4-trimethyl-2,5-dihydro-1H-[1]benzo-pyrano[3,4-f]quinoline; 5-(3-propenyl)-9-chloro-10-difluoromethoxy-2,2,4-trimethyl-2,5-dihydro-1H-[1]benzopyrano[3,4-f]-quinoline; 9-chloro-10-difluoromethoxy-5-phenyl-2,2,4-trimethyl-2,5-dihydro-1H-[1]benzopyrano[3,4-f]-quinoline; 8-fluoro-10-methoxy-5-phenyl-2,2,4-trimethyl-2,5-dihydro-1H-[1]benzopyrano[3,4-f]quinoline; 5-(3-propenyl)-8-fluoro-10-methoxy-2,2,4-trimethyl-2,5-dihydro-1H-[1]-benzopyrano-[3,4-f]-quinoline; (10-methoxy-9-fluoro-5-(3-propenyl)-2,2,4-trimethyl-1H-2,5-dihydro-[1]benzopyrano[3,4-f]-quinoline; 10-methoxy-9-hydroxy-5-(3-propenyl)-2,2,4-trimethyl-1H-2,5-dihydro-[1]benzopyrano[3,4-f]-quinoline; (±)2,5-dihydro-9-hydroxy-10-methoxy-2,2,4-trimethyl-5-(3-cyclohexenyl)-1H-[1]benzopyrano[3,4-f]-quinoline; (±)2,5-dihydro-9-hydroxy-10-methoxy-2,2,4-trimethyl-5-(1-methylcyclohexen-3-yl)-1H-[1]benzopyrano[3,4-f]-quinoline; (−)(5S,3′S)-9-hydroxy-5-[1-methyl-3-cyclohexenyl]-10-methoxy-2,2,4-trimethyl-2,5-dihydro-1H-[1]benzopyrano[3,4-f]-quinoline; (+)(5R,3′R)-9-hydroxy-5-[1-methyl-3-cyclohexenyl]-10-methoxy-2,2,4-trimethyl-2,5-dihydro-1H-[1]benzopyrano[3,4-f]-quinoline; (+)(5R,3′S)-9-hydroxy-5-[1-methyl-3-cyclohexenyl]-10-methoxy-2,2,4-trimethyl-2,5-dihydro-1H-[1]benzopyrano-[3,4-f]quinoline; (−)(5S,3′R)-9-hydroxy-5-[1-methyl-3-cyclohexenyl]-10-methoxy-2,2,4-trimethyl-2,5-dihydro-1H-[1]benzopyrano[3,4-f]quinoline; rel-(5S,3′R)-9-hydroxy-5-[1-hydroxymethyl-3-cyclohexenyl]-10-methoxy-2,2,4-trimethyl-2,5-dihydro-1H-[1]benzo-pyrano[3,4-f]quinoline; (±)(5S,3′R)-2,5-dihydro-9-hydroxy-10-methoxy-2,2,4-trimethyl-5-(1-methylcyclohexen-3-yl)-1H-[1]benzopyrano[3,4-f]quinoline; rel-(5S,3′R)-9-hydroxy-5-[1-methoxymethyl-3-cyclohexenyl]-10-methoxy-2,2,4-trimethyl-2,5-dihydro-1H-[1]benzopyrano[3,4-f]quinoline; 2,5-dihydro-9-hydroxy-10-methoxy-5-propyl-2,2,4-trimethyl-1H-[1]benzopyrano[3,4-f]quinoline; (−)(5S,3′S)-2,5-dihydro-9-hydroxy-10-methoxy-2,2,4-trimethyl-5-(3-cycloheptenyl)-1H-[1]benzopyrano[3,4-f]-quinoline; (−)(5S,3′R)-2,5-dihydro-9-hydroxy-10-methoxy-2,2,4-trimethyl-5-(3-cycloheptenyl)-1H-[1]benzopyrano[3,4-f]quinoline; 2,5-dihydro-9-hydroxy-10-methoxy-2,2,4-trimethyl-5-phenyl-1H-[1]benzopyrano[3,4-f]quinoline; 2,5-dihydro-9-hydroxy-10-methoxy-2,2,4-trimethyl-5-(3,5-difluorophenyl)-1H-[1]benzopyrano[3,4-f]-quinoline; 2,5-dihydro-9-hydroxy-10-methoxy-2,2,4-trimethyl-5-(3,4,5-trifluoro-phenyl)-1H-[1]benzopyrano[3,4-f]quinoline; 5-butyl-2,5-dihydro-9-hydroxy-10-methoxy-2,2,4-trimethyl-1H-[1]benzopyrano[3,4-f]quinoline; (−)(5S,3′S)-2,5-dihydro-9-hydroxy-10-methoxy-2,2,4-trimethyl-5-(3-cyclopentenyl)-1H-[1]benzopyrano[3,4-f]-quinoline; (−)(5S,3′R)-2,5-dihydro-9-hydroxy-10-methoxy-2,2,4-trimethyl-5-(3-cyclopentenyl)-1H-[1]benzopyrano[3,4-f]quinoline; 2,5-dihydro-9-hydroxy-10-methoxy-2,2,4-trimethyl-5-(3,4-difluorophenyl)-1H-[1]benzopyrano[3,4-f]-quinoline; 2,5-dihydro-9-hydroxy-10-methoxy-2,2,4-trimethyl-5-(4-fluorophenyl)-1H-[1]benzo-pyrano[3,4-f]-quinoline; 2,5-dihydro-9-hydroxy-10-methoxy-2,2,4-trimethyl-5-(3-trifluoromethylphenyl)-1H-[1]benzopyrano[3,4-f]quinoline; 2,5-dihydro-9-hydroxy-10-methoxy-2,2,4-trimethyl-5-(3-5-bistrifluoromethylphenyl)-1H-[1]benzopyrano[3,4-f]-quinoline; 2,5-dihydro-9-hydroxy-10-methoxy-2,2,4-trimethyl-5-(3-trifluoromethyl-4-chlorophenyl)-1H-[1]benzopyrano[3,4-f]quinoline; 2,5-dihydro-9-hydroxy-10-methoxy-2,2,4-trimethyl-5-(2-methylpropyl)-1H-[1]benzopyrano[3,4-f]-quinoline; 2,5-dihydro-9-hydroxy-10-methoxy-2,2,4-trimethyl-5-(3-fluoro-4-chlorophenyl)-1H-[1]benzopyrano-[3,4-f]quinoline; 2,5-dihydro-9-hydroxy-10-methoxy-2,2,4-trimethyl-5-(3-butenyl)-1H-[1]benzopyrano[3,4-f]-quinoline; 2,5-dihydro-9-hydroxy-10-methoxy-5-(phenylmethyl)-2,2,4-trimethyl-1H-[1]benzopyrano[3,4-f]-quinoline; (−)(5S,3′R)-2,5-dihydro-9-hydroxy-10-methoxy-2,2,4-trimethyl-5-[1-ethyl-3-cyclohexenyl]-1H-[1]benzo-pyrano[3,4-f]quinoline; (−)(S)-5-cyclopentyl-2,5-dihydro-9-hydroxy-10-methoxy-2,2,4-trimethyl-1-[1]benzopyrano[3,4-f]-quinoline; (+)(R)-5-cyclopentyl-2,5-dihydro-9-hydroxy-10-methoxy-2,2,4-trimethyl-1H-[1]benzopyrano[3,4-f]-quinoline; 2,5-dihydro-9-hydroxy-10-methoxy-5-(3-propynyl)-2,2,4-trimethyl-1H-[1]benzopyrano[3,4-f]-quinoline; 2,5-dihydro-9-hydroxy-10-methoxy-2,2,4-trimethyl-5-(2-propyl)-1H-[1]benzopyrano[3,4-f]-quinoline; 2,5-dihydro-9-hydroxy-10-methoxy-2,2,4-trimethyl-5-(5-methoxy-2-thienyl)-1H-[1]benzopyrano[3,4-f]quinoline; (±)2,5-dihydro-9-hydroxy-10-methoxy-2,2,4-trimethyl-5-(2,3,4,5,6-pentafluorophenyl)-1H-[1]benzopyrano[3,4-f]-quinoline; (±)2,5-dihydro-9-hydroxy-10-methoxy-2,2,4-trimethyl-5(S)-(3(S)-1-hydroxymethylcyclopenten-3-yl)-1H-[1]benzopyrano[3,4-f]quinoline; (±)2,5-dihydro-9-hydroxy-10-methoxy-2,2,4-trimethyl-5(S)-(3(S)-1-methylcarboxylatecyclopenten-3-yl-1H-[1]benzopyrano[3,4-f]quinoline; (−)(5S,3′S) 2,5-dihydro-9-hydroxy-10-methoxy-2,2,4-trimethyl-5-(3-cyclohexenyl)-1H-[1]benzo-pyrano[3,4-f]quinoline; (−)(5S,3′R) 2,5-dihydro-9-hydroxy-10-methoxy-2,2,4-trimethyl-5-(3-cyclohexenyl)-1H-[1]benzo-pyrano[3,4-f]quinoline; 2,5-dihydro-9-hydroxy-10-methoxy-2,2,4-trimethyl-5-(2-thienyl)-1H-[1]benzopyrano[3,4-f]-quinoline; (+)2,5-dihydro-9-hydroxy-10-methoxy-2,2,4-trimethyl-5-(2-methylphenyl)-1H-[1]benzopyrano[3,4-f]-quinoline; 2,5-dihydro-9-hydroxy-10-methoxy-2,2,4-trimethyl-5-(2-acetoxymethyl-3propenyl)-1H-[1]benzo-pyrano[3,4-f]quinoline; (+)(5R,3′S)2,5-dihydro-9-hydroxy-10-methoxy-2,2,4-trimethyl-5-[1-ethyl-3-cyclohexenyl]-1H-[1]benzopyrano[3,4-f]quinoline; 2,5-dihydro-9-hydroxy-10-methoxy-2,2,4-trimethyl-5-cyclohexyl-1H-[1]benzopyrano[3,4-f]-quinoline; 2,5,5-trihydro-9-hydroxy-10-methoxy-2,2,4-trimethyl-1H-[1]benzopyrano[3,4-f]quinoline; 2,5-dihydro-9-hydroxy-10-methoxy-2,2,4-trimethyl-5-(2-hydroxymethyl-3-propenyl)-1H-[1]benzopyrano[3,4-f]quinoline; Methyl-2-[2,5-dihydro-9-hydroxy-10-methoxy-2,2,4-trimethyl-1H-[1]benzopyrano[3,4-f]-5-quinolinyl]acetate; (Z)-2,5-dihydro-9-hydroxy-10-methoxy-2,2,4-trimethyl-5-(2-butenyl)-1H-[1]benzopyrano[3,4-f]-quinoline; 2,5-dihydro-9-hydroxy-10-methoxy-2,2,4-trimethyl-5-(3-methyl-2-butenyl)-1H-[1]benzopyrano[3,4-f]quinoline; (+)(5S,3′S)-2,5-dihydro-9-hydroxy-10-methoxy-2,2,4-trimethyl-5-(3-cyclohexenyl)-1H-[1]-benzopyrano[3,4-f]quinoline; (+)(5R,3′R) 2,5-dihydro-9-hydroxy-10-methoxy-2,2,4-trimethyl-5-(3-cyclohexenyl)-1H-[1]benzopyrano[3,4-f]quinoline; (+)(5R,3′S)-2,5(R)-dihydro-9-hydroxy-10-methoxy-2,2,4-trimethyl-5-(3-cyclopentenyl)-1H-[1]benzopyrano[3,4-f]quinoline; (+)(5R,3′R)-2,5(R)-dihydro-9-hydroxy-10-methoxy-2,2,4-trimethyl-5-(3-cyclopentenyl)-1H-[1]benzo-pyrano[3,4-f]quinoline; rel-(5S)-9-hydroxy-5-[(3R)-(1-methoxycarbonyl)cyclohexen-3-yl]-10-methoxy-2,2,4-trimethyl-1H-[1]benzopyrano[3,4-f]quinoline; 2,5-dihydro-9-hydroxy-10-methoxy-2,2,4-trimethyl-5-(2-methyl-3-propenyl)-1H-[1]benzo-pyrano-[3,4-f]quinoline; 9,10-Dimethoxy-5-(3-propenyl)-2,2,4-trimethyl-1H-2,5-dihydro-[1]benzopyrano[3,4-f]quinoline; 9,10-Dimethoxy-5-[3-cyclohexenyl]-methoxy-2,2,4-trimethyl-2,5-dihydro-1H-[1]benzopyrano[3,4-f]quinoline; 10-methoxy-9-ethoxy-5-(3-propenyl)-2,2,4-trimethyl-1H-2,5-dihydro-[1]benzopyrano[3,4-f]-quinoline; 10-methoxy-9-(3-propenyloxy)-5-(3-propenyl)-2,2,4-trimethyl-1H-2,5-dihydro-[1]benzo-pyrano[3,4-f]-quinoline; 10-methoxy-9-(3-propynyloxy)-5-(3-propenyl)-2,2,4-trimethyl-1H-2,5-dihydro-[1]benzo-pyrano[3,4-f]-quinoline; 2,5-dihydro-9-acetoxy-10-methoxy-2,2,4-trimethyl-5-(2-propenyl)-1H-[1]benzopyrano[3,4-f]-quinoline; 2,5-dihydro-9-(4-N,N-dimethylamino-4-oxo-butanoyloxy)-10-methoxy-2,2,4-trimethyl-5-(2-propenyl)-1H-[1]benzopyrano[3,4-f]quinoline; 7-bromo-5-[3-cyclohexenyl]-10-methoxy-2,2,4-trimethyl-2,5-dihydro-1H-[1]benzopyrano[3,4-f]-quinoline; 10-methoxy-7-bromo-5-(3-propenyl)-2,2,4-trimethyl-1H-2,5-dihydro-[1]benzopyrano[3,4-f]quinoline; 7-bromo-5-[1-methyl-3-cyclohexenyl]-10-methoxy-2,2,4-trimethyl-2,5-dihydro-1H-[1]benzo-pyrano[3,4-f]quinoline; 10-methoxy-9-bromo-5-(3-propenyl)-2,2,4-trimethyl-1H-2,5-dihydro-[1]benzopyrano[3,4-f]-quinoline; 7,9-Dibromo-10-methoxy-5-(3-propenyl)-2,2,4-trimethyl-1H-2,5-dihydro-[1]benzopyrano[3,4-f]-quinoline; 7,9-Dibromo-5-[cyclohexen-3-yl]-10-methoxy-2,2,4-trimethyl-2,5-dihydro-1H-[1]benzopyrano[3,4-f]-quinoline; 7,9-Dibromo-5-[1-methyl-3-cyclohexenyl]-10-methoxy-2,2,4-trimethyl-2,5-dihydro-1H-[1]benzopyrano[3,4-f]quinoline; 10-methoxy-7-(2-ethenyl)-5-(3-propenyl)-2,2,4-trimethyl-1H-2,5-dihydro-[1]benzopyrano[3,4-f]-quinoline; 10-methoxy-7-methyl-5-(3-propenyl)-2,2,4-trimethyl-1H-2,5-dihydro-[1]benzopyrano[3,4-f]-quinoline; 10-methoxy-7-acetyl-5-(3-propenyl)-2,2,4-trimethyl-1H-2,5-dihydro-[1]benzopyrano-[3,4-f]-quinoline; (±)2,5-dihydro-9-methyl-10-methoxy-2,2,4-trimethyl-5-(1-methyl-cyclohexen-3-yl)-1H-[1]benzopyrano[3,4-f]-quinoline; 10-methoxy-7-methyl-9-methyl-5-(3-propenyl)-2,2,4-trimethyl-1H-2,5-dihydro-[1]benzo-pyrano[3,4-f]quinoline; 2,5-dihydro-10-methoxy-5-(3-(N-methyl-N-(carbomethoxymethyl)aminocarbanyloxy)-phenyl)-2,2,4-trimethyl-1H-[1]benzopyrano[3,4-f]quinoline; 2,5-dihydro-10-methoxy-5-(3-(N-methyl-N—(N-methylcarbonyl)aminocarbonyloxy)phenyl)-2,2,4-trimethyl-1H-[1]benzopyrano[3,4-f]quinoline; 2,5-dihydro-10-methoxy)-5-(3-(N-methylamino-carbonyloxy)phenyl)-2,2,4-trimethyl-1H-[1]benzopyrano[3,4-f]quinoline; 2,5-dihydro-10-methoxy-5-(3-(2-hydroxyethyl)phenyl)-2,2,4-trimethyl-1H-[1]benzopyrano[3,4-f]-quinoline; 2,5-dihydro-10-methoxy-5-(3-(2-methanesulfonyloxyethyl)phenyl)-2,2,4-trimethyl-1H-[1]benzopyrano[3,4-f]quinoline; 2,5-dihydro-10-methoxy-5-(3-(2-methylhioethyl)phenyl)-2,2,4-trimethyl-1H-[1]benzopyrano[3,4-f]-quinoline; 2,5-dihydro-10-methoxy-5-(3-(2-(N,N-dimethylaminocarbonyloxy)ethyl)phenyl)-2,2,4-trimethyl-1H-[1]benzopyrano[3,4-f]quinoline; 2,5-dihydro-10-methoxy-5-(3-(2-(N,N-dimethylamino)ethyl)phenyl)-2,2,4-trimethyl-1H-[1]benzopyrano[3,4-f]quinoline; 2,5-dihydro-10-methoxy-5-cyclopropyl-2,2,4-trimethyl-1H-[1]benzopyrano[3,4-f]-quinoline; 2,5-dihydro-10-methoxy-5-ethenyl-2,2,4-trimethyl-1H-[1]benzopyrano-[3,4-f]quinoline; trans-2,5-dihydro-10-methoxy-5-(2-phenylethenyl)-2,2,4-trimethyl-1H-[1]benzopyrano[3,4-f]-quinoline; 2,5-dihydro-10-methoxy-5-(2-phenylethynyl)-2,2,4-trimethyl-1H-[1]benzopyrano[3,4-f]quinoline; cis 2,5-dihydro-10-methoxy-5-(2-phenylethenyl)-2,2,4-trimethyl-1H-[1]benzopyrano[3,4-f]-quinoline; 2,5-dihydro-10-methoxy-5-(2-methylpropenyl)-2,2,4-trimethyl-1H-[1]benzopyrano[3,4-f]quinoline; trans-2,5-dihydro-10-methoxy-5-(1-cyclohexenyl)-2,2,4-trimethyl-1H-[1]benzo-pyrano[3,4-f]-quinoline; 2,5-dihydro-5-(3-propenyl)-10-methylthio-2,2,4-trimethyl-1H-[1]benzopyrano[3,4-f]quinoline; (±)-2,5-dihydro-9-(4-acetamidobutanoyloxy)-10-methoxy-2,2,4-trimethyl-5-allyl-1H-[1]benzo-pyrano[3,4-f]quinoline; 10-(difluoromethoxy)-2,5-dihydro-5-phenyl-2,2,4-trimethyl-1H-[1]benzopyrano[3,4-f]-quinoline; 10-(bromodifluoromethoxy)-2,5-dihydro-5-phenyl-2,2,4-trimethyl-1H-[1]benzopyrano[3,4-f]-quinoline; 2,5-dihydro-9-hydroxy-10-methoxy-2,2,4-trimethyl-5-((2-fluorophenyl)methyl)-1H-[1]benzopyrano[3,4-f]quinoline; 10-methoxy-5-(5-methylisoxazol-3-yl)methylidene-2,5-dihydro-5-phenyl-2,2,4-trimethyl-1H-[1]benzo-pyrano[3,4-f]quinoline; 10-methoxy-5-(3-methylisoxazol-5-yl)methylidene-2,5-dihydro-5-phenyl-2,2,4-trimethyl-1H-[1]benzopyrano[3,4-f]quinoline; 10-methoxy-5-(4,5-dimethyl-1,3-oxazol-2-yl)-methylidene-2,5-dihydro-5-phenyl-2,2,4-trimethyl-1H-[1]benzopyrano[3,4-f]quinoline; 10-methoxy-5-(6-chloropyridin-2-methylidene-2,5-dihydro-5-phenyl-2,2,4-trimethyl-1H-[1]benzopyrano[3,4-f]quinoline; 10-methoxy-5-(pyridin-2-yl)methylidene-2,5-dihydro-5-phenyl-2,2,4-trimethyl-1H-[1-f]quinoline; 10-methoxy-5-(but-3-enylidene)-2,5-dihydro-5-phenyl-2,2,4-trimethyl-1H-[1-f]quinoline; 10-methoxy-5-(1-methylpropylidene)-2,5-dihydro-5-phenyl-2,2,4-trimethyl-1H-[1-f]-quinoline; 10-methoxy-5-(1-butylidene)-2,5-dihydro-5-phenyl-2,2,4-trimethyl-1H-[1-f]-quinoline; Z-5-(benzylidenyl)-9-hydroxy-10-methoxy-2,2,4-trimethyl-1H-2,5-dihydro-[1]benzopyrano[3,4-f]-quinoline; Z-5-(2,5-difluorobenzylidenyl)-9-hydroxy-10-methoxy-2,2,4-trimethyl-1H-2,5-dihydro-[1]benzo-pyrano[3,4-f]quinoline; Z-9-hydroxy-10-methoxy-5-(2-picolinylidenyl)-2,2,4-trimethyl-2,5-dihydro-1H-[1]benzo-pyrano[3,4-f]quinoline; 9-hydroxy-10-methoxy-5-(3,5-difluorophenyl)methylidene-2,5-dihydro-5-phenyl-2,2,4-trimethyl-1H-[1]benzopyrano[3,4-f]quinoline; 9-hydroxy-10-methoxy-5-(3,4-difluorophenyl)methylidene-2,5-dihydro-5-phenyl-2,2,4-trimethyl-1H-[1]benzopyrano[3,4-f]quinoline; (Z) 9-hydroxy-10-methoxy-5-((4-fluorophenyl)-methylene)-2,2,4-trimethyl-1H-2,5-dihydro-[1]benzopyrano[3,4-f]quinoline; (Z)-9-hydroxy-10-methoxy-5-([2,3-difluorophenyl]methylene)-2,2,4-trimethyl-1H-2,5-dihydro-[1]benzopyrano[3,4-f]quinoline; Z-5-(3-fluorobenzylidenyl)-10-methoxy-9-hydroxy-2,2,4-trimethyl-2,5-dihydro-1H-[1]benzo-pyrano[3,4-f]quinoline; 9-hydroxy-10-methoxy-5-ethyl-2,2,4-trimethyl-2,5-dihydro-1H-[1]benzopyrano[3,4-f]quinoline; (±)-2,5-dihydro-9-cyanomethoxy-10-methoxy-2,2,4-trimethyl-5-allyl-1H-[1]benzo-pyrano[3,4-f]-quinoline; 2,5-dihydro-9-(4-N,N-diethylamino-4-oxo-butanoyloxy)-10-methoxy-2,2,4-trimethyl-5-(2-propenyl)-1H-[1]benzopyrano[3,4-f]-quinoline; 2,5-dihydro-9-(4-N-piperidino-4-oxo-butanoyloxy)-10-methoxy-2,2,4-trimethyl-5(2-propenyl)-1H-[1]benzopyrano[3,4-f]quinoline; 2,5-dihydro-9-(4-N-morpholino4-oxo-butanoyloxy)-10-methoxy-2,2,4-trimethyl-5-(2-propenyl)-1H-[1]benzopyrano[3,4-f]-quinoline; 2,5-dihydro-9-(4-N,N-dimethylamino-4-oxo-butanoyloxy)-10-methoxy-2,2,4-trimethyl-5-(3,4,5-trifluorophenyl)-1H-[1]benzopyrano[3,4-f]quinoline; 2,5-dihydro-9-hydroxy-10-methoxy-2,2,4-trimethyl-5-(3-5-difluorophenylmethyl)-1H-[1]benzopyrano[3,4-f]quinoline; 2,5-dihydro-9-hydroxy-10-methoxy-2,2,4-trimethyl-5-cyclopentyl-1H-[1]benzopyrano[3,4-f]-quinoline; 2,5-dihydro-9-hydroxy-10-methoxy-2,2,4-trimethyl-5-((2-fluorophenyl)methyl)-1H-[1]benzopyrano[3,4-f]quinoline; 2,5-dihydro-9-hydroxymethyl-10-methoxy-2,2,4-trimethyl-5-allyl-1H-[1]benzopyrano-[3,4-f]quinoline; 2,5-dihydro-9-hydroxy-10-methoxy-2,2,4-trimethyl-5-(1-pentenyl)-1H-[1]benzopyrano[3,4-f]-quinoline; 2,5-dihydro-9-methylcarboxylate-10-methoxy-2,2,4-trimethyl-5-allyl-1H-[1]benzopyrano[3,4-f]-quinoline; 2,5-dihydro-9-hydroxy-10-methoxy-2,2,4-trimethyl-5-allenyl-1H-[1]benzopyrano[3,4-f]quinoline; (−)(5S,3′S)-2,5-dihydro-10-methoxy-2,2,4-trimethyl-5-(cyclopenten-3-yl)-1H-[1]benzopyrano[3,4-f]-quinoline; (−)(5S,3′S)-2,5-dihydro-10-methoxy-2,2,4-trimethyl-5-(cyclohexen-3-yl)-1H-[1]benzopyrano[3,4-f]-quinoline; (−)(5S,3′R)-2,5-dihydro-10-methoxy-2,2,4-trimethyl-5-(cyclohexen-3-yl)-1H-[1]benzopyrano[3,4-f]-quinoline; (−)(5S,3′R)-2,5-dihydro-10-methoxy-2,2,4-trimethyl-5-(cyclopenten-3-yl)-1H-[1]benzopyrano[3,4-f]quinoline; 2,5-dihydro-9-hydroxy-10-methoxy-2,2,4-trimethyl-5-(3(Z)-pentenyl)-1H-[1]benzo-pyrano[3,4-f]-quinoline; 2,5-dihydro-9-hydroxy-10-methoxy-2,2,4-trimethyl-5-(3-acetoxyphenyl)-1H-[1]benzopyrano[3,4-f]-quinoline; 10-difluoromethoxy-5-[[3-(methylthio)methoxy]phenyl]-2,2,4-trimethyl-1H-2,5-dihydro-[1]benzopyrano[3,4-f]-quinoline; 2,5-dihydro-9-hydroxy-10-methoxy-2,2,4-trimethyl-5-(3-hydroxyphenyl)-1H-[1]benzopyrano[3,4-f]-quinoline; 2,5-dihydro-9-methylthiomethoxy-10-methoxy-2,2,4-trimethyl-5-(3(methylthio)methoxyphenyl)-1H-[1]benzopyrano[3,4-f]-quinoline; 2,5-dihydro-9-hydroxy-10-methoxy-2,2,4-trimethyl-5-(3(methylthiomethoxy)phenyl)-1H-[1]benzopyrano[3,4-f]quinoline; 2,5-dihydro-9-N,N-dimethylcarbamoyloxy-10-methoxy-2,2,4-trimethyl-5-([2-N,N-dimethyl-carbamoyloxy]phenyl)-1H-[1]benzo-pyrano[3,4-f]quinoline; 9-hydroxy-10-methoxy-5-(phenylmethylene)-2,2,4-trimethyl-1H-2,5-dihydro-[1]benzopyrano[3,4-f]-quinoline; 9-hydroxy-10-methoxy-5-([3-fluorophenyl]methylene)-2,2,4-trimethyl-1H-2,5-dihydro-[1]benzopyrano[3,4-f]-quinoline; rel-(5S)-9-hydroxy-5-[(3S)-(1-methoxycarbonyl)cyclohexen-3-yl]-10-methoxy-2,2,4-trimethyl-2,5-dihydro-1H-[1]benzopyrano[3,4-f]quinoline; 2,5-dihydro-9-hydroxy-10-methoxy-2,2,4-trimethyl-5-(3,5-dichlorophenyl)-1H-[1]benzopyrano-[3,4-f]quinoline; 2,5-dihydro-9-(4-N,N-dimethylamino-4-oxo-butanoyloxy)-10-methoxy-2,2,4-trimethyl-5-(1-methylethyl)-1H-[1]benzopyrano[3,4-f]quinoline; 2,5-dihydro-9-(4-N,N-dimethylamino-4-oxo-butanoyloxy) 10-methoxy-5-(phenylmethyl)-2,2,4-trimethyl-1H-[1]benzopyrano[3,4-f]quinoline; 2,5-dihydro-9-(4-N,N-dimethyl-amino-4-oxo-butanoyloxy)-10-methoxy-2,2,4-trimethyl-5-(2-thienyl)-1H-[1]benzo-pyrano[3,4-f]quinoline; 2,5-dihydro-9-(4-N,N-dimethylaminobutanoyloxy)-10-methoxy-2,2,4-trimethyl-5-(2-propenyl)-1H-[1]benzopyrano[3,4-f]quinoline; 9-(2-ethoxy-2-oxo-ethylaminocarbonyl)-oxy-10-methoxy-5-(3-propenyl)-2,2,4-trimethyl-1H-2,5-dihydro-[1]benzopyrano[3,4-f]quinoline; (±)2,5-dihydro-9-(3-acetamido-propanoyloxy)-10-methoxy-2,2,4-trimethyl-5-allyl-1H-[1]benzopyrano[3,4-f]quinoline; 9-hydroxy-10-methoxy-5-(phenylmethylene)-2,2,4-trimethyl-1H-2,5-dihydro-[1]benzo-pyrano[3,4-f]-quinoline; 9-(dimethylaminothiocarbonyl)-oxy-10-methoxy-5-(3-propenyl)-2,2,4-trimethyl-1H-2,5-dihydro-[1]benzopyrano[3,4-f]quinoline; (±)2,5-dihydro-9-(N-carbamoyl-2-aminoacetoxy)-10-methoxy-2,2,4-trimethyl-5-allyl-1H-[1]benzopyrano[3,4-f]quinoline; (±)2,5-dihydro-9-(4-ethoxy-4-oxo-butoxy)-10-methoxy-2,2,4-trimethyl-5-allyl-1H-[1]benzopyrano-[3,4-f]-quinoline; (±)2,5-dihydro-9-(4-oxo-pentanoyloxy)-10-methoxy-2,2,4-trimethyl-5-allyl-1H-[1]benzopyrano-[3,4-f]-quinoline; 2,5-dihydro-9-methylthiomethoxy-10-methoxy-2,2,4-trimethyl-5-allyl-1H-[1]benzopyrano[3,4-f]-quinoline; 2,5-dihydro-9-(4-N,N-diethylamino-4-oxo-pentanoyloxy)-10-methoxy-2,2,4-trimethyl-5-(2-propenyl)-1H-[1]benzopyrano[3,4-f]-quinoline; 2,5-dihydro-9-(4-N,N-dimethylamino-4-oxo-pentanoyloxy)-10-methoxy-2,2,4-trimethyl-5-(2-propenyl)-1H-[1]benzopyrano[3,4-f]quinoline; 2,5-dihydro-9-(4-N-piperidino-4-oxo-pentanoyloxy)-10-methoxy-2,2,4-trimethyl-5-(2-propenyl)-1H-[1]benzopyrano[3,4-f]quinoline; 2,5-dihydro-9-(4-N-morpholino-4-oxo-pentanoyloxy)-10-methoxy-2,2,4-trimethyl-5-(2-propenyl)-1H-[1]benzopyrano[3,4-f]quinoline; (−)2,5-dihydro-9-(4-N,N-dimethylamino-4-oxo-butanoyloxy)-10-methoxy-2,2,4-trimethyl-5(S)-(3(S)-1-cyclopenten-3-yl)-1H-[1]benzopyrano[3,4-f]quinoline; 10-methoxy-9-(allylaminocarbonyl)oxy-5-(3-propenyl)-2,2,4-trimethyl-1H-2,5-dihydro-[1]benzo-pyrano[3,4-f]quinoline; 10-methoxy-9-(cyclohexylaminocarbonyl)-oxy-5-(3-propenyl)-2,2,4-trimethyl-1H-2,5-dihydro-[1]benzopyrano[3,4-f]quinoline; 2,5-dihydro-9-hydroxy-10-methoxy-2,2,4-trimethyl-5-(3-thienyl)-1H-[1]benzopyrano-[3,4-f]-quinoline; 2,5-dihydro-9-hydroxy-10-methoxy-2,2,4-trimethyl-5-(4-(fluoro-phenyl)methyl)-1H-[1]benzo-pyrano[3,4-f]quinoline; 10-benzyloxy-5-(2-propenyl)-2,5-dihydro-2,2,4-trimethyl-1H-[1]benzopyrano[3,4-f]quinoline; rel-(5S,3′R)-9-hydroxy-10-methoxy-5-{1-hydroxymethyl-cyclohexyl}-2,2,4-trimethyl-2,5-dihydro-1H-[1]benzo-pyrano[3,4-f]quinoline; 2,5-dihydro-9-hydroxy-10-methoxy-2,2,4-trimethyl-5-(1-thiazol-2-yl)-1H-[1]benzopyrano[3,4-f]-quinoline; 1,2-Dihydro-2,2,4-trimethyl-6-methoxymethyl-8-pyranono[5,6-g]quinoline; 1,2-Dihydro-2,2,4-trimethyl-6-trifluoro-methyl-8-pyranono[5,6-g]quinoline; 1,2-Dihydro-2,2,4-trimethyl-10-isocoumarino-[4,3-g]quinoline; 1,2-Dihydro-2,2,4-trimethyl-10-isoquinolono[4,3-g]quinoline; 1,2-Dihydro-2,2,4,6-tetramethyl-8-pyridono[5,6-g]quinoline; 1,2-Dihydro-10-hydroxy-2,2,4-trimethyl-10H-isochromeno[4,3-g]quinoline; 1,2-Dihydro-2,2,4,6-tetramethyl-8H-pyrano[3,2-g]quinoline; (R/S)-1,2,3,4-Tetrahydro-2,2,4-trimethyl-10-isoquinolono-[4,3-g]quinoline; 1,2-Dihydro-2,2,4-trimethyl-10-thioisoquinolono[4,3-g]quinoline; (+)-1,2,3,4-Tetrahydro-2,2,4-trimethyl-10-isoquinolono[4,3-g]quinoline; 1,2-Dihydro-2,2,4-trimethyl-6-trifluoromethyl-8-pyridono[5,6-f]quinoline; (R/S)-1,2,3,4-Tetrahydro-2,2,4-trimethyl-6-trifluoromethyl-8-pyranono[5,6-g]quinoline; 1,2-Dihydro-2,2,4-trimethyl-6-trifluoromethyl-8-thiopyranono[5,6-g]quinoline; (R/S)-1,2,3,4-Tetrahydro-2,2,4-trimethyl-6-trifluoromethyl-8-thiopyranono[5,6-g]quinoline; 6-Chloro(difluoro)-methyl-1,2-dihydro-2,2,4-trimethyl-8-pyranono[5,6-g]quinoline; 9-Acetyl-1,2-dihydro-2,2,4-trimethyl-6-trifluoromethyl-8-pyridono[5,6-g]quinoline; 1,2-Dihydro-2,2,4,10-tetramethyl-6-trifluoromethyl-8-pyridono[5,6-g]quinoline; 1,2-Dihydro-2,2,4-trimethyl-6-(1,1,2,2,2-pentafluoroethyl)-8-pyranono[5,6-g]quinoline; (R/S)-6-Chloro(difluoro)-methyl-1,2,3,4-tetrahydro-2,2,4-trimethyl-8-pyranono[5,6-g]quinoline; (Compound 257); 7-Chloro-1,2-dihydro-2,2,4-trimethyl-6-trifluoromethyl-8-pyranono[5,6-g]-quinoline; (R/S)-7-Chloro-1,2,3,4-tetrahydro-2,2,4-trimethyl-6-trifluoromethyl-8-pyranono[5,6-g]quinoline; 1,2,3,4-Tetrahydro-2,2,4-trimethyl-6-trifluoromethyl-8-pyridono[5,6-g]quinoline; 1,2-Dihydro-2,2,4,9-tetramethyl-6-trifluoromethyl-8-pyridono[5,6-g]quinoline; 1,2-Dihydro-2,2,4-trimethyl-8-trifluoromethyl-6-pyridono[5,6-g]quinoline; 6-[Dichloro(ethoxy)methyl]-1,2-dihydro-2,2,4-trimethyl-8-pyranono[5,6-g]quinoline; 5-(3-Furyl)-1,2-dihydro-2,2,4-trimethyl-8-pyranono[5,6-g]-quinoline; 1,2-Dihydro-1,2,2,4-tetramethyl-6-trifluoromethyl-8-pyranono[5,6-g]-quinoline; 1,2-Dihydro-6-trifluoromethyl-2,2,4-trimethyl-9-thiopyran-8-ono[5,6-g]-quinoline; 1,2-Dihydro-1,2,2,4,9-pentamethyl-6-trifluoromethyl-8-pyridono[5,6-g]-quinoline; 7-Chloro-1,2-dihydro-2,2,4-trimethyl-6-trifluoromethyl-8-pyridono[5,6-g]-quinoline; 6-Chloro(difluoro)methyl-1,2-dihydro-2,2,4-trimethyl-8-pyridono[5,6-g]-quinoline; (R/S)-1,2,3,4-Tetrahydro-1,2,2,4-tetramethyl-6-trifluoromethyl-8-pyranono[5,6-g]quinoline; (R/S)-5-(3-Furyl)-1,2,3,4-tetrahydro-2,2,4-trimethyl-8-pyranono[5,6-g]quinoline; 5-(3-Furyl)-1,2-dihydro-1,2,2,4-tetramethyl-8-pyranono[5,6-g]quinoline; 5-(3-Furyl)-1,2-dihydro-1,2,2,4-tetramethyl-8-thiopyranono[5,6-g]-quinoline; 6-Chloro-5-(3-furyl)-1,2-dihydro-1,2,2,4-tetramethyl-8-pyranono[5,6-g]-quinoline; 1,2,3,4-Tetrahydro-2,2,4,10-tetramethyl-6-trifluoromethyl-8-pyridono[5,6-g]-quinoline; (R/S)-1,2,3,4-Tetrahydro-4-methyl-6-trifluoromethyl-8-pyranono[5,6-g]-quinoline; 1,2-Dihydro-2,2-dimethyl-6-trifluoromethyl-8-pyranono[5,6-g]quinoline; 1,2,3,4-Tetrahydro-2,2-dimethyl-6-trifluoromethyl-8-pyranono[5,6-g]quinoline; 1,2,3,4-Tetrahydro-6-trifluoromethyl-8-pyranono[5,6-g]quinoline; (R/S)-4-Ethyl-1,2,3,4-tetrahydro-6-trifluoromethyl-8-pyranono[5,6-g]quinoline; (R/S)-1,2,3,4-Tetrahydro-1,4-dimethyl-8-pyranono[5,6-g]quinoline; (R/S)-4-Ethyl-1,2,3,4-tetrahydro-1-methyl-8-pyranono[5,6-g]quinoline; 2,2-Dimethyl-1,2,3,4-tetrahydro-6-trifluoromethyl-8-pyridono[5,6-f]quinoline; (R/S)-1,2,3,4-tetrahydro-6-trifluoromethyl-2,2,4-trimethyl-8-pyridono[5,6-f]-3-quinolinone; 5-Trifluoromethyl-7-pyridono[5,6-e]indoline; 8-(4-Chlorobenzoyl)-5-trifluoromethyl-7-pyridono[5,6-e]indoline; 7-tert-Butyloxy-carbamoyl-1,2-dihydro-2,2,8-trimethylquinoline; 1,2,3,4-Tetrahydro-6-trifluoromethyl-8-pyridono[5,6-f]quinoline; 1,2-Dihydro-6-trifluoromethyl-1,2,2,4-tetramethyl-8-pyridono[5,6-f]quinoline; 3,3-Dimethyl-5-trifluoromethyl-7-pyridono[5,6-e]indoline; (R/S)-1,2,3,4-Tetrahydro-4-methyl-6-(trifluoromethyl)-8-pyridono[5,6-g]quinoline; (R/S)-1,2,3,4-Tetrahydro-4-methyl-6-(trifluoromethyl)-8-pyridono[5,6-g]quinoline; 1,2,2-Trimethyl-1,2,3,4-tetrahydro-6-trifluoromethyl-8-pyranono[5,6-g]quinoline; (R/S)-1,2,3,4-Tetrahydro-4-propyl-6-trifluoromethyl-8-pyranono[5,6-g]quinoline; 1,2,3,4-Tetrahydro-2,2,4-trimethyl-6-trifluoromethyl-9-thiopyran-8-ono[5,6-g]-quinoline; 1,2-Dihydro-1,2,2,4-tetramethyl-6-trifluoromethyl-9-thiopyran-8-ono[5,6-g]-quinoline; 1,2,3,4-Tetrahydro-1,2,2-trimethyl-6-trifluoromethyl-8-pyridono[5,6-g]-quinoline; 1,2,3,4-Tetrahydro-1-methyl-4-propyl-6-trifluoromethyl-8-pyranono[5,6-g]-quinoline; 1,2,3,4-Tetrahydro-10-hydroxymethyl-2,2,4-trimethyl-6-trifluoromethyl-8-pyridono[5,6-g]-quinoline; 1,2,3,4-Tetrahydro-1,2,2,4-tetramethyl-6-trifluoromethyl-9-thiopyran-8-ono[5,6-g]quinoline; 1,2,3,4-Tetrahydro-2,2,9-trimethyl-6-trifluoromethyl-8-pyridono[5,6-g]quinoline; (R/S)-1,2,3,4-Tetrahydro-3-methyl-6-trifluoromethyl-8-pyridono[5,6-g]quinoline; 1,2,3,4-Tetrahydro-3,3-dimethyl-6-trifluoromethyl-8-pyridono[5,6-g]quinoline; (R/S) 1,2,3,4-Tetrahydro-2,2,3-trimethyl-6-trifluoromethyl-8-pyridono[5,6-g]quinoline; (R/S-21,4u)-1,2,3,4-Tetrahydro-2,4-dimethyl-6-trifluoromethyl-8-pyridono[5,6-g]quinoline; (R/S-21,4u)-4-Ethyl-1,2,3,4-tetrahydro-2-methyl-6-trifluoromethyl-8-pyranono[5,6-g]quinoline (Compound 440); (R/S-21,3u)-1,2,3,4-Tetrahydro-2,3-dimethyl-6-trifluoromethyl-8-pyridono[5,6-g]-quinoline; (R/S-21,31)-1,2,3,4-Tetrahydro-2,3-dimethyl-6-trifluoromethyl-8-pyridono[5,6-g]-quinoline; (R/S)-1,2,3,4-Tetrahydro-2,3,3-trimethyl-6-trifluoromethyl-8-pyridono[5,6-g]quinoline; (R/S)-1,2,3,4-Tetrahydro-2-methyl-6-trifluoromethyl-8-pyridono[5,6-g]quinoline; (R/S)-4-Ethyl-1,2,3,4-tetrahydro-6-trifluoromethyl-8-pyridono[5,6-g]quinoline; (R/S-21,3u)-1,2,3,4-Tetrahydro-2,3,9-trimethyl-6-trifluoromethyl-8-pyridono[5,6-g]quinoline; (R/S)-1,2,3,4-Tetrahydro-4-propyl-6-trifluoromethyl-8-pyridono[5,6-g]quinoline; (R/S)-3-Ethyl-1,2,3,4-tetrahydro-2,2-dimethyl-6-trifluoromethyl-8-pyridono[5,6-g]quinoline; (R/S)-1,2,3,4-Tetrahydro-2,2-dimethyl-6-trifluoromethyl-3-propyl-8-pyridono[5,6-g]quinoline; 1-Methyl-5-trifluoromethyl-7-pyridono[5,6-f]indoline; 1,2-Dihydro-2,2,4-trimethyl-6-methoxymethyl-8-pyranono[5,6-g]quinoline; 1,2-Dihydro-2,2,4-trimethyl-6-trifluoromethyl-8-pyranono[5,6-g]quinoline; 1,2-Dihydro-2,2,4-trimethyl-10-isocoumarino[4,3-g]quinoline; 1,2-Dihydro-2,2,4-trimethyl-10-isoquinolono[4,3-g]quinoline; 1,2-Dihydro-2,2,4,6-tetramethyl-8-pyridono[5,6-g]quinoline; 1,2-Dihydro-10-hydroxy-2,2,4-trimethyl-10H-isochromeno[4,3-g]quinoline; 1,2-Dihydro-2,2,4,6-tetramethyl-8H-pyrano[3,2-g]-quinoline; (R/S)-1,2,3,4-Tetrahydro-2,2,4-trimethyl-10-isoquinolono[4,3-g]quinoline; 1,2-Dihydro-2,2,4-trimethyl-10-thioisoquinolono[4,3-g]quinoline; (+)-1,2,3,4-Tetrahydro-2,2,4-trimethyl-10-isoquinolono[4,3-g]quinoline; 1,2-Dihydro-2,2,4-trimethyl-6-trifluoromethyl-8-pyridono[5,6-g]quinoline; (R/S)-1,2,3,4-Tetrahydro-2,2,4-trimethyl-6-trifluoromethyl-8-pyranono[5,6-g]quinoline; 1,2-Dihydro-2,2,4-trimethyl-6-trifluoromethyl-8-thiopyranono[5,6-g]quinoline; (R/S)-1,2,3,4-Tetrahydro-2,2,4-trimethyl-6-trifluoromethyl-8-thiopyranono[5,6-g]quinoline; 6-Chloro(difluoro)methyl-1,2-dihydro-2,2,4-trimethyl-8-pyranono[5,6-g]quinoline; 9-Acetyl-1,2-dihydro-2,2,4-trimethyl-6-trifluoromethyl-8-pyridono[5,6-g]quinoline; 1,2-Dihydro-2,2,4,10-tetramethyl-6-trifluoromethyl-8-pyridono[5,6-g]quinoline; 1,2-Dihydro-2,2,4-trimethyl-6-(1,1,2,2,2-pentafluoroethyl)-8-pyranono[5,6-g]quinoline; (R/S)-6-Chloro(difluoro)-methyl-1,2,3,4-tetrahydro-2,2,4-trimethyl-8-pyranono[5,6-g]quinoline; 7-Chloro-1,2-dihydro-2,2,4-trimethyl-6-trifluoromethyl-8-pyranono[5,6-g]quinoline; (R/S)-7-Chloro-1,2,3,4-tetrahydro-2,2,4-trimethyl-6-trifluoromethyl-8-pyranono[5,6-g]quinoline; 1,2,3,4-Tetrahydro-2,2,4-trimethyl-6-trifluoromethyl-8-pyridono[5,6-g]quinoline; 1,2-Dihydro-2,2,4,9-tetramethyl-6-trifluoromethyl-8-pyridono[5,6-g]quinoline; 1,2-Dihydro-2,2,4-trimethyl-g-trifluoromethyl-6-pyridono[5,6-g]quinoline; 6-[Dichloro(ethoxy)methyl]-1,2-dihydro-2,2,4-trimethyl-8-pyranono[5,6-g]quinoline; 5-(3-Furyl)-1,2-dihydro-2,2,4-trimethyl-8-pyranono[5,6-g]quinoline; 1,2-Dihydro-1,2,2,4-tetramethyl-6-trifluoromethyl-8-pyranono[5,6-g]quinoline; 1,2-Dihydro-6-trifluoromethyl-2,2,4-trimethyl-9-thiopyran-8-ono[5,6-g]quinoline; 1,2-Dihydro-1,2,2,4,9-pentamethyl-6-trifluoromethyl-8-pyridono[5,6-g]quinoline; 7-Chloro-1,2-dihydro-2,2,4-trimethyl-6-trifluoromethyl-8-pyridono[5,6-g]quinoline; 6-Chloro(difluoro)methyl-1,2-dihydro-2,2,4-trimethyl-8-pyridono[5,6-g]quinoline; (R/S)-2,3,4-Tetrahydro-2,2,4-tetramethyl-6-trifluoromethyl-8-pyranono[5,6-g]-quinoline; (R/S)-5-(3-Furyl)-1,2,3,4-tetrahydro-2,2,4-trimethyl-8-pyranono[5,6-g]-quinoline; 5-(3-Furyl)-1,2-dihydro-1,2,2,4-tetramethyl-8-pyranono[5,6-g]quinoline; 5-(3-Furyl)-1,2-dihydro-1,2,2,4-tetramethyl-8-thiopyranono[5,6-g]quinoline; 6-Chloro-5-(3-furyl)-1,2-dihydro-1,2,2,4-tetramethyl-8-pyranono[5,6-g]quinoline; 1,2,3,4-Tetrahydro-2,2,4,10-tetramethyl-6-trifluoromethyl-8-pyridono[5,6-g]quinoline; (R/S)-1,2,3,4-Tetrahydro-4-methyl-6-trifluoromethyl-8-pyranono[5,6-g]quinoline; 1,2-Dihydro-2,2-dimethyl-6-trifluoromethyl-8-pyranono[5,6-g]quinoline; 1,2,3,4-Tetrahydro-2,2-dimethyl-6-trifluoromethyl-8-pyranono[5,6-g]quinoline; 1,2,3,4-Tetrahydro-6-trifluoromethyl-8-pyranono[5,6-g]quinoline; (R/S)-4-Ethyl-1,2,3,4-tetrahydro-6-trifluoromethyl-8-pyranono[5,6-g]quinoline; (R/S)-1,2,3,4-Tetrahydro-1,4-dimethyl-8-pyranono[5,6-g]quinoline; (R/S)-4-Ethyl-1,2,3,4-tetrahydro-1-methyl-8-pyranono[5,6-g]quinoline; 2,2-Dimethyl-1,2,3,4-tetrahydro-6-trifluoromethyl-8-pyridono[5,6-f]quinoline; (R/S)-1,2,3,4-tetrahydro-6-trifluoromethyl-2,2,4-trimethyl-8-pyridono[5,6-f]-3-quinolinone; 5-Trifluoromethyl-7-pyridono[5,6-e]indoline; 8-(4-Chlorobenzoyl)-5-trifluoromethyl-7-pyridono[5,6-e]indoline; 7-tert-Butyloxy-carbamoyl-1,2-dihydro-2,2,8-trimethylquinoline; 1,2,3,4-Tetrahydro-6-trifluoromethyl-8-pyridono[5,6-f]quinoline; 1,2-Dihydro-6-trifluoromethyl-1,2,2,4-tetramethyl-8-pyridono[5,6-f]quinoline; 3,3-Dimethyl-5-trifluoromethyl-7-pyridono[5,6-e]indoline; (R/S)-1,2,3,4-Tetrahydro-4-methyl-6-(trifluoromethyl)-8-pyridono[5,6-g]quinoline; (R/S)-1,2,3,4-Tetrahydro-4-methyl-6-(trifluoromethyl)-8-pyridono[5,6-g]quinoline; 1,2,2-Trimethyl-1,2,3,4-tetrahydro-6-trifluoromethyl-8-pyranono[5,6-g]quinoline; (R/S)-1,2,3,4-Tetrahydro-4-propyl-6-trifluoromethyl-8-pyranono[5,6-g]quinoline; 1,2,3,4-Tetrahydro-2,2,4-trimethyl-6-trifluoromethyl-9-thiopyran-8-ono[5,6-g]-quinoline; 1,2-Dihydro-1,2,2,4-tetramethyl-6-trifluoromethyl-9-thiopyran-8-ono[5,6-g]quinoline; 1,2,3,4-Tetrahydro-1,2,2-trimethyl-6-trifluoromethyl-8-pyridono[5,6-g]-quinoline; 1,2,3,4-Tetrahydro-1-methyl-4-propyl-6-trifluoromethyl-8-pyranono[5,6-g]-quinoline; 1,2,3,4-Tetrahydro-10-hydroxymethyl-2,2,4-trimethyl-6-trifluoromethyl-8-pyridono[5,6-g]-quinoline; 1,2,3,4-Tetrahydro-1,2,2,4-tetramethyl-6-trifluoromethyl-9-thiopyran-8-ono[5,6-g]quinoline; 1,2,3,4-Tetrahydro-2,2,9-trimethyl-6-trifluoromethyl-8-pyridono[5,6-g]quinoline; (R/S)-1,2,3,4-Tetrahydro-3-methyl-6-trifluoromethyl-8-pyridono[5,6-g]quinoline; 1,2,3,4-Tetrahydro-3,3-dimethyl-6-trifluoromethyl-8-pyridono[5,6-g]quinoline; (R/S)-1,2,3,4-Tetrahydro-2,2,3-trimethyl-6-trifluoromethyl-8-pyridono[5,6-g]quinoline; (R/S-21,4u)-1,2,3,4-Tetrahydro-2,4-dimethyl-6-trifluoromethyl-8-pyridono[5,6-g]quinoline; (R/S-21,4u)-4-Ethyl-1,2,3,4-tetrahydro-2-methyl-6-trifluoromethyl-8-pyrano[5,6-g]-quinoline; (R/S-21,3u)-1,2,3,4-Tetrahydro-2,3-dimethyl-6-trifluoromethyl-8-pyridono[5,6-g]quinoline; (R/S-21,31)-1,2,3,4-Tetrahydro-2,3-dimethyl-6-trifluoromethyl-8-pyridono[5,6-g]quinoline; (R/S)-1,2,3,4-Tetrahydro-2,3,3-trimethyl-6-trifluoromethyl-8-pyridono[5,6-g]quinoline; (R/S)-1,2,3,4-Tetrahydro-2-methyl-6-trifluoromethyl-8-pyridono[5,6-g]quinoline; (R/S)-4-Ethyl-1,2,3,4-tetrahydro-6-trifluoromethyl-8-pyridono[5,6-g]quinoline; (R/S-21,3u)-1,2,3,4-Tetrahydro-2,3,9-trimethyl-6-trifluoromethyl-8-pyridono[5,6-g]quinoline; (R/S)-1,2,3,4-Tetrahydro-4-propyl-6-trifluoromethyl-8-pyridono[5,6-g]quinoline; (R/S)-3-Ethyl-1,2,3,4-tetrahydro-2,2-dimethyl-6-trifluoromethyl-8-pyridono[5,6-g]quinoline; (R/S)-1,2,3,4-Tetrahydro-2,2-dimethyl-6-trifluoromethyl-3-propyl-8-pyridono[5,6-g]quinoline; 1-Methyl-5-trifluoromethyl-7-pyridono[5,6-f]indoline; 3-chloro-4-cyano-N-(3-ethylthio-2-hydroxy-2-methylpropionyl)aniline; 3-chloro-4-cyano-N-(3-ethylsulphonyl-2-hydroxy-2-methylpropionyl)aniline; 4-cyano-3-trifluoromethyl-N-(2-hydroxy-2-methyl-3-phenylsulphonylpropionyl)aniline; 4-cyano-3-trifluoromethyl-N-(3-ethylsulphonyl-2-hydroxy-2-methylpropionyl)aniline; 4-nitro-3-trifluoromethyl-N-(2-hydroxy-3-phenylsulphonyl-2-methylpropionyl)aniline; 4-nitro-3-trifluoromethyl-N-(3-ethylsulphonyl-2-hydroxy-2-methylpropionyl)aniline; 3-chloro-4-nitro-N-(2-hydroxy-3-phenylthio-2-methylpropionyl)aniline; 4-nitro-3-trifluoromethyl-N-[2-hydroxy-2-methyl-3-(thiazol-2-ylthio)propionyl]aniline; 4-nitro-3-trifluoromethyl-N-[3-allylthio-2-hydroxy-2-methylpropionyl)aniline; 4-nitro-3-trifluoromethyl-N-(3-p-fluorophenylthio-2-hydroxy-2-methylpropionyl)aniline; 4-nitro-3-trifluoromethyl-N-[2-hydroxy-2-methyl-3-(pyrid-2-ylthio)propionyl]aniline; 4-nitro-3-trifluoromethyl-N-[2-hydroxy-2-methyl-3-(5-methyl-1,3,4-thiadiazol-2-ylthio)propionyl]aniline; 4-nitro-3-trifluoromethyl-N-[2-hydroxy-2-methyl-3-(4-methylthiazol-2-ylthio)propionyl]aniline; 4-nitro-3-trifluoromethyl-N-[2-hydroxy-2-methyl-3-(pyrid-2-ylsulphonyl)propionyl]-aniline; 4-nitro-3-trifluoromethyl-N-(3-p-fluorophenylsulphonyl-2-hydroxy-2-methylpropionyl)aniline; 4-cyano-3-trifluoromethyl-N-[2-hydroxy-2-methyl-3-(thiazol-2-ylthio)propionyl]aniline; 4-cyano-3-trifluoromethyl-N-[2-hydroxy-2-methyl-3-(pyrid-2-ylthio)propionyl]aniline; 4-cyano-3-trifluoromethyl-N-(2-hydroxy-2-methyl-3-methylthiopropionyl)aniline; 4-cyano-3-trifluoromethyl-N-(3-p-fluorophenylthio-2-hydroxy-2-methylpropionyl)aniline; and 4-cyano-3-trifluoromethyl-N-(3-p-fluorophenylsulphonyl-2-hydroxy-2-methylpropionyl)aniline.

In certain embodiments, the compounds provided herein are selective androgen receptor modulators. In certain embodiments, the compounds provided herein are selective androgen receptor binding compounds. In certain embodiments, the compounds provided herein are androgen receptor reducing compounds. In certain embodiments, the compounds provided herein are selective androgen receptor degrading compounds.

In certain embodiments, provided herein are methods of making and methods of using androgen receptor modulators, androgen binding compounds, and or selective androgen receptor reducing compounds provided herein. In certain embodiments, selective androgen modulators are agonists, partial agonists, and/or antagonists for the androgen receptor.

In certain embodiments, the compounds provided herein have a structure selected from Formula I, Formula Ia, Formula II, Formula IIa, Formula IIb, Formula III, Formula IV, Formula V and Formula VI:

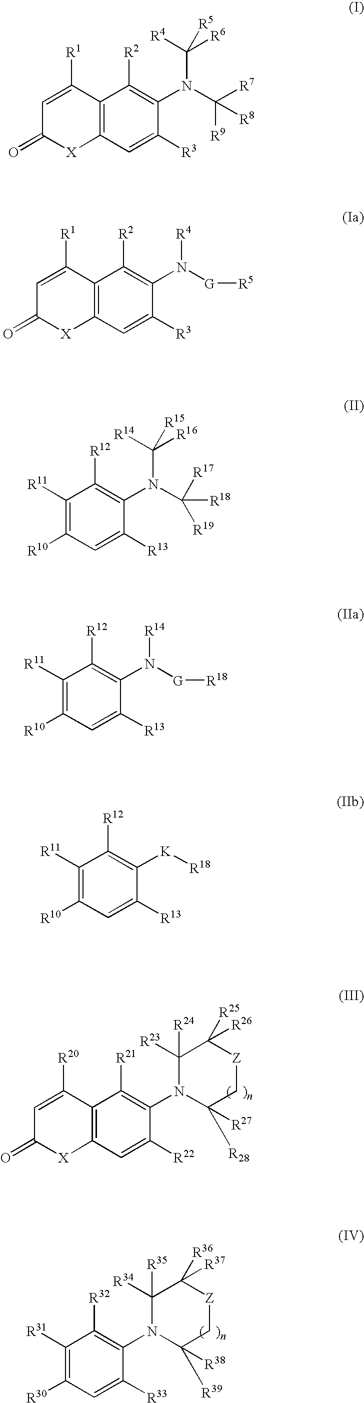

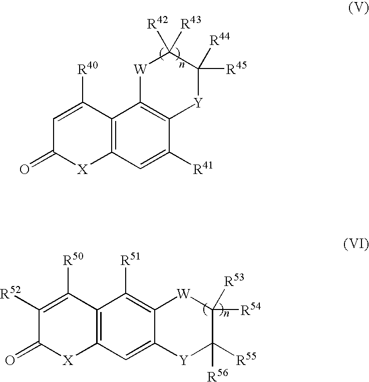

and pharmaceutically acceptable salts and prodrugs thereof.

In certain embodiments, R¹, R² and R³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R¹ is selected from halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R¹ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R¹ is selected from halogen, OR^{A}, SR^{A}, NR^{A}R^{B}, substituted C₂-C₄ alkyl, substituted C₂-C₄ haloalkyl, substituted C₂-C₄ heteroalkyl, substituted C₂-C₄ heterohaloalkyl and QT.

In certain embodiments, R¹ is selected from halogen and QT.

In certain embodiments, R² is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R² is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R² is selected from halogen, OR^{A}, SR^{A}, NR^{A}R^{B}, substituted C₂-C₄ alkyl, substituted C₂-C₄ haloalkyl, substituted C₂-C₄ heteroalkyl, substituted C₂-C₄ heterohaloalkyl and QT.

In certain embodiments, R² is selected from halogen and QT.

In certain embodiments, R³ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R³ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R³ is selected from halogen, OR^{A}, SR^{A}, NR^{A}R^{B}, substituted C₂-C₄ alkyl, substituted C₂-C₄ haloalkyl, substituted C₂-C₄ heteroalkyl, substituted C₂-C₄ heterohaloalkyl and QT.

In certain embodiments, R³ is selected from halogen and QT.

In certain embodiments, R⁴, R⁵, R⁶, R⁷, R⁸ and R⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT.

In certain embodiments, R⁴ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴ is selected from C₂-C₆ alkyl, C₂-C₆ haloalkyl, C₂-C₆ heteroalkyl, C₂-C₆ heterohaloalkyl and QT.

In certain embodiments, R⁴ is selected from substituted C₂-C₆ alkyl, substituted C₂-C₆ haloalkyl, substituted C₂-C₆ heteroalkyl, substituted C₂-C₆ heterohaloalkyl and QT.

In certain embodiments, R⁴ is QT.

In certain embodiments, R⁵ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵ is selected from C₂-C₆ alkyl, C₂-C₆ haloalkyl, C₂-C₆ heteroalkyl, C₂-C₆ heterohaloalkyl and QT.

In certain embodiments, R⁵ is selected from substituted C₂-C₆ alkyl, substituted C₂-C₆ haloalkyl, substituted C₂-C₆ heteroalkyl, substituted C₂-C₆ heterohaloalkyl and QT.

In certain embodiments, R⁵ is QT.

In certain embodiments, R⁶ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁶ is selected from C₂-C₆ alkyl, C₂-C₆ haloalkyl, C₂-C₆ heteroalkyl, C₂-C₆ heterohaloalkyl and QT.

In certain embodiments, R⁶ is selected from substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁶ is QT.

In certain embodiments, R⁷ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁷ is selected from C₂-C₆ alkyl, C₂-C₆ haloalkyl, C₂-C₆ heteroalkyl, C₂-C₆ heterohaloalkyl and QT.

In certain embodiments, R⁷ is selected from substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁷ is QT.

In certain embodiments, R⁸ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁸ is selected from C₂-C₆ alkyl, C₂-C₆ haloalkyl, C₂-C₆ heteroalkyl, C₂-C₆ heterohaloalkyl and QT.

In certain embodiments, R⁸ is selected from substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁸ is QT.

In certain embodiments, R⁹ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁹ is selected from C₂-C₆ alkyl, C₂-C₆ haloalkyl, C₂-C₆ heteroalkyl, C₂-C₆ heterohaloalkyl and QT.

In certain embodiments, R⁹ is selected from substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁹ is QT.

In certain embodiments, R¹⁰ and R¹¹ are independently selected from NO₂, CN, halogen, C₁-C₄ alkyl, C₁-C₄ haloalkyl, C₁-C₄ heteroalkyl and C₁-C₄ heterohaloalkyl.

In certain embodiments, R¹⁰ is selected from NO₂, CN, halogen, C₂-C₄ alkyl, C₂-C₄ haloalkyl, C₂-C₄ heteroalkyl and C₂-C₄ heterohaloalkyl.

In certain embodiments, R¹⁰ is selected from NO₂, CN and halogen.

In certain embodiments, R¹⁰ is selected from C₂-C₄ alkyl, C₂-C₄ haloalkyl, C₂-C₄ heteroalkyl and C₂-C₄ heterohaloalkyl.

In certain embodiments, R¹⁰ is selected from C₂-C₄ alkyl and C₂-C₄ haloalkyl.

In certain embodiments, R¹¹ is selected from NO₂, CN, halogen, C₂-C₄ alkyl, C₂-C₄ haloalkyl, C₂-C₄ heteroalkyl and C₂-C₄ heterohaloalkyl.

In certain embodiments, R¹¹ is selected from NO₂, CN and halogen.

In certain embodiments, R¹¹ is selected from C₂-C₄ alkyl, C₂-C₄ haloalkyl, C₂-C₄ heteroalkyl and C₂-C₄ heterohaloalkyl.

In certain embodiments, R¹¹ is selected from C₂-C₄ alkyl and C₂-C₄ haloalkyl.

In certain embodiments, R¹² and R¹³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R¹² is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R¹² is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R¹² is selected from halogen, OR^{A}, SR^{A}, NR^{A}R^{B}, substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R¹² is selected from halogen and QT.

In certain embodiments, R¹³ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R¹³ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R¹³ is selected from halogen, OR^{A}, SR^{A}, NR^{A}R^{B}, substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R¹³ is selected from halogen and QT.

In certain embodiments, R¹⁴, R¹⁵, R¹⁶, R¹⁷, R¹⁸ and R¹⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT.

In certain embodiments, R¹⁴ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁴ is selected from C₂-C₆ alkyl, C₂-C₆ haloalkyl, C₂-C₆ heteroalkyl, C₂-C₆ heterohaloalkyl and QT.

In certain embodiments, R¹⁴ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁴ is QT.

In certain embodiments, R¹⁵ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁵ is selected from C₂-C₆ alkyl, C₂-C₆ haloalkyl, C₂-C₆ heteroalkyl, C₂-C₆ heterohaloalkyl and QT.

In certain embodiments, R¹⁵ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁵ is QT.

In certain embodiments, R¹⁶ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁶ is selected from C₂-C₆ alkyl, C₂-C₆ haloalkyl, C₂-C₆ heteroalkyl, C₂-C₆ heterohaloalkyl and QT.

In certain embodiments, R¹⁶ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁶ is QT.

In certain embodiments, R¹⁷ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁷ is selected from C₂-C₆ alkyl, C₂-C₆ haloalkyl, C₂-C₆ heteroalkyl, C₂-C₆ heterohaloalkyl and QT.

In certain embodiments, R¹⁷ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁷ is QT.

In certain embodiments, R¹⁸ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁸ is selected from C₂-C₆ alkyl, C₂-C₆ haloalkyl, C₂-C₆ heteroalkyl, C₂-C₆ heterohaloalkyl and QT.

In certain embodiments, R¹⁸ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁸ is QT.

In certain embodiments, R¹⁹ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁹ is selected from C₂-C₆ alkyl, C₂-C₆ haloalkyl, C₂-C₆ heteroalkyl, C₂-C₆ heterohaloalkyl and QT.

In certain embodiments, R¹⁹ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R¹⁹ is QT.

In certain embodiments, R²⁰, R²¹ and R²² are independently selected from hydrogen, halogen, OR^{A}, SR^{A}, NR^{A}R^{B}, optionally substituted C₁-C₄ alkyl optionally substituted C₁-C₄ haloalkyl, optionally substituted C₁-C₄ heteroalkyl, optionally substituted C₁-C₄ heterohaloalkyl and QT.

In certain embodiments, R²⁰ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²⁰ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R²⁰ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²⁰ is selected from halogen and QT.

In certain embodiments, R²¹ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²¹ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R²¹ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²¹ is selected from halogen and QT.

In certain embodiments, R²² is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²² is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R²² is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R²² is selected from halogen and QT.

In certain embodiments, R²³, R²⁴, R²⁵, R²⁶, R²⁷, R²⁸ and R²⁹ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT.

In certain embodiments, R²³ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²³ is selected from OR^{A}, C₂-C₆ alkyl, C₂-C₆ haloalkyl, C₂-C₆ heteroalkyl, C₂-C₆ heterohaloalkyl and QT.

In certain embodiments, R²³ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²³ is selected from OR^{A} and QT.

In certain embodiments, R²⁴ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁴ is selected from OR^{A}, C₂-C₆ alkyl, C₂-C₆ haloalkyl, C₂-C₆ heteroalkyl, C₂-C₆ heterohaloalkyl and QT.

In certain embodiments, R²⁴ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁴ is selected from OR^{A} and QT.

In certain embodiments, R²⁵ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁵ is selected from OR^{A}, C₂-C₆ alkyl C₂-C₆ haloalkyl, C₂-C₆ heteroalkyl, C₂-C₆ heterohaloalkyl and QT.

In certain embodiments, R²⁵ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁵ is selected from OR^{A} and QT.

In certain embodiments, R²⁶ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁶ is selected from OR^{A}, C₂-C₆ alkyl C₂-C₆ haloalkyl, C₂-C₆ heteroalkyl, C₂-C₆ heterohaloalkyl and QT.

In certain embodiments, R²⁶ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁶ is selected from OR^{A} and QT.

In certain embodiments, R²⁷ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁷ is selected from OR^{A}, C₂-C₆ alkyl, C₂-C₆ haloalkyl, C₂-C₆ heteroalkyl, C₂-C₆ heterohaloalkyl and QT.

In certain embodiments, R²⁷ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁷ is selected from OR^{A} and QT.

In certain embodiments, R²⁸ is selected from OR^{A}, optionally substituted C₂-C₆ alkyl, optionally substituted C₂-C₆ haloalkyl, optionally substituted C₂-C₆ heteroalkyl, optionally substituted C₁-C₆ heterohaloalkyl and QT.

In certain embodiments, R²⁸ is selected from OR^{A}, C₂-C₆ alkyl C₂-C₆ haloalkyl, C₂-C₆ heteroalkyl, C₂-C₆ heterohaloalkyl and QT.

In certain embodiments, R²⁸ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁸ is selected from OR^{A} and QT.

In certain embodiments, R²⁹ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁹ is selected from OR^{A}, C₂-C₆ alkyl C₂-C₆ haloalkyl, C₂-C₆ heteroalkyl, C₂-C₆ heterohaloalkyl and QT.

In certain embodiments, R²⁹ is selected from C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R²⁹ is selected from OR^{A} and QT.

In certain embodiments, R³⁰ and R³¹ are independently selected from NO₂, CN, halogen, C₁-C₄ alkyl, C₁-C₄ haloalkyl, C₁-C₄ heteroalkyl and C₁-C₄ heterohaloalkyl.

In certain embodiments, R³⁰ is selected from NO₂, CN, halogen, C₂-C₄ alkyl, C₂-C₄ haloalkyl, C₂-C₄ heteroalkyl and C₂-C₄ heterohaloalkyl.

In certain embodiments, R³⁰ is selected from NO₂, CN and halogen.

In certain embodiments, R³⁰ is selected from halogen, C₂-C₄ alkyl, C₂-C₄ haloalkyl, C₂-C₄ heteroalkyl and C₂-C₄ heterohaloalkyl.

In certain embodiments, R³¹ is selected from NO₂, CN, halogen, C₂-C₄ alkyl, C₂-C₄ haloalkyl, C₂-C₄ heteroalkyl and C₂-C₄ heterohaloalkyl.

In certain embodiments, R³¹ is selected from NO₂, CN and halogen.

In certain embodiments, R³¹ is selected from halogen, C₂-C₄ alkyl, C₂-C₄ haloalkyl, C₂-C₄ heteroalkyl and C₂-C₄ heterohaloalkyl.

In certain embodiments, R³² and R³³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R³² is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R³² is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R³² is selected from halogen, OR^{A}, SR^{A}, NR^{A}R^{B} and QT.

In certain embodiments, R³² is selected from halogen, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R³³ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R³³ is selected from halogen, OR^{A}, SR^{A}, NR^{A}R^{B}, C₂-C₄ alkyl, C₂-C₄ haloalkyl, C₂-C₄ heteroalkyl, C₂-C₄ heterohaloalkyl and QT.

In certain embodiments, R³³ is selected from halogen, OR^{A}, SR^{A}, NR^{A}R^{B} and QT.

In certain embodiments, R³³ is selected from halogen, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R³⁴, R³⁵, R³⁶, R³⁷, R³⁸, R³⁹, R^{39a} and R^{39b} are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT.

In certain embodiments, R³⁴ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁴ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁴ is selected from OR^{A} and QT.

In certain embodiments, R³⁴ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁵ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁵ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁵ is selected from OR^{A} and QT.

In certain embodiments, R³⁵ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁶ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁶ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁶ is selected from OR^{A} and QT.

In certain embodiments, R³⁶ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁷ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁷ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁷ is selected from OR^{A} and QT.

In certain embodiments, R³⁷ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁸ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁸ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R³⁸ is selected from OR^{A} and QT.

In certain embodiments, R³⁸ is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39a} is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39a} is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39a} is selected from OR^{A} and QT.

In certain embodiments, R^{39a} is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39b} is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39b} is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R^{39b} is selected from OR^{A} and QT.

In certain embodiments, R^{39b} is selected from optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁰ and R⁴¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R⁴⁰ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴⁰ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴⁰ is selected from halogen, OR^{A}, SR^{A}, NR^{A}R^{B} and QT.

In certain embodiments, R⁴⁰ is selected from halogen, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴¹ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴¹ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴¹ is selected from halogen, OR^{A}, SR^{A}, NR^{A}R^{B} and QT.

In certain embodiments, R⁴¹ is selected from halogen, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁴², R⁴³, R⁴⁴, R⁴⁵, R⁴⁶ and R⁴⁷ are independently selected from hydrogen, OR^{A}, optionally substituted C₁-C₆ alkyl optionally substituted C₁-C₆ haloalkyl, optionally substituted C₁-C₆ heteroalkyl, optionally substituted C₁-C₆ heterohaloalkyl and QT; or R⁴² and R⁴⁴, and/or R⁴² and R⁴⁷, and/or R⁴⁴ and R⁴⁶ form a bond. R⁴⁴ and R⁴⁷ can optionally form a bond when n is 0.

In certain embodiments, R⁴² is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴² is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴² is selected from OR^{A}, optionally substituted C₂-C₆ alkyl, optionally substituted C₂-C₆ haloalkyl and QT.

In certain embodiments, R⁴² and R⁴⁴ form a bond.

In certain embodiments, R⁴² and R⁴⁷ form a bond.

In certain embodiments, R⁴³ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴³ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴³ is selected from OR^{A}, optionally substituted C₂-C₆ alkyl, optionally substituted C₂-C₆ haloalkyl and QT.

In certain embodiments, R⁴⁴ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁴ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁴ is selected from OR^{A}, optionally substituted C₂-C₆ alkyl, optionally substituted C₂-C₆ haloalkyl and QT.

In certain embodiments, R⁴⁴ and R⁴⁶ form a bond.

In certain embodiments, R⁴⁴ and R⁴⁷ form a bond when n is 0.

In certain embodiments, R⁴⁵ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁵ is selected from OR^{A}, C₂-C₆ alkyl optionally substituted C₂-C₆ haloalkyl, optionally substituted C₂-C₆ heteroalkyl, optionally substituted C₂-C₆ heterohaloalkyl and QT.

In certain embodiments, R⁴⁵ is selected from OR^{A}, optionally substituted C₂-C₆ alkyl, optionally substituted C₂-C₆ haloalkyl and QT.

In certain embodiments, R⁴⁶ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁶ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁶ is selected from OR^{A}, optionally substituted C₂-C₆ alkyl, optionally substituted C₂-C₆ haloalkyl and QT.

In certain embodiments, R⁴⁷ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁷ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁴⁷ is selected from OR^{A}, optionally substituted C₂-C₆ alkyl, optionally substituted C₂-C₆ haloalkyl and QT.

In certain embodiments, R⁵⁰ and R⁵¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT.

In certain embodiments, R⁵⁰ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁵⁰ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁵⁰ is selected from halogen, OR^{A}, SR^{A}, NR^{A}R^{B} and QT.

In certain embodiments, R⁵⁰ is selected from halogen and QT.

In certain embodiments, R⁵¹ is selected from halogen, OR^{A}, SR^{A}, NR^{A}R^{B}, optionally substituted C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁵¹ is selected from halogen, OR^{A}, SR^{A}, NR^{A}R^{B}, C₂-C₄ alkyl, optionally substituted C₂-C₄ haloalkyl, optionally substituted C₂-C₄ heteroalkyl, optionally substituted C₂-C₄ heterohaloalkyl and QT.

In certain embodiments, R⁵¹ is selected from halogen, OR^{A}, SR^{A}, NR^{A}R^{B} and QT.

In certain embodiments, R⁵¹ is selected from halogen and QT.

In certain embodiments, R⁵² is selected from hydrogen, F, Cl, Br, CH₃ and CF₃.

In certain embodiments, R⁵² is selected from F, Cl, Br, CH₃ and CF₃.

In certain embodiments, R⁵² is selected from F, Cl, Br and CF₃.

In certain embodiments, R⁵³, R⁵⁴, R⁵⁵, R⁵⁶, R¹⁷ and R⁵⁸ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT.

In certain embodiments, R⁵³ and R⁵⁵, R⁵³ and R⁵⁷, or R⁵⁵ and R⁵⁸ can optionally form a bond.

In certain embodiments, R⁵⁵ and R⁵⁷ can optionally form a bond when n is 0.

In certain embodiments, R⁵³ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵³ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵³ is selected from OR^{A}, optionally substituted C₂-C₆ alkyl and QT.

In certain embodiments, R⁵³ is selected from OR^{A} and QT.

In certain embodiments, R⁵³ and R⁵⁵ form a bond.

In certain embodiments, R⁵³ and R⁵⁷ form a bond.

In certain embodiments, R⁵⁴ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁴ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁴ is selected from OR^{A}, optionally substituted C₂-C₆ alkyl and QT.

In certain embodiments, R⁵⁴ is selected from OR^{A} and QT.

In certain embodiments, R⁵⁵ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁵ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁵ is selected from OR^{A}, optionally substituted C₂-C₆ alkyl and QT.

In certain embodiments, R⁵⁵ is selected from OR^{A} and QT.

In certain embodiments, R⁵⁵ and R⁵⁸ form a bond.

In certain embodiments, R⁵⁵ and R⁵⁷ form a bond when n is 0.

In certain embodiments, R⁵⁶ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁶ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁶ is selected from OR^{A}, optionally substituted C₂-C₆ alkyl and QT.

In certain embodiments, R⁵⁶ is selected from OR^{A} and QT.

In certain embodiments, R⁵⁷ is selected from OR^{A}, optionally substituted C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁷ is selected from OR^{A}, C₂-C₆ alkyl, optionally substituted C₂-C₆ haloalkyl, optionally substituted C₂-C₆ heteroalkyl, optionally substituted C₂-C₆ heterohaloalkyl and QT.

In certain embodiments, R⁵⁷ is selected from OR^{A}, optionally substituted C₂-C₆ alkyl and QT.

In certain embodiments, R⁵⁷ is selected from OR^{A} and QT.

In certain embodiments, R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring.

In certain embodiments, R^{A} is selected from C₂-C₆ alkyl, C₂-C₆ haloalkyl, C₂-C₆ heteroalkyl, C₂-C₆ heterohaloalkyl and QT.

In certain embodiments, R^{A} is QT.

In certain embodiments, R^{B} is selected from C₂-C₆ alkyl, C₂-C₆ haloalkyl, C₂-C₆ heteroalkyl, C₂-C₆ heterohaloalkyl and QT.

In certain embodiments, R^{B} is QT.

In certain embodiments, R^{A} and R^{B} are linked to form non-aromatic ring.

In certain embodiments, R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring.

In certain embodiments, R^{C} is selected from C₂-C₆ alkyl, C₂-C₆ haloalkyl, C₂-C₆ heteroalkyl and C₂-C₆ heterohaloalkyl.

In certain embodiments, R^{D} is selected from C₂-C₆ alkyl, C₂-C₆ haloalkyl, C₂-C₆ heteroalkyl and C₂-C₆ heterohaloalkyl.

In certain embodiments, R^{C} and R^{D} are linked to form a non-aromatic ring.

In certain embodiments, R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl.

In certain embodiments, R^{E} is selected from OR^{A}, NR^{A}, COR^{A}, C₂-C₆ alkyl, C₂-C₆ haloalkyl, C₂-C₆ heteroalkyl and C₂-C₆ heterohaloalkyl.

In certain embodiments, R^{E} is selected from OR^{A}, NR^{A} and COR^{A}.

In certain embodiments, R^{E} is selected from OR^{A}, C₂-C₆ alkyd C₂-C₆ haloalkyl, C₂-C₆ heteroalkyl and C₂-C₆ heterohaloalkyl.

In certain embodiments, m is selected from 0, 1 and 2.

In certain embodiments, m is 0.

In certain embodiments, m is 1.

In certain embodiments, m is 2.

In certain embodiments, n is selected from 0, 1, 2 and 3.

In certain embodiments, n is 0. In certain embodiments, n is 1. In certain embodiments, n is 2. In certain embodiments, n is 3.

In certain embodiments, G is selected from —CO—, —CS—, —SO₂— and a bond.

In certain embodiments, G is —CO—. In certain embodiments, G is —CS—.

In certain embodiments, G is —SO₂. In certain embodiments, G is a bond.

In certain embodiments, K is selected from —OP(S)OR^{A}O—, —OP(O)OR^{A}O—, —NR^{B}P(S)OR^{A}O— and —NR^{B}P(O)OR^{A}O—.

In certain embodiments, K is —OP(S)OR^{A}O—. In certain embodiments, K is —OP(O)OR^{A}O—. In certain embodiments, K is —NR^{B}P(S)OR^{A}O—. In certain embodiments, K is —NR^{B}P(O)OR^{A}O—.

In certain embodiments, V is selected from O, S and NR^{E}.

In certain embodiments, V is O. In certain embodiments, V is S. In certain embodiments, V is NR^{E}.

In certain embodiments, X is selected from O, S and NR^{A}.

In certain embodiments, X is O. In certain embodiments, X is S. In certain embodiments, X is NR^{A}.

In certain embodiments, Y is selected from O, S, NR⁴⁶ and a bond.

In certain embodiments, Y is O. In certain embodiments, Y is S. In certain embodiments, Y is NR⁴⁶. In certain embodiments, Y is a bond.

In certain embodiments, Z is selected from O, S, NR²⁹ and CR²⁹R^{29a}.

In certain embodiments, Z is O. In certain embodiments, Z is S. In certain embodiments, Z is NR²⁹. In certain embodiments, Z is CR²⁹R^{29a}.

In certain embodiments, W is selected from O, S, NR⁴⁷ and a bond.

In certain embodiments, W is O. In certain embodiments, W is S. In certain embodiments, W is NR⁴⁷. In certain embodiments, W is a bond.

In certain embodiments, Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl.

In certain embodiments, Q is selected from C₂-C₁₂ alkyl, C₂-C₁₂ haloalkyl, C₂-C₁₂ arylalkyl, C₂-C₁₂ arylhaloalkyl and C₂-C₁₂ arylheteroalkyl.

In certain embodiments, Q is selected from C₂-C₁₂ heteroalkyl, heteroarylalkyl, C₂-C₁₂ heteroarylhaloalkyl and C₂-C₁₂ heteroarylheteroalkyl.

In certain embodiments, T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, T is selected from C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D} and SO₂NR^{C}.

In certain embodiments, T is selected from NO₂, CN, C₇-C₁₀ haloalkyl and C₇-C₁₀ heteroalkyl.

In certain embodiments, the compound is selected from Formula III, where

R²⁰ is halogen;

R²¹ and R²² are hydrogen;

R²⁸ is selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

X is N;

n=0; and

Z is CR²⁹R^{29a}; provided that at least one of R^{A}, R²³, R²⁴, R²⁵, R²⁶, R²⁷, R²⁸, R²⁹ and R^{29a} is QT, where Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl;

provided that, if R²³, R²⁴, R²⁵, R²⁶, R²⁷, R²⁸, R²⁹, or R^{29a} is QT, then T is not C₇-C₈ haloalkyl or C₇-C₈ heteroalkyl;

In certain embodiments, the compound is selected from Formula V, where

R⁴⁰ is halogen;

R⁴¹ is selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R⁴² and R⁴⁴ form a bond;

R⁴³ and R⁴⁵ are independently selected from optionally substituted C₁-C₆ alkyl, optionally substituted C₁-C₆ haloalkyl, optionally substituted C₁-C₆ heteroalkyl, optionally substituted C₁-C₆ heterohaloalkyl and QT;

R⁴⁶ is selected from halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;

R^{A} and R^{B} are independently selected from hydrogen, C₁-C₆ alkyl, C₁-C₆ haloalkyl, C₁-C₆ heteroalkyl, C₁-C₆ heterohaloalkyl and QT;

X is NR^{A};

Y is NR⁴⁶; and

W is a bond;

provided that at least one of R^{A}, R⁴¹, R⁴³, R⁴⁵ and R⁴⁶ is QT,

Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and

T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

In certain embodiments, if the compound is selected from Formula I and Formula Ia, then least one of R^{A}, R¹, R², R³, R⁴, R⁵, R⁶, R⁷, R⁸ and R⁹ is QT.

In certain embodiments, if the compound is selected from Formula II, Formula IIa and Formula IIb, then at least one of the R^{A}, R¹², R¹³, R¹⁴, R¹⁵, R¹⁶, R¹⁷, R¹⁸ and R¹⁹ is QT.

In certain embodiments, if the compound is selected from Formula III, then at least one of the R^{A}, R²⁰, R²¹, R²², R²³, R²⁴, R²⁵, R²⁶, R²⁷, R²⁸, R²⁹ and R^{29a} is QT.

In certain embodiments, if the compound is selected from compound IV, then at least one of the R^{A}, R³², R³³, R³⁴, R³⁵, R³⁶, R³⁷, R³⁸, R³⁹, R^{39a} and R^{39b} is QT.

In certain embodiments, if the compound is selected from Formula V, then at least one of R^{A}, R⁴⁰, R⁴¹, R⁴², R⁴³, R⁴⁴, R⁴⁵, R⁴⁶ and R⁴⁷ is QT.

In certain embodiments, if the compound is selected from Formula VI, then at least one of the R^{A}, R⁵⁰, R⁵¹, R⁵³, R⁵⁴, R⁵⁵, R⁵⁶, R⁵⁷ and R⁵⁸ is QT.

In certain embodiments, if the compound is selected from Formula I and Formula Ia and if R⁴, R⁵, R⁶, R⁷, R⁸, or R⁹ is QT, then T is not S(O)ₘR^{C}.

In certain embodiments, if the compound is selected from Formula II, Formula IIa and Formula IIb, and if Q is selected from C₂-C₁₂ alkyl, C₂-C₁₂ haloalkyl, C₂-C₁₂ alkoxy, C₂-C₁₂ arylalkyl, C₂-C₁₂ arylhaloalkyl, and C₂-C₁₂ heteroarylhaloalkyl, then T is not NO₂, CN, COR^{C}, S(O)₂R^{C}; and if Q is selected from noncyclic C₂-C₈ alkyl, and noncyclic C₂-C₈ haloalkyl, then T is not NR^{A}COR^{B}, NR^{A}CO₂R^{B}, or NR^{C}SO₂R^{D};

and pharmaceutically acceptable salts and prodrugs thereof.

In certain embodiments, if the compound is selected from Formula III and if R²³, R²⁴, R²⁵, R²⁶, R²⁷, R²⁸, R²⁹, or R^{29a} is QT, then T is not C₇-C₈ haloalkyl or C₇-C₈ heteroalkyl.

In certain embodiments, if the compound is selected from Formula IV, and if R³⁴, R³⁵, R³⁶, R³⁷, R³⁸, R³⁹, R^{39a}, or R^{39b} is QT, then T is not C₃-C₄ haloalkyl or C₃-C₄ heteroalkyl.

In certain embodiments, if the compound is selected from Formula VI and if W is a bond, and Y is NH, and n is 3, and each of R⁵¹, R⁵², R⁵⁵, and R⁵⁶ is hydrogen, and X is either O or NR^{A}, and either the R⁵³ or the R⁵⁴ bound to the carbon that is closest to Y is QT, then T is not NO₂, CN, C₇-C₈ cycloheteroalkyl, NHCOR^{C}, COR^{C}, O₂CR^{C}, CO₂R^{C}, S(O)ₘR^{C}, CONR^{C}R^{D}, NHCO₂R^{C}, OC(O)NR^{C}R^{D}, NR^{C}C(O)NR^{C}R^{D} and NR^{C}SO₂R^{D}.

In certain embodiments, if the compound is selected from Formula VI and if W is a bond, and Y is NH, and n is 3, and each of R⁵¹, R⁵², R⁵⁵, and R⁵⁶ is hydrogen, and X is NR^{A} where R^{A} is QT, and where Q is saturated noncyclic alkyl, then T is not CO₂H.

In embodiments in which two or more of a particular group are present, the identities of those two or more particular groups are selected independently and, thus, may be the same or different from one another. For example, certain compounds provided herein include two or more R⁵³ groups. The identities of those two or more R⁵³ groups are each selected independently. Thus, in certain embodiments, those R⁵³ groups are all the same as one another; in certain embodiments, those R⁵³ groups are all different from one another; and in certain embodiments, some of those R⁵³ groups are the same as one another and some are different from one another. This independent selection applies to any group that is present in a compound more than once.

In certain embodiments, a compound of Formula I, Formula Ia, Formula II, Formula IIa, Formula IIb, Formula III, Formula IV, Formula V, or Formula VI is a selective androgen receptor modulator. In certain embodiments, a compound of Formula I, Formula Ia, Formula II, Formula IIa, Formula IIb, Formula III, Formula IV, Formula V, or Formula VI is a selective androgen receptor agonist. In certain embodiments, a compound of Formula I, Formula Ia, Formula II, Formula IIa, Formula IIb, Formula III, Formula IV, Formula V, or Formula VI is a selective androgen receptor antagonist. In certain embodiments, a compound of Formula I, Formula Ia, Formula II, Formula IIa, Formula IIb, Formula III, Formula IV, Formula V, or Formula VI is a selective androgen receptor partial agonist. In certain embodiments, a compound of Formula I, Formula Ia, Formula II, Formula IIa, Formula IIb, Formula III, Formula IV, Formula V, or Formula VI is a tissue-specific selective androgen modulator. In certain embodiments, a compound of Formula I, Formula Ia, Formula II, Formula IIa, Formula IIb, Formula III, Formula IV, Formula V, or Formula VI is a selective androgen receptor binding compound. In certain embodiments, a compound of Formula I, Formula Ia, Formula II, Formula IIa, Formula IIb, Formula III, Formula IV, Formula V, or Formula VI is a selective androgen receptor reducing compound. In certain embodiments, a compound of Formula I, Formula Ia, Formula II, Formula IIa, Formula IIb, Formula III, Formula IV, Formula V, or Formula VI is a selective androgen receptor degrading compound.

Certain compounds provided herein can exist as stereoisomers including optical isomers. The present disclosure is intended to include all stereoisomers and both the racemic mixtures of such stereoisomers as well as the individual enantiomers that can be separated according to methods that are known in the art.

### C. Methods of Making

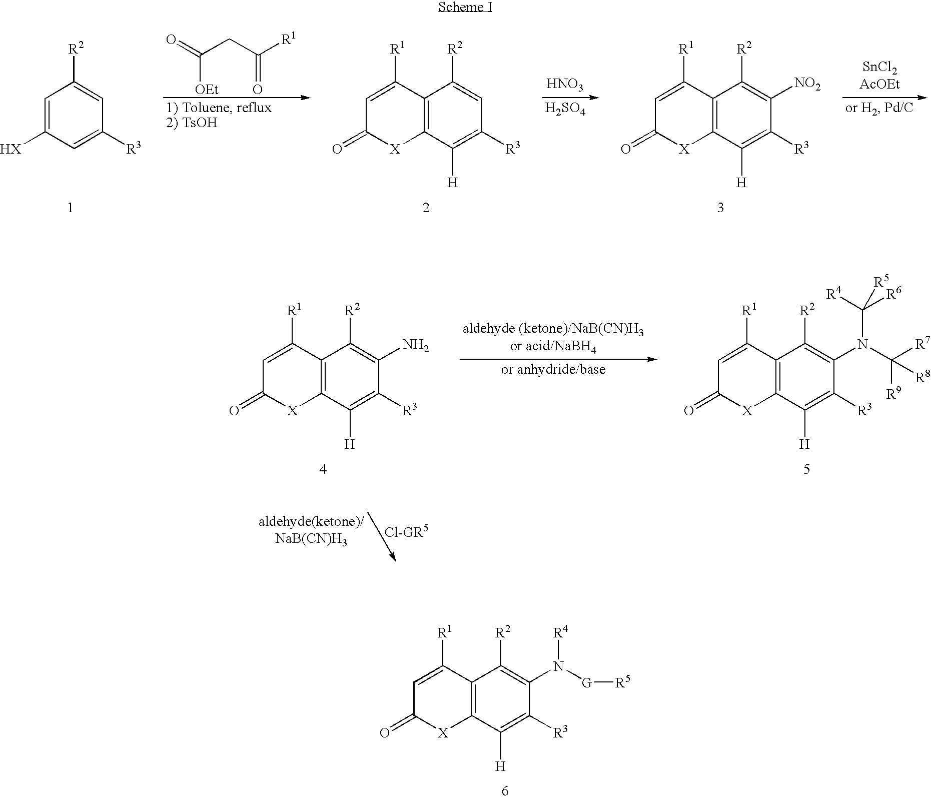

Scheme I describes the methods to prepare the compounds of structure 5 from compounds of structure 1. Compounds of structure 1 are commercially available and known in the art (for example, see U.S. Pat. No. 6,566,372).

A thermal condensation of a compound of structure 1 with a 3-ketoester, for example, ethyl 4,4,4-trifluoroacetoacetate in toluene, affords the intermediates of structure 2. Classic nitration of intermediates 2 selectively provides the nitro compound 3, which is converted to amino intermediates 3 under standard reduction conditions such as metal catalyzed hydrogenation or tin chloride reduction. A two-step sequential reductive alkylation of the amino compounds (structure 4) with an aldehyde or ketone or acid in the presence of a reducing agent, such as sodium cyanoborohydride or sodium borohydride to afford compounds of structure 5 (Formula I). The monoalkylation, monoacylation, monosulfonylation, or any combinations thereof affords the products of structure 6 (Formula Ia).

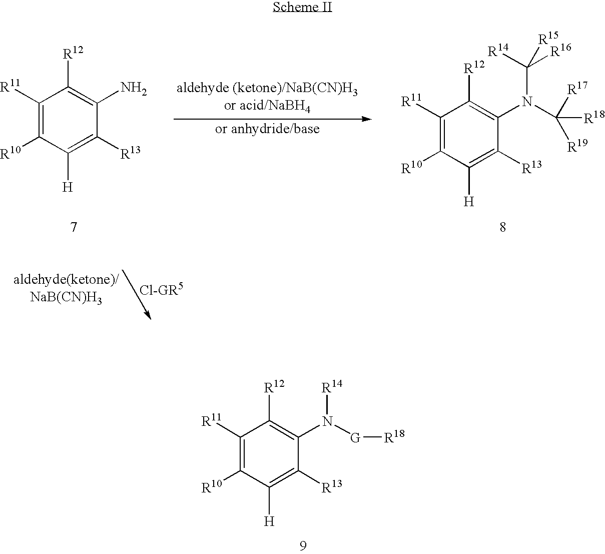

Scheme II describes the methods to prepare the compounds of structures 8 and 9 from compounds of structure 7. A two-step sequential reductive alkylation of the substituted aniline compounds (structure 7) with an aldehyde or ketone or acid in the presence of a reducing agent, such as sodium cyanoborohydride or sodium borohydride to afford compounds of structure 8 (Formula II). Alternatively, treatment of the substituted anilines of Structure 7 with an organohalide in the presence of a base provides the compounds of structure 8 (Formula II). The monoalkylation, monoacylation, monosulfonylation, or any combinations thereof affords the products of structure 9 (Formula IIa).

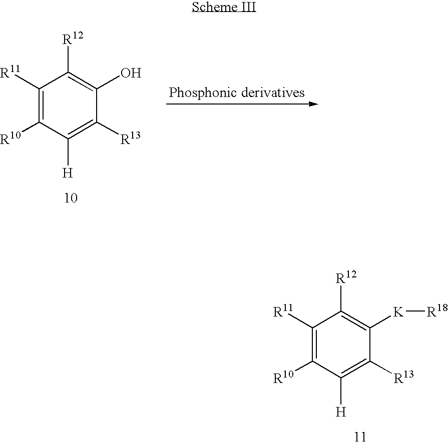

Scheme III describes the methods to prepare the compounds of structure 11 (Formula IIb) from the phenolic derivatives of structure 10. Treatment of 10 with an activated phosphorylation reagent provides the products of structure 11 (Formula IIb).

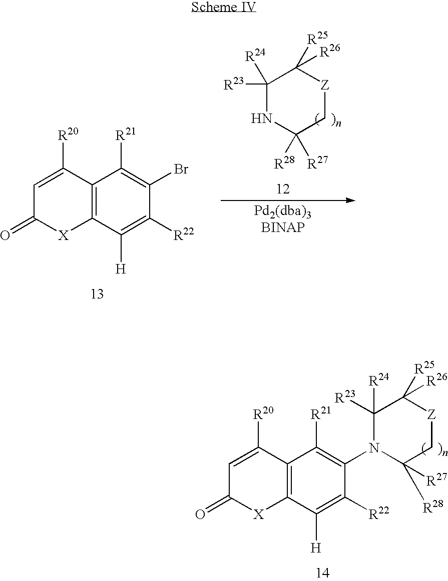

Scheme IV describes a metal-catalyzed, such as palladium or copper, coupling reaction between the bromo compound 13 and a cyclic amine 12 to provide compounds of structure 14 (Formula III). The bromo compound 13 is known in the art (for example, see U.S. Pat. No. 6,566,372). Further modification of the R groups of structure 14 (Formula III) generates more functionalized compounds. When X is nitrogen, the quinolinone moiety of structure 13 needs to be protected as enolethers during the coupling reaction.

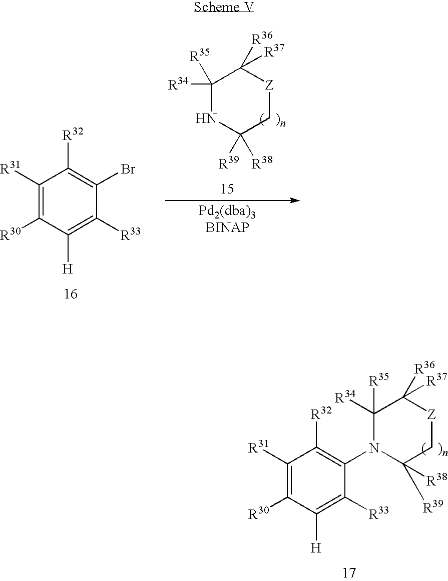

Scheme V describes a metal-catalyzed, such as palladium or copper, coupling reaction between the bromo compound 16 and a cyclic amine 15 followed to provide compounds of structure 17 (Formula IV). The bromo compound 16 is commercially available and is known in the art (for example, see U.S. Pat. App. Publication Nos. 2002-0183346 and 2002-0183314). Further modification of the R groups of structure 17 generates more functionalized compounds.

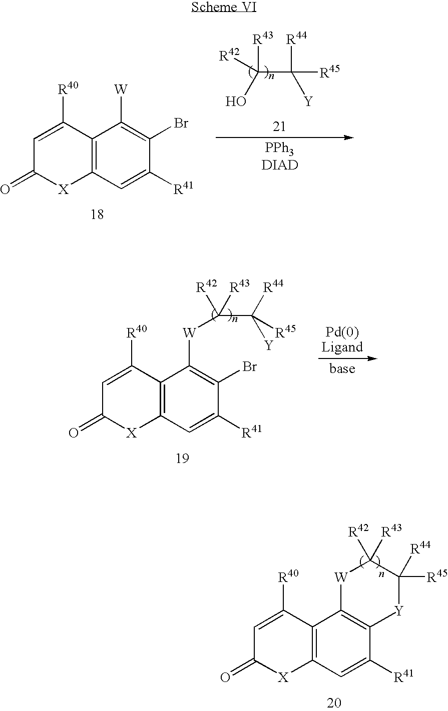

Scheme VI describes the synthesis of compounds of structure 20 (Formula V). Compounds of structure 18 are known in the art (for example, see U.S. Pat. App. Publication No. 2002-0183314). Coupling reaction of compounds of structure 18 and structure 21 provides the first bond connection represented by W of structure 19. The second connection represented by Y is accomplished by a metal-catalyzed, such as palladium or copper, intramolecular coupling reaction of compound 19. Further structure modification of the Y group affords the functionalized compounds of structure 20 (Formula V). When X is nitrogen, the quinolinone moiety of structure 18 needs to be protected as enolethers during the coupling reaction.

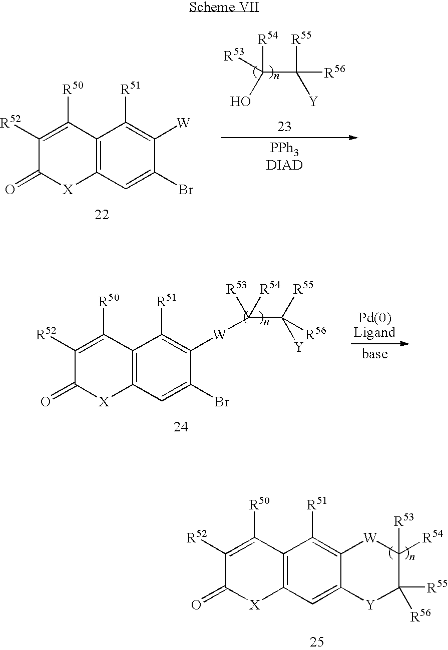

Scheme VII describes the synthesis of compounds of structure 25 (Formula VI). Compounds of structure 22 are known in the art (for example, see U.S. Pat. No. 6,566,372). A coupling reaction of compounds of structures 22 and 23 provides the first bond connection represented by W of structure 24. The second connection represented by Y is accomplished by a metal-catalyzed, such as palladium or copper, intra molecular coupling reaction of compound 24. Further structure modification of Y group affords the functionalized compounds of structure 25 (Formula VI). When X is nitrogen, the quinolinone moiety of structure 22 needs to be protected as enolethers during the coupling reaction.

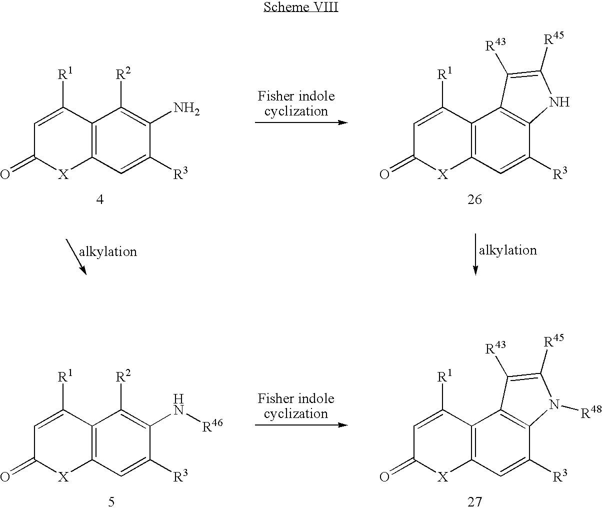

Scheme VIII describes the synthesis of compounds of structure 26 and 27. A Fisher indole cyclization of compounds of structure 4 affords indoles of structure 26. Those indoles can be alkylated to compounds of structure 27. Alternatively, alkylation of compounds of structure 4 affords compounds of structure 5, which can be subjected to Fisher indole cyclization conditions to afford compounds of structure 27. When X is nitrogen, the quinolinone moiety of structure 18 is protected as an enolether during the alkylation reaction.

### D. Formulation of Pharmaceutical Compositions

The pharmaceutical compositions provided herein include therapeutically effective amounts of one or more of the androgen receptor activity modulators provided herein that are useful in the prevention, treatment, or amelioration of one or more of the symptoms of diseases or disorders associated with androgen receptor activity. Such prevention, treatment, or amelioration of diseases or disorders include, but are not limited to, maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic myalgia; treatment of acute fatigue syndrome and muscle loss following elective surgery (e.g., post-surgical rehabilitation); accelerating of wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells including the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength.

In certain embodiments, compositions are therapeutically effective for treating prostate cancer. Prostate cancer, behind lung cancer, is the second main cause of death by cancer in men. In men over 55 years of age, 4% of deaths are attributed to a prostate tumor disease, and it is suggested that the proportion in men over 80 increases up to 80% of deaths. The mortality rate is still always relatively low; but it increases annually to about 14%. The number of men in whom a prostate tumor was diagnosed increased in recent years by 30%, which can be attributed, however, less to an increasing number of new diseases but rather to that the population is generally older, that the diagnostic processes have improved and that systematic screening programs were introduced (E. J. Small, D. M. Reese, Curr. Opi. Oncol. 2000, 12, 265-272).

The prostate tumor grows in an androgen-dependent manner in the early stages. As long as the tumor is limited locally to the prostate, it can be treated by surgical intervention or by radiation therapy. Both of these methods are associated with corresponding risks. In the cases in which the tumor is no longer locally limited, and has already formed metastases, the tumor is treated in a palliative manner by reduction of the testosterone level in the blood. This is carried out either surgically by castration or medicinally by treatment with antiandrogens (bicalutamide, cyproterone acetate, flutamide), LHRH agonists (buserelin, zoladex), LHRH antagonists (cetrorelix) or 5α-reductase inhibitors (finasteride). Since the adrenal androgen synthesis remains unaffected in surgical castration, more recently a combined surgical and medicinal treatment is frequently performed. This treatment, however, has only temporary success, since renewed growth of the tumor generally occurs after two years at the latest, and said renewed growth in most cases is then hormone-independent (L. J. Denis, K. Griffith, Semin. in Surg. Onc. 2000, 18, 52-74).

There are various indications that show that the androgen receptor plays an important role in the development and the growth of the prostate tumor not only in the early hormone-dependent stages but also in late hormone-independent stages of the tumor progression. The androgen receptor belongs to the family of steroid hormone receptors that act as transcription factors. The androgen receptor binds androgens, by which it is stabilized and protected from proteolytic degradation. After hormone binding, it is transported into the nucleus where it activates certain genes by binding to androgen-responsive DNA elements that are in promoter regions (D. J. Lamb et al., Vitamn. Horm. 2001, 62, 199-230).

Studies on prostate tumors show that an amplification of the androgen receptor locus was detected in 30% of the advanced tumors. In other cases, a number of mutations were found in the androgen receptor gene that are located in various domains of the androgen receptor molecule and result in altered receptor properties. Mutated receptors can have either a higher affinity for androgens, can be constitutively active, can change their ligand specificity, such that they are activated by other steroid hormones or even antiandrogens, can be activated via interactions with molecules from other growth-promoting signal-transmission methods, which change interaction with co-factors, or can activate other target genes (J. P. Elo, T. Visakorpi, Ann. Med. 2001, 33, 130-41).

The identification of antiandrogens that inhibit not only the natural androgen receptor but also its mutated forms and in addition change the receptor molecule so that it is destabilized, is beneficial in treating prostate tumors in various stages. Such compounds could prevent a recurrence of tumor growth or at least considerably delay such recurrence. In the case of the estrogen receptor, ligands have been identified that destabilize the receptor and result in a reduction of the receptor content in vitro and in vivo (S. Dauvois et al., Proc. Natl. Acad. Sci. USA 1992, 89, 4037-41; R. A. McClelland et al., Eur. J. Cancer 1996, 32A, 413-6).

In certain embodiments, the prostate cancer is androgen dependant prostate cancer. In certain embodiments, the prostate cancer is androgen independent prostate cancer. In certain embodiments, the prostate cancer is androgen independent, but androgen receptor dependant prostate cancer. See e.g., U.S. Pat. No. 6,861,432. In certain such embodiments, administration of compositions provided herein results in a decrease in the amount of functional androgen receptor present in cells. In certain embodiments, administration of compositions provided herein results in degradation of androgen receptors.

The compositions include one or more compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In certain embodiments, a pharmaceutical composition including one or more compounds provided herein is prepared using known techniques, including, but not limited to mixing, dissolving, granulating, dragee-making, levigating, emulsifying, encapsulating, entrapping or tabletting processes.

In the compositions, effective concentrations of one or more compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with androgen activity or in which androgen activity is implicated.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. Liposomal suspensions, including tissue-targeted liposomes, such as tumor-targeted liposomes, can also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation and then resuspended in PBS.

The active compound is included in the pharmaceutically acceptable carrier in an amount sufficient to exert a therapeutically useful effect in the absence of undesirable side effects on the patient treated.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with androgen activity or in which androgen activity is implicated, as described herein.

The effective amount of a compound of provided herein can be determined by one of ordinary skill in the art, and includes exemplary dosage amounts for a mammal of from about 1 μg/kg to 50 mg/kg and from about 0.05 to 100 mg/kg of body weight of active compound per day, which can be administered in a single dose or in the form of individual divided doses, such as from 1 to 4 times per day. It will be understood that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination and severity of the particular condition.

The active ingredient can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific-dosage regimens should be adjusted over time according to the individual need and the professional judgment of the person administering or supervising the administration of the compositions, and that the concentration ranges set forth herein are exemplary only and are not intended to limit the scope or practice of the compounds, compositions, methods and other subject matter provided herein.

Pharmaceutically acceptable derivatives include acids, bases, enol ethers and esters, salts, esters, hydrates, solvates and prodrug forms. The derivative is selected such that its pharmacokinetic properties are superior to the corresponding neutral corm pound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with androgen receptor activity or in which androgen receptor activity is implicated, as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally in form of capsules, tablets, granules, powders or liquid formulations including syrups; parenterally, such as subcutaneously, intravenously, intramuscularly, with inteasternal injection or infusion techniques (as sterile injectable aqueous or non-aqueous solutions or suspensions); nasally such as by inhalation spray; topically, such as in the form of a cream or ointment; rectally such as in the form of suppositories; liposomally; and locally. The compositions can be in liquid, semi-liquid or solid form and are formulated in a manner suitable for each route of administration. In certain embodiments, administration of the formulation include parenteral and oral modes of administration. In one embodiment, the compositions are administered orally.

In certain embodiments, the pharmaceutical compositions provided herein including one or more compounds provided herein is a solid (e.g., a powder, tablet, and/or capsule). In certain of such embodiments, a solid the pharmaceutical composition including one or more compounds provided herein is prepared using ingredients known in the art, including, but not limited to, starches, sugars, diluents, granulating agents, lubricants, binders and disintegrating agents.

In certain embodiments, a pharmaceutical composition including one or more compounds provided herein is formulated as a depot preparation. Certain of such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition including one or more compounds provided herein includes a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, a pharmaceutical composition including one or more compounds provided herein includes one or more tissue-specific delivery molecules designed to deliver the pharmaceutical composition to specific tissues or cell types. For example, in certain embodiments, pharmaceutical compositions include liposomes coated with a tissue-specific antibody.

In certain embodiments, a pharmaceutical composition including one or more compounds provided herein includes a co-solvent system. Certain of such co-solvent systems include, for example, benzyl alcohol, a nonpolar surfactant, a water-miscible organic polymer and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, solutions or suspensions used for parenteral, intradermal, subcutaneous, or topical application can include any of the following components: a sterile diluent, such as water for injection, saline solution, fixed oil, polyethylene glycol, glycerin, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds can be used. Such methods are known to those of skill in this art, and include, but are not limited to, using co-solvents, such as dimethylsulfoxide (DMSO), using surfactants, such as TWEEN®, or dissolution in aqueous sodium bicarbonate. Derivatives of the compounds, such as prodrugs of the compounds can also be used in formulating effective pharmaceutical compositions.

In certain embodiments, a pharmaceutical composition including one or more compounds provided herein includes a sustained release system. A non-limiting example of such a sustained-release system is a semipermeable matrix of solid hydrophobic polymers. In certain embodiments, sustained release systems may, depending on their chemical nature, release compounds over a period of hours, days, weeks or months.

In certain embodiments, upon mixing or addition of the compound(s), the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and can be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions including suitable quantities of the compounds or pharmaceutically acceptable derivatives thereof. The pharmaceutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose includes a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can include along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, xanthan gum, gellan gum, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols and ethanol, to thereby form a solution or suspension. If desired, the pharmaceutical composition to be administered can also include minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, include a quantity of the active compound in an amount sufficient to alleviate the symptoms of the treated subject.

Dosage forms or compositions including active ingredient in the range of 0.005% to 100% with the balance made up from non-toxic carrier can be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, xanthan gum, gellan gum, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions can include 0.001%-100% active ingredient, in one embodiment 0.1-85%, in another embodiment 75-95%.

In certain embodiments, the compounds can be administered in a form suitable for immediate release or extended release. Immediate release or extended release can be achieved with suitable pharmaceutical compositions or, particularly in the case of extended release, with devices such as subcutaneous implants or osmotic pumps. The compounds can be administered in a form suitable for controlled release. Methods for preparation of these compositions are known to those skilled in the art. For example, see Ansel, Introduction to Pharmaceutical Dosage Forms, Fourth Edition (1985), pages 167-174; and Controlled Release Delivery Systems (Roseman and Mansdorf, editors; Marcel Dekker, Inc. (1983).

In certain embodiments, the compounds can be administered in a form suitable for topical administration. Exemplary compositions for topical administration include a topical carrier such as PLASTIBASE® (mineral oil gelled with polyethylene).

In certain embodiments, compounds used in the pharmaceutical compositions may be provided as pharmaceutically acceptable salts with pharmaceutically compatible counterions. Pharmaceutically compatible salts may be formed with many acids, including but not limited to hydrochloric, sulfuric, acetic, lactic, tartaric, malic and succinic.

In certain embodiments, the pharmaceutical compositions include a compound provided herein in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

The compositions can include other active compounds to obtain desired combinations of properties. The compounds provided herein, or pharmaceutically acceptable derivatives thereof as described herein, can also be advantageously administered for therapeutic or prophylactic purposes together with another pharmacological agent known in the general art to be of value in treating one or more of the diseases or medical conditions referred to hereinabove, such as diseases or disorders associated with androgen receptor activity or in which androgen receptor activity is implicated. It is to be understood that such combination therapy constitutes a further aspect of the compositions and methods of treatment provided herein.

In certain embodiments, a pharmaceutical composition including one or more compounds provided herein is formulated as a prodrug. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, a prodrug is an ester. In certain embodiments, such prodrugs are less water soluble than the corresponding active form. In certain instances, such prodrugs possess superior transmittal across cell membranes, where water solubility is detrimental to mobility. In certain embodiments, the ester in such prodrugs is metabolically hydrolyzed to carboxylic acid. In certain instances the carboxylic acid-containing compound is the corresponding active form. In certain embodiments, a prodrug includes a short peptide (polyaminoacid) bound to an acid group. In certain of such embodiments, the peptide is metabolized to form the corresponding active form.

In certain embodiments, a pharmaceutical composition including one or more compounds provided herein is useful for treating a conditions or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary and subcutaneous). In certain embodiments, pharmaceutical compositions are administered to achieve local rather than systemic exposures. For example, pharmaceutical compositions may be injected directly in the area of desired effect (e.g., in the renal or cardiac area). In certain embodiments in which the pharmaceutical composition is administered locally, the dosage regimen is adjusted to achieve a desired local concentration of a compound provided herein.

In certain embodiments, a pharmaceutical composition including one or more compounds provided herein is administered in the form of a dosage unit (e.g., tablet, capsule, bolus, etc.). In certain embodiments, such dosage units include a selective androgen receptor modulator in a dose from about 1 μg/kg of body weight to about 50 mg/kg of body weight. In certain embodiments, such dosage units include a selective androgen receptor modulator in a dose from about 2 μg/kg of body weight to about 25 mg/kg of body weight. In certain embodiments, such dosage units include a selective androgen receptor modulator in a dose from about 10 μg/kg of body weight to about 5 mg/kg of body weight. In certain embodiments, pharmaceutical compositions are administered as needed, once per day, twice per day, three times per day, or four or more times per day. It is recognized by those skilled in the art that the particular dose, frequency and duration of administration depends on a number of factors, including, without limitation, the biological activity desired, the condition of the patient and tolerance for the pharmaceutical composition.

In certain embodiments, a pharmaceutical composition provided herein is administered for a period of continuous therapy. For example, a pharmaceutical composition provided herein may be administered over a period of days, weeks, months, or years.

Dosage amount, interval between doses and duration of treatment may be adjusted to achieve a desired effect. In certain embodiments, dosage amount and interval between doses are adjusted to maintain a desired concentration on compound in a patient. For example, in certain embodiments, dosage amount and interval between doses are adjusted to provide plasma concentration of a compound provided herein at an amount sufficient to achieve a desired effect. In certain of such embodiments the plasma concentration is maintained above the minimal effective concentration (MEC). In certain embodiments, pharmaceutical compositions provided herein are administered with a dosage regimen designed to maintain a concentration above the MEC for 10-90% of the time, between 30-90% of the time, or between 50-90% of the time.

1. Compositions for Oral Administration

In certain embodiments, oral pharmaceutical dosage forms are either solid, gel or liquid. The solid dosage forms are tablets, capsules, granules and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art.

In certain embodiments, the formulations are solid dosage forms, preferably capsules or tablets. The tablets, pills, capsules, troches and the like can include any of the following ingredients, or compounds of a similar nature: a binder; a diluent; a disintegrating agent; a lubricant; a glidant; a sweetening agent; and a flavoring agent.

In certain embodiments, pharmaceutical compositions for oral administration are push fit capsules made of gelatin. Certain of such push fit capsules include one or more compounds provided herein in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more compounds provided are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

Examples of binders for use in the compositions provided herein include microcrystalline cellulose, gum tragacanth, xanthan gum, gellan gum, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, sodium alginate, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, aspartame and sucralose, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition can also be formulated in combination with an antacid or other such ingredient.

When the dosage unit form is a capsule, it can include, in addition to material of the above type, a liquid carrier such as a fatty oil. In addition, dosage unit forms can include various other materials which modify the physical form of the dosage unit, for example, coatings of sugar and other enteric agents. The compounds can also be administered as a component of an elixir, suspension, syrup, wafer, sprinkle, chewing gum or the like. A syrup can include, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers and diuretics. The active ingredient is a compound or pharmaceutically acceptable derivative thereof as described herein. Higher concentrations, up to about 98% by weight of the active ingredient can be included.

Pharmaceutically acceptable carriers included in tablets are binders, lubricants, diluents, disintegrating agents, coloring agents, flavoring agents and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents can also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose and can include a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin, aspartame and sucralose. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes and mixtures thereof. Flavoring agents include natural flavors extracted from plants such fruits and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those including a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations can be coated as known by those of skill in the art in order to modify or sustain dissolution of the active ingredient. Thus, for example, they can be coated with a conventional enterically digestible coating, such as phenylsalicylate, waxes and cellulose acetate phthalate.

Exemplary compositions can include fast-dissolving diluents such as mannitol, lactose, sucrose and/or cyclodextrins. Also included in such formulations can be high molecular weight excipients such as celluloses (AVICEL®), xanthan gum (KELTROL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZO®); and agents to control release such as polyacrylic copolymer (e.g., CARBOPOL 9340). Lubricants, glidants, flavors, coloring agents and stabilizers can also be added for ease of fabrication and use.

In certain of such embodiments, a pharmaceutical composition for oral administration is formulated by combining one or more compounds provided herein with one or more pharmaceutically acceptable carriers. Certain of such carriers enable compounds provided herein to be formulated as tablets, pills, dragees, capsules, liquids, gels, syrups, slurries, suspensions and the like, for oral ingestion by a patient. In certain embodiments, pharmaceutical compositions for oral use are obtained by mixing one or more compounds provided herein and one or more solid excipient. Suitable excipients include, but are not limited to, fillers, such as sugars, including lactose, sucrose, mannitol, or sorbitol; polyglucans such as, for example, maize starch, wheat starch, rice starch, potato starch, gelatin, gum tragacanth, xanthan gum, gellan gum, cellulose preparations such as, for example, methyl cellulose, hydroxypropylmethyl cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross linked polyvinyl pyrrolidone, agar, or alginic acid or a salt thereof, such as sodium alginate) are added.

In certain embodiments, dragee cores are provided with coatings. In certain of such embodiments, concentrated sugar solutions may be used, which may optionally include gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, a daily dosage regimen for a patient includes an oral dose of between 0.1 mg and 2000 mg of a compound provided herein. In certain embodiments, a daily dosage regimen is administered as a single daily dose. In certain embodiments, a daily dosage regimen is administered as two, three, four, or more than four doses.

2. Injectables, Solutions and Emulsions

In certain embodiments, the pharmaceutical composition is prepared for transmucosal administration. In certain of such embodiments penetrants appropriate to the barrier to be permeated are used in the formulation. Such penetrants are generally known in the art.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol, mannitol, 1,3-butanediol, Ringer's solution, an isotonic sodium chloride solution or ethanol. In addition, if desired, the pharmaceutical compositions to be administered can also include minor amounts of non-toxic auxiliary substances such as wetting or emulsifying agents, pH buffering agents, stabilizers, solubility enhancers, and other such agents, such as for example, mono- or diglycerides, fatty acids, such as oleic acid, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound included in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions including thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzylalcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution including an active compound is an effective mode of administration. Another embodiment is a sterile aqueous or oily solution or suspension including an active material injected as necessary to produce the desired pharmacological effect.

Injectables are designed for local and systemic administration. Typically a therapeutically effective dosage is formulated to include a concentration of at least about 0.1% w/w up to about 90% w/w or more, preferably more than 1% w/w of the active compound to the treated tissue(s). The active ingredient can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of formulations provided herein.

The compounds can be formulated in any suitable vehicle or form. For example, they can be in micronized or other suitable form and/or can be derivatized to produce a more soluble active product or to produce a prodrug or for other purposes. The form of the resulting mixture depends upon a number of factors, including, for example, an intended mode of administration and the solubility of the compound in the selected carrier or vehicle. The effective concentration is sufficient for ameliorating the symptoms of the condition and can be empirically determined.

In certain embodiments, a pharmaceutical composition is prepared for administration by injection wherein the pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampules or in multi dose containers. Certain pharmaceutical compositions for injection are suspensions, solutions or emulsions in oily or aqueous vehicles, and may include formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides and liposomes. Aqueous injection suspensions may include substances that increase the viscosity of the suspension, such as sodium carboxymethyl cellulose, sorbitol, or dextran. Optionally, such suspensions may also include suitable stabilizers or agents that increase the solubility of the compounds to allow for the preparation of highly concentrated solutions.

In certain embodiments, the pharmaceutical composition is prepared for administration by inhalation. Certain of such pharmaceutical compositions for inhalation are prepared in the form of an aerosol spray in a pressurized pack or a nebulizer. Certain of such pharmaceutical compositions include a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations include a powder mixture of a compound provided herein and a suitable powder base such as lactose or starch.

In certain embodiments, the pharmaceutical compositions provided are administered by continuous intravenous infusion. In certain of such embodiments, from 0.1 mg to 500 mg of the composition is administered per day.

3. Lyophilized Powders

Of interest herein are also lyophilized powders, which can be reconstituted for administration as solutions, emulsions and other mixtures. They can also be reconstituted and formulated as solids or gels.

The sterile, lyophilized powder is prepared by dissolving a compound provided herein, or a pharmaceutically acceptable derivative thereof, in a suitable solvent. The solvent can include an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also include a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will include a single dosage of 10-1000 mg, in one embodiment, 100-500 mg or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture can be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof can be formulated as aerosols for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

In certain embodiments, the pharmaceutical compositions for inhalation are prepared in the form of an aerosol spray in a pressurized pack or a nebulizer. Certain of such pharmaceutical compositions include a propellant, e.g., dichlorodifluoromethane, trichlorofluoromethane, dichlorotetrafluoroethane, carbon dioxide or other suitable gas. In certain embodiments using a pressurized aerosol, the dosage unit can be determined with a valve that delivers a metered amount. In certain embodiments, capsules and cartridges for use in an inhaler or insufflator can be formulated. Certain of such formulations include a powder mixture of a compound provided herein and a suitable powder base such as lactose or starch.

Exemplary compositions for nasal aerosol or inhalation administration include solutions which can include, for example, benzyl alcohol or other suitable preservatives, absorption promoters to enhance absorption and/or bioavailability, and/or other solubilizing or dispersing agents such as those known in the art.

The compounds can be formulated for local or topical application, such as for topical application to the skin and mucous membranes, such as in the eye, in the form of gels, creams and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, can be formulated as 0.01%-10% isotonic solutions, pH about 5-7, with appropriate salts. In certain embodiments in which the compositions is administered locally, the dosage regimen is adjusted to achieve a desired local concentration of a compound provided herein.

In certain embodiments, the pharmaceutical composition is prepared for topical administration. Certain of such pharmaceutical compositions include bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for the pharmaceutical composition provided herein can be chosen in view of a particular patient's condition. (See e.g., Fingi et al. 1975, in “The Pharmacological Basis of Therapeutics”, Ch. 1 p. 1). In certain embodiments, the pharmaceutical composition is administered as a single dose. In certain embodiments, a pharmaceutical composition is administered as a series of two or more doses administered over one or more days.

5. Compositions for Other Routes of Administration

Other routes of administration, such as topical application, transdermal patches and rectal administration are also contemplated herein.

In certain embodiments, the pharmaceutical composition is prepared for topical administration such as rectal administration. The pharmaceutical dosage forms for rectal administration include, but are not limited to rectal suppositories, capsules and tablets for systemic effect. In certain embodiments, a pharmaceutical agent is prepared for rectal administration, such as a suppositories or retention enema. Certain of such pharmaceutical agents include known ingredients, such as cocoa butter and/or other glycerides. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles- and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases can be used. In certain embodiments, the pharmaceutical compositions include bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.). Agents to raise the melting point of suppositories include spermaceti and wax. Rectal suppositories can be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The compounds or pharmaceutically acceptable derivatives can be packaged as articles of manufacture including packaging material, within the packaging material a compound or pharmaceutically acceptable derivative thereof provided herein, which is effective for modulating the activity of androgen receptor, or for treatment, prevention or amelioration of one or more symptoms of androgen receptor mediated diseases or disorders, or diseases or disorders in which androgen receptor activity is implicated, and a label that indicates that the compound or composition, or pharmaceutically acceptable derivative thereof, is used for modulating the activity of androgen receptor or for treatment, prevention or amelioration of one or more symptoms of androgen receptor mediated diseases or disorders, or diseases or disorders in which androgen receptor activity is implicated.

The articles of manufacture provided herein include packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles and any packaging material suitable for a selected formulation and intended mode of administration and treatment. A wide array of formulations of the compounds and compositions provided herein are contemplated as are a variety of treatments for any disease or disorder in which androgen receptor activity is implicated as a mediator or contributor to the symptoms or cause.

In certain embodiments, the pharmaceutical compositions can be presented in a pack or dispenser device which can include one or more unit dosage forms including a compound provided herein. The pack can, for example, include metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions including a compound provided herein formulated in a compatible pharmaceutical carrier can also be prepared, placed in an appropriate container and labeled for treatment of an indicated condition.

### E. Evaluation of the Activity of the Compounds

Standard physiological, pharmacological and biochemical procedures are available for testing the compounds provided herein to identify those that possess activity as androgen receptor modulators. In vitro and in vivo assays known in the art can be used to evaluate the activity of the compounds provided herein as androgen receptor modulators. Exemplary assays include, but are not limited to fluorescence polarization assay, luciferase assay and co-transfection assay. In certain embodiments, the compounds provided herein are capable of modulating activity of androgen receptor in a “co-transfection” assay (also called a “cis-trans” assay), which is known in the art. See e.g., Evans et al., Science, 240:889-95 (1988); U.S. Pat. Nos. 4,981,784 and 5,071,773; Pathirana et al., “Nonsteroidal Human Progesterone Receptor Modulators from the Marie Alga Cymopolia Barbata,” Mol. Pharm. 47:630-35 (1995)). Modulating activity in a co-transfection assay has been shown to correlate with in vivo modulating activity. Thus, in certain embodiments, such assays are predictive of in vivo activity. See, e.g., Berger et al., J. Steroid Biochem. Molec. Biol. 41:773 (1992).

In certain co-transfection assays, two different co-transfection plasmids are prepared. In the first co-transfection plasmid, cloned cDNA encoding an intracellular receptor (e.g., androgen receptor) is operatively linked to a constitutive promoter (e.g., the SV 40 promoter). In the second co-transfection plasmid, cDNA encoding a reporter protein, such as firefly luciferase (LUC), is operatively linked to a promoter that is activated by a receptor-dependant activation factor. Both co-transfection plasmids are co-transfected into the same cells. Expression of the first co-transfection plasmid results in production of the intracellular receptor protein. Activation of that intracellular receptor protein (e.g., by binding of an agonist) results in production of a receptor-dependant activation factor for the promoter of the second co-transfection plasmid. That receptor-dependant activation factor in turn results in expression of the reporter protein encoded on the second co-transfection plasmid. Thus, reporter protein expression is linked to activation of the receptor. Typically, that reporter activity can be conveniently measured (e.g., as increased luciferase production).

Certain co-transfection assays can be used to identify agonists, partial agonists, and/or antagonists of intracellular receptors. In certain embodiments, to identify agonists, co-transfected cells are exposed to a test compound. If the test compound is an agonist or partial agonist, reporter activity is expected to increase compared to co-transfected cells in the absence of the test compound. In certain embodiments, to identify antagonists, the cells are exposed to a known agonist (e.g., androgen for the androgen receptor) in the presence and absence of a test compound. If the test compound is an antagonist, reporter activity is expected to decrease relative to that of cells exposed only to the known agonist.

In certain embodiments, compounds provided herein are used to detect the presence, quantity and/or state of receptors in a sample. In certain of such embodiments, samples are obtained from a patient. In certain embodiments, compounds are radio- or isotopically-labeled. For example, compounds provided herein that selectively bind androgen receptors may be used to determine the presence of such receptors in a sample, such as cell homogenates and lysates.

### F. Methods of Use of the Compounds and Compositions

Methods of use of the compounds and compositions provided herein also are provided. The methods include in vitro and in vivo uses of the compounds and compositions for altering androgen receptor activity and for treatment, prevention, or amelioration of one or more symptoms of diseases or disorder that are modulated by androgen receptor activity, or in which androgen receptor activity, is implicated. In certain embodiments, provided herein are methods of treating a patient by administering a compound provided herein. In certain embodiments, such patient exhibits symptoms or signs of a androgen receptor mediated condition. In certain embodiments, a patient is treated prophylactically to reduce or prevent the occurrence of a condition.

The compounds provided herein can be used in the treatment of a variety of conditions including, but not limited to, maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic myalgia; treatment of acute fatigue syndrome and muscle loss following elective surgery (e.g., post-surgical rehabilitation); accelerating of wound healing; accelerating bone fracture repair (such as accelerating the recovery of hip fracture patients); accelerating healing of complicated fractures, e.g., distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells including the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength. The term treatment is also intended to include prophylactic treatment.

In certain embodiments, the compounds provided herein are used to treat acne, male-pattern baldness, wasting diseases, hirsutism, hypogonadism, osteoporoses, infertility, impotence, obesity and cancer. In certain embodiments, one or more compounds provided herein are used to stimulate hematopoiesis. In certain embodiments, one or more compounds provided herein are used for contraception.

In certain embodiments, one or more compounds provided herein are used to treat cancer. Certain exemplary cancers include, but are not limited to, breast cancer, colorectal cancer, gastric carcinoma, glioma, head and neck squamous cell carcinoma, papillary renal carcinoma, leukemia, lymphoma, Li-Fraumeni syndrome, malignant pleural mesothelioma, melanoma, multiple myeloma, non-small cell lung cancer, synovial sarcoma, thyroid carcinoma, transitional cell carcinoma of urinary bladder and prostate cancer, including, but not limited to prostatic hyperplasia.

In certain embodiments, one or more compounds provided herein are used to improve athletic performance. In certain such embodiments, one or more compounds provided herein are used, for example to shorten the time normally needed to recover from physical exertion or to increase muscle strength. Athletes to whom one or more compounds provided herein can be administered include, but are not limited to, horses, dogs and humans. In certain embodiments, one or more compounds provided herein are administered to an athlete engaged in a professional or recreational competition, including, but not limited to weight-lifting, body-building, track and field events and any of various team sports.

In certain embodiments, provided are methods for treating a patient by administering one or more selective androgen receptor agonists and/or partial agonists. Exemplary conditions that can be treated with such selective androgen receptor agonists and/or partial agonist include, but are not limited to, hypogonadism, wasting diseases, cancer cachexia, frailty, infertility and osteoporosis. In certain embodiments, a selective androgen receptor agonist or partial agonist is used for male hormone replacement therapy. In certain embodiments, one or more selective androgen receptor agonists and/or partial agonists are used to stimulate hematopoiesis. In certain embodiments, a selective androgen receptor agonist or partial agonist is used as an anabolic agent. In certain embodiments, a selective androgen receptor agonist and/or partial agonist is used to improve athletic performance.

In certain embodiments, provided herein are methods for treating a patient by administering one or more selective androgen receptor antagonists and/or partial agonists. Exemplary conditions that may be treated with such one or more selective androgen receptor antagonists and/or partial agonists include, but are not limited to, hirsutism, acne, male-pattern baldness, prostatic hyperplasia and cancer, including, but not limited to, various hormone-dependent cancers, including, without limitation, prostate and breast cancer.

In certain embodiments, provided herein are methods for treating a patient with prostate cancer. In certain such embodiments, the prostate cancer is androgen dependant prostate cancer. In certain embodiments, the prostate cancer is androgen independent prostate cancer. In certain embodiments, the prostate cancer is androgen independent, but androgen receptor dependant prostate cancer. See e.g., U.S. Pat. No. 6,861,432. In certain such embodiments, administration of compositions provided herein results in a decrease in the amount of functional androgen receptor present in cells. In certain embodiments, administration of compositions provided herein results in degradation of androgen receptors.

### G. Combination Therapies

In certain embodiments, one or more compounds or compositions provided herein can be co-administered with one or more other pharmaceutical agents. In certain embodiments, such one or more other pharmaceutical agents are designed to treat the same disease or condition as the one or more compounds or pharmaceutical compositions provided herein. In certain embodiments, such one or more other pharmaceutical agents are designed to treat a different disease or condition as the one or more compounds or compositions provided herein. In certain embodiments, such one or more other pharmaceutical agents are designed to treat an undesired effect of one or more compounds or compositions provided herein. In certain embodiments, one or more compounds or compositions provided herein is co-administered with another pharmaceutical agent to treat an undesired effect of that other pharmaceutical agent.

In certain embodiments, compounds or compositions provided herein and one or more other pharmaceutical agents are administered at the same time. In certain embodiments, compounds or compositions provided herein and one or more other pharmaceutical agents are administered at the different times. In certain embodiments, compounds or compositions provided herein and one or more other pharmaceutical agents are prepared together in a single formulation. In certain embodiments, compounds or compositions provided herein and one or more other pharmaceutical agents are prepared separately.

Examples of pharmaceutical agents that may be co-administered with compounds or compositions provided herein include, but are not limited to, analgesics (e.g., acetaminophen); anti-inflammatory agents, including, but not limited to non-steroidal anti-inflammatory drugs (e.g., ibuprofen, COX-1 inhibitors and COX-2, inhibitors); salicylates; antibiotics; antivirals; antifungal agents; antidiabetic agents (e.g., biguanides, glucosidase inhibitors, insulins, sulfonylureas and thiazolidenediones); adrenergic modifiers; diuretics; hormones (e.g., anabolic steroids, androgen, estrogen, calcitonin, progestin, somatostatin and thyroid hormones); immunomodulators; muscle relaxants; antihistamines; osteoporosis agents (e.g., biphosphonates, calcitonin and estrogens); prostaglandins, anti-neoplastic agents; psychotherapeutic agents; sedatives; poison oak or poison sumac products; antibodies; and vaccines.

In other embodiments, pharmaceutical agents that may be co-administered with compounds or compositions provided herein include, but are not limited to, other modulators of nuclear hormone receptors or other suitable therapeutic agents useful in the treatment of the aforementioned disorders including: anti-diabetic agents; anti-osteoporosis agents; anti-obesity agents; anti-inflammatory agents; anti-anxiety agents; anti-depressants; anti-hypertensive agents; anti-platelet agents; anti-thrombotic and thrombolytic agents; cardiac glycosides; cholesterol/lipid lowering agents; mineralocorticoid receptor antagonists; phosphodiesterase inhibitors; protein tyrosine kinase inhibitors; thyroid mimetics (including thyroid receptor agonists); anabolic agents; HIV or AIDS therapies; therapies used in the treatment of Alzheimer's and other cognitive disorders; therapies used in the treatment of sleeping disorders; anti-proliferative agents; and anti-tumor agents.

### EXAMPLES

The following examples, including experiments and results achieved, are provided for illustrative purposes only and are not to be construed as limiting the claimed subject matter.

### Example 1

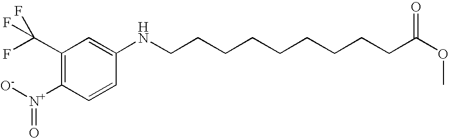

### 10-(4-Nitro-3-trifluoromethyl-phenylamino)-decanoic acid methyl ester

(Compound 120, Structure 9 of Scheme II, where R¹¹=Trifluoromethyl, R¹⁰=Nitro, R¹⁴=Hydrogen, G=a Bond, R¹⁸=Methyldecanoate)

4-Nitro-3-trifluoromethylaniline (100 mg) was dissolved in 5 mL DMF with stirring. To this was added 10-bromodecanoic acid methyl ester (1 eq., 128 mg) followed by potassium carbonate (2 eq., 134 mg). After stirring at room temperature and seeing no change by TLC (50% ethyl acetate/hexanes), the reaction mixture was heated to 50° C. After another 2 days, the reaction was judged complete by TLC, and the mixture was allowed to cool to room temperature, HCl_{aq} added and extracted into ethyl acetate (EA). The organic layer was washed with water then concentrated. Chromatography (silica, 25% EA/hexanes). Trituration of the recovered material afforded 72 mg (38%). ¹H NMR (500 MHz, CDCl₃) δ 8.03 (d, J=9.0 Hz, 1H); 6.87 (d, J=2.6 Hz, 1H); 6.64 (dd, J=9.0, 2.6 Hz, 1H); 4.58 (t, J=5.2 Hz, 1H); 3.67 (s, 3H); 3.21 (td, J=7.1, 5.2 Hz, 2H); 2.31 (t, J=7.5 Hz, 2H); 1.70-1.59 (m, 4H); 1.46-1.29 (m, 10H).

### Example 2

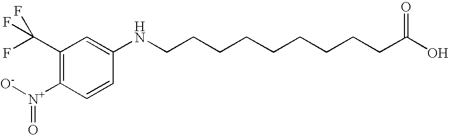

### 10-(4-Nitro-3-trifluoromethyl-phenylamino)-decanoic acid

(Compound 121, Structure 9 of Scheme II, where R¹¹=Trifluoromethyl, R¹⁰=Nitro, R¹⁴=Hydrogen, G=a Bond, R¹⁸=10-Decanoic Acid)

¹H NMR (500 MHz, CDCl₃) δ 9.33 (s, 1H); 8.03 (d, J=9.1 Hz, 1H); 6.87 (d, J=2.6 Hz, 1H); 6.64 (dd, J=9.1, 2.6 Hz, 1H); 4.57 (br s, 1H); 3.22 (t, J=6.7 Hz, 2H); 2.36 (t, J=7.5 Hz, 2H); 1.70-1.60 (m, 4H); 1.44-1.29 (m, 10H).

### Example 3

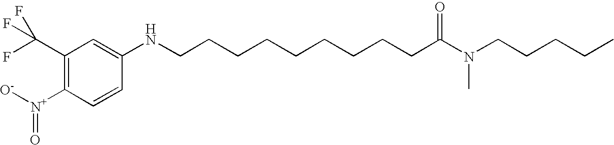

### 10-(4-Nitro-3-trifluoromethyl-phenylamino)-decanoic acid N-methyl-N-pentyl-amide

(Compound 114, Structure 9 of Scheme II, where R¹¹=Trifluoromethyl, R¹⁰=Nitro, R¹⁴=Hydrogen, G=a Bond, R¹⁸=N-Methyl-N-Pentyl Decanoic Acid Amide)

¹H NMR (500 MHz, CDCl₃) δ 8.03 (d, J=9.0 Hz, 1H); 6.88 (d, J=2.7 Hz, 1H); 6.64 (dd, J=9.0, 2.7 Hz, 1H); 4.74 (m, 1H); 3.35 (t, J=7.5 Hz, 1H); 3.27-3.19 (m, 3H); 2.97 (s, 1.5H); 2.91 (s, 1.5H); 2.30 (t, J=7.2 Hz, 1H); 2.29 (t, J=7.3 Hz, 1H); 1.69-1.61 (m, 4H); 1.44-1.22 (m, 16H); 0.92 (t, J=7.2 Hz, 1.5H); 0.89 (t, J=7.2 Hz, 1.5H).

### Example 4

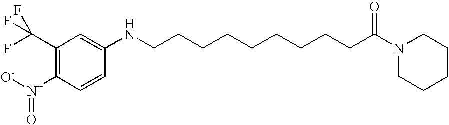

### 10-(4-Nitro-3-trifluoromethyl-phenylamino)-1-piperidin-1-yl-decan-1-one

(Compound 115, Structure 9 of Scheme II, where R¹¹=Trifluoromethyl, R¹⁰=Nitro, R¹⁴=Hydrogen, G=a Bond)

¹H NMR (500 MHz, CDCl₃) δ 8.03 (d, J=9.0 Hz, 1H); 6.88 (d, J=2.6 Hz, 1H); 6.64 (dd, J=9.0, 2.6 Hz, 1H); 4.76 (t, J=5.2 Hz, 1H); 3.55 (m, 2H); 3.39 (m, 2H); 3.21 (td, J=7.0, 5.2 Hz, 2H); 2.31 (t, J=7.6 Hz, 2H); 1.69-1.60 (m, 6H); 1.57-1.50 (m, 4H); 1.44-1.31 (m, 10H).

### Example 5

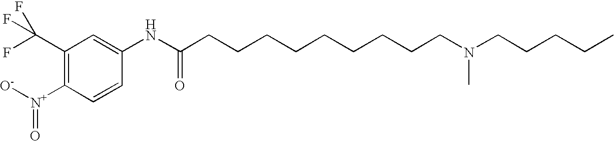

### 10-(N-Methyl-N-pentylamino)-decanoic acid (4-nitro-3-trifluoromethyl-phenyl)-amide

(Compound 122, Structure 9 of Scheme II, where R¹¹=Trifluoromethyl, R¹⁰=Nitro, R¹⁴=Hydrogen, G=(C═O))

10-Bromodecanoic acid (120 mg) was dissolved in 20 mL DCM with stirring. To this was added oxalyl chloride (0.2 mL). After 2 h, the mixture was concentrated to dryness and redissolved in dioxane (10 mL) and added 4-Nitro-3-trifluoromethylaniline (100 mg). After 1 day, the reaction was judged complete by TLC, the mixture was partitioned between water and EA and then concentrated. Chromatography (silica, 25-50% EA/hexanes) afforded 200 mg (quant). 30 mg of the resulting bromide was dissolved in N-methylpyrrolidone (3 mL) followed by N-methyl pentyl amine (0.25 mL) and potassium carbonate and heated to 50° C. After 12 hours, the reaction mixture was partitioned between HCl_{aq} and EA and concentrated, Prep. LC (60/40 acetonitrile/water w/0.1% formic acid) afforded 5 mg (16%). ¹H NMR (500 MHz, CDCl₃) δ 10.36 (s, 1H); 8.38 (d, J=2.2 Hz, 1H); 8.28 (dd, J=8.9, 2.2 Hz, 1H); 7.94 (d, J=8.9 Hz, 1H); 3.05-2.86 (m, 4H); 2.76 (s, 3H); 2.55 (t, J=7.6 Hz, 2H); 1.88-1.76 (m, 6H); 1.50-1.22 (m, 14H); 0.93 (t, J=6.9 Hz, 3H).

### Example 6

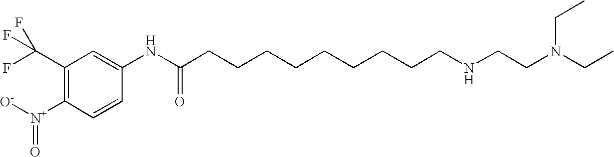

### 10-(2-Diethylamino-ethylamino)-decanoic acid (4-nitro-3-trifluoromethyl-phenyl)-amide

(Compound 123, Structure 9 of Scheme II, where R¹¹=Trifluoromethyl, R¹⁰=Nitro, R¹⁴=Hydrogen, G=(C═O))

¹H NMR (500 MHz, CDCl₃) δ 10.84 (s, 1H); 8.59 (m, 1H); 8.28 (dd, J=8.0, 2.3 Hz, 1H); 8.17 (d, J=2.3 Hz, 1H); 7.95 (d, J=8.0 Hz, 1H); 3.57 (dq, J=7.5, 5.6 Hz, 4H); 3.07-2.82 (m, 6H); 2.76 (q, J=7.2 Hz, 4H); 2.47 (m, 2H); 1.82-1.70 (m, 4H); 1.45-1.27 (m, 10H); 1.14 (t, J=7.2 Hz, 6H).

### Example 7

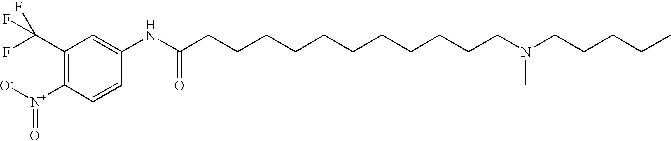

### 12-(N-Methyl-N-pentylamino)-dodecanoic acid (4-nitro-3-trifluoromethyl-phenyl)-amide

(Compound 124, Structure 9 of Scheme II, where R¹¹=Trifluoromethyl, R¹⁰=Nitro, R¹⁴=Hydrogen, G=(C═O))

¹H NMR (500 MHz, CDCl₃) δ 10.20 (s, 1H); 8.31 (dd, J=8.9, 2.2 Hz, 1H); 8.29 (d, J=2.2 Hz, 1H); 7.95 (d, J=8.9 Hz, 1H); 3.02-2.92 (m, 4H); 2.75 (s, 3H); 2.54 (t, J=7.6 Hz, 2H); 1.90-1.73 (m, 8H); 1.44-1.28 (m, 16H); 0.92 (t, J=7.0 Hz, 3H).

### Example 8

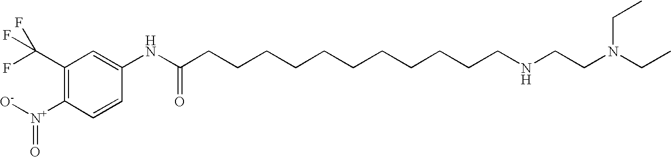

### 12-(2-Diethylamino-ethylamino)-dodecanoic acid (4-nitro-3-trifluoromethyl-phenyl)-amide

(Compound 125, Structure 9 of Scheme II, where R¹¹=Trifluoromethyl, R¹⁰=Nitro, R¹⁴=Hydrogen, G=(C═O))

¹H NMR (500 MHz, CDCl₃) δ 10.35 (s, 1H); 8.61 (s, 1H); 8.31 (dd, J=9.0, 2.2 Hz, 1H); 8.22 (d, J=2.2 Hz, 1H); 7.95 (d, J=9.0 Hz, 1H); 3.07-2.82 (m, 6H); 2.76 (q, J=7.2 Hz, 4H); 2.52 (t, J=7.5 Hz, 2H); 1.82-1.72 (m, 4H); 1.45-1.27 (m, 14H); 1.14 (t, J=7.2 Hz, 6H).

### Example 9

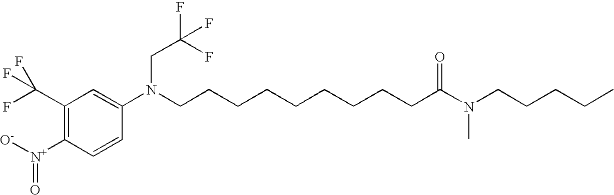

### 10-[(4-Nitro-3-trifluoromethyl-phenyl)-(2,2,2-trifluoro-ethyl)-amino]-decanoic acid N-methyl-N-pentyl-amide

(Compound 116, Structure 9 of Scheme II, where R¹¹=Trifluoromethyl, R¹⁰=Nitro, R¹⁴=Trifluoroethyl, G=a Bond)

20 mg of compound 114 was dissolved in 5 mL TFA with stirring and heated to 50° C. To this was added about 200 mg of NaBH₄ and heated to 60° C. After 12 hours, cooled to room temperature and added water, basified using 1N NaOH and extracted into EA. The organic layer was washed with water and brine and concentrated. Chromatography (silica, 25-50% EA/hexanes) afforded 5 mg (23%). ¹H NMR (500 MHz, CDCl₃) δ 8.05 (d, J=9.3 Hz, 1H); 7.06 (d, J=2.8 Hz, 1H); 6.87 (dd, J=9.3, 2.8 Hz, 1H); 3.99 (q, J=8.5 Hz, 2H); 3.50 (t, J=8.0 Hz, 2H); 3.35 (t, J=7.6 Hz, 1H); 3.25 (t, J=7.6 Hz, 1H); 2.96 (s, 1.5H); 2.91 (s, 1.5H); 2.30 (t, J=7.1 Hz, 1H); 2.28 (t, J=6.9 Hz, 1H); 1.67-1.59 (m, 6H); 1.58-1.47 (m, 2H); 1.38-1.22 (m, 12H); 0.91 (t, J=7:2 Hz, 1.5H), 0.89 (t, J=7.2 Hz, 1.5H).

### Example 10

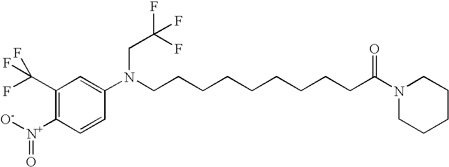

### 10-[(4-Nitro-3-trifluoromethyl-phenyl)-(N-2,2,2-trifluoroethyl)amino]-1-piperidin-1-yl-decan-1-one

(Compound 117, Structure 9 of Scheme II, where R¹¹=Trifluoromethyl, R¹⁰=Nitro, R¹⁴=Trifluoroethyl, G=a Bond)

¹H NMR (500 MHz, CDCl₃) δ 8.05 (d, J=9.2 Hz, 1H); 7.06 (d, J=2.9 Hz, 1H); 6.87 (dd, J=9.2, 2.9 Hz, 1H); 3.99 (q, J=8.5 Hz, 2H); 3.55 (t, J=5.5 Hz, 2H); 3.50 (t, J=7.9 Hz, 2H); 3.39 (t, J=5.5 Hz, 2H); 2.31 (t, J=7.7 Hz, 2H); 1.67-1.60 (m, 6H); 1.58-1.50 (m, 4H); 1.37-1.30 (m, 10H).

### Example 11

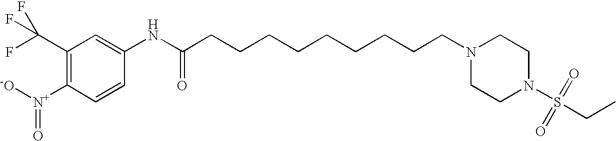

### 10-(4-Ethanesulfonyl-piperazin-1-yl)-decanoic acid (4-nitro-3-trifluoromethyl-phenyl)-amide

(Compound 126, Structure 9 of Scheme II, where R¹¹=Trifluoromethyl, R¹⁰=Nitro, R¹⁴=Hydrogen, G=(C═O))

¹H NMR (500 MHz, CDCl₃) 8.06 (dd, J=8.8, 2.3 Hz, 1H); 8.00 (d, J=2.3 Hz, 1H); 7.98 (d, J=8.8 Hz, 1H); 3.43-3.37 (m, 4H); 2.98 (q, J=7.4 Hz, 2H); 2.64 (m, 4H); 2.48 (m, 2H); 2.43 (t, J=7.6 Hz, 2H); 1.74 (m, 2H); 1.38 (t, J=7.4 Hz, 3H); 1.31 (m, 12H).

### Example 12

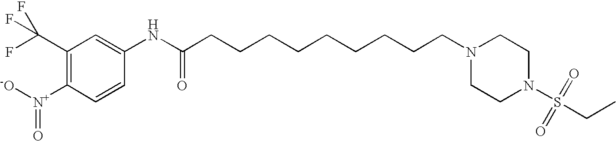

### 12-(4-Ethanesulfonyl-piperazin-1-yl)-dodecanoic acid (4-nitro-3-trifluoromethyl-phenyl)-amide

(Compound 127, Structure 9 of Scheme II, where R¹¹=Trifluoromethyl, R¹⁰=Nitro, R¹⁴=Hydrogen, G=(C═O))

¹H NMR (500 MHz, CDCl₃) δ 8.04 (dd, J=8.8, 2.3 Hz, 1H); 7.98 (d, J=8.8 Hz, 1H); 7.98 (d, J=2.3 Hz, 1H); 3.44-3.34 (m, 4H); 2.98 (q, J=7.4 Hz, 2H); 2.71-2.54 (m, 4H); 2.48 (m, 2H); 2.43 (t, J=7.6 Hz, 2H); 1.74 (m, 2H); 1.69-1.57 (m, 4H); 1.38 (t, J=7.4 Hz, 3H); 1.34-1.26 (m, 12H).

### Example 13

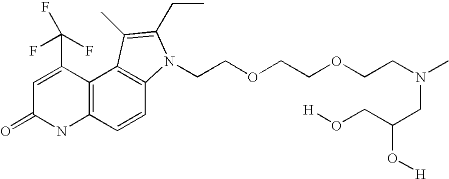

### (±)-3-[2-(2-{2-[(N-2,3-Dihydroxy-propyl)-N-methyl-amino]-ethoxy}-ethoxy)-ethyl]-2-ethyl-1-methyl-9-trifluoromethyl-3,6-dihydro-pyrrolo[3,2-f]quinolin-7-one

(Compound 342, Structure 27 of Scheme VIII, where R¹=Trifluoromethyl, R³=Hydrogen, R⁴³=Methyl, R⁴⁵=Ethyl, R⁴⁶=2-(2-{2-[(2,3-Dihydroxy-Propyl)-Methyl-Amino]-Ethoxy}-Ethoxy)-Ethyl)

¹H NMR (500 MHz, CD₃OD) δ 7.84 (d, J=8.8 Hz, 1H), 7.17 (d, J=8.8 Hz, 1H), 7.00 (s, 1H), 4.61 (m, 2H), 4.46 (t, J=5.4 Hz, 2H), 3.80 (t, J=5.4 Hz, 2H), 3.80 (m, 1H), 3.52 (s, 3H), 3.56-3.51 (m, 4H), 3.49 (d, J=5.0 Hz, 2H), 2.92 (q, J=7.6 Hz, 2H), 2.81 (m, 2H), 2.51 (m, 2H), 2.32 (q, J=2.0 Hz, 3H), 1.24 (t, J=7.6 Hz, 3H).

### Example 14

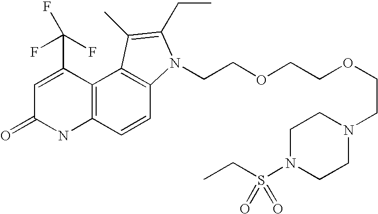

### 3-(2-{2-[2-(4-Ethanesulfonyl-piperazin-1-yl)-ethoxy]-ethoxy}-ethyl)-2-ethyl-1-methyl-9-trifluoromethyl-3,6-dihydro-pyrrolo[3,2-f]quinolin-7-one

(Compound 341, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.83 (d, J=8.8 Hz, 1H), 7.16 (d, J=8.8 Hz, 1H), 7.00 (s, 1H), 4.45 (t, J=5.4 Hz, 2H), 3.79 (t, J=5.4 Hz, 2H), 3.51-3.45 (m, 4H), 3.40 (t, J=5.4 Hz, 2H), 3.16 (m, 4H), 2.97 (q, J=7.5 Hz, 2H), 2.92 (q, J=7.5 Hz, 2H), 2.42 (m, 4H), 2.41 (t, J=5.4 Hz, 2H), 2.32 (q, J=2.0 Hz, 3H), 1.29 (t, J=7.5 Hz, 3H), 1.24 (t, J=7.5 Hz, 3H).

### Example 15

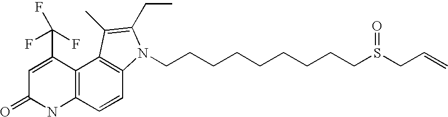

### 2-Ethyl-1-methyl-3-[9-(prop-2-ene-1-sulfinyl)-nonyl]-9-trifluoromethyl-3,6-dihydro-pyrrolo[3,2-f]quinolin-7-one

(Compound 340, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 5.91 (dddd, J=17.1, 10.2, 7.7, 7.1 Hz, 1H), 5.43 (ddt, J=10.2, 1.4, 0.7 Hz, 1H), 5.41 (dq, J=17.1, 1.4 Hz, 1H), 4.24 (t, J=7.5 Hz, 2H), 3.61 (m, 1H), 3.46 (m, 1H), 2.87 (q, J=7.6 Hz, 2H), 2.75 (m, 2H), 2.32 (q, J=2.0 Hz, 3H), 1.80-1.66 (m, 4H), 1.50-1.30 (m, 10H), 1.24 (t, J=7.6 Hz, 3H).

### Example 16

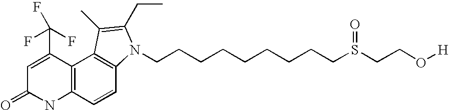

### 2-Ethyl-3-[9-(2-hydroxy-ethanesulfinyl)-nonyl]-1-methyl-9-trifluoromethyl-3,6-dihydro-pyrrolo[3,2-f]quinolin-7-one

(Compound 339, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 7.78 (d, J=8.8 Hz, 1H), 7.27 (d, J=8.8 Hz, 1H), 6.92 (s, 1H), 4.28 (t, J=7.6 Hz, 2H), 4.01-3.94 (m, 2H), 2.91 (m, 2H), 2.74 (m, 2H), 2.32 (q, J=2.1 Hz, 3H), 1.78 (m, 2H), 1.71 (m, 2H), 1.50-1.28 (m, 10H), 1.24 (t, J=7.6 Hz, 3H).

### Example 17

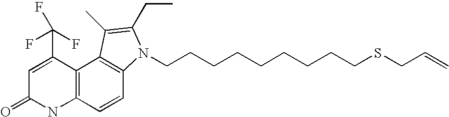

### 2-Ethyl-1-methyl-3-[9-(prop-2-ene-1-sulfinyl)-nonyl]-9-trifluoromethyl-3,6-dihydro-pyrrolo[3,2-f]quinolin-7-one

(Compound 338, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 11.18 (s, 1H), 7.78 (d, J=8.7 Hz, 1H), 7.25 (d, J=8.7 Hz, 1H), 6.92 (s, 1H), 5.77 (ddt, J=16.9, 10.0, 7.1 Hz, 1H), 5.09 (ddt, J=16.9, 1.7, 1.1 Hz, 1H), 5.03 (dd, J=10.0, 1.7, 1.1 Hz, 1H), 4.28 (t, J=7.8 Hz, 2H), 3.12 (dt, J=7.1, 1.1 Hz, 2H), 2.89 (q, J=7.5 Hz, 2H), 2.43 (t, J=7.3 Hz, 2H), 2.32 (q, J=2.1 Hz, 2H), 1.83-1.79 (m, 2H), 1.54 (m, 2H), 1.47-1.40 (m, 2H), 1.40-1.33 (m, 4H), 1.33-1.28 (m, 4H), 1.24 (t, J=7.5 Hz, 3H).

### Example 18

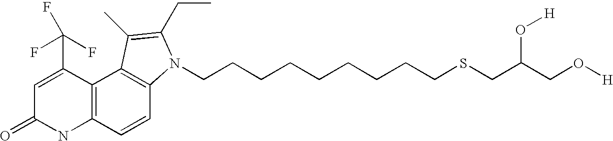

### 3-[9-(2,3-Dihydroxy-propylsulfanyl)-nonyl]-2-ethyl-1-methyl-9-trifluoromethyl-3,6-dihydro-pyrrolo[3,2-f]quinolin-7-one

(Compound 337, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 7.78 (d, J=8.7 Hz, 1H), 7.25 (d, J=8.7 Hz, 1H), 6.91 (s, 1H), 4.28 (t, J=7.8 Hz, 2H), 3.71 (m, 1H), 3.58 (m, 1H), 3.51 (m, 1H), 2.89 (q, J=7.4 Hz, 2H), 2.68 (dd, J=13.6, 5.7 Hz, 1H), 2.55 (m, 2H), 2.54 (dd, J=13.6, 6.6 Hz, 1H), 2.32 (q, J=2.1 Hz, 3H), 1.78 (m, 2H), 1.55 (m, 2H), 1.50-1.28 (m, 10H), 1.24 (t, J=7.4 Hz, 3H).

### Example 19

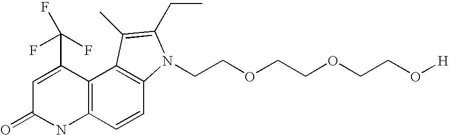

### 2-Ethyl-3-{2-[2-(2-hydroxy-ethoxy)-ethoxy]-ethyl}-1-methyl-9-trifluoromethyl-3,6-dihydro-pyrrolo[3,2-f]quinolin-7-one

(Compound 336, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.75 (d, J=8.8 Hz, 1H), 7.38 (d, J=8.8 Hz, 1H), 7.22 (s, 1H), 4.35 (t, J=5.7 Hz, 2H), 3.72 (t, J=5.7 Hz, 2H), 3.64 (m, 2H), 3.52-3.44 (m, 6H), 2.80 (q, J=7.6 Hz, 2H), 2.27 (q, J=2.0 Hz, 3H), 1.17 (t, J=7.6 Hz, 3H).

### Example 20

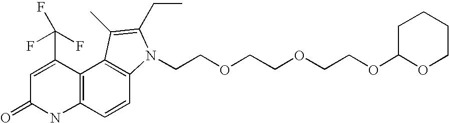

### 2-Ethyl-1-methyl-3-(2-{2-[2-(tetrahydro-pyran-2-yloxy)-ethoxy]-ethoxy}-ethyl)-9-trifluoromethyl-3,6-dihydro-pyrrolo[3,2-f]quinolin-7-one

(Compound 335, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.67 (d, J=8.8 Hz, 1H), 7.33 (d, J=8.8 Hz, 1H), 7.19 (s, 1H), 4.60 (dd, J=4.4, 3.2 Hz, 1H), 4.38 (t, J=6.1 Hz, 2H), 3.84 (m, 2H), 3.77 (t, J=6.1 Hz, 2H), 3.62-3.53 (m, 8H), 2.86 (q, J=7.6 Hz, 2H), 2.33 (q, J=2.0 Hz, 3H), 1.81 (m, 1H), 1.69 (m, 1H), 1.61-1.46 (m, 4H), 1.23 (t, J=7.6 Hz, 3H).

### Example 21

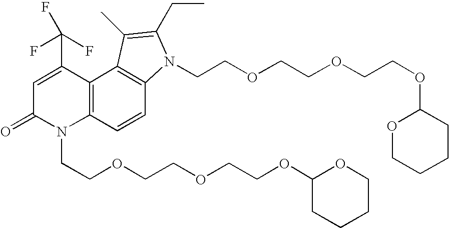

### 2-Ethyl-1-methyl-3,6-bis-(2-{2-[2-(tetrahydro-pyran-2-yloxy)-ethoxy]-ethoxy}-ethyl)-9-trifluoromethyl-3,6-dihydro-pyrrolo[3,2-f]quinolin-7-one

(Compound 334, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.72 (d, J=8.8 Hz, 1H), 7.60 (d, J=8.8 Hz, 1H), 7.28 (s, 1H), 4.69 (m, 2H), 4.63 (dd, J=4.3, 3.1 Hz, 1H), 4.59 (dd, J=4.4, 3.0 Hz, 1H), 4.41 (t, J=6.3 Hz, 2H), 3.94 (m, 2H), 3.89-3.74 (m, 8H), 3.73-3.68 (m, 4H), 3.64-3.53 (m, 8H), 3.52-3.45 (m, 2H), 2.86 (q, J=7.6 Hz, 2H), 2.35 (q, J=2.0 Hz, 3H), 1.87-1.76 (m, 2H), 1.74-1.66 (m, 2H), 1.64-1.46 (m, 8H), 1.23 (t, J=7.6 Hz, 3H).

### Example 22

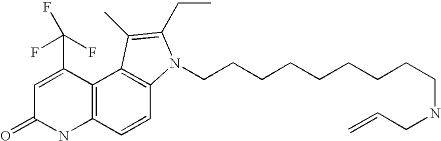

### 3-(9-Allylamino-nonyl)-2-ethyl-1-methyl-9-trifluoromethyl-3,6-dihydro-pyrrolo[3,2-f]-quinolin-7-one

(Compound 333, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 5.89 (ddt, J=17.1, 10.3, 6.6 Hz, 1H), 5.39 (dq, J=17.1, 1.2 Hz, 1H), 5.34 (dq, J=10.3, 1.2 Hz, 1H), 4.24 (t, J=7.4 Hz, 2H), 3.45 (dt, J=6.6, 1.2 Hz, 2H), 2.87 (q, J=7.6 Hz, 2H), 2.78 (t, J=7.7 Hz, 2H), 2.32 (q, J=2.0 Hz, 3H), 1.76 (m, 2H), 1.57 (m, 2H), 1.41-1.28 (m, 10H), 1.24 (t, J=7.6 Hz, 3H).

### Example 23

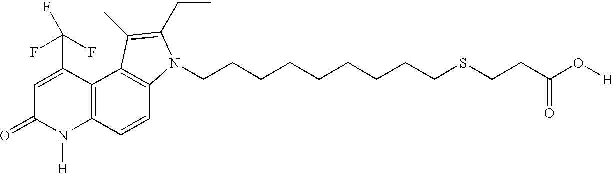

### 3-[9-(2-Ethyl-1-methyl-7-oxo-9-trifluoromethyl-6,7-dihydro-pyrrolo[3,2-f]quinolin-3-yl)-nonylsulfanyl]-propionic acid

(Compound 332, Structure 27 of Scheme VIII, where R¹=Trifluoromethyl, R³=Hydrogen, R⁴³=Methyl, R⁴⁵=Ethyl)

¹H NMR (500 MHz, DMSO) δ 7.79 (d, J=8.8 Hz, 1H), 7.18 (d, J=8.8 Hz, 1H), 6.92 (s, 1H), 4.18 (t, J=7.6 Hz, 2H), 2.79 (q, J=7.5 Hz, 2H), 2.62 (t, J=7.4 Hz, 2H), 2.45 (t, J=7.4 Hz, 2H), 2.33 (m, 2H), 2.22 (q, J=1.6 Hz, 3H), 1.64 (m, 2H), 1.46 (m, 2H), 1.34-1.19 (m, 10H), 1.16 (t, J=7.5 Hz, 3H).

### Example 24

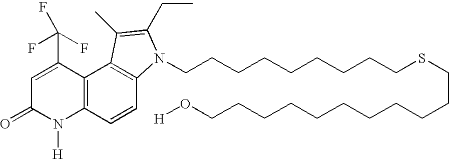

### 2-Ethyl-3-[9-(11-hydroxy-undecylsulfanyl)-nonyl]-1-methyl-9-trifluoromethyl-3,6-dihydro-pyrrolo[3,2-f]quinolin-7-one

(Compound 331, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.17 (d, J=8.9 Hz, 1H), 6.99 (s, 1H), 4.24 (t, J=7.6 Hz, 2H), 3.53 (m, 2H), 2.87 (q, J=7.6 Hz, 2H), 2.46 (t, J=7.0 Hz, 2H), 2.45 (t, J=7.0 Hz, 2H), 2.32 (q, J=2.2 Hz, 3H), 1.76 (m, 2H), 1.57-1.47 (m, 6H), 1.41-1.27 (m, 24H), 1.24 (t, J=7.6 Hz, 3H).

### Example 25

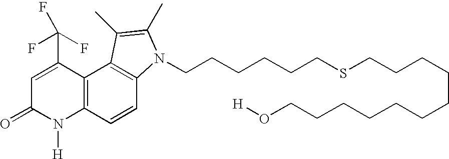

### 2-Ethyl-3-[6-(11-hydroxy-undecylsulfanyl)-hexyl]-1-methyl-9-trifluoromethyl-3,6-dihydro-pyrrolo[3,2-f]quinolin-7-one

(Compound 330, Structure 27 of Scheme VIII, where R¹=Trifluoromethyl, R³=Hydrogen, R⁴³=Methyl, R⁴⁵=Methyl)

¹H NMR (300 MHz, CD₃OD) δ 7.75 (d, J=8.8 Hz, 1H), 7.15 (d, J=8.8 Hz, 1H), 6.99 (s, 1H), 4.23 (t, J=7.4 Hz, 2H), 3.52 (t, J=6.5 Hz, 2H), 2.47-2.40 (m, 7H), 2.30 (s, 3H), 1.74 (t, J=7.0 Hz, 2H), 1.60-1.45 (m, 6H), 1.44-1.25 (m, 18H).

### Example 26

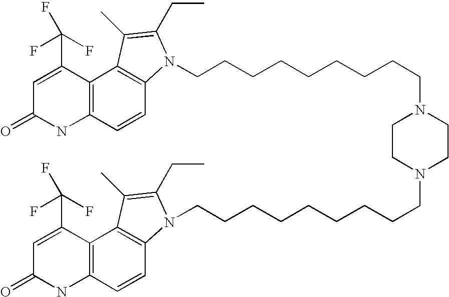

### N,N-1,4-Bis-(2-Ethyl-1-methyl-9-trifluoromethyl-3,6-dihydro-pyrrolo[3,2-f]quinolin-7-one-3-(9-nonanyl))piperazine

(Compound 329, Structure 27 of Scheme VIII, where R¹=Trifluoromethyl, R³=Hydrogen, R⁴³=Methyl, R⁴⁵=Ethyl)

¹H NMR (500 MHz, DMSO) δ 12.27 (s, 2H), 7.78 (d, J=8.9 Hz, 2H), 7.14 (d, J=8.9 Hz, 2H), 6.91 (s, 2H), 4.16 (t, J=7.3 Hz, 4H), 3.61 (t, J=6.3 Hz, 2H), 3.53 (t, J=6.3 Hz, 2H), 3.51 (t, J=6.3 Hz, 2H), 2.87 (t, J=6.3 Hz, 2H), 2.77 (q, J=7.5 Hz, 4H), 2.60 (m, 4H), 2.21 (m, 6H), 1.62 (m, 4H), 1.45 (m, 4H), 1.33-1.17 (m, 20H), 1.14 (t, J=7.5 Hz, 6H).

### Example 27

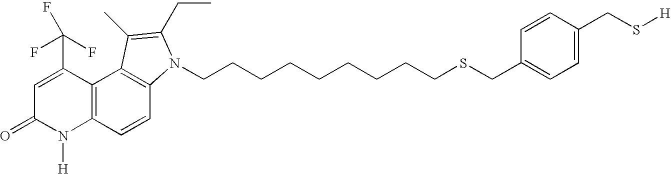

### 2-Ethyl-3-[9-(4-mercaptomethyl-benzylsulfanyl)-nonyl]-1-methyl-9-trifluoromethyl-3,6-dihydro-pyrrolo[3,2-f]quinolin-7-one

(Compound 328, Structure 27 of Scheme VIII, where R¹=Trifluoromethyl, R³=Hydrogen, R⁴³=Methyl, R⁴⁵=Ethyl)

¹H NMR (500 MHz, DMSO) δ 12.27 (s, 1H), 7.79 (d, J=8.9 Hz, 1H), 7.25 (d, J=8.2 Hz, 2H), 7.22 (d, J=8.2 Hz, 2H), 7.15 (d, J=8.9 Hz, 1H), 6.92 (s, 1H), 4.17 (t, J=7.5 Hz, 2H), 3.69 (d, J=7.8 Hz, 2H), 3.67 (s, 2H), 2.79 (q, J=7.5 Hz, 2H), 2.35 (t, J=7.3 Hz, 2H), 2.22 (q, J=1.5 Hz, 3H), 1.64 (m, 2H), 1.45 (m, 2H), 1.34-1.17 (m, 10H), 1.15 (t, J=7.5 Hz, 3H).

### Example 28

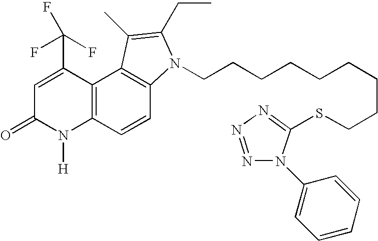

### 2-Ethyl-1-methyl-3-[9-(1-phenyl-1H-tetrazol-5-ylsulfanyl)-nonyl]-9-trifluoromethyl-3,6-dihydro-pyrrolo[3,2-f]quinolin-7-one

(Compound 327, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 11.23 (s, 1H), 7.78 (d, J=8.8 Hz, 1H), 7.71-7.63 (m, 5H), 7.26 (d, J=8.8 Hz, 1H), 6.92 (s, 1H), 4.28 (t, J=7.1 Hz, 2H), 3.37 (t, J=7.2 Hz, 2H), 2.89 (q, J=7.6 Hz, 2H), 2.32 (m, 3H), 1.84-1.74 (m, 4H), 1.48-1.30 (m, 10H), 1.23 (t, J=7.6 Hz, 3H).

### Example 29

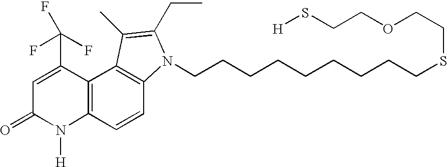

### 2-Ethyl-3-{9-[2-(2-mercapto-ethoxy)-ethylsulfanyl]-nonyl}-1-methyl-9-trifluoromethyl-3,6-dihydro-pyrrolo[3,2-f]quinolin-7-one

(Compound 326, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 7.78 (d, J=8.9 Hz, 1H), 7.25 (d, J=8.9 Hz, 1H), 6.91 (s, 1H), 4.28 (t, J=7.7 Hz, 2H), 3.61 (t, J=6.7 Hz, 2H), 3.57 (t, J=6.3 Hz, 2H), 2.89 (q, J=7.6 Hz, 3H), 2.67 (t, J=6.7 Hz, 2H), 2.66 (dt, J=8.0, 6.3 Hz, 2H), 2.56 (t, J=7.3 Hz, 2H), 2.32 (q, J=2.1 Hz, 3H), 1.86 (t, J=8.0 Hz, 1H), 1.78 (m, 2H), 1.56 (m, 2H), 1.47-1.28 (m, 10H), 1.24 (t, J=7.6 Hz, 3H).

### Example 30

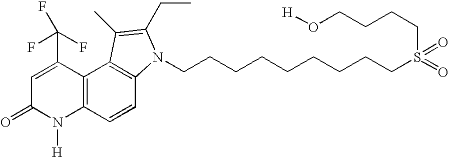

### 2-Ethyl-3-[9-(4-hydroxy-butane-1-sulfonyl)-nonyl]-1-methyl-9-trifluoromethyl-3,6-dihydro-pyrrolo[3,2-f]quinolin-7-one

(Compound 325, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.54 (s, 1H), 7.76 (d, J=8.8 Hz, 1H), 7.17 (d, J=8.8 Hz, 1H), 6.99 (s, 1H), 4.60 (s, 1H), 4.24 (t, J=7.6 Hz, 2H), 3.59 (t, J=6.2 Hz, 2H), 3.08 (t, J=7.9 Hz, 2H), 3.03 (t, J=7.9 Hz, 2H), 2.87 (q, J=7.6 Hz, 2H), 2.32 (q, J=2.0 Hz, 3H), 1.87 (m, 2H), 1.76 (m, 4H), 1.66 (m, 2H), 1.47-1.30 (m, 10H), 1.24 (t, J=7.6 Hz, 3H).

### Example 31

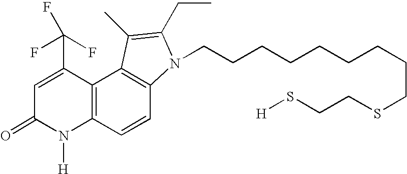

### 2-Ethyl-3-[9-(2-mercapto-ethylsulfanyl)-nonyl]-1-methyl-9-trifluoromethyl-3,6-dihydro-pyrrolo[3,2-f]quinolin-7-one

(Compound 324, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.7 Hz, 2H), 2.91-2.84 (m, 3H), 2.79 (m, 1H), 2.72-2.63 (m, 2H), 2.49 (q, J=7.4 Hz, 2H), 2.32 (q, J=2.0 Hz, 3H), 1.76 (m, 2H), 1.52 (m, 2H), 1.41-1.25 (m, 10H), 1.24 (t, J=7.4 Hz, 3H).

### Example 32

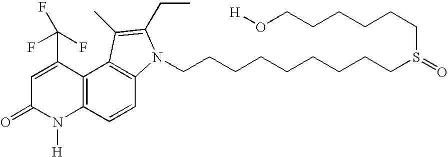

### 2-Ethyl-3-[9-(6-hydroxy-hexane-1-sulfinyl)-nonyl]-1-methyl-9-trifluoromethyl-3,6-dihydro-pyrrolo[3,2-f]quinolin-7-one

(Compound 323, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.6 Hz, 2H), 3.55 (t, J=6.5 Hz, 2H), 2.87 (q, J=7.6 Hz, 2H), 2.82-2.68 (m, 4H), 2.32 (q, J=2.0 Hz, 3H), 1.81-1.68 (m, 8H), 1.59-1.30 (m, 14H), 1.24 (t, J=7.6 Hz, 3H).

### Example 33

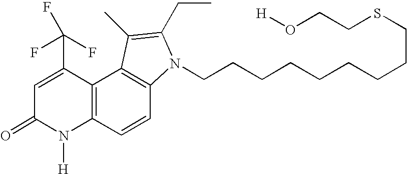

### 2-Ethyl-3-[9-(2-hydroxy-ethylsulfanyl)-nonyl]-1-methyl-9-trifluoromethyl-3,6-dihydro-pyrrolo[3,2-f]quinolin-7-one

(Compound 322, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.17 (d, J=8.9 Hz, 1H), 6.99 (s, 1H), 4.24 (t, J=7.6 Hz, 2H), 3.64 (t, J=6.8 Hz, 2H), 2.87 (q, J=7.5 Hz, 2H), 2.61 (t, J=6.8 Hz, 2H), 2.50 (t, J=7.3 Hz, 2H), 2.32 (q, J=2.0 Hz, 3H), 1.76 (m, 2H), 1.53 (m, 2H), 1.41-1.25 (m, 10H), 1.24 (t, J=7.5 Hz, 3H).

### Example 34

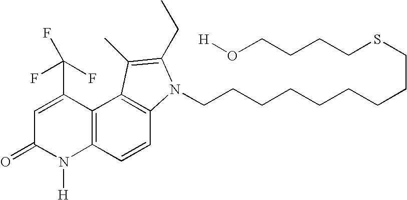

### 2-Ethyl-3-[9-(4-hydroxy-butylsulfanyl)-nonyl]-1-methyl-9-trifluoromethyl-3,6-dihydro-pyrrolo[3,2-f]quinolin-7-one

(Compound 321, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.6 Hz, 2H), 3.55 (m, 2H), 2.87 (q, J=7.6 Hz, 2H), 2.50 (m, 2H), 2.46 (t, J=7.3 Hz, 2H), 2.32 (q, J=2.0 Hz, 3H), 1.76 (m, 2H), 1.65-1.59 (m, 4H), 1.52 (m, 2H), 1.41-1.25 (m, 10H), 1.24 (t, J=7.6 Hz, 3H).

### Example 35

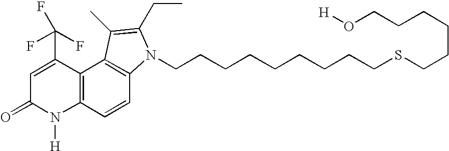

### 2-Ethyl-3-[9-(4-hydroxy-butylsulfanyl)-nonyl]-1-methyl-9-trifluoromethyl-3,6-dihydro-pyrrolo[3,2-f]quinolin-7-one

(Compound 320, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.7 Hz, 1H), 7.16 (d, J=8.7 Hz, 1H), 6.99 (s, 1H), 4.24 (t, J=7.5 Hz, 2H), 3.53 (t, J=6.6 Hz, 2H), 2.87 (q, J=7.5 Hz, 2H), 2.48 (t, J=7.2 Hz, 2H), 2.45 (t, J=7.2 Hz, 2H), 2.32 (q, J=2.1 Hz, 3H), 1.76 (m, 2H), 1.60-1.48 (m, 6H), 1.44-1.26 (m, 14H), 1.24 (t, J=7.5 Hz, 3H).

### Example 36

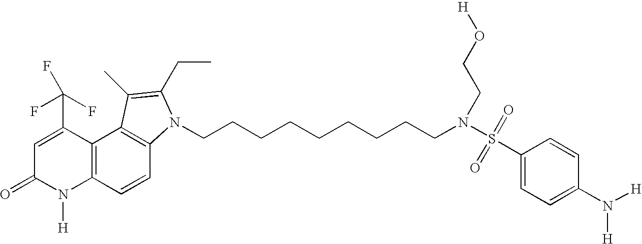

### 4-Amino-N-[9-(2-ethyl-1-methyl-7-oxo-9-trifluoromethyl-6,7-dihydro-pyrrolo[3,2-f]-quinolin-3-yl)-nonyl]-N-(2-hydroxy-ethyl)-benzenesulfonamide

(Compound 319, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.55 (s, 1H), 7.75 (d, J=8.8 Hz, 1H), 7.54 (d, J=9.1 Hz, 2H), 7.16 (d, J=8.8 Hz, 1H), 6.99 (s, 1H), 6.61 (d, J=9.1 Hz, 2H), 4.24 (t, J=7.5 Hz, 2H), 3.52 (t, J=6.0 Hz, 2H), 3.09 (t, J=7.1 Hz, 2H), 2.89 (t, J=6.0 Hz, 2H), 2.87 (q, J=7.4 Hz, 2H), 2.32 (q, J=2.1 Hz, 3H), 1.76 (m, 2H), 1.58 (m, 2H), 1.42-1.28 (m, 10H), 1.24 (t, J=7.4 Hz, 3H).

### Example 37

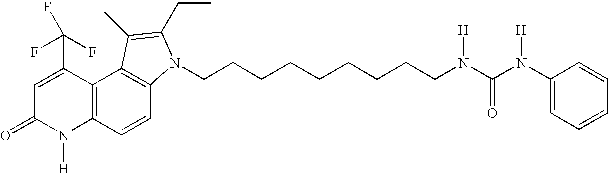

### 1-[9-(2-Ethyl-1-methyl-7-oxo-9-trifluoromethyl-6,7-dihydro-pyrrolo[3,2-f]quinolin-3-yl)-nonyl]-3-phenyl-urea

(Compound 318, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.32 (dd, J=8.6, 1.2 Hz, 2H), 7.22 (dd, J=8.6, 7.4 Hz, 2H), 7.16 (d, J=8.8 Hz, 1H), 6.99 (s, 1H), 6.94 (tt, J=7.4, 1.2 Hz, 1H), 4.23 (t, J=7.4 Hz, 2H), 3.16 (t, J=7.0 Hz, 2H), 2.86 (q, J=7.5 Hz, 2H), 2.31 (q, J=2.0 Hz, 3H), 1.75 (m, 2H), 1.49 (m, 2H), 1.40-1.30 (m, 10H), 1.23 (t, J=7.5 Hz, 3H).

### Example 38

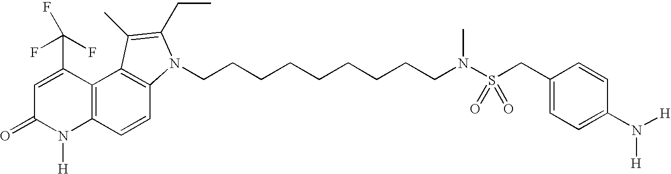

### C-(4-Aminophenyl)-N-[9-(2-ethyl-1-methyl-7-oxo-9-trifluoromethyl-6,7-dihydro-pyrrolo[3,2-f]quinolin-3-yl)-nonanyl]-N-methyl-methanesulfonamide

(Compound 317, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.28 (s, 1H), 7.75 (d, J=8.8 Hz, 1H), 7.16 (d, J=8.8 Hz, 1H), 7.12 (d, J=8.6 Hz, 2H), 6.98 (s, 1H), 6.58 (d, J=8.6 Hz, 2H), 4.23 (t, J=7.4 Hz, 2H), 4.14 (s, 2H), 3.02 (t, J=7.2 Hz, 2H), 2.87 (q, J=7.6 Hz, 2H), 2.60 (s, 3H), 2.32 (q, J=2.0 Hz, 3H), 1.75 (m, 2H), 1.55 (m, 2H), 1.41-1.28 (m, 10H), 1.24 (t, J=7.6 Hz, 3H).

### Example 39

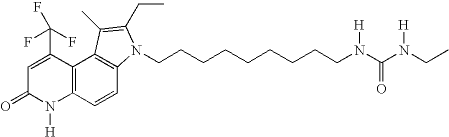

### 1-Ethyl-3-[9-(2-ethyl-1-methyl-7-oxo-9-trifluoromethyl-6,7-dihydro-pyrrolo[3,2-f]-quinolin-3-yl)-nonyl]-urea

(Compound 316, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 5.82 (m, 1H), 4.24 (t, J=7.6 Hz, 2H), 3.12 (q, J=7.1 Hz, 2H), 3.06 (m, 2H), 2.87 (q, J=7.6 Hz, 2H), 2.32 (q, J=2.2 Hz, 3H), 1.75 (m, 2H), 1.43 (m, 2H), 1.40-1.27 (m, 10H), 1.24 (t, J=7.6 Hz, 3H), 1.07 (t, J=7.1 Hz, 3H).

### Example 40

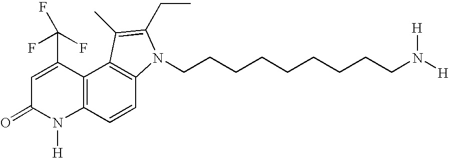

### 3-(9-Amino-nonyl)-2-ethyl-1-methyl-9-trifluoromethyl-3,6-dihydro-pyrrolo[3,2-f]-quinolin-7-one

(Compound 315, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.65 (d, J=8.8 Hz, 1H), 7.06 (d, J=8.8 Hz, 1H), 6.90 (s, 1H), 4.14 (t, J=7.6 Hz, 2H), 2.77 (q, J=7.6 Hz, 2H), 2.62 (t, J=7.4 Hz, 2H), 2.22 (q, J=2.0 Hz, 3H), 1.65 (m, 2H), 1.41 (m, 2H), 1.31-1.16 (m, 10H), 1.14 (t, J=7.6 Hz, 3H).

### Example 41

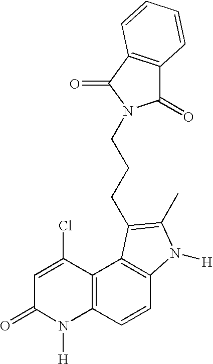

### 2-[3-(9-Chloro-2-methyl-7-oxo-6,7-dihydro-3H-pyrrolo[3,2-f]quinolin-1-yl)-propyl]-isoindole-1,3-dione

(Compound 194, Structure 26 of Scheme VIII, where R¹=Chloro, R³=Hydrogen, R⁴⁵=Methyl)

¹H NMR (500 MHz, CD₃OD) δ 7.73-7.70 (m, 2H), 7.65-7.62 (m, 2H), 7.24 (d, J=8.5 Hz, 1H), 6.79 (d, J=8.5 Hz, 1H), 6.68 (s, 1H), 3.61 (t, J=6.3 Hz, 2H), 3.15 (t, J=7.0 Hz, 2H), 2.44 (s, 3H), 2.05 (m, 2H).

### Example 42

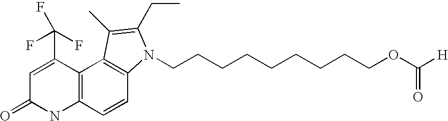

### Formic acid 9-(2-ethyl-1-methyl-7-oxo-9-trifluoromethyl-6,7-dihydro-pyrrolo[3,2-f]-quinolin-3-yl)-nonyl ester

(Compound 314, Structure 27 of Scheme VIII, where R¹=Trifluoromethyl, R³=Hydrogen, R⁴³=Methyl, R⁴⁵=Ethyl)

¹H NMR (500 MHz, Acetone-d₆) δ 11.25 (s, 1H), 8.11 (s, 1H), 7.79 (d, J=8.8 Hz, 1H), 7.25 (d, J=8.8 Hz, 1H), 6.93 (s, 1H), 4.28 (t, J=7.8 Hz, 2H), 4.11 (t, J=6.8 Hz, 2H), 2.89 (q, J=7.6 Hz, 2H), 2.32 (q, J=2.1 Hz, 3H), 1.79 (m, 2H), 1.63 (m, 2H), 1.48-1.28 (m, 10H), 1.24 (t, J=7.6 Hz, 3H).

### Example 43

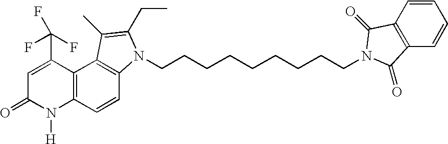

### 2-[9-(2-Ethyl-1-methyl-7-oxo-9-trifluoromethyl-6,7-dihydro-pyrrolo[3,2-f]quinolin-3-yl)-nonyl]-isoindole-1,3-dione

(Compound 313, Structure 27 of Scheme VIII, where R¹=Trifluoromethyl, R³=Hydrogen, R⁴³=Methyl, R⁴⁵=Ethyl)

¹H NMR (500 MHz, DMSO) δ 12.27 (s, 1H), 7.87-7.81 (m, 4H), 7.79 (d, J=8.8 Hz, 1H), 7.14 (d, J=8.8 Hz, 1H), 6.91 (s, 1H), 4.16 (t, J=7.6 Hz, 2H), 3.53 (t, J=7.1 Hz, 2H), 2.78 (q, J=7.6 Hz, 2H), 2.22 (q, J=1.7 Hz, 3H), 1.62 (m, 2H), 1.55 (m, 2H), 1.34-1.19 (m, 10H), 1.14 (t, J=7.6 Hz, 3H).

### Example 44

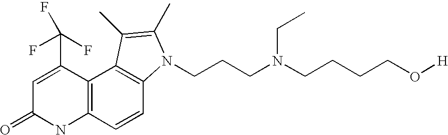

### 3-{3-[Ethyl-(4-hydroxy-butyl)-amino]-propyl}-1,2-dimethyl-9-trifluoromethyl-3,6-dihydro-pyrrolo[3,2-f]quinolin-7-one

(Compound 312, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.83 (d, J=8.8 Hz, 1H), 7.18 (d, J=8.8 Hz, 1H), 7.00 (s, 1H), 4.33 (t, J=7.2 Hz, 2H), 3.52 (t, J=6.0 Hz, 2H), 2.85-2.77 (m, 4H), 2.74 (m, 2H), 2.46 (s, 3H), 2.30 (q, J=1.5 Hz, 3H), 2.02 (m, 2H), 1.62-1.48 (m, 4H), 1.10 (t, J=7.2 Hz, 3H).

### Example 45

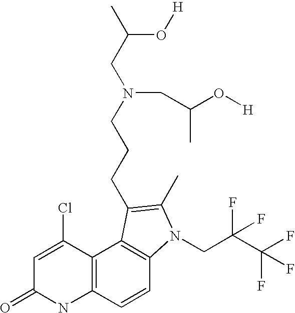

### 1-{3-[Bis-(2-hydroxy-propyl)-amino]-propyl}-9-chloro-2-methyl-3-(2,2,3,3,3-pentafluoro-propyl)-3,6-dihydro-pyrrolo[3,2-f]quinolin-7-one

(Compound 193, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, CD₃OD) δ 7.78 (d, J=8.8 Hz, 1H), 7.22 (d, J=8.8 Hz, 1H), 6.80 (s, 1H), 5.14 (t, J=15.7 Hz, 2H), 3.68 (m, 2H), 3.15-3.04 (m, 2H), 2.59-2.36 (m, 4H), 2.49 (s, 3H), 2.31 (m, 2H), 1.72 (m, 2H), 1.03 (d, J=6.2 Hz, 3H), 1.02 (d, J=6.2 Hz, 3H).

### Example 46

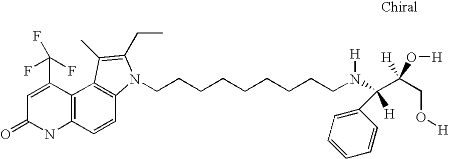

### 3-[9-((1S,2R)-2,3-Dihydroxy-1-phenyl-propylamino)-nonyl]-2-ethyl-1-methyl-9-trifluoromethyl-3,6-dihydro-pyrrolo[3,2-f]quinolin-7-one

(Compound 311, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.42 (m, 2H), 7.37 (m, 2H), 7.31 (tt, J=7.2, 1.4 Hz, 1H), 7.16 (d, J=8.8 Hz, 1H), 6.99 (s, 1H), 4.76 (d, J=8.5 Hz, 1H), 4.24 (t, J=7.6 Hz, 2H), 3.58 (dd, J=12.0, 3.9 Hz, 1H), 3.36 (dd, J=12.0, 4.7 Hz, 1H), 3.10 (m, 1H), 2.96 (ddd, J=11.7, 9.4, 6.2 Hz, 1H), 2.87 (m, 1H), 2.87 (q, J=7.6 Hz, 2H), 2.32 (q, J=2.0 Hz, 3H), 1.76 (m, 2H), 1.61 (m, 2H), 1.41-1.28 (m, 10H), 1.24 (t, J=7.6 Hz, 3H).

### Example 47

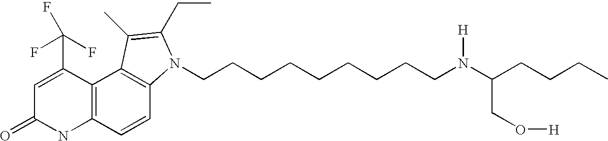

### 2-Ethyl-3-[9-(1-hydroxymethyl-pentylamino)-nonyl]-1-methyl-9-trifluoromethyl-3,6-dihydro-pyrrolo[3,2-f]quinolin-7-one

(Compound 310, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.5 Hz, 2H), 3.79 (dd, J=12.2, 3.6 Hz, 1H), 3.61 (dd, J=12.2, 4.9 Hz, 1H), 3.06 (m, 1H), 2.95 (m, 2H), 2.87 (q, J=7.5 Hz, 2H), 2.32 (q, J=2.1 Hz, 3H), 1.76 (qn, J=7.5 Hz, 2H), 1.69-1.57 (m, 4H), 1.43-1.30 (m, 14H), 1.24 (t, J=7.5 Hz, 3H), 0.95 (t, J=7.0 Hz, 3H).

### Example 48

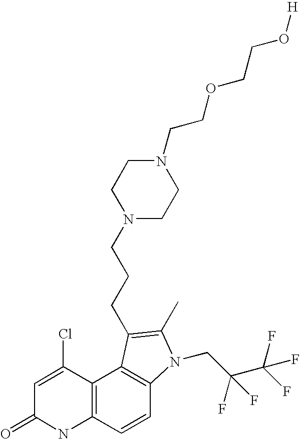

### 9-Chloro-1-(3-{4-[2-(2-hydroxy-ethoxy)-ethyl]-piperazin-1-yl}-propyl)-2-methyl-3-(2,2,3,3,3-pentafluoro-propyl)-3,6-dihydro-pyrrolo[3,2-f]quinolin-7-one

(Compound 192, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, CD₃OD) δ 7.78 (d, J=9.1 Hz, 1H), 7.22 (d, J=9.1 Hz, 1H), 6.80 (s, 1H), 5.15 (t, J=15.7 Hz, 2H), 3.64 (m, 2H), 3.60 (t, J=5.4 Hz, 2H), 3.51 (m, 2H), 3.16 (t, J=7.5 Hz, 2H), 2.74-2.33 (m, 8H), 2.60 (m, 2H), 2.49 (s, 3H), 2.30 (m, 2H), 1.76 (m, 2H).

### Example 49

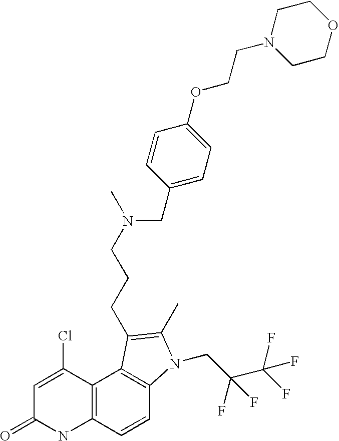

### 9-Chloro-2-methyl-1-(3-{methyl-[4-(2-morpholin-4-yl-ethoxy)-benzyl]-amino}-propyl)-3-(2,2,3,3,3-pentafluoro-propyl)-3,6-dihydro-pyrrolo[3,2-f]quinolin-7-one

(Compound 191, Structure 27 of Scheme VIII, where R¹=Chloro, R³=Hydrogen, R⁴⁵=methyl, R⁴⁶=2,2,3,3,3-Pentafluoropropyl)

¹H NMR (500 MHz, CD₃OD) δ 7.77 (d, J=8.8 Hz, 1H), 7.21 (d, J=8.8 Hz, 1H), 7.09 (d, J=8.5 Hz, 2H), 6.83 (d, J=8.5 Hz, 2H), 6.80 (s, 1H), 5.13 (t, J=15.9 Hz, 2H), 4.12 (t, J=5.5 Hz, 2H), 3.72 (m, 4H), 3.44 (s, 2H), 3.13 (t, J=7.4 Hz, 2H), 2.81 (t, J=5.5 Hz, 2H), 2.61 (m, 4H), 2.46 (s, 3H), 2.32 (m, 2H), 2.18 (s, 3H), 1.77 (m, 2H).

### Example 50

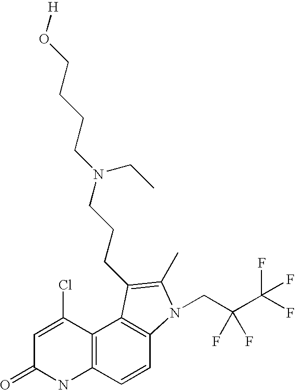

### 9-Chloro-1-{3-[ethyl-(4-hydroxy-butyl)-amino]-propyl}-2-methyl-3-(2,2,3,3,3-pentafluoro-propyl)-3,6-dihydro-pyrrolo[3,2-f]quinolin-7-one

(Compound 190, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, CD₃OD) δ 7.82 (d, J=8.9 Hz, 1H), 7.25 (d, J=8.9 Hz, 1H), 6.84 (s, 1H), 5.18 (t, J=15.9 Hz, 2H), 3.52 (t, J=6.0 Hz, 2H), 3.23 (t, J=7.7 Hz, 2H), 2.99 (m, 2H), 2.91 (m, 4H), 2.52 (s, 3H), 1.88 (m, 2H), 1.59 (m, 2H), 1.49 (m, 2H), 1.14 (t, J=7.2 Hz, 3H).

### Example 51

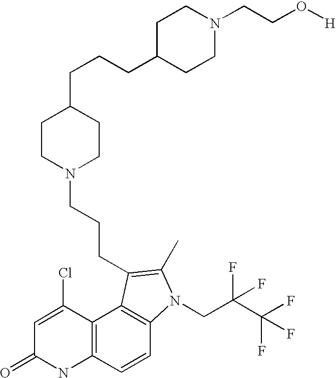

### 9-Chloro-1-[3-(4-{3-[1-(2-hydroxy-ethyl)-piperidin-4-yl]-propyl}-piperidin-1-yl)-propyl]-2-methyl-3-(2,2,3,3,3-pentafluoro-propyl)-3,6-dihydro-pyrrolo[3,2-f]quinolin-7-one

(Compound 189, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, DMSO) δ 11.99 (s, 1H), 7.83 (d, J=9.0 Hz, 1H), 7.17 (d, J=9.0 Hz, 1H), 6.70 (s, 1H), 5.32 (t, J=16.5 Hz, 2H), 4.31 (t, J=5.3 Hz, 1H), 4.04 (m, 4H), 3.45 (dt, J=5.3, 6.4 Hz, 2H), 3.02 (t, J=7.5 Hz, 2H), 2.80 (m, 2H), 2.69 (m, 2H), 2.42 (s, 3H), 2.32 (t, J=6.4 Hz, 2H), 2.08 (t, J=6.9 Hz, 2H), 1.86 (m, 2H), 1.71 (m, 2H), 1.61-1.52 (m, 6H), 1.24 (m, 2H), 1.16-0.98 (m, 6H).

### Example 52

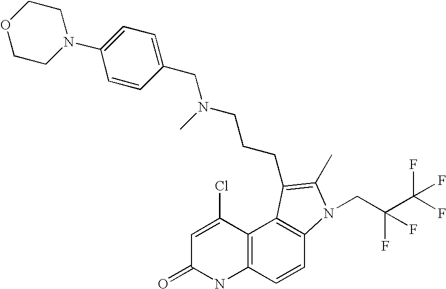

### 9-Chloro-2-methyl-1-{3-[methyl-(4-morpholin-4-yl-benzyl)-amino]-propyl}-3-(2,2,3,3,3-pentafluoro-propyl)-3,6-dihydro-pyrrolo[3,2-f]quinolin-7-one

(Compound 188, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, CD₃OD) δ 7.77 (d, J=8.9 Hz, 1H), 7.22 (d, J=8.9 Hz, 1H), 7.07 (d, J=8.6 Hz, 2H), 6.86 (d, J=8.6 Hz, 2H), 6.80 (s, 1H), 5.12 (t, J=15.7 Hz, 2H), 3.83 (m, 4H), 3.43 (s, 2H), 3.14-3.10 (m, 6H), 2.46 (s, 3H), 2.34 (m, 2H), 2.19 (s, 3H), 1.77 (m, 2H).

### Example 53

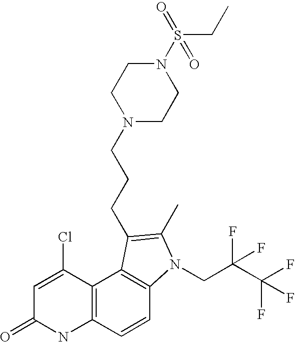

### 9-Chloro-1-[3-(4-ethanesulfonyl-piperazin-1-yl)-propyl]-2-methyl-3-(2,2,3,3,3-pentafluoro-propyl)-3,6-dihydro-pyrrolo[3,2-f]quinolin-7-one

(Compound 187, Structure 27 of Scheme VIII, where R¹=Chloro, R³=Hydrogen, R⁴⁵=Methyl, R⁴⁶=2,2,3,3,3-Pentafluoropropyl)

¹H NMR (500 MHz, CD₃OD) δ 7.78 (d, J=9.0 Hz, 1H), 7.22 (d, J=9.0 Hz, 1H), 6.81 (s, 1H), 5.15 (t, J=15.9 Hz, 2H), 3.21 (m, 4H), 3.18 (t, J=7.4 Hz, 2H), 3.00 (q, J=7.4 Hz, 2H), 2.49 (s, 3H), 2.43 (m, 4H), 2.29 (m, 2H), 1.75 (qn, J=7.4 Hz, 2H), 1.29 (t, J=7.4 Hz, 3H).

### Example 54

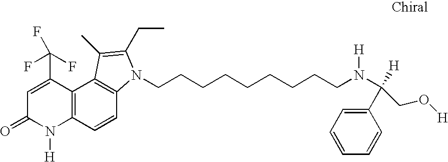

### 2-Ethyl-3-[9-(2-hydroxy-1-phenyl-ethylamino)-nonyl]-1-methyl-9-trifluoromethyl-3,6-dihydro-pyrrolo[3,2-f]quinolin-7-one

(Compound 309, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.35-7.30 (m, 4H), 7.26 (m, 1H), 7.16 (d, J=8.8 Hz, 1H), 6.99 (s, 1H), 4.23 (t, J=7.4 Hz, 2H), 3.79 (m, 1H), 3.67 (dd, J=10.9, 4.6 Hz, 1H), 3.61 (dd, J=10.9, 8.3 Hz, 1H), 2.86 (q, J=7.5 Hz, 2H), 2.45 (m, 2H), 2.31 (q, J=2.2 Hz, 3H), 1.74 (m, 2H), 1.47 (m, 2H), 1.39-1.24 (m, 10H), 1.23 (t, J=7.5 Hz, 3H).

### Example 55

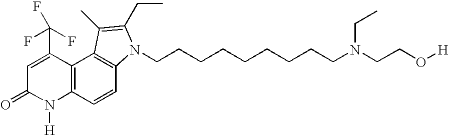

### 2-Ethyl-3-{9-[ethyl-(2-hydroxy-ethyl)-amino]-nonyl}-1-methyl-9-trifluoromethyl-3,6-dihydro-pyrrolo[3,2-f]quinolin-7-one

(Compound 308, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 7.00 (s, 1H), 4.25 (t, J=7.4 Hz, 2H), 3.78 (t, J=5.4 Hz, 2H), 3.16-3.03 (m, 4H), 2.98 (m, 2H), 2.87 (q, J=7.6 Hz, 2H), 2.32 (q, J=2.0 Hz, 3H), 1.76 (qn, J=7.4 Hz, 2H), 1.62 (m, 2H), 1.41-1.30 (m, 10H), 1.24 (t, J=7.6 Hz, 6H).

### Example 56

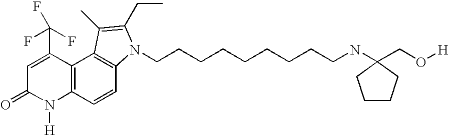

### 2-Ethyl-3-[9-(1-hydroxymethyl-cyclopentylamino)-nonyl]-1-methyl-9-trifluoromethyl-3,6-dihydro-pyrrolo[3,2-f]quinolin-7-one

(Compound 307, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 7.00 (s, 1H), 4.25 (t, J=7.6 Hz, 2H), 3.53 (s, 2H), 2.90 (t, J=7.9 Hz, 2H), 2.87 (q, J=7.6 Hz, 2H), 2.32 (q, J=2.2 Hz, 3H), 1.88-1.74 (m, 6H), 1.73-1.59 (m, 6H), 1.42-1.30 (m, 10H), 1.24 (t, J=7.6 Hz, 3H).

### Example 57

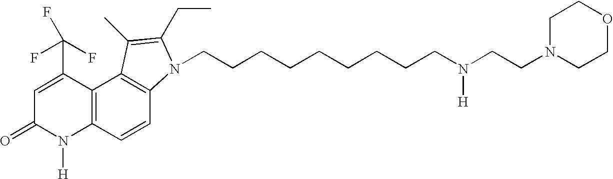

### 2-Ethyl-1-methyl-3-[9-(2-morpholin-4-yl-ethylamino)-nonyl]-9-trifluoromethyl-3,6-dihydro-pyrrolo[3,2-f]quinolin-7-one

(Compound 306, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.9 Hz, 1H), 7.18 (d, J=8.9 Hz, 1H), 6.99 (s, 1H), 4.24 (t, J=7.5 Hz, 2H), 3.68 (m, 4H), 2.87 (q, J=7.6 Hz, 2H), 2.82 (t, J=6.3 Hz, 2H), 2.69 (t, J=7.7 Hz, 2H), 2.54 (t, J=6.3 Hz, 2H), 2.47 (m, 4H), 2.31 (q, J=2.0 Hz, 3H), 1.75 (qn, J=7.5 Hz, 2H), 1.55 (m, 2H), 1.41-1.27 (m, 10H), 1.24 (t, J=7.6 Hz, 3H).

### Example 58

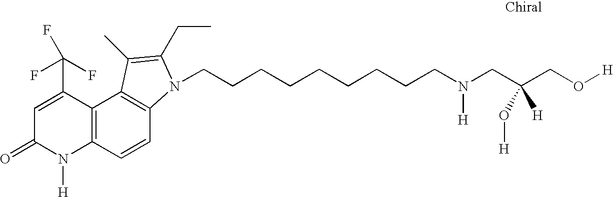

### 3-[9-(2,3-Dihydroxy-propylamino)-nonyl]-2-ethyl-1-methyl-9-trifluoromethyl-3,6-dihydro-pyrrolo[3,2-f]quinolin-7-one

(Compound 305, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 4.24 (t, J=7.4 Hz, 2H), 3.87 (m, 1H), 3.58 (dd, J=11.2, 4.6 Hz, 1H), 3.52 (dd, J=11.2, 5.6 Hz, 1H), 3.11 (dd, J=12.7, 3.2 Hz, 1H), 2.98-2.92 (m, 3H), 2.87 (q, J=7.6 Hz, 2H), 2.32 (q, J=2.0 Hz, 3H), 1.76 (qn, J=7.4 Hz, 2H), 1.65 (m, 2H), 1.41-1.30 (m, 10H), 1.24 (t, J=7.6 Hz, 3H).

### Example 59

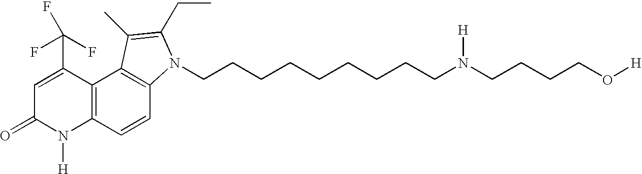

### 2-Ethyl-3-[9-(4-hydroxy-butylamino)-nonyl]-1-methyl-9-trifluoromethyl-3,6-dihydro-pyrrolo[3,2-f]quinolin-7-one

(Compound 304, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 7.00 (s, 1H), 4.25 (t, J=7.6 Hz, 2H), 3.60 (t, J=6.1 Hz, 2H), 2.98 (m, 2H), 2.93 (m, 2H), 2.87 (q, J=7.6 Hz, 2H), 2.32 (q, J=2.0 Hz, 3H), 1.80-1.71 (m, 4H), 1.66-1.57 (m, 4H), 1.41-1.29 (m, 10H), 1.24 (t, J=7.6 Hz, 3H).

### Example 60

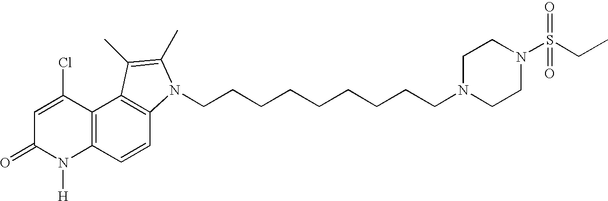

### 9-Chloro-3-[9-(4-ethanesulfonyl-piperazin-1-yl)-nonyl]-1,2-dimethyl-3,6-dihydro-pyrrolo[3,2-f]quinolin-7-one

(Compound 303, Structure 27 of Scheme VIII, where R¹=chloro, R³=Hydrogen, R⁴³=Methyl, R⁴⁵=Methyl)

¹H NMR (500 MHz, CD₃OD) δ 7.73 (d, J=8.8 Hz, 1H), 7.13 (d, J=8.8 Hz, 1H), 6.74 (s, 1H), 4.24 (t, J=7.3 Hz, 2H), 3.27 (m, 4H), 3.03 (q, J=7.3 Hz, 2H), 2.54 (s, 3H), 2.50 (m, 4H), 2.45 (s, 3H), 2.34 (m, 2H), 1.73 (qn, J=7.3 Hz, 2H), 1.46 (m, 2H), 1.37-1.25 (m, 10H), 1.31 (t, J=7.3 Hz, 3H).

### Example 61

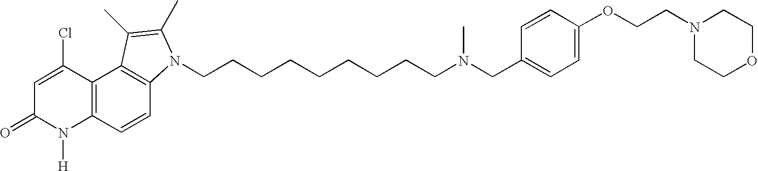

### 9-Chloro-2-dimethyl-3-(9-{methyl-[4-(2-morpholin-4-yl-ethoxy)-benzyl]-amino}-nonyl)-3,6-dihydro-pyrrolo[3,2-f]quinolin-7-one

(Compound 302, Structure 27 of Scheme VIII, where R¹=Chloro, R³=Hydrogen, R⁴³=Methyl, R⁴⁵=Methyl)

¹H NMR (500 MHz, CD₃OD) δ 7.71 (d, J=8.8 Hz, 1H), 7.24 (d, J=8.5 Hz, 2H), 7.12 (d, J=8.8 Hz, 1H), 6.91 (d, J=8.5 Hz, 2H), 6.72 (s, 1H), 4.22 (t, J=7.2 Hz, 2H), 4.11 (t, J=5.5 Hz, 2H), 3.69 (m, 4H), 3.64 (s, 2H), 2.77 (t, J=5.5 Hz, 2H), 2.57 (m, 4H), 2.53 (s, 3H), 2.51 (m, 2H), 2.44 (s, 3H), 2.32 (s, 3H), 1.71 (qn, J=7.2 Hz, 2H), 1.53 (m, 2H), 1.36-1.22 (m, 10H).

### Example 62

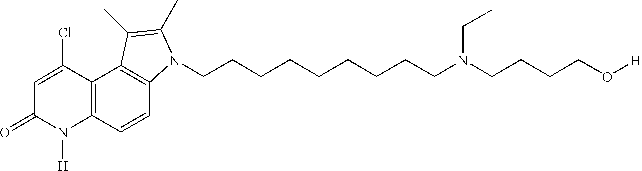

### 9-Chloro-3-{9-[ethyl-(4-hydroxy-butyl)-amino]-nonyl}-1,2-dimethyl-3,6-dihydro-pyrrolo[3,2-f]quinolin-7-one

(Compound 301, Structure 27 of Scheme VIII, where R¹=Chloro, R³=Hydrogen, R⁴³=Methyl, R⁴⁵=Methyl)

¹H NMR (500 MHz, CD₃OD) δ 7.73 (d, J=8.8 Hz, 1H), 7.13 (d, J=8.8 Hz, 1H), 6.74 (s, 1H), 4.25 (t, J=7.2 Hz, 2H), 3.60 (t, J=6.0 Hz, 2H), 3.12 (q, J=7.3 Hz, 2H), 3.05 (m, 2H), 3.00 (m, 2H), 2.54 (s, 3H), 2.45 (s, 3H), 1.79-1.70 (m, 4H), 1.66-1.56 (m, 4H), 1.37-1.29 (m, 10H), 1.26 (t, J=7.3 Hz, 3H).

### Example 63

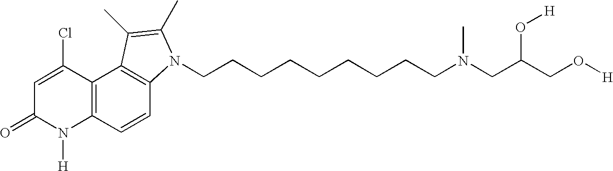

### (±)-9-Chloro-3-{9-[(2,3-dihydroxy-propyl)-methyl-amino]-nonyl}-1,2-dimethyl-3,6-dihydro-pyrrolo[3,2-f]quinolin-7-one

(Compound 300, Structure 27 of Scheme VIII, where R¹=Chloro, R³=Hydrogen, R⁴³=Methyl, R⁴⁵=Methyl)

¹H NMR (500 MHz, CD₃OD) δ 7.73 (d, J=8.8 Hz, 1H), 7.14 (d, J=8.8 Hz, 1H), 6.74 (s, 1H), 4.24 (t, J=7.3 Hz, 2H), 3.96 (m, 1H), 3.56 (dd, J=11.2, 4.8 Hz, 1H), 3.52 (dd, J=11.2, 5.5 Hz, 1H), 3.13-2.98 (m, 4H), 2.80 (s, 3H), 2.53 (s, 3H), 2.44 (s, 3H), 1.77-1.61 (m, 4H), 1.37-1.29 (m, 10H).

### Example 64

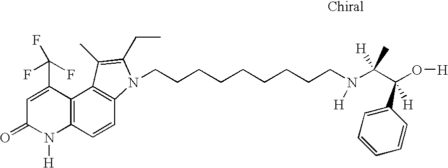

### 2-Ethyl-3-[9-((1S,2S)-2-hydroxy-1-methyl-2-phenyl-ethylamino)-nonyl]-1-methyl-9-trifluoromethyl-3,6-dihydro-pyrrolo[3,2-f]quinolin-7-one

(Compound 299, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.8 Hz, 1H), 7.42-7.35 (m, 4H), 7.29 (m, 1H), 7.17 (d, J=8.8 Hz, 1H), 7.00 (s, 1H), 5.08 (d, J=3.2 Hz, 1H), 4.25 (t, J=7.4 Hz, 2H), 3.40 (m, 1H), 3.03 (m, 2H), 2.87 (q, J=7.5 Hz, 2H), 2.32 (q, J=2.2 Hz, 3H), 1.77 (m, 2H), 1.69 (m, 2H), 1.43-1.32 (m, 10H), 1.24 (t, J=7.5 Hz, 3H), 1.04 (d, J=6.6 Hz, 3H).

### Example 65

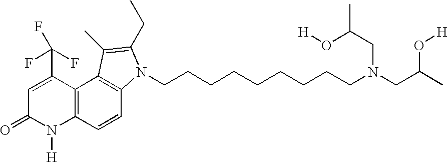

### (±)-3-{9-[Bis-(2-hydroxy-propyl)-amino]-nonyl}-2-ethyl-1-methyl-9-trifluoromethyl-3,6-dihydro-pyrrolo[3,2-f]quinolin-7-one

(Compound 298, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.17 (d, J=8.8 Hz, 1H), 7.00 (s, 1H), 4.24 (t, J=7.6 Hz, 1H), 4.08-3.89 (m, 2H), 3.04 (m, 1H), 2.95-2.60 (m, 5H), 2.87 (q, J=7.5 Hz, 2H), 2.32 (q, J=2.2 Hz, 3H), 1.76 (m, 2H), 1.57 (m, 2H), 1.40-1.28 (m, 10H), 1.24 (t, J=7.5 Hz, 3H), 1.16 (d, J=6.1 Hz, 6H).

### Example 66

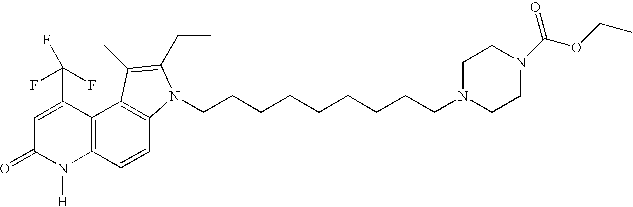

### 4-[9-(2-Ethyl-1-methyl-7-oxo-9-trifluoromethyl-6,7-dihydro-pyrrolo[3,2-f]quinolin-3-yl)-nonyl]-piperazine-1-carboxylic acid ethyl ester

(Compound 297, Structure 27 of Scheme VIII, where R¹=Trifluoromethyl, R³=Hydrogen, R⁴³=Methyl, R⁴⁵=Ethyl)

¹H NMR (500 MHz, CD₃OD) 7.75 (d, J=8.8 Hz, 1H), 7.16 (d, J=8.8 Hz, 1H), 6.99 (s, 1H), 4.24 (t, J=7.4 Hz, 2H), 4.11 (q, J=7.1 Hz, 2H), 3.46 (m, 4H), 2.87 (q, J=7.5 Hz, 2H), 2.40 (m, 4H), 2.31 (q, J=2.2 Hz, 3H), 2.31 (m, 2H), 1.75 (qn, J=7.1 Hz, 2H), 1.47 (m, 2H), 1.40-1.26 (m, 10H), 1.25 (t, J=7.1 Hz, 3H), 1.24 (t, J=7.5 Hz, 3H).

### Example 67

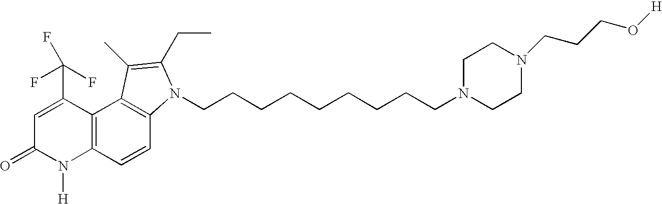

### 2-Ethyl-3-{9-[4-(3-hydroxy-propyl)-piperazin-1-yl]-nonyl}-1-methyl-9-trifluoromethyl-3,6-dihydro-pyrrolo[3,2-f]quinolin-7-one

(Compound 296, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.6 Hz, 2H), 3.61 (t, J=6.2 Hz, 2H), 2.87 (q, J=7.6 Hz, 2H), 2.79-2.35 (m, 8H), 2.48 (m, 2H), 2.32 (m, 2H), 2.31 (q, J=2.0 Hz, 3H), 1.79-1.69 (m, 4H), 1.46 (m, 2H), 1.40-1.26 (m, 10H), 1.24 (t, J=7.6 Hz, 3H).

### Example 68

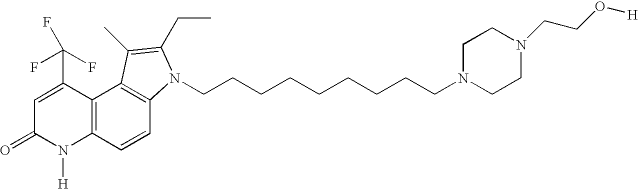

### 2-Ethyl-3-{9-[4-(2-hydroxy-ethyl)-piperazin-1-yl]-nonyl}-1-methyl-9-trifluoromethyl-3,6-dihydro-pyrrolo[3,2-f]quinolin-7-one

(Compound 295, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.17 (d, J=8.8 Hz, 1H), 7.00 (s, 1H), 4.24 (t, J=7.4 Hz, 2H), 3.68 (t, J=6.0 Hz, 2H), 2.87 (q, J=7.6 Hz, 2H), 2.71-2.36 (m, 8H), 2.53 (t, J=6.0 Hz, 2H), 2.32 (q, J=1.9 Hz, 3H), 2.31 (m, 2H), 1.75 (m, 2H), 1.46 (m, 2H), 1.40-1.26 (m, 10H), 1.24 (t, J=7.6 Hz, 3H).

### Example 69

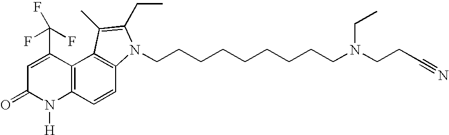

### 3-{Ethyl-[9-(2-ethyl-1-methyl-7-oxo-9-trifluoromethyl-6,7-dihydro-pyrrolo[3,2-f]-quinolin-3-yl)-nonyl]-amino}-propionitrile

(Compound 294, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.17 (d, J=8.9 Hz, 1H), 7.00 (s, 1H), 4.24 (t, J=7.5 Hz, 2H), 2.87 (q, J=7.6 Hz, 2H), 2.77 (t, J=6.8 Hz, 2H), 2.54 (m, 4H), 2.44 (m, 2H), 2.32 (q, J=2.1 Hz, 3H), 1.76 (m, 2H), 1.46-1.26 (m, 12H), 1.24 (t, J=7.6 Hz, 3H), 1.03 (t, J=7.1 Hz, 3H).

### Example 70

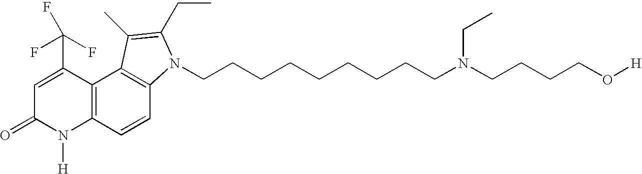

### 2-Ethyl-3-{9-[ethyl-(4-hydroxy-butyl)-amino]-nonyl}-1-methyl-9-trifluoromethyl-3,6-dihydro-pyrrolo[3,2-f]quinolin-7-one

(Compound 293, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.8 Hz, 1H), 7.17 (d, J=8.8 Hz, 1H), 7.00 (s, 1H), 4.24 (t, J=7.4 Hz, 2H), 3.55 (t, J=6.0 Hz, 2H), 2.87 (q, J=7.6 Hz, 2H), 2.64 (m, 2H), 2.58-2.49 (m, 4H), 2.32 (q, J=2.1 Hz, 3H), 1.76 (m, 2H), 1.63-1.43 (m, 6H), 1.40-1.26 (m, 10H), 1.24 (t, J=7.6 Hz, 3H), 1.07 (t, J=7.1 Hz, 3H).

### Example 71

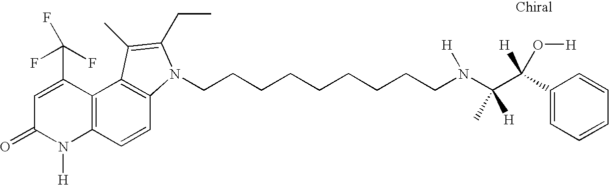

### 2-Ethyl-3-[9-(2-hydroxy-1-methyl-2-phenyl-ethylamino)-nonyl]-1-methyl-9-trifluoromethyl-3,6-dihydro-pyrrolo[3,2-f]quinolin-7-one

(Compound 292, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.8 Hz, 1H), 7.42-7.36 (m, 4H), 7.30 (m, 1H), 7.17 (d, J=8.8 Hz, 1H), 7.00 (s, 1H), 5.09 (d, J=3.2 Hz, 1H), 4.25 (t, J=7.5 Hz, 2H), 3.41 (dq, J=3.2, 6.8 Hz, 1H), 3.04 (m, 2H), 2.87 (q, J=7.6 Hz, 2H), 2.32 (q, J=2.1 Hz, 3H), 1.77 (m, 2H), 1.69 (m, 2H), 1.42-1.32 (m, 10H), 1.24 (t, J=7.6 Hz, 3H), 1.04 (d, J=6.8 Hz, 3H).

### Example 72

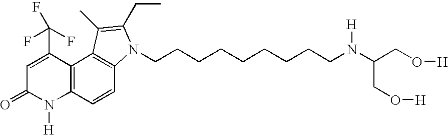

### 2-Ethyl-3-[9-(2-hydroxy-1-hydroxymethyl-ethylamino)-nonyl]-1-methyl-9-trifluoromethyl-3,6-dihydro-pyrrolo[3,2-f]quinolin-7-one

(Compound 291, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.17 (d, J=8.9 Hz, 1H), 7.00 (s, 1H), 4.24 (t, J=7.5 Hz, 2H), 3.61 (dd, J=11.2, 5.6 Hz, 2H), 3.53 (dd, J=11.2, 5.8 Hz, 2H), 2.87 (q, J=7.6 Hz, 2H), 2.70 (qn, J=5.6 Hz, 1H), 2.64 (t, J=7.5 Hz, 2H), 2.32 (q, J=2.0 Hz, 3H), 1.76 (qn, J=7.1 Hz, 1H), 1.49 (m, 2H), 1.41-1.28 (m, 10H), 1.24 (t, J=7.6 Hz, 3H).

### Example 73

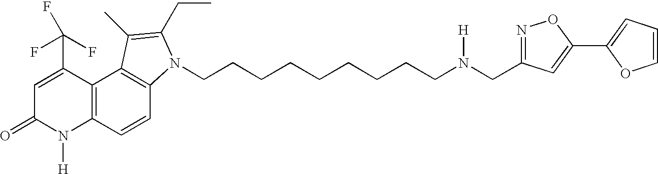

### 2-Ethyl-3-{9-[(5-furan-2-yl-isoxazol-3-yl-methyl)-amino]-nonyl}-1-methyl-9-trifluoromethyl-3,6-dihydro-pyrrolo[3,2-f]quinolin-7-one

(Compound 290, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.68 (dd, J=1.7, 0.7 Hz, 1H), 7.16 (d, J=8.8 Hz, 1H), 6.99 (s, 1H), 6.96 (dd, J=3.5, 0.7 Hz, 1H), 6.62 (s, 1H), 6.61 (dd, J=3.5, 1.7 Hz, 1H), 4.23 (t, J=7.6 Hz, 2H), 3.84 (s, 2H), 2.86 (q, J=7.6 Hz, 2H), 2.59 (t, J=7.4 Hz, 2H), 2.31 (q, J=2.0 Hz, 3H), 1.75 (m, 2H), 1.51 (m, 2H), 1.40-1.27 (m, 10H), 1.23 (t, J=7.6 Hz, 3H).

### Example 74

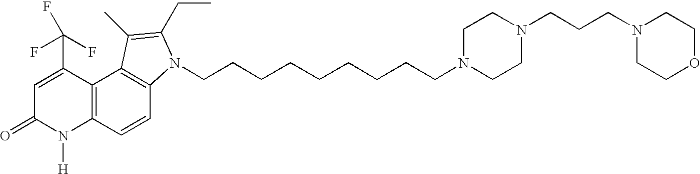

### 2-Ethyl-1-methyl-3-{9-[4-(3-morpholin-4-yl-propyl)-piperazin-1-yl]-nonyl}-9-trifluoromethyl-3,6-dihydro-pyrrolo[3,2-f]quinolin-7-on

(Compound 289 Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.61 (m, 4H), 4.24 (t, J=7.5 Hz, 2H), 3.69 (t, J=4.7 Hz, 4H), 2.87 (q, J=7.6 Hz, 2H), 2.72-2.52 (m, 4H), 2.52-2.37 (m, 10H), 2.32 (q J=2.1 Hz, 3H), 1.79-1.69 (m, 4H), 1.49 (m, 2H), 1.40-1.26 (m, 10H), 1.24 (t, J=7.6 Hz, 3H).

### Example 75

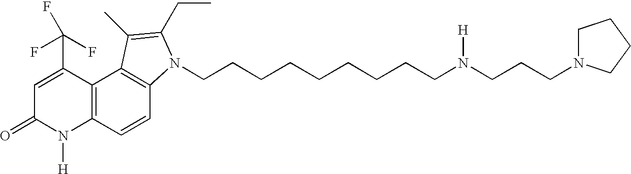

### 2-Ethyl-1-methyl-3-[9-(3-pyrrolidin-1-yl-propylamino)-nonyl]-9-trifluoromethyl-3,6-dihydro-pyrrolo[3,2-f]quinolin-7-one

(Compound 288, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.5 Hz, 2H), 2.87 (q, J=7.6 Hz, 2H), 2.65 (t, J=7.3 Hz, 2H), 2.60-2.50 (m, 8H), 2.32 (q, J=2.0 Hz, 3H), 1.82-1.70 (m, 8H), 1.49 (m, 2H), 1.40-1.27 (m, 10H), 1.24 (t, J=7.6 Hz, 3H).

### Example 76

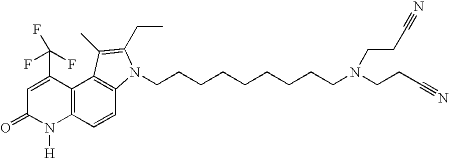

### 3-{(2-Cyanoethyl)-[9-(2-ethyl-1-methyl-7-oxo-9-trifluoromethyl-6,7-dihydro-pyrrolo[3,2-f]quinolin-3-yl)-nonyl]-amino}-propionitrile

(Compound 287, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.8 Hz, 1H), 7.17 (d, J=8.8 Hz, 1H), 6.99 (s, 1H), 4.24 (t, J=7.6 Hz, 2H), 3.19 (t, J=7.1 Hz, 2H), 2.90 (t, J=6.8 Hz, 4H), 2.87 (q, J=7.5 Hz, 2H), 2.60 (t, J=6.8 Hz, 4H), 2.32 (q, J=2.2 Hz, 3H), 1.80-1.71 (m, 4H), 1.41-1.26 (m, 10H), 1.24 (t, J=7.5 Hz, 3H).

### Example 77

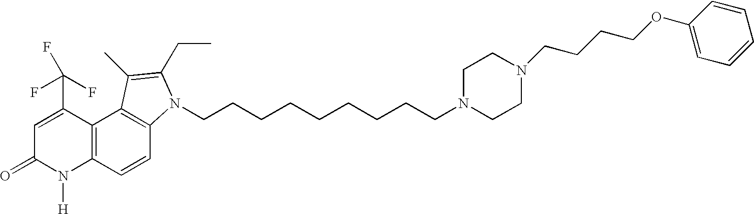

### 2-Ethyl-1-methyl-3-{9-[4-(4-phenoxy-butyl)-piperazin-1-yl]-nonyl}-9-trifluoromethyl-3,6-dihydro-pyrrolo[3,2-f]quinolin-7-one

(Compound 285, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.24 (m, 2H), 7.16 (d, J=8.9 Hz, 1H), 6.99 (s, 1H), 6.91-6.87 (m, 3H), 4.24 (t, J=7.5 Hz, 2H), 3.99 (t, J=6.1 Hz, 2H), 2.87 (q, J=7.6 Hz, 2H), 2.74-2.27 (m, 8H), 2.45 (m, 2H), 2.35 (m, 2H), 2.32 (q, J=2.1 Hz, 3H), 1.82-1.66 (m, 6H), 1.47 (m, 2H), 1.41-1.26 (m, 10H), 1.24 (t, J=7.6 Hz, 3H).

### Example 78

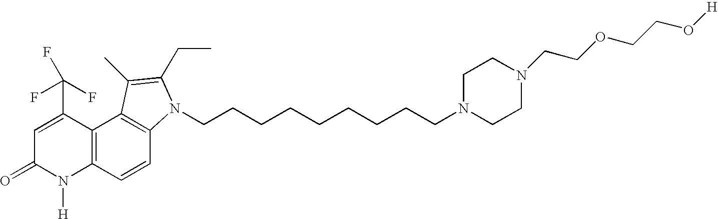

### 2-Ethyl-3-(9-{-4-[2-(2-hydroxy-ethoxy)-ethyl]-piperazin-1-yl}-nonyl)-1-methyl-9-trifluoromethyl-3,6-dihydro-pyrrolo[3,2-f]quinolin-7-one

(Compound 284, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.23 (t, J=7.5 Hz, 2H), 3.65 (m, 2H), 3.62 (t, J=5.5 Hz, 2H), 3.52 (m, 2H), 2.87 (q, J=7.6 Hz, 2H), 2.70-2.38 (m, 8H), 2.60 (t, J=5.5 Hz, 2H), 2.31 (q, J=2.0 Hz, 3H), 2.31 (m, 2H), 1.75 (m, 2H), 1.46 (m, 2H), 1.40-1.26 (m, 10H), 1.23 (t, J=7.6 Hz, 3H).

### Example 79

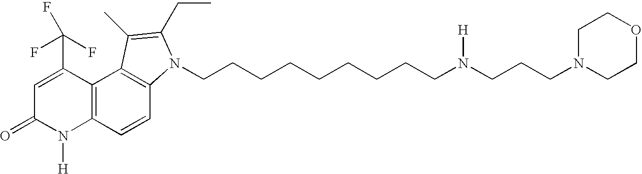

### 2-Ethyl-1-methyl-3-[9-(3-morpholin-4-yl-propylamino)-nonyl]-9-trifluoromethyl-3,6-dihydro-pyrrolo[3,2-f]quinolin-7-one

(Compound 283, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.9 Hz, 1H), 7.16 (d, J=8.9 Hz, 1H), 6.99 (s, 1H), 4.25 (t, J=7.5 Hz, 2H), 3.81 (m, 2H), 3.68 (m, 4H), 3.10 (m, 2H), 3.08 (t, J=7.0 Hz, 2H), 2.98 (t, J=7.8 Hz, 2H), 2.87 (q, J=7.6 Hz, 2H), 2.50 (t, J=7.0 Hz, 2H), 2.32 (q, J=2.1 Hz, 3H), 1.85 (qn, J=7.0 Hz, 2H), 1.76 (m, 2H), 1.65 (m, 2H), 1.41-1.30 (m, 10H), 1.24 (t, J=7.6 Hz, 3H).

### Example 80

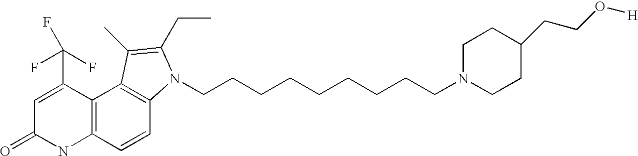

### 2-Ethyl-3-{9-[4-(2-hydroxy-ethyl)-piperidin-1-yl]-nonyl}-1-methyl-9-trifluoromethyl-3,6-dihydro-pyrrolo[3,2-f]quinolin-7-one

(Compound 286, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.76 (d, J=8.8 Hz, 1H), 7.16 (d, J=8.8 Hz, 1H), 7.00 (s, 1H), 4.25 (t, J=7.4 Hz, 2H), 3.62 (t, J=6.4 Hz, 2H), 3.46-3.39 (m, 2H), 2.93 (m, 2H), 2.87 (q, J=7.6 Hz, 2H), 2.79 (m, 2H), 2.32 (q, J=2.0 Hz, 3H), 1.95 (m, 2H), 1.76 (m, 2H), 1.65 (m, 2H), 1.52 (m, 2H), 1.46-1.28 (m, 13H), 1.24 (t, J=7.6 Hz, 3H).

### Example 81

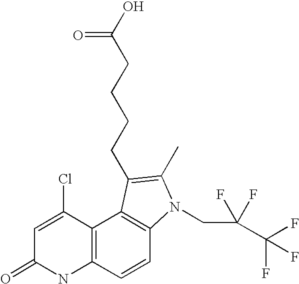

### 5-[9-Chloro-2-methyl-7-oxo-3-(2,2,3,3,3-pentafluoro-propyl)-6,7-dihydro-3H-pyrrolo-[3,2-f]quinolin-1-yl]-pentanoic acid

(Compound 186, Structure 27 of Scheme VIII, where R¹=Chloro, R³=Hydrogen, R⁴⁵=Methyl, R⁴⁶=2,2,3,3,3,3-Pentafluoropropyl)

¹H NMR (500 MHz, DMSO) δ 11.99 (s, 1H), 7.83 (d, J=8.8 Hz, 1H), 7.20 (d, J=8.8 Hz, 1H), 6.68 (s, 1H), 5.22 (t, J=16.6 Hz, 2H), 3.17 (d, J=5.1 Hz, 1H), 3.01 (t, J=5.8 Hz, 2H), 2.81 (t, J=6.2 Hz, 2H), 2.52 (s, 3H), 1.88 (m, 2H), 1.68 (m, 2H).

### Example 82

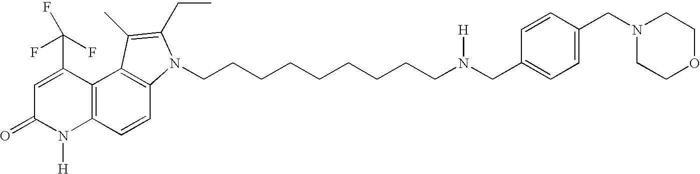

### 2-Ethyl-1-methyl-3-[9-(4-morpholin-4-ylmethyl-benzylamino)-nonyl]-9-trifluoromethyl-3,6-dihydro-pyrrolo[3,2-f]quinolin-7-one

(Compound 282, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.44-7.39 (m, 4H), 7.16 (d, J=8.8 Hz, 1H), 6.99 (s, 1H), 4.24 (t, J=7.5 Hz, 2H), 4.08 (s, 2H), 3.67 (m, 4H), 3.53 (s, 2H), 2.91 (t, J=8.0 Hz, 2H), 2.87 (q, J=7.6 Hz, 2H), 2.44 (m, 4H), 2.32 (q, J=2.0 Hz, 3H), 1.76 (m, 2H), 1.62 (m, 2H), 1.41-1.28 (m, 10H), 1.23 (t, J=7.6 Hz, 3H).

### Example 83

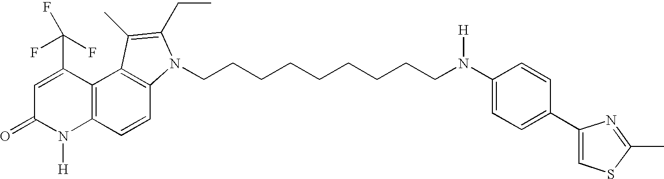

### 2-Ethyl-1-methyl-3-{9-[4-(2-methyl-thiazol-4-yl)-phenylamino]-nonyl}-9-trifluoromethyl-3,6-dihydro-pyrrolo[3,2-f]quinolin-7-one

(Compound 281, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.73 (d, J=8.8 Hz, 1H), 7.59 (d, J=8.8 Hz, 2H), 7.24 (s, 1H), 7.15 (d, J=8.8 Hz, 1H), 6.99 (s, 1H), 6.63 (d, J=8.8 Hz, 2H), 4.22 (t, J=7.6 Hz, 2H), 3.08 (t, J=7.1 Hz, 2H), 2.86 (q, J=7.6 Hz, 2H), 2.71 (s, 3H), 2.31 (q, J=2.0 Hz, 3H), 1.75 (m, 2H), 1.59 (m, 2H), 1.43-1.30 (m, 10H), 1.23 (t, J=7.6 Hz, 3H).

### Example 84

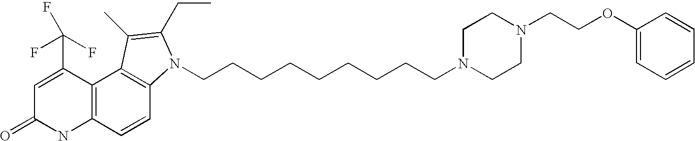

### 2-Ethyl-1-methyl-3-{9-[4-(2-phenoxy-ethyl)-piperazin-1-yl]-nonyl}-9-trifluoromethyl-3,6-dihydro-pyrrolo[3,2-f]quinolin-7-one

(Compound 280, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.26 (m, 2H), 7.16 (d, J=8.9 Hz, 1H), 6.99 (s, 1H), 6.94-6.90 (m, 3H), 4.24 (t, J=7.5 Hz, 2H), 4.13 (t, J=5.4 Hz, 2H), 2.94-2.60 (m, 10H), 2.89 (m, 2H), 2.87 (q, J=7.6 Hz, 2H), 2.31 (q, J=2.1 Hz, 3H), 1.75 (qn, J=7.4 Hz, 2H), 1.56 (m, 2H), 1.40-1.28 (m, 10H), 1.23 (t, J=7.6 Hz, 3H).

### Example 85

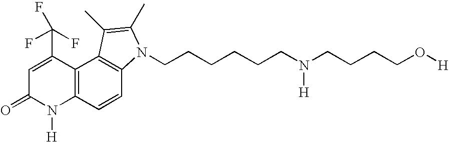

### 3-[6-(4-Hydroxy-butylamino)-hexyl]-1,2-dimethyl-9-trifluoromethyl-3,6-dihydro-pyrrolo[3,2-f]quinolin-7-one

(Compound 279, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.76 (d, J=8.8 Hz, 1H), 7.16 (d, J=8.8 Hz, 1H), 7.00 (s, 1H), 4.27 (t, J=7.3 Hz, 2H), 3.59 (t, J=6.0 Hz, 2H), 2.96 (t, J=7.7 Hz, 2H), 2.91 (t, J=7.8 Hz, 2H), 2.43 (s, 3H), 2.30 (q, J=2.0 Hz, 3H), 1.82-1.69 (m, 4H), 1.66-1.56 (m, 4H), 1.42-1.38 (m, 4H).

### Example 86

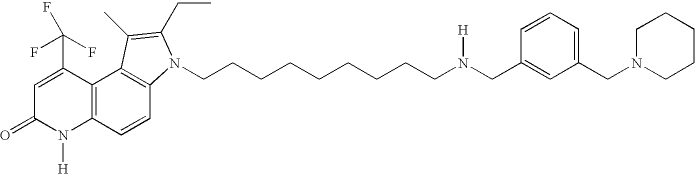

### 2-Ethyl-1-methyl-3-[9-(3-piperidin-1-ylmethyl-benzylamino)-nonyl]-9-trifluoromethyl-3,6-dihydro-pyrrolo[3,2-f]quinolin-7-one

(Compound 278, Structure 27 of Scheme VIII, where R¹=Trifluoromethyl, R³=Hydrogen, R⁴³=Methyl, R⁴⁵=Ethyl)

¹H NMR (300 MHz, CD₃OD) δ 7.75 (d, J=8.9 Hz, 1H), 7.44-7.35 (m, 4H), 7.16 (d, J=8.9 Hz, 1H), 6.99 (s, 1H), 4.24 (t, J=7.3 Hz, 2H), 4.05 (m, 2H), 3.60 (m, 2H), 2.87 (m, 3H), 2.57-2.46 (m, 6H), 2.31 (m, 3H), 1.75 (m, 2H), 1.68-1.56 (m, 8H), 1.49 (m, 2H), 1.41-1.28 (m, 8H), 1.23 (t, J=7.5 Hz, 3H).

### Example 87

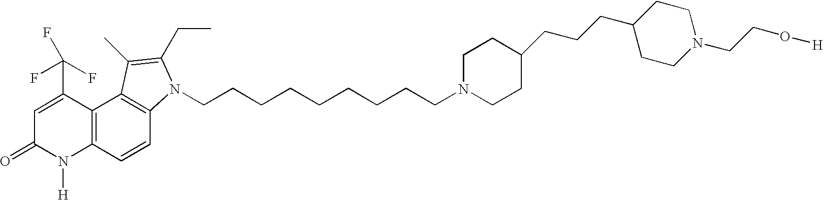

### 2-Ethyl-3-[9-(4-{3-[1-(2-hydroxy-ethyl)-piperidin-4-yl]-propyl}-piperidin-1-yl)-nonyl]-1-methyl-9-trifluoromethyl-3,6-dihydro-pyrrolo[3,2-f]quinolin-7-one

(Compound 277, Structure 27 of Scheme VIII, where R¹=Trifluoromethyl, R³=Hydrogen, R⁴³=Methyl, R⁴⁵=Ethyl)

¹H NMR (300 MHz, CD₃OD) δ 7.75 (d, J=8.9 Hz, 1H), 7.16 (d, J=8.9 Hz, 1H), 6.99 (s, 1H), 4.24 (t, J=7.5 Hz, 2H), 3.68 (t, J=6.2 Hz, 2H), 3.02-2.82 (m, 8H), 2.53 (t, J=6.2 Hz, 2H), 2.32 (m, 3H), 2.30 (m, 2H), 2.15-1.64 (m, 11H), 1.53-1.42 (m, 2H), 1.40-1.15 (m, 22H).

### Example 88

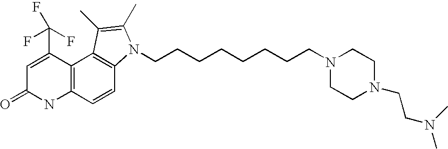

### 3-{8-[4-(2-Dimethylamino-ethyl)-piperazin-1-yl]-octyl}-1,2-dimethyl-9-trifluoromethyl-3,6-dihydro-pyrrolo[3,2-f]quinolin-7-one

(Compound 276, Structure 27 of Scheme VIII, where R¹=Trifluoromethyl, R³=Hydrogen, R⁴³=Methyl, R⁴⁵=Methyl)

¹H NMR (300 MHz, CD₃OD) δ 7.74 (d, J=8.7 Hz, 1H), 7.15 (d, J=8.7 Hz, 1H), 6.98 (s, 1H), 4.23 (t, J=7.3 Hz, 2H), 2.86 (t, J=5.1 Hz, 2H), 2.60-2.41 (m, 10H), 2.32-2.24 (m, 8H), 1.73 (m, 2H), 1.45 (m, 2H), 1.39-1.20 (m, 8H).

### Example 89

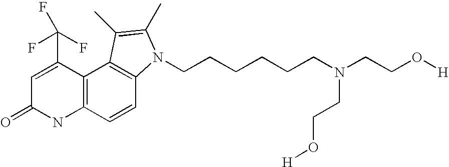

### 3-{6-[Bis-(2-hydroxy-ethyl)-amino]-hexyl}-1,2-dimethyl-9-trifluoromethyl-3,6-dihydro-pyrrolo[3,2-f]quinolin-7-one

(Compound 275, Structure 27 of Scheme VIII, where R¹=Trifluoromethyl, R³=Hydrogen, R⁴³=Methyl, R⁴⁵=Methyl)

¹H NMR (3001 MHz, CD₃OD) δ 7.76 (d, J=8.8 Hz, 1H), 7.16 (d, J=8.8 Hz, 1H), 6.99 (s, 1H), 4.25 (t, J=7.3 Hz, 2H), 3.58 (t, J=5.9 Hz, 4H), 2.64 (m, 4H), 2.52 (m, 2H), 2.43 (s, 3H), 2.30 (s, 3H), 1.75 (m, 2H), 1.47 (m, 2H), 1.42-1.27 (m, 4H).

### Example 90

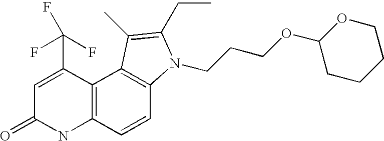

### (±)-2-Ethyl-1-methyl-3-[3-(tetrahydro-pyran-2-yloxy)-propyl]-9-trifluoromethyl-3,6-dihydro-pyrrolo[3,2-f]quinolin-7-one

(Compound 274, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.80 (d, J=8.8 Hz, 1H), 7.16 (d, J=8.8 Hz, 1H), 6.99 (s, 1H), 4.56 (m, 1H), 4.43-4.32 (m, 2H), 3.85 (m, 1H), 3.78 (m, 1H), 3.49 (m, 1H), 3.39 (m, 1H), 2.90 (q, J=7.6 Hz, 2H), 2.32 (q, J=2.1 Hz, 3H), 2.03 (m, 2H), 1.87 (m, 1H), 1.73 (m, 1H), 1.63-1.50 (m, 4H), 1.24 (t, J=7.6 Hz, 3H).

### Example 91

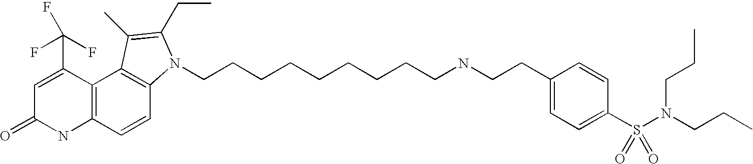

### 4-{2-[9-(2-Ethyl-1-methyl-7-oxo-9-trifluoromethyl-6,7-dihydro-pyrrolo[3,2-f]quinolin-3-yl)-nonylamino]-ethyl}-N,N-dipropyl-benzenesulfonamide

(Compound 273, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.46 (s, 1H), 7.79 (d, J=8.5 Hz, 2H), 7.75 (d, J=8.7 Hz, 1H), 7.49 (d, J=8.5 Hz, 2H), 7.16 (d, J=8.7 Hz, 1H), 6.99 (s, 1H), 4.24 (t, J=7.5 Hz, 2H), 3.28-3.24 (m, 2H), 3.09-3.04 (m, 6H), 2.99 (t, J=7.9 Hz, 2H), 2.87 (q, J=7.6 Hz, 2H), 2.32 (q, J=2.1 Hz, 3H), 1.76 (m, 2H), 1.65 (m, 2H), 1.55 (sext, J=7.4 Hz, 4H), 1.41-1.30 (m, 10H), 1.23 (t, J=7.6 Hz, 3H), 0.87 (t, J=7.4 Hz, 6H).

### Example 92

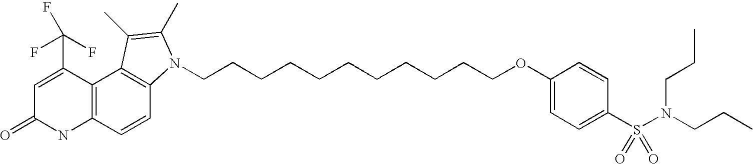

### 4-[9-(2-Ethyl-1-methyl-7-oxo-9-trifluoromethyl-6,7-dihydro-pyrrolo[3,2-f]quinolin-3-yl)-nonyloxy]-N,N-dipropyl-benzenesulfonamide

(Compound 272, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.63 (d, J=8.9 Hz, 2H), 7.16 (d, J=8.8 Hz, 1H), 6.99 (s, 1H), 6.89 (d, J=8.9 Hz, 2H), 4.24 (t, J=7.5 Hz, 2H), 3.18 (t, J=6.9 Hz, 2H), 3.03 (t, J=7.5 Hz, 4H), 2.87 (q, J=7.5 Hz, 2H), 2.32 (q, J=2.1 Hz, 3H), 1.80-1.71 (m, 4H), 1.54 (sext, J=7.5 Hz, 4H), 1.41-1.26 (m, 10H), 1.24 (t, J=7.5 Hz, 3H), 0.87 (t, J=7.5 Hz, 6H).

### Example 93

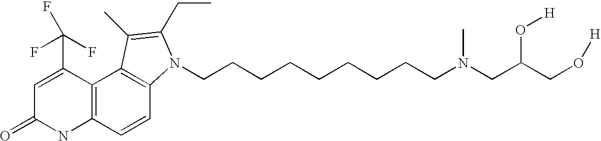

### (±)-3-{9-[(2,3-Dihydroxy-propyl)-methyl-amino]-nonyl}-2-ethyl-1-methyl-9-trifluoromethyl-3,6-dihydro-pyrrolo[3,2-f]quinolin-7-one

(Compound 271, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 4.24 (t, J=7.5 Hz, 2H), 3.80 (m, 1H), 3.54-3.46 (m, 2H), 2.87 (q, J=7.5 Hz, 2H), 2.65-2.52 (m, 4H), 2.40 (s, 3H), 2.32 (q, J=1.9 Hz, 3H), 1.75 (m, 2H), 1.51 (m, 2H), 1.42-1.26 (m, 10H), 1.24 (t, J=7.5 Hz, 3H).

### Example 94

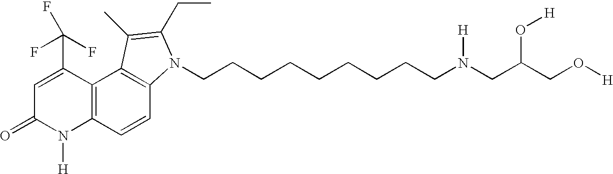

### (±)-3-[9-(2,3-Dihydroxy-propylamino)-nonyl]-2-ethyl-1-methyl-9-trifluoromethyl-3,6-dihydro-pyrrolo[3,2-f]quinolin-7-one

(Compound 270, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 4.24 (t, J=7.5 Hz, 2H), 3.74 (m, 1H), 3.53-3.46 (m, 2H), 2.87 (q, J=7.6 Hz, 2H), 2.73 (m, 1H), 2.66-2.55 (m, 3H), 2.32 (q, J=2.1 Hz, 3H), 1.76 (m, 2H), 1.50 (m, 2H), 1.41-1.28 (m, 10H), 1.24 (t, J=7.6 Hz, 3H).

### Example 95

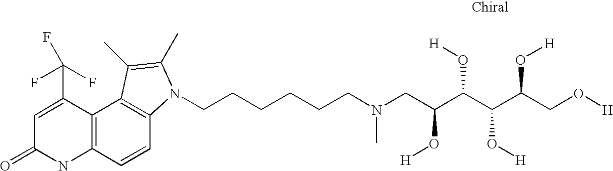

### 1,2-Dimethyl-3-{6-[methyl-((2S,3S,4S,5S)-2,3,4,5,6-pentahydroxy-hexyl)-amino]-hexyl}-9-trifluoromethyl-3,6-dihydro-pyrrolo[3,2-f]quinolin-7-one

(Compound 269, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.77 (d, J=8.8 Hz, 1H), 7.16 (d, J=8.8 Hz, 1H), 6.99 (s, 1H), 4.30-4.23 (m, 2H), 4.18 (ddd, J=9.2, 3.4, 1.9 Hz, 1H), 3.89 (m, 2H), 3.65 (m, 1H), 3.55 (dd, J=9.1, 1.9 Hz, 1H), 3.49 (dd, J=9.0, 1.8 Hz, 1H), 3.23 (m, 2H), 3.11 (m, 2H), 2.87 (s, 3H), 2.44 (s, 3H), 2.30 (q, J=1.7 Hz, 3H), 1.83-1.65 (m, 4H), 1.46-1.37 (m, 4H).

### Example 96

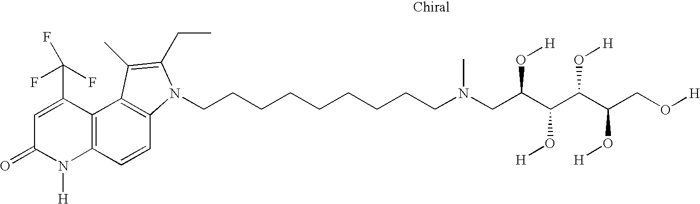

### 2-Ethyl-1-methyl-3-{9-[methyl-((2R,3R,4R,5R)-2,3,4,5,6-pentahydroxy-hexyl)-amino]-nonyl}-9-trifluoromethyl-3,6-dihydro-pyrrolo[3,2-f]quinolin-7-one

(Compound 268, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.17 (d, J=8.8 Hz, 1H), 6.99 (s, 1H), 4.23 (t, J=7.5 Hz, 2H), 4.03 (m, 1H), 3.92 (m, 1H), 3.66-3.63 (m, 3H), 3.54 (m, 1H), 2.86 (q, J=7.5 Hz, 2H), 2.70 (m, 1H), 2.54 (m, 1H), 2.50-2.39 (m, 2H), 2.32 (s, 3H), 2.31 (q, J=2.1 Hz, 3H), 1.75 (m, 2H), 1.48 (m, 2H), 1.40-1.26 (m, 10H), 1.24 (t, J=7.5 Hz, 3H).

### Example 97

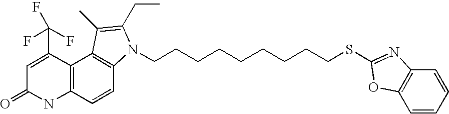

### 3-[9-(Benzooxazol-2-ylsulfanyl)-nonyl]-2-ethyl-1-methyl-9-trifluoromethyl-3,6-dihydro-pyrrolo[3,2-f]quinolin-7-one

(Compound 267, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.05 (s, 1H), 7.59 (ddd, J=7.8, 1.4, 0.6 Hz, 1H), 7.55 (d, J=8.7 Hz, 1H), 7.42 (ddd, J=7.8, 1.2, 0.6 Hz, 1H), 7.29-7.21 (m, 2H), 7.19 (d, J=8.7 Hz, 1H), 7.15 (s, 1H), 4.13 (t, J=7.7 Hz, 2H), 3.31 (t, J=7.3 Hz, 2H), 2.82 (q, J=7.6 Hz, 2H), 2.33 (q, J=2.0 Hz, 3H), 1.82 (m, 2H), 1.75 (m, 2H), 1.47 (m, 2H), 1.41-1.31 (m, 8H), 1.24 (t, J=7.6 Hz, 3H).

### Example 98

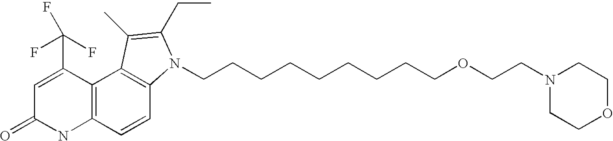

### 2-Ethyl-1-methyl-3-[9-(2-morpholin-4-yl-ethoxy)-nonyl]-9-trifluoromethyl-3,6-dihydro-pyrrolo[3,2-f]quinolin-7-one

(Compound 266, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.61 (s, 1H), 7.55 (d, J=8.8 Hz, 1H), 7.22 (d, J=8.8 Hz, 1H), 7.15 (s, 1H), 4.13 (t, J=7.7 Hz, 2H), 3.72 (m, 4H), 3.56 (t, J=5.8 Hz, 2H), 3.41 (t, J=6.7 Hz, 2H), 2.82 (q, J=7.5 Hz, 2H), 2.59 (t, J=5.8 Hz, 2H), 2.52 (m, 4H), 2.33 (q, J=2.0 Hz, 3H), 1.75 (m, 2H), 1.55 (m, 2H), 1.41-1.26 (m, 10H), 1.24 (t, J=7.5 Hz, 3H).

### Example 99

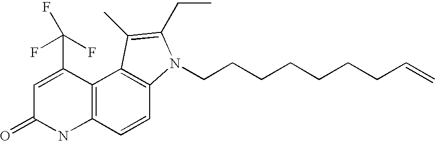

### 2-Ethyl-1-methyl-3-non-8-enyl-9-trifluoromethyl-3,6-dihydro-pyrrolo[3,2-f]quinolin-7-one

(Compound 265, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 11.49 (s, 1H), 7.54 (d, J=8.8 Hz, 1H), 7.13 (s, 1H), 7.11 (d, J=8.8 Hz, 1H), 5.80 (ddt, J=17.1, 10.2, 6.8 Hz, 1H), 4.99 (ddt, J=17.1, 2.1, 1.4 Hz, 1H), 4.94 (ddt, J=10.2, 2.1, 1.4 Hz, 1H), 4.13 (t, J=7.7 Hz, 2H), 2.82 (q, J=7.6 Hz, 2H), 2.33 (d, J=2.0 Hz, 3H), 2.04 (qt, J=6.8, 1.4 Hz, 2H), 1.76 (m, 2H), 1.42-1.28 (m, 8H), 1.24 (t, J=7.6 Hz, 3H).

### Example 100

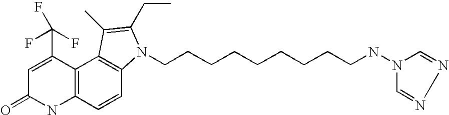

### 2-Ethyl-1-methyl-3-[9-[(1,2,4]triazol-4-ylamino)-nonyl]-9-trifluoromethyl-3,6-dihydro-pyrrolo[3,2-f]quinolin-7-one

(Compound 264, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.91 (s, 1H), 7.75 (d, J=8.8 Hz, 1H), 7.16 (d, J=8.8 Hz, 1H), 6.99 (s, 1H), 6.20 (s, 1H), 4.33 (t, J=7.3 Hz, 2H), 4.25 (t, J=7.5 Hz, 2H), 2.87 (q, J=7.5 Hz, 2H), 2.32 (d, J=2.1 Hz, 3H), 1.90 (m, 2H), 1.76 (m, 2H), 1.39-1.28 (m, 10H), 1.23 (t, J=7.5 Hz, 3H).

### Example 101

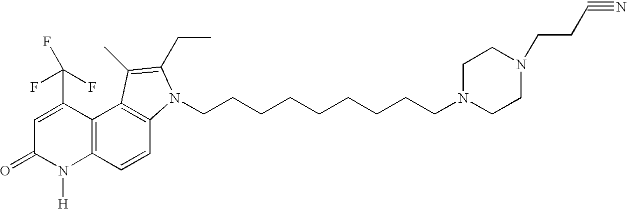

### 3-{-4-[9-(2-Ethyl-1-methyl-7-oxo-9-trifluoromethyl-6,7-dihydro-pyrrolo[3,2-f]quinolin-3-yl)-nonyl]-piperazin-1-yl}-propionitrile

(Compound 263, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.5 Hz, 2H), 2.87 (q, J=7.5 Hz, 2H), 2.68-2.60 (m, 6H), 2.60-2.44 (m, 6H), 2.32 (d, J=1.9 Hz, 3H), 2.31 (t, J=7.8 Hz, 2H), 1.76 (m, 2H), 1.46 (m, 2H), 1.39-1.26 (m, 10H), 1.24 (t, J=7.5 Hz, 3H).

### Example 102

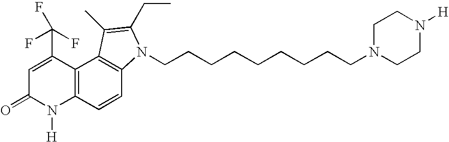

### 2-Ethyl-1-methyl-3-(9-piperazin-1-yl-nonyl)-9-trifluoromethyl-3,6-dihydro-pyrrolo[3,2-f]-quinolin-7-one

(Compound 262, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.16 (d, J=8.9 Hz, 1H), 6.99 (s, 1H), 4.24 (t, J=7.5 Hz, 2H), 2.91-2.88 (m, 4H), 2.86 (q, J=7.6 Hz, 2H), 2.47 (m, 4H), 2.32 (q, J=2.1 Hz, 3H), 2.31 (t, J=7.6 Hz, 2H), 1.76 (m, 2H), 1.46 (m, 2H), 1.40-1.26 (m, 10H), 1.24 (t, J=7.6 Hz, 3H).

### Example 103

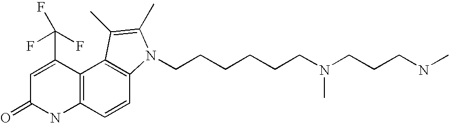

### 1,2-Dimethyl-3-{6-[methyl-(3-methylamino-propyl)-amino]-hexyl}-9-trifluoromethyl-3,6-dihydro-pyrrolo[3,2-f]quinolin-7-one

(Compound 260, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.76 (d, J=8.8 Hz, 1H), 7.15 (d, J=8.8 Hz, 1H), 7.00 (s, 1H), 4.26 (t, J=7.3 Hz, 2H), 2.97 (t, J=7.0 Hz, 2H), 2.62 (s, 3H), 2.52 (t, J=7.0 Hz, 2H), 2.43 (s, 3H), 2.38 (t, J=7.6 Hz, 2H), 2.30 (d, J=1.8 Hz, 3H), 2.24 (s, 3H), 1.77 (m, 4H), 1.48 (m, 2H), 1.42-1.28 (m, 4H).

### Example 104

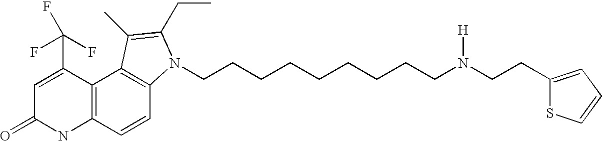

### 2-Ethyl-1-methyl-3-[9-(2-thiophen-2-yl-ethylamino)-nonyl]-9-trifluoromethyl-3,6-dihydro-pyrrolo[3,2-f]quinolin-7-one

(Compound 259, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.9 Hz, 1H), 7.20 (dd, J=5.2, 1.0 Hz, 1H), 7.16 (d, J=8.9 Hz, 1H), 6.99 (s, 1H), 6.92 (dd, J=5.2, 3.4 Hz, 1H), 6.85 (dd, J=3.4, 1.0 Hz, 1H), 4.24 (t, J=7.5 Hz, 2H), 3.03 (t, J=7.4 Hz, 2H), 2.90-2.84 (m, 4H), 2.60 (t, J=7.4 Hz, 2H), 2.32 (d, J=2.0 Hz, 3H), 1.75 (m, 2H), 1.47 (m, 2H), 1.40-1.26 (m, 10H), 1.24 (t, J=7.5 Hz, 3H).

### Example 105

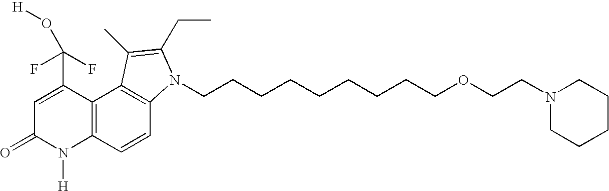

### 9-(Difluoro-hydroxy-methyl)-2-ethyl-1-methyl-3-[9-(2-piperidin-1-yl-ethoxy)-nonyl]-3,6-dihydro-pyrrolo[3,2-f]quinolin-7-one

(Compound 261, Structure 27 of Scheme VIII, where R¹=Difluorohydroxymethyl, R³=Hydrogen, R⁴³=Methyl, R⁴⁵=Ethyl)

¹H NMR (500 MHz, CD₃OD) δ 8.09 (t, J=53.1 Hz, 1H), 7.79 (d, J=8.9 Hz, 1H), 7.25 (d, J=8.9 Hz, 1H), 7.00 (s, 1H), 4.28 (t, J=7.6 Hz, 2H), 3.78 (t, J=5.6 Hz, 2H), 3.61 (t, J=5.6 Hz, 2H), 3.42 (t, J=6.5 Hz, 2H), 2.99 (q, J=7.5 Hz, 2H), 2.94-2.77 (m, 7H), 1.82-1.66 (m, 6H), 1.62-1.48 (m, 4H), 1.42-1.24 (m, 10H), 1.30 (t, J=7.5 Hz, 3H).

### Example 106

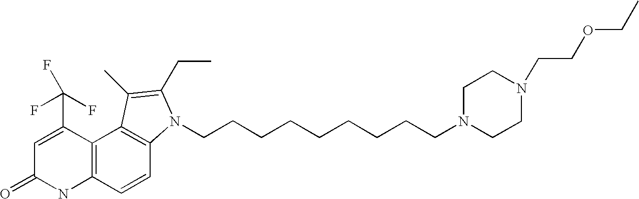

### 3-{9-[4-(2-Ethoxy-ethyl)-piperazin-1-yl]-nonyl}-2-ethyl-1-methyl-9-trifluoromethyl-3,6-dihydro-pyrrolo[3,2-f]quinolin-7-one

(Compound 258, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.16 (d, J=8.8 Hz, 1H), 6.99 (s, 1H), 4.24 (t, J=7.5 Hz, 2H), 3.57 (t, J=5.7 Hz, 2H), 3.49 (q, J=7.0 Hz, 2H), 2.87 (q, J=7.5 Hz, 2H), 2.72-2.42 (m, 8H), 2.59 (t, J=5.7 Hz, 2H), 2.34 (m, 2H), 2.32 (q, J=2.0 Hz, 3H), 1.76 (m, 2H), 1.47 (m, 2H), 1.40-1.26 (m, 10H), 1.24 (t, J=7.6 Hz, 3H), 1.18 (t, J=7.0 Hz, 3H).

### Example 107

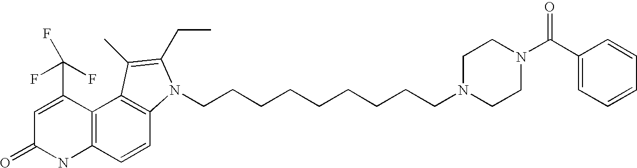

### 3-[9-(4-Benzoyl-piperazin-1-yl)-nonyl]-2-ethyl-1-methyl-9-trifluoromethyl-3,6-dihydro-pyrrolo[3,2-f]quinolin-7-one

(Compound 257, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.48-7.43 (m, 3H), 7.40 (m, 2H), 7.16 (d, J=8.8 Hz, 1H), 6.99 (s, 1H), 4.23 (t, J=7.5 Hz, 2H), 3.75 (m, 2H), 3.46 (m, 2H), 2.87 (q, J=7.5 Hz, 2H), 2.53 (m, 2H), 2.40 (m, 2H), 2.34 (t, J=7.8 Hz, 2H), 2.31 (q, J=1.9 Hz, 3H), 1.75 (m, 2H), 1.47 (m, 2H), 1.40-1.26 (m, 10H), 1.23 (t, J=7.5 Hz, 3H).

### Example 108

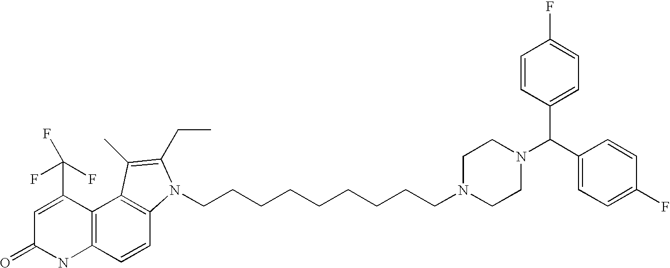

### 3-(9-{-4-[Bis-(4-fluoro-phenyl)-methyl]-piperazin-1-yl}-nonyl)-2-ethyl-1-methyl-9-trifluoromethyl-3,6-dihydro-pyrrolo[3,2-f]quinolin-7-one

(Compound 256, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.43 (dd, J=8.7, 5.4 Hz, 4H), 7.15 (d, J=8.8 Hz, 1H), 7.02 (t, J=8.7 Hz, 4H), 6.99 (s, 1H), 4.35 (s, 1H), 4.23 (t, J=7.5 Hz, 2H), 3.00-2.33 (m, 10H), 2.86 (q, J=7.6 Hz, 2H), 2.31 (q, J=2.0 Hz, 3H), 1.75 (m, 2H), 1.54 (m, 2H), 1.40-1.27 (m, 10H), 1.23 (t, J=7.6 Hz, 3H).

### Example 109

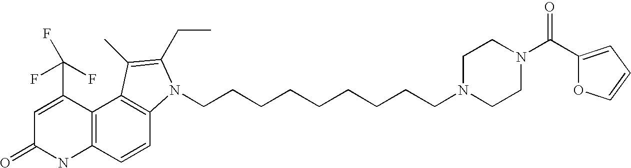

### 2-Ethyl-3-{9-[4-(furan-2-carbonyl)-piperazin-1-yl]-nonyl}-1-methyl-9-trifluoromethyl-3,6-dihydro-pyrrolo[3,2-f]quinolin-7-one

(Compound 255, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.75 (d, J=8.8 Hz, 1H), 7.67 (dd, J=1.8, 0.8 Hz, 1H), 7.16 (d, J=8.8 Hz, 1H), 7.02 (dd, J=3.5, 0.8 Hz, 1H), 6.99 (s, 1H), 6.58 (dd, J=3.5, 1.8 Hz, 1H), 4.24 (t, J=7.5 Hz, 2H), 3.80 (m, 4H), 2.87 (q, J=7.6 Hz, 2H), 2.50 (m, 4H), 2.35 (t, J=7.8 Hz, 2H), 2.32 (q, J=2.0 Hz, 3H), 1.76 (m, 2H), 1.49 (m, 2H), 1.40-1.27 (m, 10H), 1.24 (t, J=7.6 Hz, 3H).

### Example 110

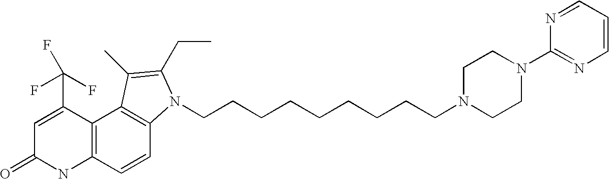

### 2-Ethyl-1-methyl-3-[9-(4-pyrimidin-2-yl-piperazin-1-yl)-nonyl]-9-trifluoromethyl-3,6-dihydro-pyrrolo[3,2-f]quinolin-7-one

(Compound 254, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.31 (d, J=4.8 Hz, 2H), 7.75 (d, J=8.8 Hz, 1H), 7.16 (d, J=8.8 Hz, 1H), 6.99 (s, 1H), 6.59 (t, J=4.8 Hz, 1H), 4.24 (t, J=7.5 Hz, 2H), 3.79 (t, J=5.1 Hz, 4H), 2.87 (q, J=7.6 Hz, 2H), 2.49 (t, J=5.1 Hz, 4H), 2.35 (t, J=7.8 Hz, 2H), 2.32 (q, J=2.0 Hz, 3H), 1.76 (m, 2H), 1.51 (m, 2H), 1.41-1.28 (m, 10H), 1.24 (t, J=7.6 Hz, 3H).

### Example 111

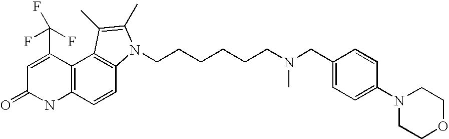

### 1,2-Dimethyl-3-{6-[methyl-(4-morpholin-4-yl-benzyl)-amino]-hexyl}-9-trifluoromethyl-3,6-dihydro-pyrrolo[3,2-f]quinolin-7-one

(Compound 253, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.75 (d, J=8.9 Hz, 1H), 7.19 (d, J=8.7 Hz, 2H), 7.15 (d, J=8.9 Hz, 1H), 6.98 (s, 1H), 6.93 (d, J=8.7 Hz, 2H), 4.23 (t, J=7.3 Hz, 2H), 3.80 (t, J=4.8 Hz, 4H), 3.64 (s, 2H), 3.12 (t, J=4.8 Hz, 4H), 2.54 (m, 2H), 2.42 (s, 3H), 2.33 (s, 3H), 2.30 (d, J=1.8 Hz, 3H), 1.74 (m, 2H), 1.55 (m, 2H), 1.40-1.27 (m, 4H).

### Example 112

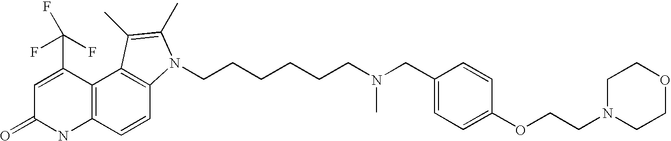

### 1,2-Dimethyl-3-(6-{methyl-[4-(2-morpholin-4-yl-ethoxy)-benzyl]-amino}-hexyl)-9-trifluoromethyl-3,6-dihydro-pyrrolo[3,2-f]quinolin-7-one

(Compound 252, Structure 27 of Scheme VIII, where R¹=Trifluoromethyl, R³=Hydrogen, R⁴³=Methyl, R⁴⁵=Methyl)

¹H NMR (500 MHz, CD₃OD) δ 7.73 (d, J=8.8 Hz, 1H), 7.20 (d, J=8.7 Hz, 2H), 7.15 (d, J=8.8 Hz, 1H), 6.98 (s, 1H), 6.89 (d, J=8.7 Hz, 2H), 4.22 (t, J=7.3 Hz, 2H), 4.11 (t, J=5.5 Hz, 2H), 3.69 (t, J=4.6 Hz, 4H), 3.51 (s, 2H), 2.79 (t, J=5.5 Hz, 2H), 2.58 (t, J=4.6 Hz, 4H), 2.42 (s, 3H), 2.40 (m, 2H), 2.30 (d, J=1.8 Hz, 3H), 2.23 (s, 3H), 1.74 (m, 2H), 1.52 (m, 2H), 1.38-1.27 (m, 4H).

### Example 113

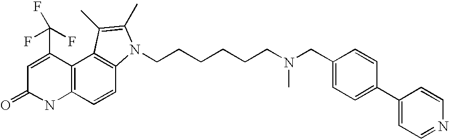

### 1,2-Dimethyl-3-{6-[methyl-(4-pyridin-4-yl-benzyl)-amino]-hexyl}-9-trifluoromethyl-3,6-dihydro-pyrrolo[3,2-f]quinolin-7-one

(Compound 251, Structure 27 of Scheme VIII, where R¹=Trifluoromethyl, R³=Hydrogen, R⁴³=Methyl, R⁴⁵=Methyl)

¹H NMR (500 MHz, CD₃OD) δ 8.57-8.55 (m, 2H), 7.74 (d, J=8.8 Hz, 1H), 7.70 (d, J=8.2 Hz, 2H), 7.69 (m, 2H), 7.42 (d, J=8.2 Hz, 2H), 7.14 (d, J=8.8 Hz, 1H), 6.97 (s, 1H), 4.22 (t, J=7.3 Hz, 2H), 3.53 (s, 2H), 2.41 (s, 3H), 2.36 (t, J=7.6 Hz, 2H), 2.29 (q, J=2.0 Hz, 3H), 2.20 (s, 3H), 1.74 (m, 2H), 1.52 (m, 2H), 1.40-1.28 (m, 4H).

### Example 114

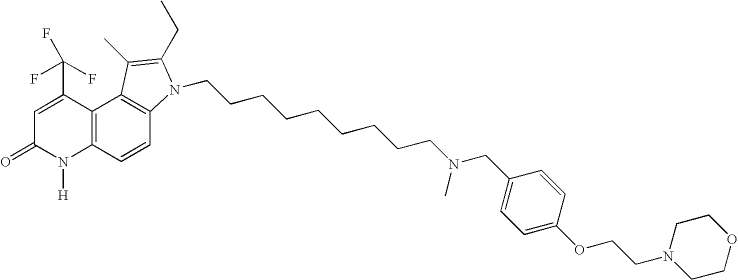

### 2-Ethyl-1-methyl-3-(9-{methyl-[4-(2-morpholin-4-yl-ethoxy)-benzyl]-amino}-nonyl)-9-trifluoromethyl-3,6-dihydro-pyrrolo[3,2-f]quinolin-7-one

(Compound 250, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.8 Hz, 1H), 7.24 (d, J=8.7 Hz, 2H), 7.16 (d, J=8.8 Hz, 1H), 6.99 (s, 1H), 6.91 (d, J=8.7 Hz, 2H), 4.23 (t, J=7.6 Hz, 2H), 4.11 (t, J=5.5 Hz, 2H), 3.69 (t, J=4.5 Hz, 4H), 3.61 (s, 2H), 2.86 (q, J=7.6 Hz, 2H), 2.77 (t, J=5.5 Hz, 2H), 2.57 (t, J=4.5 Hz, 4H), 2.48 (t, J=7.6 Hz, 2H), 2.31 (q, J=1.9 Hz, 3H), 2.29 (s, 3H), 1.75 (m, 2H), 1.53 (m, 2H), 1.41-1.25 (m, 10H), 1.23 (t, J=7.6 Hz, 3H).

### Example 115

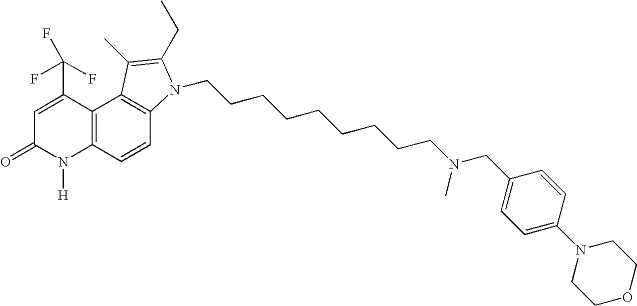

### 2-Ethyl-1-methyl-3-{9-[methyl-(4-morpholin-4-yl-benzyl)-amino]-nonyl}-9-trifluoromethyl-3,6-dihydro-pyrrolo[3,2-f]quinolin-7-one

(Compound 249, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 7.74 (d, J=8.9 Hz, 1H), 7.21 (d, J=8.7 Hz, 2H), 7.16 (d, J=8.9 Hz, 1H), 6.99 (s, 1H), 6.93 (d, J=8.7 Hz, 2H), 4.23 (t, J=7.5 Hz, 2H), 3.80 (t, J=4.8 Hz, 4H), 3.63 (s, 2H), 3.12 (t, J=4.8 Hz, 4H), 2.86 (q, J=7.6 Hz, 2H), 2.52 (m, 2H), 2.32 (s, 3H), 2.31 (q, J=2.2 Hz, 3H), 1.75 (m, 2H), 1.54 (m, 2H), 1.40-1.25 (m, 10H), 1.23 (t, J=7.6 Hz, 3H).

### Example 116

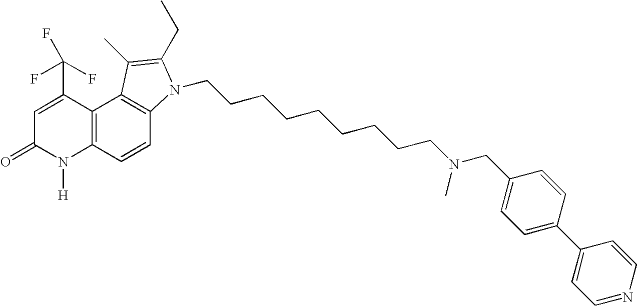

### 2-Ethyl-1-methyl-3-{9-[methyl-(4-pyridin-4-yl-benzyl)-amino]-nonyl}-9-trifluoromethyl-3,6-dihydro-pyrrolo[3,2-f]quinolin-7-one

(Compound 248, Structure 27 of Scheme VIII, where R¹=Trifluoromethyl, R³=Hydrogen, R⁴³=Methyl, R⁴⁵=Ethyl)

¹H NMR (500 MHz, CD₃OD) δ 8.61 (m, 2H), 7.88 (d, J=8.4 Hz, 2H), 7.78-7.74 (m, 3H), 7.70 (d, J=8.4 Hz, 2H), 7.20 (d, J=8.5 Hz, 1H), 7.01 (s, 1H), 4.39 (s, 2H), 4.23 (t, J=7.5 Hz, 2H), 3.14 (t, J=8.2 Hz, 2H), 2.86 (q, J=7.5 Hz, 2H), 2.80 (s, 3H), 2.31 (q, J=2.0 Hz, 3H), 1.82-1.70 (m, 4H), 1.40-1.28 (m, 10H), 1.23 (t, J=7.5 Hz, 3H).

### Example 117

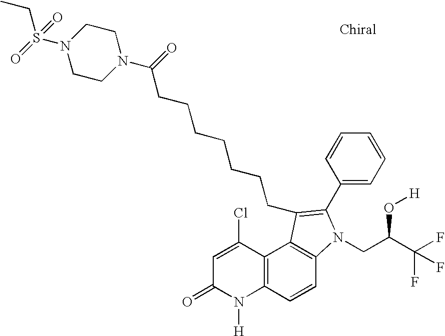

### 9-Chloro-1-[8-(4-ethanesulfonyl-piperazin-1-yl)-8-oxo-octyl]-2-phenyl-3-((R)-3,3,3-trifluoro-2-hydroxy-propyl)-3,6-dihydro-pyrrolo[3,2-f]quinolin-7-one

(Compound 185, Structure 27 of Scheme VIII, where R¹=Chloro, R³=Hydrogen, R⁴⁵=Phenyl R⁴⁶=2-Hydroxy-3,3,3-Trifluoromethyl)

¹H NMR (500 MHz, CDCl₃) 11.55 (s, 1H), 7.79 (d, J=8.8 Hz, 1H), 7.53-7.48 (m, 3H), 7.47-7.39 (m, 2H), δ 7.12 (d, J=8.8 Hz, 1H), 6.71 (s, 1H), 4.31 (dd, J=15.0, 9.6 Hz, 1H), 4.25 (dd, J=15.0, 2.7 Hz, 1H), 4.20 (m, 1H), 3.66 (m, 2H), 3.48 (m, 2H), 3.31-3.22 (m, 4H), 2.97 (m, 1H), 2.96 (q, J=7.4 Hz, 2H), 2.89 (m, 1H), 2.11 (m, 2H), 1.42 (m, 2H), 1.36 (t, J=7.4 Hz, 3H), 1.31 (m, 2H), 1.10-0.98 (m, 6H).

### Example 118

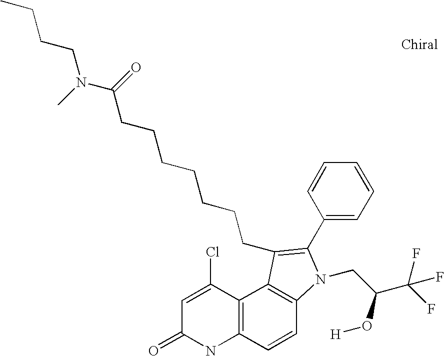

### 8-[9-Chloro-7-oxo-2-phenyl-3-((S)-3,3,3-trifluoro-2-hydroxy-propyl)-6,7-dihydro-3H-pyrrolo[3,2-f]quinolin-1-yl]-octanoic acid butyl-methyl-amide

(Compound 184, Structure 27 of Scheme VIII, where R¹=Chloro, R³=Hydrogen, R⁴⁵=Phenyl, R⁴⁶=2-Hydroxy-3,3,3-Trifluoromethyl)

¹H NMR (500 MHz, CDCl₃) 7.80 (m, 1H), 7.48 (m, 5H), 7.12 (d, J=8.8 Hz, 1H), 6.73 (s, 1H), 4.33-4.20 (m, 3H), 3.30 (m, 1H), 3.17 (m, 1H), 3.01-2.86 (m, 2H), 2.89 (s, 1.5H), 2.85 (s, 1.5H), 2.02 (m, 2H), 1.52-1.39 (m, 4H), 1.32-1.17 (m, 4H), 1.04-0.93 (m, 6H), 0.92 (t, J=7.2 Hz, 1.5H), 0.91 (t, J=7.2 Hz, 1.5H).

### Example 119

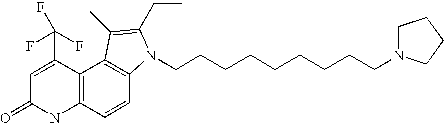

### 2-Ethyl-1-methyl-3-(9-pyrrolidin-1-yl-nonyl)-9-trifluoromethyl-3,6-dihydro-pyrrolo[3,2-f]-quinolin-7-one

(Compound 247, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.54 (d, J=8.7 Hz, 1H), 7.18 (d, J=8.7 Hz, 1H), 7.14 (s, 1H), 4.13 (t, J=7.7 Hz, 2H), 2.82 (q, J=7.6 Hz, 2H), 2.56 (m, 4H), 2.45 (m, 2H), 2.33 (q, J=1.8 Hz, 3H), 1.81 (m, 4H), 1.75 (m, 2H), 1.53 (m, 2H), 1.41-1.27 (m, 10H), 1.24 (t, J=7.6 Hz, 3H).

### Example 120

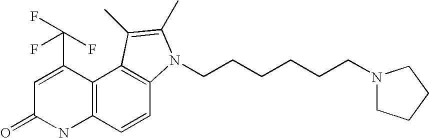

### 1,2-Dimethyl-3-(6-pyrrolidin-1-yl-hexyl)-9-trifluoromethyl-3,6-dihydro-pyrrolo[3,2-f]-quinolin-7-one

(Compound 246, Structure 27 of Scheme VIII, where R¹=Trifluoromethyl, R³=Hydrogen, R⁴³=Methyl, R⁴⁵=Methyl)

¹H NMR (500 MHz, CDCl₃) δ 7.54 (d, J=8.8 Hz, 1H), 7.14 (d, J=8.8 Hz, 1H), 7.14 (s, 1H), 4.13 (t, J=7.6 Hz, 2H), 2.51 (m, 4H), 2.43 (m, 2H), 2.41 (s, 3H), 2.32 (q, J=1.6 Hz, 3H), 1.81-1.71 (m, 6H), 1.53 (m, 2H), 1.42-1.35 (m, 4H).

### Example 121

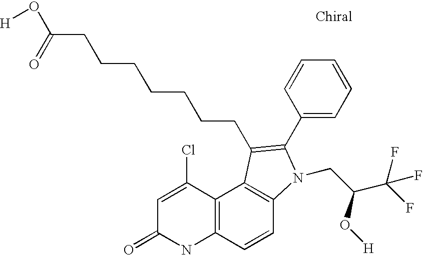

### 8-[9-Chloro-7-oxo-2-phenyl-3-((S)-3,3,3-trifluoro-2-hydroxy-propyl)-6,7-dihydro-3H-pyrrolo[3,2-f]quinolin-1-yl]-octanoic acid

(Compound 183, Structure 27 of Scheme VIII, where R¹=Chloro, R³=Hydrogen, R⁴³=, R⁴⁵=Phenyl, R⁴⁶=2-Hydroxy-3,3,3-Trifluoromethyl)

¹H NMR (500 MHz, CD₃OD) δ 7.92 (d, J=8.9 Hz, 1H), 7.56 (m, 2H), 7.53 (tt, J=7.3, 1.6 Hz, 1H), 7.45 (m, 2H), 7.27 (d, J=8.9 Hz, 1H), 6.80 (s, 1H), 4.35 (dd, J=15.2, 4.4 Hz, 1H), 4.31 (d, J=15.2 Hz, 1H), 4.00 (m, 1H), 2.99 (ddd, J=14.7, 8.9, 6.2 Hz, 1H), 2.90 (ddd, J=14.7, 8.8, 6.6 Hz, 1H), 2.16 (t, J=7.4 Hz, 2H), 1.47-1.36 (m, 4H), 1.17-1.02 (m, 6H).

### Example 122

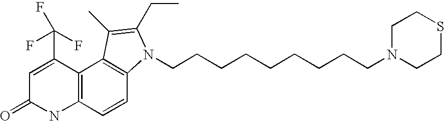

### 2-Ethyl-1-methyl-3-(9-thiomorpholin-4-yl-nonyl)-9-trifluoromethyl-3,6-dihydro-pyrrolo[3,2-f]quinolin-7-one

(Compound 245, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.49 (s, 1H), 7.55 (d, J=8.8 Hz, 1H), 7.21 (d, J=8.8 Hz, 1H), 7.15 (s, 1H), 4.13 (t, J=7.7 Hz, 2H), 2.82 (q, J=7.6 Hz, 2H), 2.71-2.66 (m, 8H), 2.33 (q J=1.7 Hz, 3H), 2.33 (m, 2H), 1.75 (m, 2H), 1.45 (m, 2H), 1.41-1.25 (m, 10H), 1.24 (t, J=7.6 Hz, 3H).

### Example 123

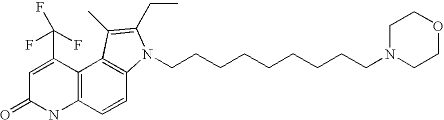

### 2-Ethyl-1-methyl-3-(9-morpholin-4-yl-nonyl)-9-trifluoromethyl-3,6-dihydro-pyrrolo[3,2-f]quinolin-7-one

(Compound 244, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.35 (s, 1H), 7.55 (d, J=8.8 Hz, 1H), 7.20 (d, J=8.8 Hz, 1H), 7.15 (s, 1H), 4.13 (t, J=7.7 Hz, 2H), 3.72 (m, 4H), 2.82 (q, J=7.6 Hz, 2H), 2.44 (m, 4H), 2.33 (q, J=2.0 Hz, 3H), 2.32 (m, 2H), 1.75 (m, 2H), 1.47 (m, 2H), 1.41-1.27 (m, 10H), 1.24 (t, J=7.6 Hz, 3H).

### Example 124

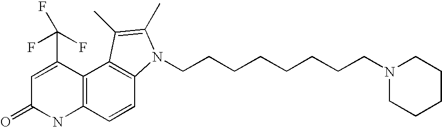

### 1,2-Dimethyl-3-(8-piperidin-1-yl-octyl)-9-trifluoromethyl-3,6-dihydro-pyrrolo[3,2-f]-quinolin-7-one

(Compound 243, Structure 27 of Scheme VIII, where R¹=Trifluoromethyl, R³=Hydrogen, R⁴³=Methyl, R⁴⁵=Methyl)

¹H NMR (500 MHz, CDCl₃) δ 7.53 (d, J=8.7 Hz, 1H), 7.13 (s, 1H), 7.11 (d, J=8.7 Hz, 1H), 4.12 (t, J=7.6 Hz, 2H), 2.41 (s, 3H), 2.37 (m, 2H), 2.32 (q, J=1.9 Hz, 3H), 2.27 (m, 2H), 1.72 (m, 2H), 1.62-1.55 (m, 6H), 1.52-1.40 (m, 4H), 1.39-1.22 (m, 8H).

### Example 125

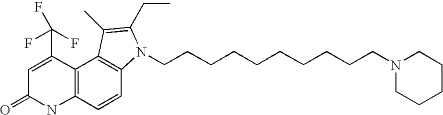

### 2-Ethyl-1-methyl-3-(10-piperidin-1-yl-decyl)-9-trifluoromethyl-3,6-dihydro-pyrrolo[3,2-f]quinolin-7-one

(Compound 242, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.55 (d, J=8.9 Hz, 1H), 7.25 (d, J=8.9 Hz, 1H), 7.12 (s, 1H), 4.14 (t, J=7.6 Hz, 2H), 2.82 (q, J=7.6 Hz, 2H), 2.85-2.75 (m, 4H), 2.33 (q, J=2.0 Hz, 3H), 2.18-1.48 (m, 10H), 1.37-1.16 (m, 14H), 1.24 (t, J=7.6 Hz, 3H).

### Example 126

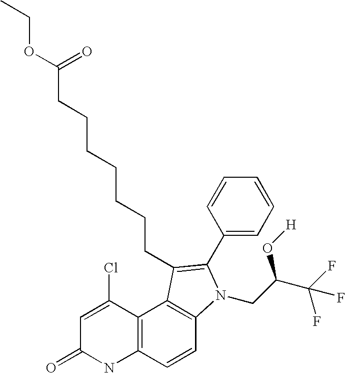

### 8-[9-Chloro-7-oxo-2-phenyl-3-((R)-3,3,3-trifluoro-2-hydroxy-propyl)-6,7-dihydro-3H-pyrrolo[3,2-f]quinolin-1-yl]-octanoic acid ethyl ester

(Compound 181, Structure 27 of Scheme VIII, where R¹=Chloro, R³=Hydrogen, R⁴⁵=Phenyl, R⁴⁶=2-Hydroxy-3,3,3-Trifluoromethyl)

¹H NMR (500 MHz, CDCl₃) δ 11.47 (s, 1H), 7.92 (m, 1H), 7.50 (m, 3H), 7.41 (m, 2H), 7.06 (d, J=8.9 Hz, 1H), 6.23 (s, 1H), 4.34 (dd, J=14.7, 9.8 Hz, 1H), 4.27 (dd, J=14.7, 2.0 Hz, 1H), 4.09 (q, J=7.2 Hz, 2H), 4.09 (m, 1H), 3.04 (m, 1H), 2.78 (m, 1H), 2.17 (t, J=7.5 Hz, 2H), 1.45 (m, 3H), 1.32 (m, 1H), 1.23 (t, J=7.2 Hz, 3H), 1.15-1.04 (m, 6H).

### Example 127

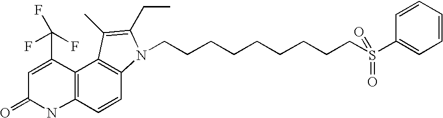

### 3-(9-Benzenesulfonyl-nonyl)-2-ethyl-1-methyl-9-trifluoromethyl-3,6-dihydro-pyrrolo[3,2-f]quinolin-7-one

(Compound 240, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.91 (m, 2H), 7.65 (tt, J=7.4, 1.2 Hz, 1H), 7.57 (dd, J=8.0, 7.4 Hz, 2H), 7.53 (d, J=8.8 Hz, 1H), 7.12 (s, 1H), 7.08 (d, J=8.8 Hz, 1H), 4.12 (t, J=7.7 Hz, 2H), 3.07 (m, 2H), 2.81 (q, J=7.6 Hz, 2H), 2.33 (q, J=2.0 Hz, 3H), 1.77-1.68 (m, 4H), 1.39-1.24 (m, 10H), 1.23 (t, J=7.6 Hz, 3H).

### Example 128

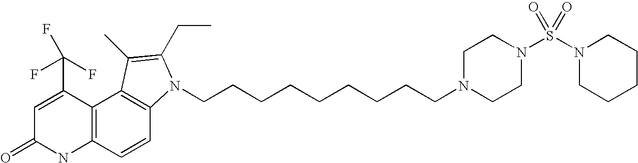

### 2-Ethyl-1-methyl-3-{9-[4-(piperidine-1-sulfonyl)-piperazin-1-yl]-nonyl}-9-trifluoromethyl-3,6-dihydro-pyrrolo[3,2-f]quinolin-7-one

(Compound 239, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.67 (s, 1H), 7.55 (d, J=8.8 Hz, 1H), 7.23 (d, J=8.8 Hz, 1H), 7.15 (s, 1H), 4.13 (m, 2H), 3.25 (m, 4H), 3.21 (m, 4H), 2.82 (q, J=7.6 Hz, 2H), 2.47 (m, 4H), 2.33 (t, J=7.7 Hz, 2H), 2.33 (q, J=1.8 Hz, 3H), 1.75 (m, 2H), 1.54 (m, 2H), 1.46 (m, 2H), 1.41-1.25 (m, 14H), 1.24 (t, J=7.6 Hz, 3H).

### Example 129

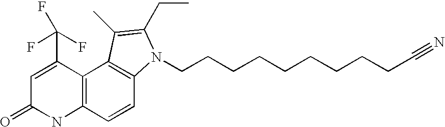

### 10-(2-Ethyl-1-methyl-7-oxo-9-trifluoromethyl-6,7-dihydro-pyrrolo[3,2-f]quinolin-3-yl)-decanenitrile

(Compound 238, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.36 (s, 1H), 7.55 (d, J=8.8 Hz, 1H), 7.20 (d, J=8.8 Hz, 1H), 7.15 (s, 1H), 4.14 (t, J=7.7 Hz, 2H), 2.82 (q, J=7.6 Hz, 2H), 2.33 (q, J=2.0 Hz, 3H), 2.33 (t, J=7.1 Hz, 2H), 1.76 (m, 2H), 1.65 (m, 2H), 1.48-1.28 (m, 10H), 1.24 (t, J=7.6 Hz, 3H).

### Example 130

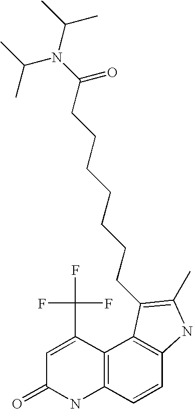

### 8-(2-Methyl-7-oxo-9-trifluoromethyl-6,7-dihydro-3H-pyrrolo[3,2-f]quinolin-1-yl)-octanoic acid diisopropylamide

(Compound 180, Structure 26 of Scheme VIII, where R¹=Trifluoromethyl, R³=Hydrogen, R⁴⁵=Methyl)

¹H NMR (500 MHz, Acetone-d₆) δ 11.23 (s, 1H), 10.51 (s, 1H), 7.64 (d, J=8.6 Hz, 1H), 7.19 (d, J=8.6 Hz, 1H), 6.92 (s, 1H), 4.03 (m, 1H), 3.44 (m, 1H), 2.86 (t, J=7.7 Hz, 2H), 2.46 (s, 3H), 2.21 (t, J=7.6 Hz, 2H), 1.47 (m, 2H), 1.38 (m, 2H), 1.31 (d, J=6.8 Hz, 6H), 1.24-1.19 (m, 6H), 1.16 (d, J=6.6 Hz, 6H).

### Example 131

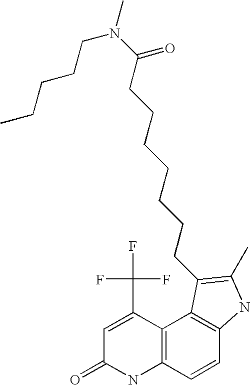

### 8-(2-Methyl-7-oxo-9-trifluoromethyl-6,7-dihydro-3H-pyrrolo[3,2-f]quinolin-1-yl)-octanoic acid methyl-pentyl-amide

(Compound 179, Structure 26 of Scheme VIII, where R¹=Trifluoromethyl, R³=Hydrogen, R⁴⁵=Methyl)

¹H NMR (300 MHz, Acetone-d₆) δ 11.26 (s, 1H), 10.51 (s, 1H), 7.64 (d, J=8.6 Hz, 1H), 7.19 (d, J=8.6 Hz, 1H), 6.92 (s, 1H), 3.29 (t, J=7.4 Hz, 1H), 3.28 (t, J=7.4 Hz, 1H), 2.96 (s, 1.5H), 2.86 (t, J=7.6 Hz, 2H), 2.85 (s, 1.5H), 2.46 (s, 3H), 2.24 (t, J=7.4 Hz, 1H), 2.22 (t, J=7.4 Hz, 1H), 1.61-1.16 (m, 16H), 0.88 (t, J=7.0 Hz, 1.5H), 0.86 (t, J=7.1 Hz, 1.5H).

### Example 132

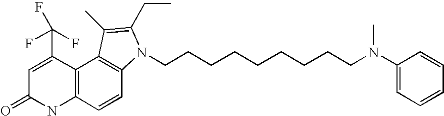

### 2-Ethyl-1-methyl-3-[9-(methyl-phenyl-amino)-nonyl]-9-trifluoromethyl-3,6-dihydro-pyrrolo[3,2-f]quinolin-7-one

(Compound 237, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.55 (d, J=8.8 Hz, 1H), 7.21 (dd, J=8.9, 7.3 Hz, 2H), 7.18 (d, J=8.8 Hz, 1H), 7.14 (s, 1H), 6.68 (m, 2H), 6.66 (tt, J=7.3, 1.0 Hz, 1H), 4.13 (t, J=7.7 Hz, 2H), 3.28 (t, J=7.5 Hz, 2H), 2.91 (s, 3H), 2.82 (q, J=7.6 Hz, 2H), 2.33 (q, J=2.0 Hz, 3H), 1.75 (m, 2H), 1.55 (m, 2H), 1.42-1.28 (m, 10H), 1.24 (t, J=7.6 Hz, 3H).

### Example 133

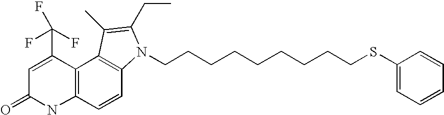

### 2-Ethyl-1-methyl-3-(9-phenylsulfanyl-nonyl)-9-trifluoromethyl-3,6-dihydro-pyrrolo[3,2-f]quinolin-7-one

(Compound 236, Structure 27 of Scheme VIII, where R¹=Trifluoromethyl, R³=hydrogen R⁴³=Methyl, R⁴⁵=Ethyl)

¹H NMR (500 MHz, CDCl₃) δ 12.48 (s, 1H), 7.54 (d, J=8.8 Hz, 1H), 7.31 (m, 2H), 7.27 (dd, J=7.3, 8.0 Hz, 2H), 7.21 (d, J=8.8 Hz, 1H), 7.15 (s, 1H), 7.15 (tt, J=7.3, 1.4 Hz, 1H), 4.12 (t, J=7.7 Hz, 2H), 2.91 (t, J=7.4 Hz, 2H), 2.82 (q, J=7.6 Hz, 2H), 2.33 (q, J=2.0 Hz, 3H), 1.75 (m, 2H), 1.64 (m, 2H), 1.45-1.26 (m, 10H), 1.24 (t, J=7.6 Hz, 3H).

### Example 134

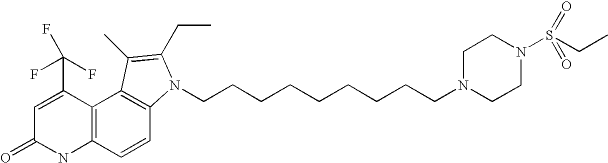

### 3-[9-(4-Ethanesulfonyl-piperazin-1-yl)-nonyl]-2-ethyl-1-methyl-9-trifluoromethyl-3,6-dihydro-pyrrolo[3,2-f]quinolin-7-one

(Compound 235, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.43 (s, 1H), 7.55 (d, J=8.8 Hz, 1H), 7.21 (d, J=8.8 Hz, 1H), 7.15 (s, 1H), 4.13 (m, 2H), 3.31 (t, J=4.9 Hz, 4H), 2.94 (q, J=7.5 Hz, 2H), 2.82 (q, J=7.5 Hz, 2H), 2.50 (t, J=4.9 Hz, 4H), 2.34 (m, 2H), 2.33 (m, 3H), 1.75 (m, 2H), 1.46 (m, 2H), 1.37 (t, J=7.5 Hz, 3H), 1.40-1.27 (m, 10H), 1.24 (t, J=7.5 Hz, 3H).

### Example 135

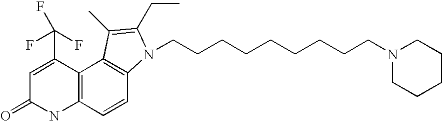

### 2-Ethyl-1-methyl-3-(9-piperidin-1-yl-nonyl)-9-trifluoromethyl-3,6-dihydro-pyrrolo[3,2-f]quinolin-7-one

(Compound 234, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.55 (d, J=8.8 Hz, 1H), 7.25 (d, J=8.8 Hz, 1H), 7.13 (s, 1H), 4.13 (t, J=7.6 Hz, 2H), 2.82 (q, J=7.5 Hz, 2H), 2.81-2.73 (m, 4H), 2.33 (d, J=2.0 Hz, 3H), 2.06-1.71 (m, 12H), 1.38-1.26 (m, 10H), 1.24 (t, J=7.5 Hz, 3H).

### Example 136

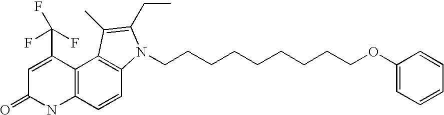

### 2-Ethyl-1-methyl-3-(9-phenoxy-nonyl)-9-trifluoromethyl-3,6-dihydro-pyrrolo[3,2-f]quinolin-7-one

(Compound 233, Structure 27 of Scheme VIII, where R¹=Trifluoromethyl, R³=Hydrogen, R⁴³=Methyl, R⁴⁵=Ethyl)

In an oven-dried amber-colored 10 ml round bottom flask equipped with a magnetic stirrer and a cold-water condenser, 3-(9-iodo-nonyl)-1,2-dimethyl-9-trifluoromethyl-3,6-dihydro-pyrrolo[3,2-f]quinolin-7-one (Compound 229, Example 145) (25.0 mg) was dissolved in dry THF (5.0 ml), and Phenol (500 mg) was added. The reaction mixture was refluxed for overnight at 85° C. where TLC (2:1 ratios of Ethyl acetate in Hexanes) showed that the reaction was finished. The reaction mixture was cooled gradually to room temperature, water added and then extracted three times with ethyl acetate (15 ml). The combined organic layers were washed with saturated aqueous sodium bicarbonate solution (20 ml), deionized water (20 ml) and with brine (20 ml) consequently, then dried over anhydrous magnesium sulfate and filtered. The filtrate was then absorbed on 2.2 volume of silica gel and further purified with flash column chromatography, eluted with 40% ethyl acetate in hexanes to provide us with 8.0 mg of the title compound: ¹H NMR (500 MHz, CDCl₃) δ 7.55 (d, J=8.8 Hz, 1H), 7.27 (dd, J=8.7, 7.3 Hz, 2H), 7.19 (d, J=8.8 Hz, 1H), 7.15 (s, 1H), 6.93 (tt, J=7.3, 1.0 Hz, 1H), 6.89 (dd, J=8.7, 1.0 Hz, 2H), 4.13 (t, J=7.7 Hz, 2H), 3.95 (t, J=6.5 Hz, 2H), 2.82 (q, J=7.6 Hz, 2H), 2.33 (q, J=2.0 Hz, 3H), 1.80-1.72 (m, 4H), 1.49-1.32 (m, 10H), 1.24 (t, J=7.6 Hz, 3H).

### Example 137

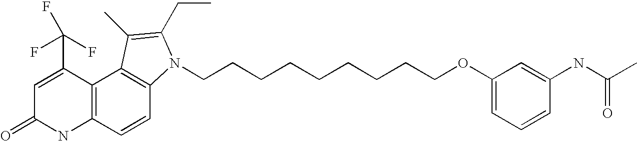

### N-{3-[9-(2-Ethyl-1-methyl-7-oxo-9-trifluoromethyl-6,7-dihydro-pyrrolo[3,2-f]quinolin-3-yl)-nonyloxy]-phenyl}-acetamide

(Compound 232, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.05 (s, 1H), 7.53 (d, J=8.8 Hz, 1H), 7.35 (t, J=1.9 Hz, 1H), 7.34 (br s, 1H), 7.19 (dd, J=8.2, 7.9 Hz, 1H), 7.17 (d, J=8.8 Hz, 1H), 7.13 (s, 1H), 6.89 (dd, J=7.9, 1.9 Hz, 1H), 6.64 (dd, J=8.2, 1.9 Hz, 1H), 4.13 (t, J=7.4 Hz, 2H), 3.94 (t, J=6.5 Hz, 2H), 2.82 (q, J=7.5 Hz, 2H), 2.33 (q, J=2.0 Hz, 3H), 2.19 (s, 3H), 1.79-1.72 (m, 4H), 1.47-1.30 (m, 10H), 1.24 (t, J=7.5 Hz, 3H).

### Example 138

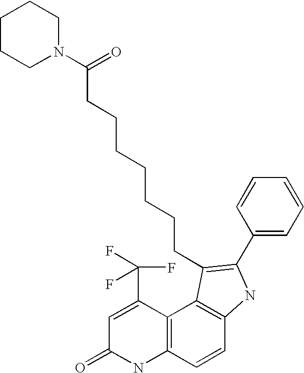

### 1-(8-Oxo-8-piperidin-1-yl-octyl)-2-phenyl-9-trifluoromethyl-3,6-dihydro-pyrrolo[3,2-f]quinolin-7-one

(Compound 178, Structure 26 of Scheme VIII, where R¹=Trifluoromethyl, R³=Hydrogen, R⁴⁵=Phenyl)

¹H NMR (500 MHz, CDCl₃) δ 8.46 (s, 1H), 7.64 (d, J=8.6 Hz, 1H), 7.57 (m, 2H), 7.50 (t, J=7.3 Hz, 2H), 7.43 (tt, J=7.3, 1.3 Hz, 1H), 7.15 (s, 1H), 7.13 (d, J=8.6 Hz, 1H), 3.51 (m, 2H), 3.33 (m, 2H), 2.99 (t, J=7.6 Hz, 2H), 2.17 (t, J=7.8 Hz, 2H), 1.50 (m, 2H), 1.41 (m, 2H), 1.37-1.19 (m, 6H), 1.12-1.05 (m, 2H), 1.04-0.93 (m, 4H).

### Example 139

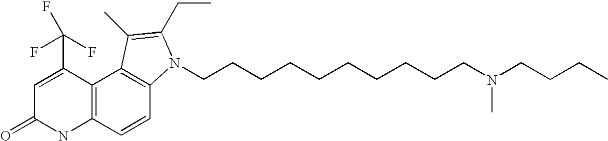

### 3-[10-(Butyl-methyl-amino)-decyl]-2-ethyl-1-methyl-9-trifluoromethyl-3,6-dihydro-pyrrolo[3,2-f]quinolin-7-one

(Compound 231, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.55 (d, J=8.8 Hz, 1H), 7.21 (d, J=8.8 Hz, 1H), 7.12 (s, 1H), 4.14 (t, J=7.6 Hz, 2H), 2.88-2.76 (m, 4H), 2.82 (q, J=7.5 Hz, 2H), 2.63 (br s, 3H), 2.33 (q, J=2.0 Hz, 3H), 1.79-1.69 (m, 6H), 1.42-1.20 (m, 14H), 1.24 (t, J=7.5 Hz, 3H), 0.96 (t, J=7.5 Hz, 3H).

### Example 140

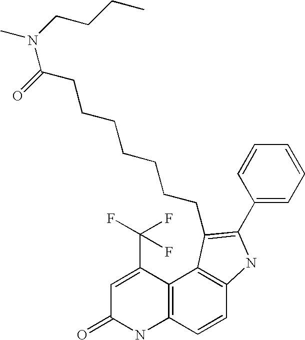

### 8-(7-Oxo-2-phenyl-9-trifluoromethyl-6,7-dihydro-3H-pyrrolo[3,2-f]quinolin-1-yl)-octanoic acid butyl-methyl-amide

(Compound 177, Structure 26 of Scheme VIII, where R¹=Trifluoromethyl, R³=Hydrogen, R⁴⁵=Phenyl)

In a 100 ml round bottom flask, equipped with a magnetic stirrer, to a solution of Compound 171 in Example 179 (16.0 g) in dry DMF (1.0 ml), 1-hydroxybenzotriazole-hydrate (7.0 mg) and EDAC-HCl (10.0 mg) were added at once and the flask was capped with a septum tightly and heated at 50° C. in an oil bath for 30 minutes. To this solution, neat methyl-pentyl-amine (0.1 ml) was added and heating continued for 24 hours where TLC (8:92 ratios of Methanol in Dichloromethane) showed that the reaction was finished. The reaction mixture was cooled to room temperature, deionized water (25.0 ml) added and extracted three times with 25.0 ml ethyl acetate. The combined organic layers were washed with 1.0 N sodium hydroxide solution (10.0 ml), 1.0 N hydrochloric acid (10.0 ml), saturated aqueous sodium bicarbonate solution (20 ml), and deionized water (20 ml) and with brine (20 ml) respectively, then dried over anhydrous magnesium sulfate and filtered. The filtrate was then absorbed on 2.2 volume of silica gel and further purified with flash column chromatography, eluted with 1% to 5% increments of methanol in 60% ethyl acetate in Hexanes solutions to provide 6.0 mg of the title compound: ¹H NMR (500 MHz, CDCl₃) δ 8.50 (s, 1H), 7.64 (d, J=8.6 Hz, 1H), 7.57 (m, 2H), 7.50 (m, 2H), 7.42 (tt, J=7.3, 1.3 Hz, 1H), 7.19 (d, J=8.6 Hz, 1H), 7.16 (s, 1H), 3.32 (t, J=7.5 Hz, 1H), 3.19 (t, J=7.5 Hz, 1H), 2.99 (t, J=7.5 Hz, 2H), 2.90 (s, 1.5H), 2.87 (s, 1.5H), 2.16 (m, 2H), 1.52-1.39 (m, 4H), 1.33-1.20 (m, 4H), 1.13-0.94 (m, 6H), 0.92 (t, J=7.4 Hz, 1.5H), 0.90 (t, J=7.4 Hz, 1.5H).

### Example 141

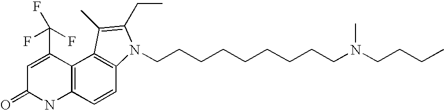

### 3-[9-(Butyl-methyl-amino)-nonyl]-2-ethyl-1-methyl-9-trifluoromethyl-3,6-dihydro-pyrrolo[3,2-f]quinolin-7-one

(Compound 230, Structure 27 of Scheme VIII, where R¹=Trifluoromethyl, R³=hydrogen R⁴³=Methyl, R⁴⁵=Ethyl)

In an oven-dried amber-colored 10 ml round bottom flask equipped with a magnetic stirrer and a cold-water condenser, 3-(9-iodo-nonyl)-1,2-dimethyl-9-trifluoromethyl-3,6-dihydro-pyrrolo[3,2-f]quinolin-7-one (Compound 229, Example 145) (25.0 mg) was dissolved in dry THF (5.0 ml), and N-butyl-methyl-amine (0.5 ml) was added and the reaction mixture was refluxed for overnight at 85° C. where TLC (2:1 ratios of Ethyl acetate in Hexanes) showed that the reaction was finished. The reaction mixture was cooled gradually to room temperature, water added and then extracted three times with ethyl acetate (15 ml). The combined organic layers were washed with 2.0 N (15 ml) hydrochloric acid, saturated aqueous sodium bicarbonate solution (20 ml), deionized water (20 ml) and with brine (20 ml) respectively, then dried over anhydrous magnesium sulfate and filtered. The filtrate was absorbed on 2.2 volume of silica gel and further purified with flash column chromatography, eluted with 5%, 10%, and 20% methanol in dichloromethane to provide 10.0 mg of the title compound: ¹H NMR (500 MHz, CDCl₃) δ 7.54 (d, J=8.8 Hz, 1H), 7.18 (d, J=8.8 Hz, 1H), 7.12 (s, 1H), 4.13 (t, J=7.6 Hz, 2H), 2.91-2.80 (m, 4H), 2.82 (q, J=7.5 Hz, 2H), 2.65 (s, 3H), 2.33 (q, J=2.1 Hz, 3H), 1.78-1.71 (m, 6H), 1.42-1.26 (m, 12H), 1.24 (t, J=7.5 Hz, 3H), 0.96 (t, J=7.5 Hz, 3H).

### Example 142

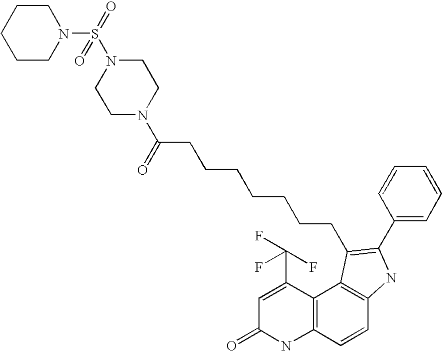

### 1-{8-Oxo-8-[4-(piperidine-1-sulfonyl)-piperazin-1-yl]-octyl}-2-phenyl-9-trifluoromethyl-3,6-dihydro-pyrrolo[3,2-f]quinolin-7-one

(Compound 176, Structure 26 of Scheme VIII, where R¹=Trifluoromethyl, R³=Hydrogen, R⁴⁵=Phenyl)

¹H NMR (500 MHz, CDCl₃) δ 12.61 (s, 1H), 8.63 (s, 1H), 7.66 (d, J=8.6 Hz, 1H), 7.57 (m, 2H), 7.50 (m, 2H), 7.42 (tt, J=7.3, 1.3 Hz, 1H), 7.29 (d, J=8.6 Hz, 1H), 7.17 (s, 1H), 3.62 (m, 2H), 3.44 (m, 2H), 3.23-3.13 (m, 8H), 3.00 (t, J=7.7 Hz, 2H), 2.18 (t, J=7.7 Hz, 2H), 1.63-1.52 (m, 6H), 1.42 (m, 2H), 1.24 (m, 2H), 1.13-0.92 (m, 6H).

### Example 143

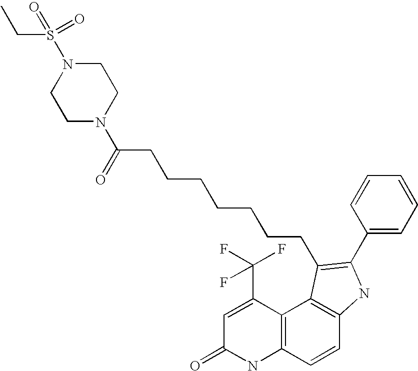

### 1-[8-(4-Ethanesulfonyl-piperazin-1-yl)-8-oxo-octyl]-2-phenyl-9-trifluoromethyl-3,6-dihydro-pyrrolo[3,2-f]quinolin-7-one

(Compound 175, Structure 26 of Scheme VIII, where R¹=Trifluoromethyl, R³=Hydrogen, R⁴⁵=Phenyl)

¹H NMR (500 MHz, CDCl₃) δ 12.57 (s, 1H), 8.61 (s, 1H), 7.66 (d, J=8.7 Hz, 1H), 7.57 (m, 2H), 7.50 (t, J=7.4 Hz, 2H), 7.42 (tt, J=7.4, 1.3 Hz, 1H), 7.29 (d, J=8.7 Hz, 1H), 7.17 (s, 1H), 3.65 (m, 2H), 3.47 (m, 2H), 3.24 (m, 4H), 3.00 (t, J=7.7 Hz, 2H), 2.95 (q, J=7.4 Hz, 2H), 2.19 (t, J=7.7 Hz, 2H), 1.43 (m, 2H), 1.35 (t, J=7.4 Hz, 3H), 1.24 (m, 2H), 1.13-0.91 (m, 6H).

### Example 144

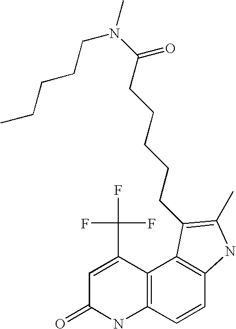

### 6-(2-Methyl-7-oxo-9-trifluoromethyl-6,7-dihydro-3H-pyrrolo[3,2-f]quinolin-1-yl)-hexanoic acid methyl-pentyl-amide

(Compound 174, Structure 26 of Scheme VIII, where R¹=Trifluoromethyl, R³=Hydrogen, R⁴⁵=Methyl)

¹H NMR (300 MHz, CD₃OD) δ 7.60 (d, J=8.6 Hz, 1H), 7.09 (d, J=8.6 Hz, 1H), 6.97 (s, 1H), 3.28 (t, J=7.5 Hz, 1H), 3.21 (t, J=7.5 Hz, 1H), 2.90 (s, 1.5H), 2.86 (t, J=7.4 Hz, 2H), 2.82 (s, 1.5H), 2.42 (s, 3H), 2.19 (t, J=7.4 Hz, 2H), 1.54-1.43 (m, 4H), 1.42-1.11 (m, 8H), 0.89 (t, J=7.0 Hz, 1.5H), 0.88 (t, J=7.0 Hz, 1.5H).

### Example 145

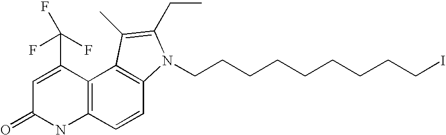

### 2-Ethyl-3-(9-iodo-nonyl)-1-methyl-9-trifluoromethyl-3,6-dihydro-pyrrolo[3,2-f]-quinolin-7-one

(Compound 229, Structure 27 of Scheme VIII, where R¹=Trifluoromethyl, R³=Hydrogen, R⁴³=Methyl, R⁴⁵=Ethyl)

¹H NMR (300 MHz, CDCl₃) δ 11.91 (s, 1H), 7.54 (d, J=8.2 Hz, 1H), 7.15 (d, J=8.2 Hz, 1H), 7.14 (s, 1H), 4.13 (t, J=7.6 Hz, 2H), 3.18 (t, J=7.0 Hz, 2H), 2.82 (q, J=7.4 Hz, 2H), 2.33 (m, 3H), 1.87-1.70 (m, 4H), 1.45-1.27 (m, 10H), 1.24 (t, J=7.4 Hz, 3H).

### Example 146

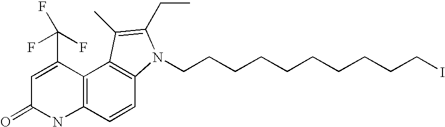

### 2-Ethyl-3-(10-iodo-decyl)-1-methyl-9-trifluoromethyl-3,6-dihydro-pyrrolo[3,2-f]quinolin-7-one

(Compound 228, Structure 27 of Scheme VIII, where R¹=Trifluoromethyl, R³=Hydrogen, R⁴³=Methyl, R⁴⁵=Ethyl)

¹H NMR (300 MHz, CDCl₃) δ 11.76 (s, 1H), 7.54 (d, J=8.7 Hz, 1H), 7.14 (s, 1H), 7.14 (d, J=8.7 Hz, 1H), 4.13 (t, J=7.1 Hz, 2H), 3.18 (t, J=7.0 Hz, 2H), 2.82 (q, J=7.5 Hz, 2H), 2.33 (m, 3H), 1.87-1.69 (m, 4H), 1.44-1.26 (m, 12H), 1.24 (t, J=7.5 Hz, 3H).

### Example 147

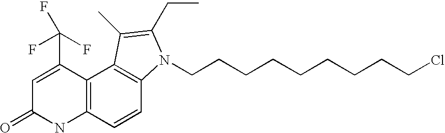

### 3-(9-Chloro-nonyl)-2-ethyl-1-methyl-9-trifluoromethyl-3,6-dihydro-pyrrolo[3,2-f]-quinolin-7-one

(Compound 227, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 7.67 (d, J=8.8 Hz, 1H), 7.31 (d, J=8.8 Hz, 1H), 4.17 (t, J=7.7 Hz, 2H), 3.52 (t, J=6.7 Hz, 2H), 2.85 (q, J=7.6 Hz, 2H), 2.35 (q, J=2.1 Hz, 3H), 1.81-1.72 (m, 4H), 1.46-1.28 (m, 10H), 1.25 (t, J=7.6 Hz, 3H).

### Example 148

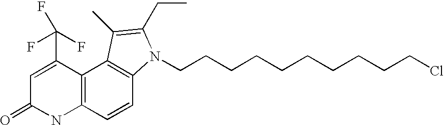

### 3-(10-Chloro-decyl)-2-ethyl-1-methyl-9-trifluoromethyl-3,6-dihydro-pyrrolo[3,2-f]-quinolin-7-one

(Compound 226, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 11.38 (s, 1H), 7.54 (d, J=8.8 Hz, 1H), 7.13 (s, 1H), 7.10 (d, J=8.8 Hz, 1H), 4.13 (t, J=7.6 Hz, 2H), 3.53 (t, J=6.8 Hz, 2H), 2.82 (q, J=7.6 Hz, 2H), 2.33 (q, J=2.1 Hz, 3H), 1.79-1.72 (m, 4H), 1.45-1.27 (m, 12H), 1.24 (t, J=7.6 Hz, 3H).

### Example 149

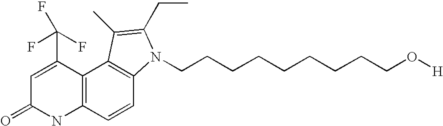

### 2-Ethyl-3-(9-hydroxy-nonyl)-1-methyl-9-trifluoromethyl-3,6-dihydro-pyrrolo[3,2-f]-quinolin-7-one

(Compound 225, Structure 27 of Scheme VIII, where R¹=Trifluoromethyl, R³=Hydrogen, R⁴³=Methyl, R⁴⁵=Ethyl)

¹H NMR (500 MHz, DMSO) δ 12.27 (s, 1H), 7.79 (d, J=8.9 Hz, 1H), 7.15 (d, J=8.9 Hz, 1H), 6.92 (s, 1H), 4.29 (s, 1H), 4.18 (t, J=7.6 Hz, 2H), 3.35 (m, 2H), 2.79 (q, J=7.5 Hz, 2H), 2.22 (q, J=1.7 Hz, 3H), 1.64 (m, 2H), 1.37 (m, 2H), 1.34-1.20 (m, 10H), 1.16 (t, J=7.5 Hz, 3H).

### Example 150

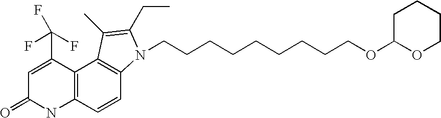

### 2-Ethyl-1-methyl-3-[9-(tetrahydro-pyran-2-yloxy)-nonyl]-9-trifluoromethyl-3,6-dihydro-pyrrolo[3,2-f]quinolin-7-one

(Compound 224, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.79 (s, 1H), 7.55 (d, J=8.8 Hz, 1H), 7.24 (d, J=8.8 Hz, 1H), 7.16 (s, 1H), 4.57 (dd, J=4.4, 2.8 Hz, 1H), 4.13 (t, J=7.7 Hz, 2H), 3.87 (m, 1H), 3.73 (dt, J=9.6, 6.9 Hz, 1H), 3.50 (m, 1H), 3.38 (dt, J=9.6, 6.7 Hz, 1H), 2.82 (q, J=7.6 Hz, 2H), 2.33 (q, J=1.9 Hz, 3H), 1.87-1.68 (m, 4H), 1.63-1.49 (m, 6H), 1.42-1.28 (m, 10H), 1.24 (t, J=7.6 Hz, 3H).

### Example 151

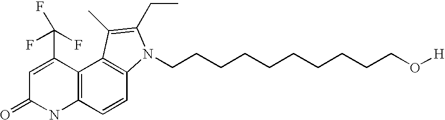

### 2-Ethyl-3-(10-hydroxy-decyl)-1-methyl-9-trifluoromethyl-3,6-dihydro-pyrrolo[3,2-f]-quinolin-7-one

(Compound 223, Structure 27 of Scheme VIII, where R¹=Trifluoromethyl, R³=Hydrogen, R⁴³=Methyl, R⁴⁵=Ethyl)

¹H NMR (500 MHz, DMSO) δ 12.28 (s, 1H), 7.80 (d, J=8.8 Hz, 1H), 7.15 (d, J=8.8 Hz, 1H), 6.92 (s, 1H), 4.18 (t, J=7.5 Hz, 2H), 3.35 (t, J=6.6 Hz, 2H), 2.79 (q, J=7.5 Hz, 2H), 2.22 (q, J=1.6 Hz, 3H), 1.64 (m, 2H), 1.37 (m, 2H), 1.32-1.20 (m, 12H), 1.15 (t, J=7.5 Hz, 3H).

### Example 152

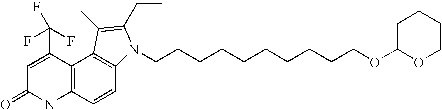

### 2-Ethyl-1-methyl-3-[10-(tetrahydro-pyran-2-yloxy)-decyl]-9-trifluoromethyl-3,6-dihydro-pyrrolo[3,2-f]quinolin-7-one

(Compound 221, Structure 27 of Scheme VIII, where R¹=Trifluoromethyl, R³=Hydrogen, R⁴³=Methyl, R⁴⁵=Ethyl)

¹H NMR (500 MHz, CDCl₃) δ 12.93 (s, 1H), 7.55 (d, J=8.7 Hz, 1H), 7.25 (d, J=8.7 Hz, 1H), 7.16 (s, 1H), 4.57 (dd, J=4.5, 2.7 Hz, 1H), 4.13 (t, J=7.7 Hz, 2H), 3.87 (m, 1H), 3.73 (dt, J=9.6, 6.9 Hz, 1H), 3.50 (m, 1H), 3.38 (dt, J=9.6, 6.7 Hz, 1H), 2.82 (q, J=7.6 Hz, 2H), 2.33 (q, J=2.0 Hz, 3H), 1.87-1.68 (m, 4H), 1.61-1.49 (m, 6H), 1.42-1.26 (m, 12H), 1.24 (t, J=7.6 Hz, 3H).

### Example 153

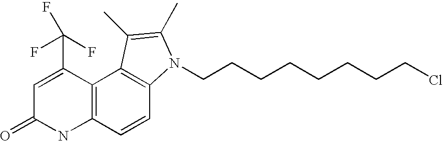

### 3-(8-Chloro-octyl)-1,2-dimethyl-9-trifluoromethyl-3,6-dihydro-pyrrolo[3,2-f]quinolin-7-one

(Compound 218, Structure 27 of Scheme VIII, where R¹=Trifluoromethyl, R³=hydrogen, R⁴³=Methyl, R⁴⁵=Methyl)

¹H NMR (500 MHz, CDCl₃) δ 12.22 (s, 1H), 7.54 (d, J=8.8 Hz, 1H), 7.18 (d, J=8.8 Hz, 1H), 7.15 (s, 1H), 4.13 (t, J=7.6 Hz, 2H), 3.52 (t, J=6.7 Hz, 2H), 2.41 (s, 3H), 2.32 (q, J=1.7 Hz, 3H), 1.79-1.70 (m, 4H), 1.46-1.29 (m, 8H).

### Example 154

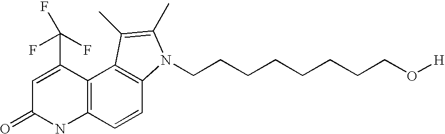

### 3-(8-Hydroxy-octyl)-1,2-dimethyl-9-trifluoromethyl-3,6-dihydro-pyrrolo[3,2-f]quinolin-7-one

(Compound 219, Structure 27 of Scheme VIII, where R¹=Trifluoromethyl, R³=Hydrogen, R⁴³=Methyl, R⁴⁵=Methyl)

¹H NMR (500 MHz, CDCl₃) δ 7.52 (d, J=8.7 Hz, 1H), 7.11 (s, 1H), 6.99 (d, J=8.7 Hz, 1H), 4.12 (t, J=7.6 Hz, 2H), 3.64 (dt, J=6.9, 5.9 Hz, 2H), 2.41 (s, 3H), 2.31 (q, J=1.8 Hz, 3H), 1.73 (m, 2H), 1.39-1.30 (m, 10H).

### Example 155

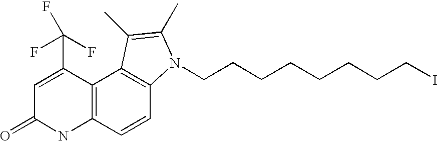

### 3-(8-Iodo-octyl)-1,2-dimethyl-9-trifluoromethyl-3,6-dihydro-pyrrolo[3,2-f]quinolin-7-one

(Compound 220, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.36 (s, 1H), 7.54 (d, J=8.8 Hz, 1H), 7.20 (d, J=8.8 Hz, 1H), 7.15 (s, 1H), 4.13 (t, J=7.5 Hz, 2H), 3.18 (t, J=7.0 Hz, 2H), 2.41 (s, 3H), 2.32 (q, J=1.7 Hz, 3H), 1.84-1.70 (m, 4H), 1.42-1.28 (m, 8H).

### Example 156

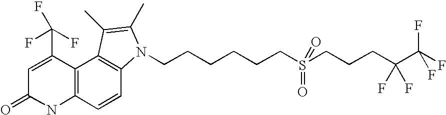

### 1,2-Dimethyl-3-[6-(4,4,5,5,5-pentafluoro-pentane-1-sulfonyl)-hexyl]-9-trifluoromethyl-3,6-dihydro-pyrrolo[3,2-f]quinolin-7-one

(Compound 217 Structure 27 of Scheme VIII; where R¹=Trifluoromethyl, R³=Hydrogen, R⁴³=Methyl, R⁴⁵=Methyl)

In a 10 ml round bottom flask equipped with a magnetic stirrer, 1,2-dimethyl-3-[6-(4,4,5,5,5-pentatluoro-pentylsulanyl)-hexyl]-9-trifluoromethyl-3,6-dihydropyrrolo-[3,2-f]quinolin-7-one (Compound 215, Example 158) (22.0 mg) was dissolved in HPLC grade methanol (5.0 ml). A solution of oxone, mono-persulfate compound (33.0 mg) in 0.2 ml water was added dropwise and the solution was stirred overnight at room temperature. TLC (2:1 ratios of Ethyl acetate in Hexanes) showed that the reaction was finished. Deionized water was added and the reaction solution was extracted three times with ethyl acetate (15 ml). The combined organic layers were washed with 1 N sodium hydroxide (20 ml), deionized water (20 ml), and brine (20 ml), then dried over anhydrous magnesium sulfate and filtered. The filtrate was absorbed on 2.2 volume of silica gel and further purified with flash column chromatography, eluted with 20%-50% ethyl acetate in hexane, followed by 1% to 5% increments of methanol in 50% ethyl acetate in hexanes solution to provide 80 mg of the title compound: ¹H NMR (500 MHz, CDCl₃) δ 11.97 (s, 1H), 7.53 (d, J=8.8 Hz, 1H), 7.16 (d, J=8.8 Hz, 1H), 7.14 (s, 1H), 4.15 (t, J=7.3 Hz, 2H), 3.03 (t, J=7.4 Hz, 2H), 2.95 (m, 2H), 2.41 (s, 3H), 2.32 (q, J=1.6 Hz, 3H), 2.30-2.14 (m, 4H), 1.85 (m, 2H), 1.78 (m, 2H), 1.50 (m, 2H), 1.41 (m, 2H).

### Example 157

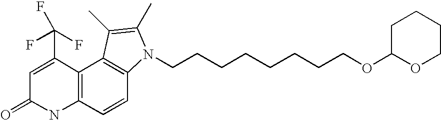

### (±)-1,2-Dimethyl-3-[8-(tetrahydro-pyran-2-yloxy)-octyl]-9-trifluoromethyl-3,6-dihydro-pyrrolo[3,2-f]quinolin-7-one

(Compound 216, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.74 (s, 1H), 7.55 (d, J=8.8 Hz, 1H), 7.23 (d, J=8.8 Hz, 1H), 7.16 (s, 1H), 4.56 (dd, J=4.5, 2.7 Hz, 1H), 4.13 (t, J=7.6 Hz, 2H), 3.87 (ddd, J=11.2, 7.5, 3.4 Hz, 1H), 3.73 (dt, J=9.6, 6.9 Hz, 1H), 3.50 (m, 1H), 3.37 (dt, J=9.6, 6.7 Hz, 1H), 2.41 (s, 3H), 2.32 (q, J=1.6 Hz, 3H), 1.82 (m, 1H), 1.76-1.68 (m, 3H), 1.58-1.49 (m, 6H), 1.40-1.30 (m, 8H).

### Example 158

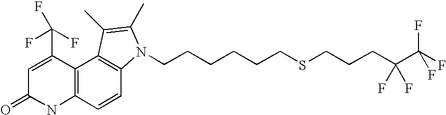

### 1,2-Dimethyl-3-[6-(4,4,5,5,5-pentafluoro-pentylsulfanyl)-hexyl]-9-trifluoromethyl-3,6-dihydro-pyrrolo[3,2-f]quinolin-7-one

(Compound 215, Structure 27 of Scheme VIII, where R¹=Trifluoromethyl, R³=Hydrogen, R⁴³=Methyl, R⁴⁵=Methyl)

In an oven-dried amber-colored 10 ml round bottom flask equipped with a magnetic stirrer, 3-(6-iodo-hexyl)-1,2-dimethyl-9-trifluoromethyl-3,6-dihydro-pyrrolo[3,2-f]quinolin-7-one (Compound 213, Example 160) (30.0 mg) was dissolved in dry THF (5.0 ml) and sodium hydride (60% dispersion in mineral oil) (100 mg) added and the solution was stirred for one hour, after which TLC (1:4 ratios of Ethyl acetate in Hexanes) showed that the reaction was finished. Water was added drop-wise very cautiously until the excess of sodium hydride was decomposed. The reaction solution was then extracted three times with ethyl acetate (15 ml). The combined organic layers were washed with 1 N sodium hydroxide (20 ml), deionized water (20 ml) and brine (20 ml), then dried over anhydrous magnesium sulfate and filtered. The filtrate was absorbed on 2.2 volume of silica gel and further purified with flash column chromatography, eluted with 20%, 50% and 75% ethyl acetate in hexane, then with pure ethyl acetate, then with 1% to 5% increments of methanol in dichloromethane solution to provide 22.0 mg of the title compound: ¹H NMR (500 MHz, CDCl₃) δ 13.15 (s, 1H), 7.55 (d, J=8.8 Hz, 1H), 7.28 (d, J=8.8 Hz, 1H), 7.17 (s, 1H), 4.14 (t, J=7.5 Hz, 2H), 2.57 (t, J=7.0 Hz, 2H), 2.49 (t, J=7.3 Hz, 2H), 2.41 (s, 3H), 2.32 (q, J=1.7 Hz, 3H), 2.16 (m, 2H), 1.87 (m, 2H), 1.75 (m, 2H), 1.58 (m, 2H), 1.48-1.35 (m, 4H).

### Example 159

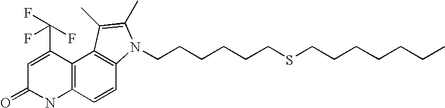

### 3-(6-Heptylsulfanyl-hexyl)-1,2-dimethyl-9-trifluoromethyl-3,6-dihydro-pyrrolo[3,2-f]quinolin-7-one

(Compound 212, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.64 (s, 1H), 7.55 (d, J=8.8 Hz, 1H), 7.22 (d, J=8.8 Hz, 1H), 7.15 (s, 1H), 4.14 (t, J=7.5 Hz, 2H), 2.51-2.46 (m, 4H), 2.41 (s, 3H), 2.32 (q, J=1.8 Hz, 3H), 1.75 (m, 2H), 1.60-1.24 (m, 16H), 0.87 (t, J=7.0 Hz, 3H)

### Example 160

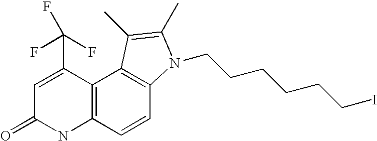

### 3-(6-Iodo-hexyl)-1,2-dimethyl-9-trifluoromethyl-3,6-dihydro-pyrrolo[3,2-f]quinolin-7-one

(Compound 213, Structure 27 of Scheme VIII, where R¹=Trifluoromethyl, R³=Hydrogen, R⁴³=Methyl, R⁴⁵=Methyl)

In a 500 ml amber-colored round bottom flask equipped with a magnetic stirrer and a cold water condenser, 3-(6-chloro-hexyl)-1,2-dimethyl-9-trifluoromethyl-3,6-dihydro-pyrrolo[3,2-f]quinolin-7-one (Compound 208, Example 163) (720 mg) was dissolved in HPLC grade acetone (250 ml) and sodium 12.0 grams was added and the reaction flask was stirred overnight (16 hrs) under reflux conditions in a pre-heated oil bath at 80° C. The following morning the reaction solution was diluted with deionized water (150 ml) and concentrated in situ using a rotovap until the reaction turned cloudy and a solid precipitate formed in the flask. The solid was filtered and washed with an excess of water, hexanes and then dried under reduced pressure to provide 340 mg of the title compound: ¹H NMR (500 MHz, CDCl₃) δ 11.79 (s, 1H), 7.54 (d, J=8.7 Hz, 1H), 7.14 (d, J=8.7 Hz, 1H), 7.14 (s, 1H), 4.14 (t, J=7.5 Hz, 2H), 3.17 (t, J=6.9 Hz, 2H), 2.41 (s, 3H), 2.32 (q, J=1.7 Hz, 3H), 1.81 (m, 2H), 1.76 (m, 2H), 1.49-1.35 (m, 4H).

### Example 161

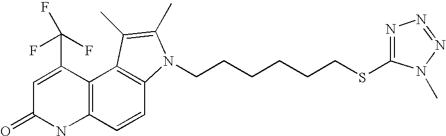

### 1,2-Dimethyl-3-[6-(1-methyl-1H-tetrazol-5-ylsulfanyl)-hexyl]-9-trifluoromethyl-3,6-dihydro-pyrrolo[3,2-f]quinolin-7-one

(Compound 210, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.24 (s, 1H), 7.54 (d, J=8.7 Hz, 1H), 7.19 (d, J=8.7 Hz, 1H), 7.15 (s, 1H), 4.14 (t, J=7.4 Hz, 2H), 3.90 (s, 3H), 3.32 (t, J=7.4 Hz, 2H), 2.41 (s, 3H), 2.32 (q, J=1.6 Hz, 3H), 1.82 (m, 2H), 1.76 (m, 2H), 1.49 (m, 2H), 1.42 (m, 2H).

### Example 162

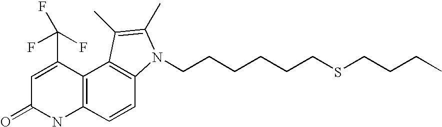

### 3-(6-Butylsulfanyl-hexyl)-1,2-dimethyl-9-trifluoromethyl-3,6-dihydro-pyrrolo[3,2-f]quinolin-7-one

(Compound 209, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.10 (s, 1H), 7.54 (d, J=8.8 Hz, 1H), 7.17 (d, J=8.8 Hz, 1H), 7.14 (s, 1H), 4.14 (t, J=7.5 Hz, 2H), 2.49 (t, J=7.4 Hz, 4H), 2.41 (s, 3H), 2.32 (q, J=1.6 Hz, 3H), 1.79-1.71 (m, 2H), 1.62-1.52 (m, 5H), 1.48-1.35 (m, 5H), 0.91 (t, J=7.4 Hz, 3H)

### Example 163

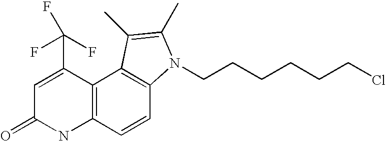

### 3-(6-Chloro-hexyl)-1,2-dimethyl-9-trifluoromethyl-3,6-dihydro-pyrrolo[3,2-f]quinolin-7-one

(Compound 208, Structure 27 of Scheme VIII, where R¹=Trifluoromethyl, R³=Hydrogen, R⁴³=Methyl, R⁴⁵=Methyl)

In a 50 ml round bottom flask equipped with a magnetic stirrer and a cold water condenser, 7-chloro-1,2-dimethyl-3-[6-(tetrahydro-pyran-2-yloxy)-hexyl]-9-trifluoro-methyl-3H-pyrrolo[3,2-f]quinoline (Example 172) (300 mg) was dispersed in conc. hydrochloric acid (10 ml) and heated to 80° C. under reflux for 48 hrs in an oil bath. After 2 days, TLC (2:1 ratios of EA to Hexanes) showed that the reaction was finished. The reaction solution was cooled to ambient temperature by removing the oil bath. The reaction mixture was extracted two times with dichloromethane (25 ml). The combined organic layers were washed with saturated aqueous sodium bicarbonate solution (25 ml), deionized water (25 ml) and brine (25 ml), then dried over anhydrous magnesium sulfate and filtered. The filtrate was evaporated in situ to obtain the crude product which was washed with a small amount of chloroform to provide 280 mg of the title compound: ¹H NMR (500 MHz, CDCl₃) δ 12.57 (s, 1H), 7.54 (d, J=8.8 Hz, 1H), 7.22 (d, J=8.8 Hz, 1H), 7.16 (s, 1H), 4.15 (t, J=7.5 Hz, 2H), 3.53 (t, J=6.6 Hz, 2H), 2.41 (s, 3H), 2.32 (q, J=1.8 Hz, 3H), 1.81-1.73 (m, 4H), 1.53-1.46 (m, 2H), 1.44-1.36 (m, 2H).

### Example 164

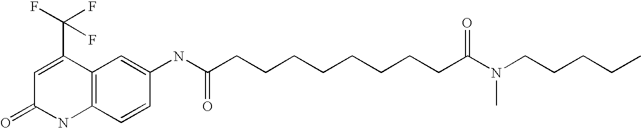

### Decanedioic acid methyl-pentyl-amide(2-oxo-4-trifluoromethyl-1,2-dihydro-quinolin-6-yl)-amide

(Compound 108, Structure 6 of Scheme I, where R¹=Trifluoromethyl, R²=Hydrogen, R³=Hydrogen, R⁴=Hydrogen, G=(C═O))

¹H NMR (500 MHz, Acetone-d₆) 9.37 (s, 1H), 8.28 (q, J=1.9 Hz, 1H), 7.94 (dd, J=9.0, 1.9 Hz, 1H), 7.47 (d, J=9.0 Hz, 1H), 6.94 (s, 1H), 3.32 (t, J=7.4 Hz, 1H), 3.31 (t, J=7.7 Hz, 1H), 2.99 (s, 1.5H), 2.84 (s, 1.5H), 2.40 (t, J=7.5 Hz, 2H), 2.31 (t, J=7.4 Hz, 1H), 2.29 (t, J=7.4 Hz, 1H), 1.70 (m, 2H), 1.61-1.54 (m, 3H), 1.48 (m, 1H), 1.41-1.27 (m, 12H), 0.90 (t, J=7.1 Hz, 1.5H), 0.88 (t, J=7.2 Hz, 1.5H).

### Example 165

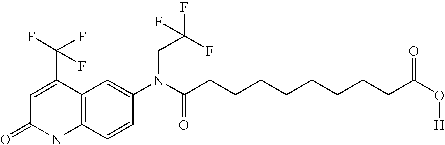

### 9-[(2-Oxo-4-trifluoromethyl-1,2-dihydro-quinolin-6-yl)-(2,2,2-trifluoro-ethyl)-carbamoyl]-nonanoic acid

(Compound 109, Structure 6 of Scheme I, where R¹=Trifluoromethyl, R²=Hydrogen, R³=Hydrogen, R⁴=Trifluoroethyl, G=(C═O))

¹H NMR (500 MHz, Acetone-d₆) δ 7.81 (br s, 1H), 7.75-7.67 (m, 2H), 7.06 (br s, 1H), 4.54 (q, J=9.2 Hz, 2H), 2.24 (t, J=7.2 Hz, 2H), 2.12 (t, J=7.1 Hz, 2H), 1.57-1.48 (m, 4H), 1.31-1.11 (m, 8H).

### Example 166

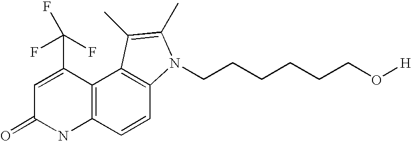

### 3-(6-Hydroxy-hexyl)-1,2-dimethyl-9-trifluoromethyl-3,6-dihydro-pyrrolo[3,2-f]-quinolin-7-one

(Compound 207, Structure 27 of Scheme VIII, where R¹=Trifluoromethyl, R³=Hydrogen, R⁴³=Methyl, R⁴⁵=Methyl)

In a 100 ml round bottom flask equipped with a magnetic stirrer, 1,2-dimethyl-3-[6-(tetrahydro-pyran-2-yloxy)-hexyl]-9-trifluoromethyl-3,6-dihydro-pyrrolo[3,2-f]-quinolin-7-one (Compound 206 in Example 172) (45.0 mg) was dissolved in methanol (20 ml) and p-toluenesulfonic acid (100 mg) was added. The reaction mixture was stirred overnight at room temperature (16 hrs). The following morning TLC (2:1 ratios of Ethyl acetate to Hexanes) showed that the reaction was finished. The reaction solution was diluted with deionized water (20 ml) and extracted three times with ethyl acetate (15 ml). The combined organic layers were washed two times with water (100 ml), and with brine (50 ml), then dried over anhydrous magnesium sulfate and filtered. The filtrate was absorbed on 2.2 volume of silica gel and further purified with flash column chromatography, eluted with 5% methanol in dichloromethane solution to provide the title compound: ¹H NMR (500 MHz, Acetone-d₆) δ 11.11 (s, 1H), 7.78 (d, J=8.8 Hz, 1H), 7.24 (d, J=8.8 Hz, 1H), 6.91 (s, 1H), 4.26 (t, J=7.6 Hz, 2H), 3.51 (t, J=6.3 Hz, 2H), 2.44 (s, 3H), 2.30 (q, J=2.0 Hz, 3H), 1.80-1.73 (m, 2H), 1.54-1.47 (m, 2H), 1.45-1.40 (m, 4H).

### Example 167

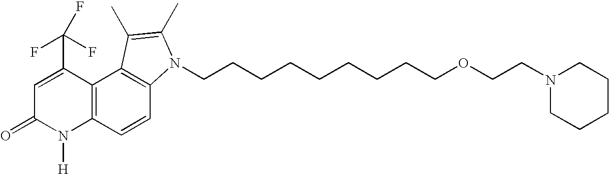

### 1,2-Dimethyl-3-[9-(2-piperidin-1-yl-ethoxy)-nonyl]-9-trifluoromethyl-3,6-dihydro-pyrrolo[3,2-f]quinolin-7-one

(Compound 203, Structure 27 of Scheme VIII where R¹=Trifluoromethyl, R³=Hydrogen, R⁴³=Methyl, R⁴⁵=Methyl)

¹H NMR (500 MHz, CDCl₃) δ 8.55 (s, 1H), 7.53 (d, J=8.6 Hz, 1H), 7.15 (d, J=8.6 Hz, 1H), 7.12 (s, 1H), 4.13 (t, J=7.4 Hz, 2H), 3.74 (t, J=5.1 Hz, 2H), 3.38 (t, J=6.7 Hz, 2H), 3.06-2.92 (m, 6H), 2.41 (s, 3H), 2.32 (q, J=1.7 Hz, 3H), 1.84 (m, 4H), 1.73 (m, 2H), 1.51 (m, 2H), 1.38-1.23 (m, 10H).

### Example 168

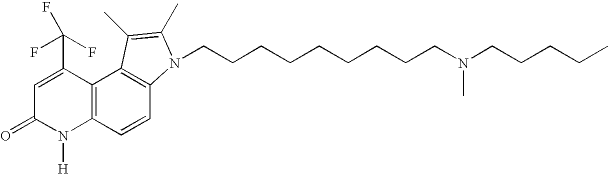

### 1,2-Dimethyl-3-[9-(methyl-pentyl-amino)-nonyl]-9-trifluoromethyl-3,6-dihydro-pyrrolo[3,2-f]quinolin-7-one

(Compound 204, Structure 27 of Scheme VIII, where R¹=Trifluoromethyl, R³=Hydrogen, R⁴³=Methyl, R⁴⁵=Methyl)

¹H NMR (500 MHz, CDCl₃) δ 7.54 (d, J=8.7 Hz, 1H), 7.23 (d, J=8.7 Hz, 1H), 7.14 (s, 1H), 4.13 (t, J=7.4 Hz, 2H), 2.68 (br, 4H), 2.52 (s, 3H), 2.41 (s, 3H), 2.32 (q, J=1.7 Hz, 3H), 1.76-1.60 (m, 4H), 1.38-1.24 (m, 16H), 0.90 (t, J=7.1 Hz, 3H).

### Example 169

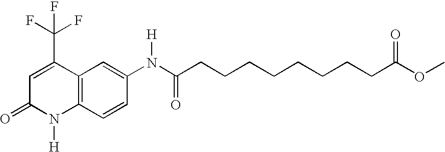

### 9-(2-Oxo-4-trifluoromethyl-1,2-dihydro-quinolin-6-ylcarbamoyl)-nonanoic acid methyl ester

(Compound 105, Structure 6 of Scheme I, where R¹=Trifluoromethyl, R²=Hydrogen, R³=Hydrogen, R⁴=Hydrogen, G=(C═O))

¹H NMR (500 MHz, CDCl₃) δ 11.57 (s, 1H), 7.94-7.89 (m, 2H), 7.36 (d, J=8.7 Hz, 1H), 7.35 (s, 1H), 7.09 (s, 1H), 3.67 (s, 3H), 2.40 (t, J=7.5 Hz, 2H), 2.31 (t, J=7.4 Hz, 2H), 1.75 (m, 2H), 1.68-1.60 (m, 2H), 1.42-1.29 (m, 8H).

### Example 170

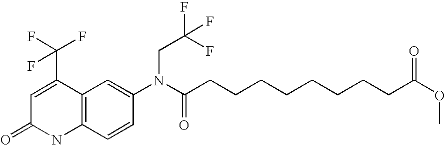

### 9-[(2-Oxo-4-trifluoromethyl-1,2-dihydro-quinolin-6-yl)-(2,2,2-trifluoro-ethyl)-carbamoyl]-nonanoic acid methyl ester

(Compound 107, Structure 6 of Scheme I, where R¹=Trifluoromethyl, R²=Hydrogen, R³=Hydrogen, R⁴=Trifluoroethyl, G=(C═O))

¹H NMR (500 MHz, CDCl₃) δ 12.78 (s, 1H), 7.71 (s, 1H), 7.60 (d, J=8.7 Hz, 1H), 7.49 (d, J=8.7 Hz, 1H), 7.18 (s, 1H), 4.40 (q, J=8.8 Hz, 2H), 3.66 (s, 3H), 2.28 (t, J=7.5 Hz, 2H), 2.05 (t, J=7.4 Hz, 2H), 1.71-1.58 (m, 3H), 1.38-1.12 (m, 9H).

### Example 171

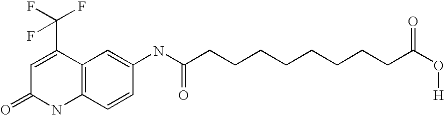

### 9-(2-Oxo-4-trifluoromethyl-1,2-dihydro-quinolin-6-ylcarbamoyl)-nonanoic acid

(Compound 106, Structure 6 of Scheme I, where R¹=Trifluoromethyl, R²=Hydrogen, R³=Hydrogen, R⁴=Hydrogen, G=(C═O))

¹H NMR (500 MHz, DMSO) δ 12.30 (s, 1H), 11.98 (s, 1H), 10.16 (s, 1H), 8.24 (m, 1H), 0.80 (m, 1H), 7.38 (d, J=8.9 Hz, 1H), 6.97 (m, 1H), 2.31 (t, J=7.2 Hz, 2H), 2.18 (t, J=7.2 Hz, 2H), 1.64-1.42 (m, 4H), 1.32-1.22 (m, 8H).

### Example 172

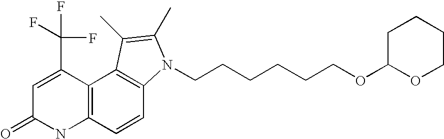

### (±)-1,2-Dimethyl-3-[6-(tetrahydro-pyran-2-yloxy)-hexyl]-9-trifluoromethyl-3,6-dihydro-pyrrolo[3,2-f]quinolin-7-one

(Compound 206, Structure 27 of Scheme VIII, where R¹=Trifluoromethyl, R³=Hydrogen, R⁴³=Methyl, R⁴⁵=Methyl)

In a 250 ml round bottom flask equipped with a magnetic stirrer and a cold water condenser, 1,2-dimethyl-9-trifluoromethyl-3,6-dihydro-pyrrolo[3,2-f]quinolin-7-one (11.5 g) was dispersed into 75 ml phosphorus oxychloride at room temperature and refluxed at 90° C. in an oil bath overnight. The following morning, TLC (8:92 ratios of Methanol in Dichloromethane) showed that the reaction was finished. The reaction mixture was cooled gradually to ambient temperature by removing the oil bath and then the reaction mixture was poured in a very slow stream over a period of 5 minutes into a 2 litter Erlenmeyer flask containing 750 ml crushed ice and stirred vigorously. The above aqueous solution was extracted with ethyl acetate (2×100 ml). The combined organic layers were washed several times with saturated aqueous sodium bicarbonate (2×75 ml) and then with water (150 ml) and brine (75 ml). The organic layer was dried over anhydrous magnesium sulfate and filtered. The organic filtrate was evaporated to dryness, hexane (200 ml) was added and stirred until a solid precipitated of the solution. The solid was filtered and washed with excess hexanes and then dried in-situ under reduced pressure to provide 8.5 g of 7-chloro-1,2-dimethyl-9-trifluoromethyl-3H-pyrrolo[3,2-f]quinoline.

In an oven-dried 20 ml round bottom flask equipped with a magnetic stirrer, 7-chloro-1,2-dimethyl-9-trifluoromethyl-3H-pyrrolo[3,2-f]quinoline (0.72 g), was dissolved in 8.0 ml dry N,N-dimethyl sulfoxide. To this solution and under a nitrogen atmosphere, powdered potassium hydroxide (0.9 g) was added quickly and stirred at room temperature for 10 min. 2-(6-Bromohexyloxy)tetrahydro-2-H-pyran (1.10 g) was added and the flask was tightly capped with a septum. The reaction flask was then heated in an oil bath at 60-62° C. overnight (16-18 hrs). The following morning, TLC (2:1 ratios of Ethyl acetate in Hexanes) showed that the reaction was finished. The reaction mixture was gradually cooled to room temperature, deionized water (60 ml) was added and extracted three times with ethyl acetate (50 ml). The combined organic layers were washed two times with water (100 ml) and brine (50 ml) then dried over anhydrous magnesium sulfate and filtered. The filtrate was then absorbed on 5 g of silica gel and further purified with flash column chromatography, eluted with 20%, 40%, 60%, 80% ethyl acetate in hexanes to provide 7-chloro-1,2-dimethyl-3-[6-(tetrahydro-pyran-2-yloxy)-hexyl]-9-trifluoromethyl-3H-pyrrolo[3,2-f]quinoline (0.8 g).

In a 20 ml round bottom flask equipped with a magnetic stirrer and a cold water condenser, 7-chloro-1,2-dimethyl-3-[6-(tetrahydro-pyran-2-yloxy)-hexyl]-9-trifluoromethyl-3H-pyrrolo[3,2-f]quinoline (50 mg) was dispersed in aqueous saturated sodium hydroxide solution (10 ml) and N,N-dimethyl sulfoxide (1.2 ml) was added and the reaction mixture was refluxed at 80° C. for 24 hrs. TLC (Etylacetate in Hexanes 2:1 ratio) showed that the reaction was finished. The reaction solution was cooled to ambient temperature by removing the oil bath, then deionized water (20 ml) was added and extracted three times with ethyl acetate (15 ml). The combined organic layers were washed two times with water (100 ml), and with brine (50 ml), then dried over anhydrous magnesium sulfate and filtered. The filtrate was absorbed on 2.2 volume of silica gel and further purified with flash column chromatography, eluted with 10%, 30%, and 50% ethyl acetate in hexanes to provide the title compound: ¹H NMR (500 MHz, CDCl₃) δ 12.01 (s, 1H), 7.54 (d, J=8.7 Hz, 1H), 7.15 (d, J=8.7 Hz, 1H), 7.14 (s, 1H), 4.55 (dd, J=4.5, 2.8 Hz, 1H), 4.13 (m, 2H), 3.85 (m, 1H), 3.73 (dt, J=9.6, 6.7 Hz, 1H), 3.49 (m, 1H), 3.37 (dt, J=9.6, 6.5 Hz, 1H), 2.41 (s, 3H), 2.32 (q, J=1.7 Hz, 3H), 1.85-1.67 (m, 4H), 1.64-1.49 (m, 6H), 1.46-1.38 (m, 4H).

### Example 173

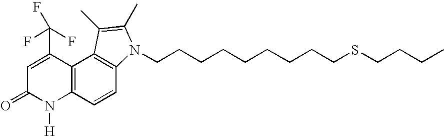

### 3-(9-Butylsulfanyl-nonyl)-1,2-dimethyl-9-trifluoromethyl-3,6-dihydro-pyrrolo[3,2-f]-quinolin-7-one

(Compound 205, Structure 27 of Scheme VIII, where R¹=Trifluoromethyl, R³=Hydrogen, R⁴³=Methyl, R⁴⁵=Methyl)

¹H NMR (500 MHz, CDCl₃) δ 11.63 (s, 1H), 7.54 (d, J=8.7 Hz, 1H), 7.13 (s, 1H), 7.12 (d, J=8.7 Hz, 1H), 4.12 (t, J=7.6 Hz, 2H), 2.52-2.47 (m, 4H), 2.41 (s, 3H), 2.32 (q, J=1.7 Hz, 3H), 1.73 (m, 2H), 1.56 (m, 4H), 1.44-1.25 (m, 12H), 0.91 (t, J=7.4 Hz, 3H).

### Example 174

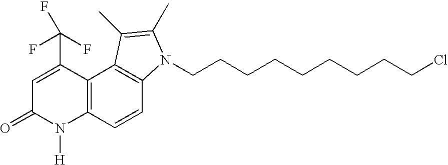

### 3-(9-Chloro-nonyl)-1,2-dimethyl-9-trifluoromethyl-3,6-dihydro-pyrrolo[3,2-f]quinolin-7-one

(Compound 201, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.55 (s, 1H), 7.55 (d, J=8.7 Hz, 1H), 7.21 (d, J=8.7 Hz, 1H), 7.15 (s, 1H), 4.13 (t, J=7.5 Hz, 2H), 3.52 (t, J=6.8 Hz, 2H), 2.41 (s, 3H), 2.32 (q, J=1.7 Hz, 3H), 1.79-1.70 (m, 4H), 1.45-1.27 (m, 10H).

### Example 175

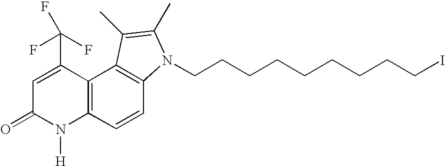

### 3-(9-Iodo-nonyl)-1,2-dimethyl-9-trifluoromethyl-3,6-dihydro-pyrrolo[3,2-f]quinolin-7-one

(Compound 202, Structure 27 of Scheme VIII, where R¹=Trifluoromethyl, R³=Hydrogen, R⁴³=Methyl, R⁴⁵=Methyl)

¹H NMR (500 MHz, CDCl₃) δ12.18 (s, 1H), 7.54 (d, J=8.8 Hz, 1H), 7.18 (d, J=8.8 Hz, 1H), 7.14 (s, 1H), 4.13 (t, J=7.5 Hz, 2H), 3.18 (t, J=7.0 Hz, 2H), 2.41 (s, 3H), 2.32 (q, J=1.3 Hz, 3H), 1.84-1.70 (m, 4H), 1.42-1.24 (m, 10H).

### Example 176

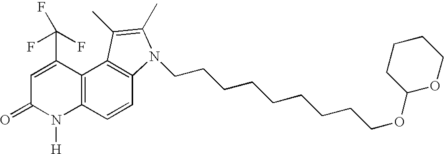

### (±)-1,2-Dimethyl-3-[9-(tetrahydro-pyran-2-yloxy)-nonyl]-9-trifluoromethyl-3,6-dihydro-pyrrolo[3,2-f]quinolin-7-one

(Compound 200, Structure 27 of Scheme VIII, where R¹=Trifluoromethyl, R³=Hydrogen, R⁴³=Methyl, R⁴⁵=Methyl)

¹H NMR (500 MHz, CDCl₃) δ 12.83 (s, 1H), 7.55 (d, J=8.8 Hz, 1H), 7.24 (d, J=8.8 Hz, 1H), 7.16 (s, 1H), 4.57 (dd, J=4.5, 2.7 Hz, 1H), 4.12 (t, J=7.6 Hz, 2H), 3.87 (ddd, J=11.4, 7.4, 3.2 Hz, 1H), 3.73 (dt, J=9.6, 6.9 Hz, 1H), 3.50 (m, 1H), 3.38 (dt, J=9.6, 6.7 Hz, 1H), 2.41 (s, 3H), 2.32 (q, J=1.5 Hz, 3H), 1.82 (m, 1H), 1.76-1.68 (m, 3H), 1.60-1.48 (m, 6H), 1.39-1.27 (m, 10H).

### Example 177

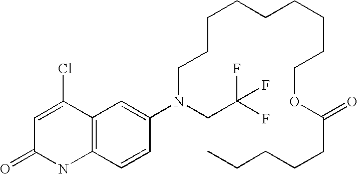

### Hexanoic acid 9-[(4-chloro-2-oxo-1,2-dihydro-quinolin-6-yl)-(2,2,2-trifluoro-ethyl)-amino]-nonyl ester

(Compound 102, Structure 5 of Scheme I, where R¹=Chloro, R²=Hydrogen, R³=Hydrogen, R⁴=R⁵=R⁷=R⁸=Hydrogen, R⁶=Trifluoromethyl, R⁹=8-Octanol Hexanoic Acid Ester)

¹H NMR (500 MHz, CDCl₃) δ 10.91 (s, 1H), 7.25 (d, J=9.0 Hz, 1H), 7.23 (d, J=2.7 Hz, 1H), 7.11 (dd, J=9.0, 2.7 Hz, 1H), 6.85 (s, 1H), 4.06 (t, J=6.8 Hz, 2H), 3.92 (q, J=8.8 Hz, 2H), 3.45 (t, J=7.7 Hz, 2H), 2.29 (t, J=7.6 Hz, 2H), 1.66-1.59 (m, 6H), 1.38-1.24 (m, 14H), 0.89 (t, J=7.0 Hz, 3H).

### Example 178

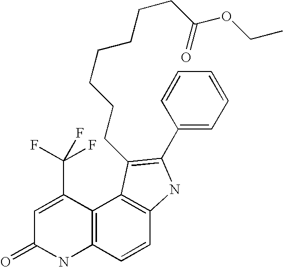

### 8-(7-Oxo-2-phenyl-9-trifluoromethyl-6,7-dihydro-3H-pyrrolo[3,2-f]quinolin-1-yl)-octanoic acid ethyl ester

(Compound 170, Structure 26 of Scheme VIII where R¹=Trifluoromethyl, R³=Hydrogen, R⁴⁵=Phenyl)

In a 200 ml 2-neck round bottom flask, equipped with a magnetic stirrer and a cold water condenser, and containing a solution of 6-amino-4-trifluoromethyl-1H-quinolin-2-one hydrazine hydrochloride (1.0 g) in 20 ml concentrated hydrochloric acid cooled to −5° C. was added a solution of 10-oxo-10-phenyl-decanoic acid methyl ester (1.0 g) in pure ethanol (5.0 ml). The reaction was heated to 98° C. in a pre-heated oil bath overnight. The following morning, TLC (2:1 ratios of Ethyl acetate in Hexanes) showed that the reaction was finished. The reaction mixture was cooled to room temperature, deionized water (25.0 ml) added and extracted three times with 25 ml ethyl acetate. The combined organic layers were washed with saturated sodium bicarbonate solution (75.0 ml), deionized water (50.0 ml) and brine solution (50.0 ml), dried over anhydrous magnesium sulfate and filtered. The filtrate was evaporated to dryness and the oily residue was absorbed on 2.2 volume of silica gel and further purified by flash column chromatography on silica gel, eluted with 50% and 75% EtOAc in Hexane solutions to provide 150 mg of the title compound: ¹H NMR (500 MHz, CDCl₃) δ 12.97 (s, 1H), 8.50 (s, 1H), 7.65 (d, J=8.7 Hz, 1H), 7.58 (m, 2H), 7.50 (t, J=7.4 Hz, 2H), 7.42 (tt, J=7.4, 1.3 Hz, 1H), 7.33 (d, J=8.7 Hz, 1H), 7.19 (s, 1H), 4.07 (q, J=7.1 Hz, 2H), 3.00 (t, J=7.7 Hz, 2H), 2.15 (t, J=7.6 Hz, 2H), 1.47-1.39 (m, 2H), 1.27-1.20 (m, 2H), 1.21 (t, J=7.1 Hz, 3H), 1.11-1.04 (m, 2H), 1.04-0.94 (m, 4H).

### Example 179

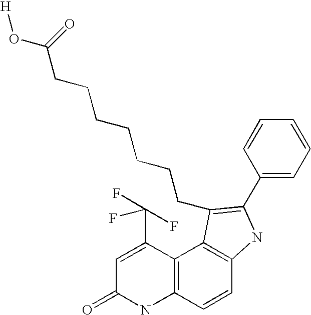

### 8-(7-Oxo-2-phenyl-9-trifluoromethyl-6,7-dihydro-3H-pyrrolo[3,2-f]quinolin-1-yl)-octanoic acid

(Compound 171, Structure 26 of Scheme VIII, where R¹=Trifluoromethyl, R³=Hydrogen, R⁴⁵=Phenyl)

In a 100 ml round bottom flask, equipped with a magnetic stirrer and a cold water condenser, containing a solution of Compound 170 in Example 178 (1.0 g) in THF (30.0 ml), aqueous sodium hydroxide solution (6.0 N, 20.0 ml) was added. The reaction was heated to 60° C. in an oil bath overnight. The following morning, TLC (66:33:5 ratio of Ethylacetate:Hexanes:Methanol) showed that the reaction was finished. The reaction mixture was cooled to room temperature, deionized water (25.0 ml) was added and then neutralized with hydrochloric acid solution (2.5 N) to a pH of 7.0. The reaction mixture was then extracted three times with 25.0 ml ethyl acetate. The combined organic layers were dried over anhydrous magnesium sulfate and filtered. The filtrate was evaporated to dryness to provide 450 mg of the title compound: ¹H NMR (500 MHz, Acetone-d₆) δ 11.02 (s, 1H), 10.94 (s, 1H), 7.80 (d, J=8.7 Hz, 1H), 7.68 (m, 2H), 7.54 (t, J=7.5 Hz, 2H), 7.43 (tt, J=7.5, 1.2 Hz, 1H), 7.34 (d, J=8.7 Hz, 1H), 7.00 (s, 1H), 3.07 (t, J=7.6 Hz, 2H), 12.14 (t, J=7.4 Hz, 2H), 1.42-1.27 (m, 4H), 1.12-0.94 (m, 6H).

### Example 180

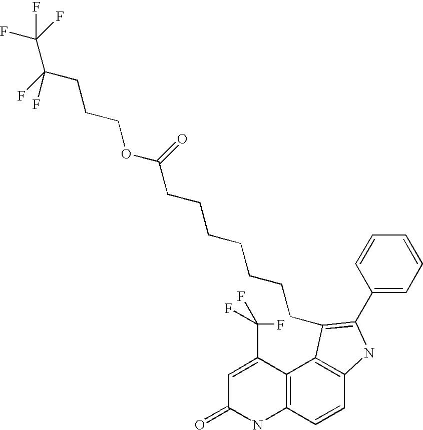

### 8-(7-Oxo-2-phenyl-9-trifluoromethyl-6,7-dihydro-3H-pyrrolo[3,2-f]quinolin-1-yl)-octanoic acid 4,4,5,5,5-pentafluoro-pentyl ester

(Compound 172, Structure 26 of Scheme VIII, where R¹=Trifluoromethyl, R³=Hydrogen, R⁴⁵=Phenyl)

¹H NMR (500 MHz, CDCl₃) δ 8.42 (s, 1H), 7.65 (d, J=8.6 Hz, 1H), 7.57 (m, 2H), 7.51 (t, J=7.4 Hz, 2H), 7.43 (tt, J=7.4, 1.3 Hz, 1H), 7.24 (d, J=8.6 Hz, 1H), 7.17 (s, 1H), 4.10 (t, J=6.3 Hz, 2H), 2.99 (t, J=7.7 Hz, 2H), 2.19 (t, J=7.6 Hz, 2H), 2.14-2.03 (m, 2H), 1.94-1.87 (m, 2H), 1.48-1.40 (m, 2H), 1.27-1.20 (m, 2H), 1.12-0.95 (m, 6H).

### Example 181

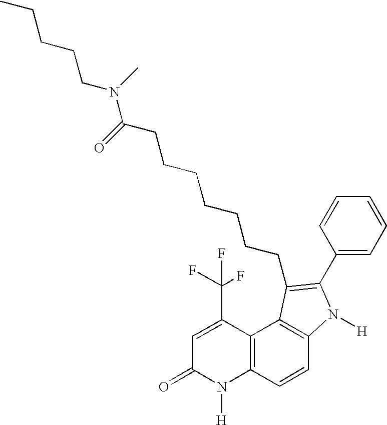

### 8-(7-Oxo-2-phenyl-9-trifluoromethyl-6,7-dihydro-3H-pyrrolo[3,2-f]quinolin-1-yl)-octanoic acid methyl-pentyl-amide

(Compound 173, Structure 26 of Scheme VIII, where R¹=Trifluoromethyl, R³=Hydrogen, R⁴⁵=Phenyl)

¹H NMR (500 MHz, CDCl₃) δ 8.56 (s, 2H), 7.64 (d, J=8.6 Hz, 2H), 7.57 (m, 4H), 7.50 (t, J=7.4 Hz, 4H), 7.42 (tt, J=7.4, 1.3 Hz, 2H), 7.26 (d, J=8.6 Hz, 2H), 7.17 (s, 2H), 3.30 (t, J=7.6 Hz, 2H), 3.18 (t, J=7.6 Hz, 2H), 3.00 (t, J=7.7 Hz, 4H), 2.90 (s, 3H), 2.86 (s, 3H), 2.16 (t, J=7.8 Hz, 2H), 2.15 (t, J=7.8 Hz, 2H), 1.54-1.39 (m, 6H), 1.34-1.18 (m, 14H), 1.12-0.94 (m, 12H), 0.88 (t, J=7.3 Hz, 3H), 0.86 (t, J=7.4 Hz, 3H).

### Example 182

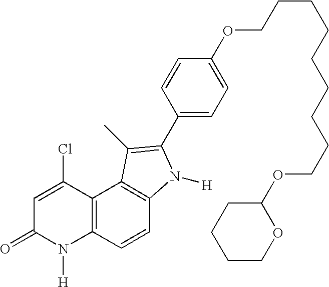

### 9-Chloro-1-methyl-2-{4-[9-(tetrahydro-pyran-2-yloxy)-nonyloxy]-phenyl}-3,6-dihydro-pyrrolo[3,2-f]quinolin-7-one

(Compound 195, Structure 26 of Scheme VIII, where R¹=Chloro, R³=Hydrogen, R⁴³=Methyl)

¹H NMR (500 MHz, CDCl₃) δ 11.94 (s, 1H), 8.57 (s, 1H), 7.61 (d, J=8.5 Hz, 1H), 7.50 (d, J=8.8 Hz, 2H), 7.22 (d, J=8.5 Hz, 1H), 7.02 (d, J=8.8 Hz, 2H), 6.91 (s, 1H), 4.58 (dd, J=4.5, 2.9 Hz, 1H), 4.02 (t, J=6.5 Hz, 2H), 3.88 (m, 1H), 3.73 (dt, J=9.6, 6.9 Hz, 1H), 3.51 (m, 1H), 3.38 (dt, J=9.6, 6.7 Hz, 1H), 2.66 (s, 3H), 1.86-1.79 (m, 3H), 1.73 (m, 1H), 1.63-1.45 (m, 8H), 1.41-1.31 (m, 8H).

### Example 183

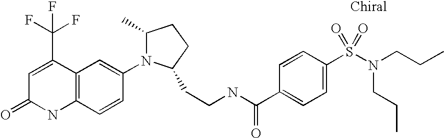

### 4-Dipropylsulfamoyl-N-{2-[(2R,5R)-5-methyl-1-(2-oxo-4-trifluoromethyl-1,2-dihydro-quinolin-6-yl)-pyrrolidin-2-yl]-ethyl}-benzamide

(Compound 141, Structure 14 of Scheme IV, where R²⁰=Trifluoromethyl, R²⁴=Methyl, R²⁷=Ethyl-2-(4-Dipropylsulfamoyl)Benzamide, Z=CH₂, n=0)

¹H NMR (300 MHz, Acetone-d₆) 8.12 (m, 1H), 8.08 (d, J=8.6 Hz, 2H), 7.91 (d, J=8.6 Hz, 2H), 7.39 (d, J=9.1 Hz, 1H), 7.16 (dm, J=9.1 Hz, 1H), 6.86 (m, 1H), 6.85 (s, 1H), 3.90-3.78 (m, 2H), 3.58 (m, 2H), 3.12 (t, J=7.5 Hz, 4H), 2.23-2.14 (m, 3H), 1.99 (m, 1H), 1.77 (m, 2H), 1.55 (sext, J=7.5 Hz, 4H), 1.32 (d, J=6.2 Hz, 3H), 0.85 (t, J=7.5 Hz, 6H).

### Example 184

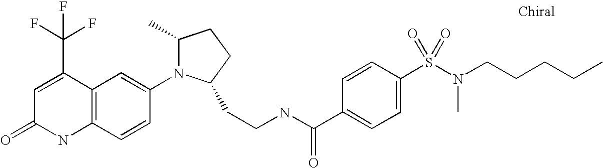

### N-{2-[(2R,5R)-5-Methyl-1-(2-oxo-4-trifluoromethyl-1,2-dihydro-quinolin-6-yl)-pyrrolidin-2-yl]-ethyl}-4-(methyl-pentyl-sulfamoyl)-benzamide

(Compound 142, Structure 14 of Scheme IV, where R²⁰=Trifluoromethyl, R²⁴=Methyl, R²⁷=Ethyl-2-(4-N-Methyl-N-Pentylsulfamoyl)Benzamide, Z=CH₁, n=0)

¹H NMR (300 MHz, Acetone-d₆) δ 8.14 (m, 1H), 8.10 (d, J=8.6 Hz, 2H), 7.88 (d, J=8.6 Hz, 2H), 7.39 (d, J=8.8 Hz, 1H), 7.17 (m, 1H), 6.86 (s, 1H), 6.86 (m, 1H), 3.91-3.76 (m, 2H), 3.63-3.52 (m, 2H), 3.02 (t, J=7.2 Hz, 2H), 2.74 (s, 3H), 2.27-2.14 (m, 3H), 2.02-1.96 (m, 1H), 1.87-1.71 (m, 2H), 1.54 (m, 2H), 1.36-1.27 (m, 4H), 1.32 (d, J=6.2 Hz, 3H), 0.88 (t, J=6.9 Hz, 3H).

### Example 185

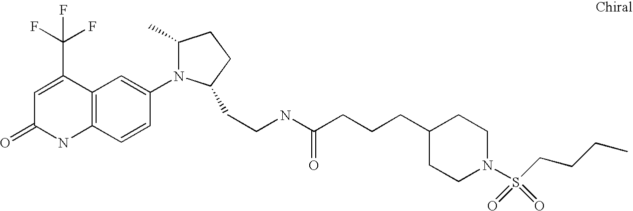

### 4-[1-(Butane-1-sulfonyl)-piperidin-4-yl]-N-{2-[(2R,5R)-5-methyl-1-(2-oxo-4-trifluoromethyl-1,2-dihydro-quinolin-6-yl)-pyrrolidin-2-yl]-ethyl}-butyramide

(Compound 143, Structure 14 of Scheme IV, where R²⁰=Trifluoromethyl, R²⁴=Methyl, Z=CH₂, n=0)

¹H NMR (300 MHz, Acetone-d₆) δ 7.40 (d, J=9.1 Hz, 1H), 7.15 (m, 1H), 7.11 (dd, J=9.1, 2.7 Hz, 1H), 6.87 (s, 1H), 6.84 (m, 1H), 3.85-3.73 (m, 2H), 3.72-3.64 (m, 2H), 3.39-3.26 (m, 2H), 2.93 (m, 2H), 2.76 (m, 2H), 2.16 (t, J=7.5 Hz, 2H), 2.14 (m, 1H), 1.93 (m, 1H), 1.82-1.58 (m, 8H), 1.52-1.38 (m, 4H), 1.30 (m, 3H), 1.29 (d, J=6.2 Hz, 3H), 1.24-1.08 (m, 2H), 0.93 (t, J=7.3 Hz, 3H).

### Example 186

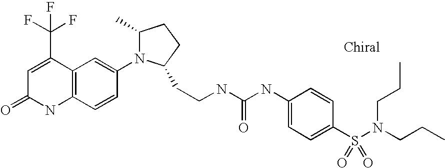

### 4-(3-{2-[(2R,5R)-5-Methyl-1-(2-oxo-4-trifluoromethyl-1,2-dihydro-quinolin-6-yl)-pyrrolidin-2-yl]-ethyl}-ureido)-N,N-dipropyl-benzenesulfonamide

(Compound 144, Structure 14 of Scheme IV, where R²⁰=Trifluoromethyl, R²⁴=Methyl, Z=CH₂, n=0)

¹H NMR (300 MHz, Acetone-d₆) δ 10.97 (s, 1H), 8.44 (s, 1H), 7.71 (d, J=9.3 Hz, 2H), 7.67 (d, J=9.3 Hz, 2H), 7.39 (d, J=9.0 Hz, 1H), 7.16 (dd, J=9.0, 2.5 Hz, 1H), 6.86 (s, 1H), 6.86 (m, 1H), 6.16 (t, J=5.4 Hz, 1H), 3.85-3.74 (m, 2H), 3.44-3.34 (m, 2H), 3.05 (t, J=7.5 Hz, 4H), 2.20-2.09 (m, 3H), 1.96 (m, 1H), 1.79 (m, 1H), 1.66 (m, 1H), 1.54 (sext, J=7.5 Hz, 4H), 1.30 (d, J=6.2 Hz, 3H), 0.86 (t, J=7.5 Hz, 6H).

### Example 187

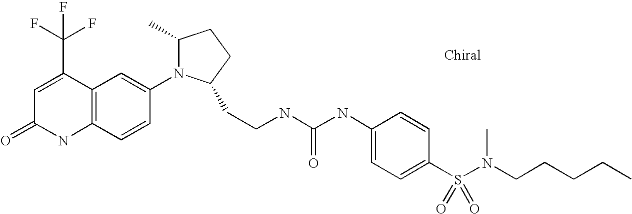

### N-Methyl-4-(3-{2-[(2R,5R)-5-methyl-1-(2-oxo-4-trifluoromethyl-1,2-dihydro-quinolin-6-yl)-pyrrolidin-2-yl]-ethyl}-ureido)-N-pentyl-benzenesulfonamide

(Compound 145, Structure 14 of Scheme IV, where R²⁰=Trifluoromethyl, R²⁴=Methyl, Z=CH₂, n=0)

¹H NMR (300 MHz, Acetone-d₆) δ 10.89 (s, 1H), 8.45 (s, 1H), 7.72 (d, J=8.7 Hz, 2H), 7.65 (d, J=8.7 Hz, 2H), 7.39 (d, J=9.0 Hz, 1H), 7.16 (dd, J=9.0, 2.6 Hz, 1H), 6.86 (m, 1H), 6.86 (s, 1H), 6.16 (t, J=5.5 Hz, 1H), 3.86-3.75 (m, 2H), 3.39 (m, 2H), 2.95 (t, J=7.1 Hz, 2H), 2.67 (s, 3H), 2.20-2.09 (m, 3H), 1.95 (m, 1H), 1.79 (m, 1H), 1.65 (m, 1H), 1.52 (m, 2H), 1.30 (d, J=6.2 Hz, 3H), 1.36-1.29 (m, 4H), 0.89 (t, J=6.9 Hz, 3H).

### Example 188

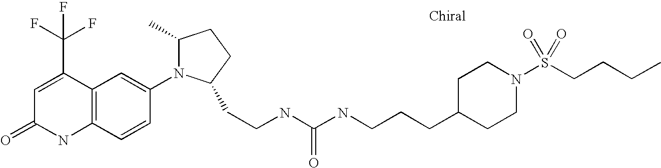

### 1-{3-[1-(Butane-1-sulfonyl)-piperidin-4-yl]-propyl}-3-{2-[(2R,5R)-5-methyl-1-(2-oxo-4-trifluoromethyl-1,2-dihydro-quinolin-6-yl)-pyrrolidin-2-yl]-ethyl}-urea

(Compound 146, Structure 14 of Scheme IV, where R²⁰=Trifluoromethyl, R²⁴=Methyl, Z=CH₂, n=0)

¹H NMR (300 MHz, Acetone-d₆) δ 7.40 (d, J=9.2 Hz, 1H), 7.15 (dd, J=9.2, 2.6 Hz, 1H), 6.87 (s, 1H), 6.84 (m, 1H), 5.54 (t, J=6.0 Hz, 1H), 5.48 (t, J=5.7 Hz, 1H), 3.83-3.63 (m, 4H), 3.26 (m, 2H), 3.13 (m, 2H), 2.98-2.90 (m, 2H), 2.75 (m, 2H), 2.14 (m, 1H), 1.90 (m, 1H), 1.82-1.66 (m, 6H), 1.61-1.37 (m, 6H), 1.30 (m, 3H), 1.29 (d, J=6.2 Hz, 3H), 1.24-1.06 (m, 2H), 0.93 (t, J=7.3 Hz, 3H).

### Example 189

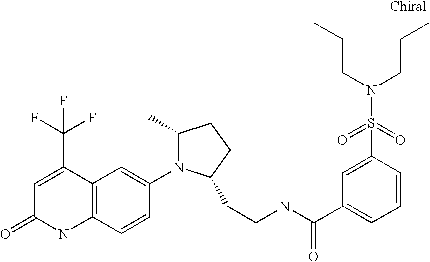

### 3-Dipropylsulfamoyl-N-{2-[(2R,5R)-5-methyl-1-(2-oxo-4-trifluoromethyl-1,2-dihydro-quinolin-6-yl)-pyrrolidin-2-yl]-ethyl}-benzamide

(Compound 148, Structure 14 of Scheme IV, where R²⁰=Trifluoromethyl, R²⁴=Methyl, Z=CH₂, n=0)

¹H NMR (300 MHz, Acetone-d₆) δ 10.95 (s, 1H), 8.31 (t, J=1.5 Hz, 1H), 8.25 (t, J=5.6 Hz, 1H), 8.17 (m, 1H), 7.98 (m, 1H), 7.71 (t, J=7.8 Hz, 1H), 7.39 (m, 1H), 7.16 (m, 1H), 6.86 (s, 1H), 6.86 (m, 1H), 3.91-3.78 (m, 2H), 3.57 (m, 2H), 3.11 (t, J=7.5 Hz, 4H), 2.28-2.12 (m, 3H), 2.00 (m, 1H), 1.79 (m, 2H), 1.55 (sext, J=7.5 Hz, 4H), 1.32 (d, J=6.2 Hz, 3H), 0.84 (t, J=7.5 Hz, 6H).

### Example 190

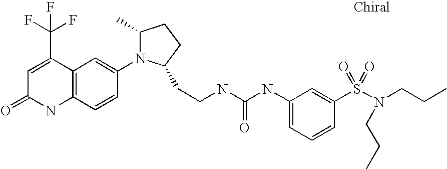

### 3-(3-{2-[(2R,5R)-5-Methyl-1-(2-oxo-4-trifluoromethyl-1,2-dihydro-quinolin-6-yl)-pyrrolidin-2-yl]-ethyl}-ureido)-N,N-dipropyl-benzenesulfonamide

(Compound 149, Structure 14 of Scheme IV, where R²⁰=Trifluoromethyl, R²⁴=Methyl, Z=CH₂, n=0)

¹H NMR (300 MHz, Acetone-d₆) δ 8.43 (s, 1H), 8.12 (t, J=2.0 Hz, 1H), 7.69 (ddd, J=7.9, 2.0, 1.2 Hz, 1H), 7.43 (t, J=7.9 Hz, 1H), 7.39 (d, J=9.0 Hz, 1H), 7.35 (ddd, J=7.9, 2.0, 1.2 Hz, 1H), 7.17 (dd, J=9.0, 2.6 Hz, 1H), 6.86 (m, 1H), 6.85 (s, 1H), 6.16 (t, J=5.7 Hz, 1H), 3.86-3.75 (m, 2H), 3.43-3.34 (m, 2H), 3.09 (t, J=7.5 Hz, 4H), 2.21-2.08 (m, 3H), 1.96 (m, 1H), 1.78 (m, 1H), 1.65 (m, 1H), 1.55 (sext, J=7.5 Hz, 4H), 1.30 (d, J=6.0 Hz, 3H), 0.85 (t, J=7.5 Hz, 6H).

### Example 191

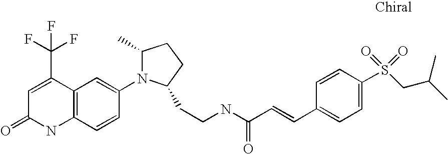

### (E)-N-{2-[(2R,5R)-5-Methyl-1-(2-oxo-4-trifluoromethyl-1,2-dihydro-quinolin-6-yl)-pyrrolidin-2-yl]-ethyl}-3-[4-(2-methyl-propane-1-sulfonyl)-phenyl]-acrylamide

(Compound 150, Structure 14 of Scheme IV, where R²⁰=Trifluoromethyl, R²⁴=Methyl, Z=CH₂, n=0)

¹H NMR (300 MHz, Acetone-d₆) δ 10.85 (s, 1H), 7.94 (d, J=8.2 Hz, 2H), 7.83 (d, J=8.2 Hz, 2H), 7.61 (d, J=15.6 Hz, 1H), 7.57 (m, 1H), 7.39 (d, J=9.1 Hz, 1H), 7.16 (dd, J=9.1, 2.6 Hz, 1H), 6.86 (m, 1H), 6.85 (s, 1H), 6.85 (d, J=15.6 Hz, 1H), 3.85-3.78 (m, 2H), 3.49 (m, 2H), 3.14 (d, J=6.6 Hz, 2H), 2.19-2.12 (m, 3H), 1.97 (m, 2H), 1.79 (m, 1H), 1.70 (m, 1H), 1.31 (d, J=6.1 Hz, 3H), 1.05 (d, J=6.8 Hz, 6H).

### Example 192

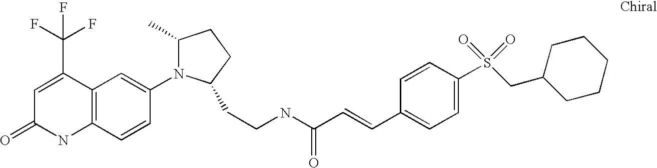

### (E)-3-(4-Cyclohexylmethanesulfonyl-phenyl)-N-{2-[(2R,5R)-5-methyl-1-(2-oxo-4-trifluoromethyl-1,2-dihydro-quinolin-6-yl)-pyrrolidin-2-yl]-ethyl}-acrylamide

(Compound 151, Structure 14 of Scheme IV, where R²⁰=Trifluoromethyl, R²⁴=Methyl, Z=CH₂, n=0)

¹H NMR (500 MHz, Acetone-d₆) δ 10.86 (s, 1H), 7.94 (d, J=8.4 Hz, 2H), 7.83 (d, J=8.4 Hz, 2H), 7.61 (d, J=15.6 Hz, 1H), 7.57 (t, J=5.7 Hz, 1H), 7.39 (d, J=9.0 Hz, 1H), 7.16 (dd, J=9.0, 2.7 Hz, 1H), 6.86 (m, 1H), 6.85 (d, J=15.6 Hz, 1H), 6.85 (s, 1H), 3.85-3.78 (m, 2H), 3.48 (m, 2H), 3.12 (d, J=6.1 Hz, 2H), 2.19-2.11 (m, 3H), 2.02-1.92 (m, 2H), 1.90-1.84 (m, 2H), 1.79 (m, 1H), 1.73-1.63 (m, 2H), 1.60 (m, 1H), 1.31 (d, J=6.1 Hz, 3H), 1.28-1.06 (m, 6H).

### Example 193

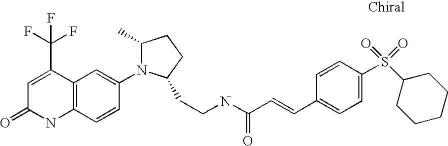

### (E)-3-(4-Cyclohexanesulfonyl-phenyl)-N-{2-[(2R,5R)-5-methyl-1-(2-oxo-4-trifluoromethyl-1,2-dihydro-quinolin-6-yl)-pyrrolidin-2-yl]-ethyl}-acrylamide

(Compound 152, Structure 14 of Scheme IV, where R²⁰=Trifluoromethyl, R²⁴=Methyl, Z=CH₂, n=0)

¹H NMR (500 MHz, Acetone-d₆) δ 10.86 (s, 1H), 7.88 (d, J=8.4 Hz, 2H), 7.83 (d, J=8.4 Hz, 2H), 7.62 (d, J=15.6 Hz, 1H), 7.58 (t, J=5.6 Hz, 1H), 7.39 (d, J=9.0 Hz, 1H), 7.16 (dd, J=9.0, 2.7 Hz, 1H), 6.86 (d, J=15.6 Hz, 1H), 6.86 (m, 1H), 6.85 (s, 1H), 3.85-3.78 (m, 2H), 3.52-3.46 (m, 2H), 3.08 (m, 1H), 2.21-2.10 (m, 3H), 2.02-1.96 (m, 3H), 1.85-1.76 (m, 3H), 1.73-1.62 (m, 1H), 1.41-1.23 (m, 5H), 1.31 (d, J=6.3 Hz, 3H), 1.16 (m, 1H)

### Example 194

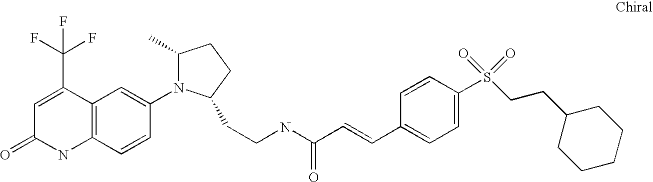

### (E)-3-[4-(2-Cyclohexyl-ethanesulfonyl)-phenyl]-N-{2-[(2R,5R)-5-methyl-1-(2-oxo-4-trifluoromethyl-1,2-dihydro-quinolin-6-yl)-pyrrolidin-2-yl]-ethyl}-acrylamide

(Compound 153, Structure 14 of Scheme IV, where R²⁰=Trifluoromethyl, R²⁴=Methyl, Z=CH₂, n=0)

¹H NMR (500 MHz, Acetone-d₆) δ 10.86 (s, 1H), 7.93 (d, J=8.3 Hz, 2H), 7.83 (d, J=8.3 Hz, 2H), 7.61 (d, J=15.6 Hz, 1H), 7.58 (t, J=5.5 Hz, 1H), 7.39 (d, J=9.1 Hz, 1H), 7.16 (dd, J=9.1, 2.6 Hz, 1H), 6.86 (m, 1H), 6.86 (d, J=15.6 Hz, 1H), 6.85 (s, 1H), 3.85-3.78 (m, 2H), 3.51-3.46 (m, 2H), 3.23 (m, 2H), 2.21-2.12 (m, 3H), 1.98 (m, 1H), 1.79 (m, 1H), 1.72-1.58 (m, 6H), 1.57-1.52 (m, 2H), 1.31 (d, J=6.3 Hz, 3H), 1.24-1.10 (m, 4H), 0.92-0.83 (m, 2H).

### Example 195

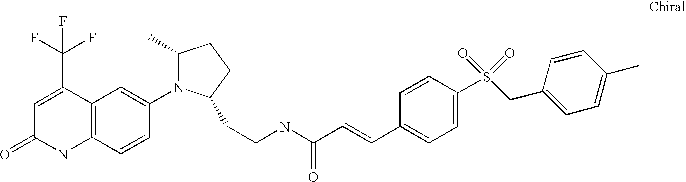

### (E)-N-{2-[(2R,5R)-5-Methyl-1-(2-oxo-4-trifluoromethyl-1,2-dihydro-quinolin-6-yl)-pyrrolidin-2-yl]-ethyl}-3-(4-p-tolylmethanesulfonyl-phenyl)-acrylamide

(Compound 154, Structure 14 of Scheme IV, where R²⁰=Trifluoromethyl, R²⁴=Methyl, Z=CH₂, n=0)

¹H NMR (500 MHz, Acetone-d₆) δ 7.74 (d, J=8.5 Hz, 2H), 7.70 (d, J=8.5 Hz, 2H), 7.58 (d, J=15.6 Hz, 1H), 7.57 (m, 1H), 7.39 (d, J=9.1 Hz, 1H), 7.16 (dd, J=9.1, 2.6 Hz, 1H), 7.10 (d, J=8.2 Hz, 2H), 7.07 (d, J=8.2 Hz, 2H), 6.86 (s, 1H), 6.86 (m, 1H), 6.83 (d, J=15.6 Hz, 1H), 4.49 (s, 2H), 3.85-3.78 (m, 2H), 3.49 (m, 2H), 2.29 (s, 3H), 2.21-2.12 (m, 3H), 1.97 (m, 1H), 1.79 (m, 1H), 1.70 (m, 1H), 1.31 (d, J=6.1 Hz, 3H).

### Example 196

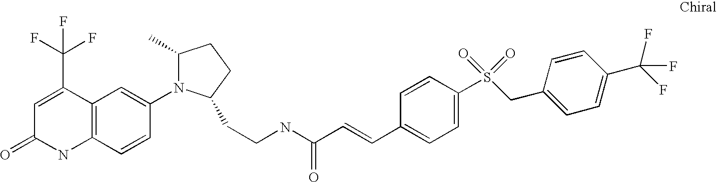

### (E)-N-{2-[(2R,5R)-5-Methyl-1-(2-oxo-4-trifluoromethyl-1,2-dihydro-quinolin-6-yl)-pyrrolidin-2-yl]-ethyl}-3-[4-(4-trifluoromethyl-phenylmethanesulfonyl)-phenyl]-acrylamide

(Compound 155, Structure 14 of Scheme IV, where R²⁰=Trifluoromethyl, R²⁴=Methyl, Z=CH₂, n=0)

¹H NMR (500 MHz, Acetone-d₆) δ 7.77 (d, J=8.7 Hz, 2H), 7.74 (d, J=8.7 Hz, 2H), 7.67 (d, J=8.0 Hz, 2H), 7.59 (d, J=15.6 Hz, 1H), 7.56 (t, J=6.1 Hz, 1H), 7.47 (d, J=8.0 Hz, 2H), 7.39 (d, J=9.2 Hz, 1H), 7.16 (dd, J=9.2, 2.6 Hz, 1H), 6.86 (s, 1H), 6.86 (m, 1H), 6.83 (d, J=15.6 Hz, 1H), 4.71 (s, 2H), 3.86-3.78 (m, 2H), 3.49 (m, 2H), 2.20-2.12 (m, 3H), 1.97 (m, 1H), 1.79 (m, 1H), 1.70 (m, 1H), 1.31 (d, J=6.1 Hz, 3H).

### Example 197

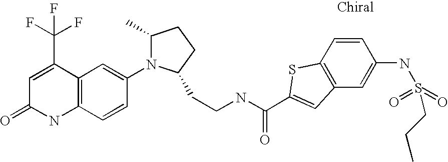

### 5-(Propane-1-sulfonylamino)-benzo[b]thiophene-2-carboxylic acid {2-[(2R,5R)-5-methyl-1-(2-oxo-4-trifluoromethyl-1,2-dihydro-quinolin-6-yl)-pyrrolidin-2-yl]-ethyl}-amide

(Compound 157, Structure 14 of Scheme IV, where R²⁰=Trifluoromethyl, R²⁴=Methyl, Z=CH₂, n=0)

¹H NMR (500 MHz, Acetone-d₆) δ 10.85 (s, 1H), 8.75 (s, 1H), 8.06 (t, J=5.3 Hz, 1H), 7.95 (d, J=8.7 Hz, 1H), 7.91 (s, 1H), 7.86 (d, J=2.1 Hz, 1H), 7.46 (dd, J=8.7, 2.1 Hz, 1H), 7.37 (d, J=9.0 Hz, 1H), 7.17 (m, 1H), 6.87 (m, 1H), 6.85 (s, 1H), 3.90-3.78 (m, 2H), 3.62-3.50 (m, 2H), 3.11 (m, 2H), 2.25-2.15 (m, 3H), 2.01 (m, 1H), 1.84-1.73 (m, 2H), 1.81 (sext, J=7.5 Hz, 2H), 1.32 (d, J=6.1 Hz, 3H), 0.98 (t, J=7.5 Hz, 3H).

### Example 198

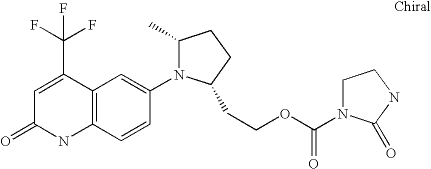

### 2-Oxo-imidazolidine-1-carboxylic acid 2-[(2R,5R)-5-methyl-1-(2-oxo-4-trifluoromethyl-1,2-dihydro-quinolin-6-yl)-pyrrolidin-2-yl]-ethyl ester

(Compound 162, Structure 14 of Scheme IV, where R²⁰=Trifluoromethyl, R²⁴=Methyl, Z=CH₂, n=0)

¹H NMR (500 MHz, Acetone-d₆) δ 10.97 (s, 1H), 7.41 (d, J=9.0 Hz, 1H), 7.37 (dd, J=9.0, 2.4 Hz, 1H), 6.87 (s, 1H), 6.84 (m, 1H), 6.35 (br s, 1H), 4.29 (m, 2H), 3.97 (m, 1H), 3.91 (m, 2H), 3.80 (m, 1H), 3.48 (m, 2H), 2.24-2.16 (m, 2H), 2.11 (m, 1H), 1.98 (m, 1H), 1.85-1.71 (m, 2H), 1.32 (d, J=6.3 Hz, 3H).

### Example 199

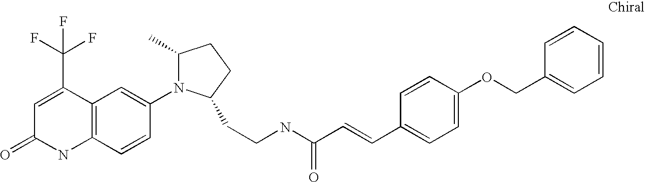

### (E)-3-(4-Benzyloxy-phenyl)-N-{2-[(2R,5R)-5-methyl-1-(2-oxo-4-trifluoromethyl-1,2-dihydro-quinolin-6-yl)-pyrrolidin-2-yl]-ethyl}-acrylamide

(Compound 163, Structure 14 of Scheme IV, where R²⁰=Trifluoromethyl, R²⁴=Methyl, Z=CH₂, n=0)

¹H NMR (500 MHz, Acetone-d₆) δ 7.53 (d, J=8.8 Hz, 2H), 7.50 (d, J=15.5 Hz, 1H), 7.49 (d, J=7.5 Hz, 2H), 7.40 (t, J=7.5 Hz, 2H), 7.38 (d, J=9.0 Hz, 1H), 7.34 (t, J=7.5 Hz, 1H), 7.15 (dd, J=9.0, 2.7 Hz, 1H), 7.05 (d, J=8.8 Hz, 2H), 6.85 (s, 1H), 6.85 (m, 1H), 6.54 (d, J=15.5 Hz, 1H), 5.17 (s, 2H), 3.83-3.77 (m, 2H), 3.48-3.43 (m, 2H), 2.14 (m, 1H), 1.97 (m, 1H), 1.82-1.76 (m, 2H), 1.70-1.62 (m, 2H), 1.31 (d, J=6.3 Hz, 3H).

### Example 200

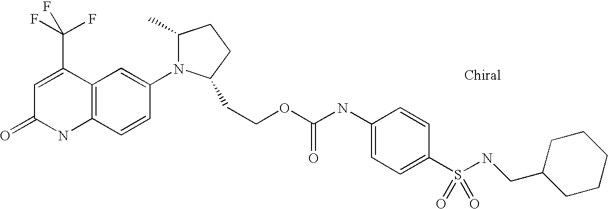

### [4-(Cyclohexylmethyl-sulfamoyl)-phenyl]-carbamic acid 2-[(2R,5R)-5-methyl-1-(2-oxo-4-trifluoromethyl-1,2-dihydro-quinolin-6-yl)-pyrrolidin-2-yl]-ethyl ester

(Compound 164, Structure 14 of Scheme IV, where R²⁰=Trifluoromethyl, R²⁴=Methyl, Z=CH₂, n=0)

¹H NMR (300 MHz, Acetone-d₆) δ 10.87 (br s, 1H), 9.14 (br s, 1H), 7.81-7.73 (m, 4H), 7.40 (d, J=9.1 Hz, 1H), 7.17 (dd, J=9.1, 2.6 Hz, 1H), 6.86 (m, 1H), 6.86 (s, 1H), 6.30 (t, J=6.5 Hz, 1H), 4.37-4.30 (m, 2H), 3.93-3.76 (m, 2H), 2.72 (t, J=6.5 Hz, 2H), 2.30-2.12 (m, 3H), 1.87-1.57 (m, 8H), 1.43 (m, 1H), 1.32 (d, J=6.2 Hz, 3H), 1.19-1.12 (m, 3H), 0.95-0.80 (m, 2H).

### Example 201

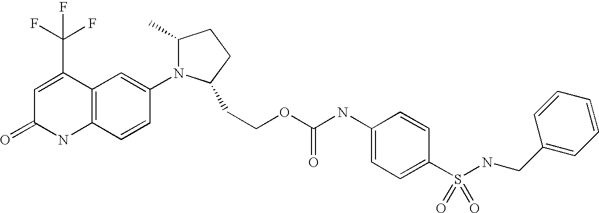

### (4-Benzylsulfamoyl-phenyl)-carbamic acid 2-[(2R,5R)-5-methyl-1-(2-oxo-4-trifluoromethyl-1,2-dihydro-quinolin-6-yl)-pyrrolidin-2-yl]-ethyl ester

(Compound 165, Structure 14 of Scheme IV, where R²⁰=Trifluoromethyl, R²⁴=Methyl, Z=CH₂, n=0)

¹H NMR (500 MHz, Acetone-d₆) δ 10.89 (s, 1H), 9.15 (s, 1H), 7.82 (d, J=8.9 Hz, 2H), 7.76 (d, J=8.9 Hz, 2H), 7.40 (d, J=9.1 Hz, 1H), 7.31-7.26 (m, 4H), 7.23 (m, 1H), 7.18 (dd, J=9.1, 2.7 Hz, 1H), 6.88-6.86 (m, 2H), 6.82 (t, J=6.3 Hz, 1H), 4.39-4.30 (m, 2H), 4.11 (d, J=6.3 Hz, 2H), 3.89 (m, 1H), 3.82 (m, 1H), 2.28-2.15 (m, 3H), 2.01-1.97 (m, 1H), 1.88-1.79 (m, 2H), 1.32 (d, J=6.3 Hz, 3H).

### Example 202

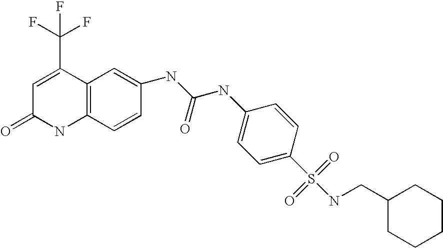

### N-Cyclohexylmethyl-4-[3-(2-oxo-4-trifluoromethyl-1,2-dihydro-quinolin-6-yl)-ureido]-benzenesulfonamide

### (Compound 111, Structure 6 of Scheme I)

¹H NMR (500 MHz, DMSO) δ 9.13 (s, 1H), 9.11 (s, 1H), 8.12 (m, 1H), 7.69 (d, J=9.0 Hz, 2H), 7.64 (d, J=9.0 Hz, 2H), 7.63 (dd, J=8.9, 2.2 Hz, 1H), 7.42 (t, J=6.2 Hz, 1H), 7.40 (d, J=8.9 Hz, 1H), 6.99 (s, 1H), 2.53 (t, J=6.2 Hz, 2H), 1.67-1.55 (m, 5H), 1.31 (m, 1H), 1.15-1.05 (m, 3H), 0.79 (m, 2H).

### Example 203

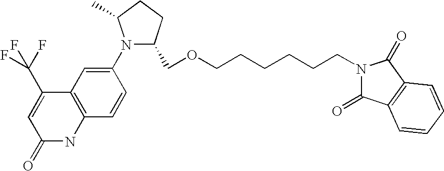

### 2-{6-[(2R,5R)-5-Methyl-1-(2-oxo-4-trifluoromethyl-1,2-dihydro-quinolin-6-yl)-pyrrolidin-2-ylmethoxy]-hexyl}-isoindole-1,3-dione

### (Compound 161, Structure 14 of Scheme IV)

¹H NMR (500 MHz, DMSO) δ 12.03 (s, 1H), 7.87-7.80 (m, 4H), 7.31 (d, J=9.1 Hz, 1H), 7.15 (dd, J=9.1, 2.4 Hz, 1H), 6.86 (s, 1H), 6.77 (m, 1H), 3.75 (m, 1H), 3.70 (m, 1H), 3.52 (t, J=7.1 Hz, 2H), 3.47-3.37 (m, 3H), 2.10 (m, 1H), 1.94-1.80 (m, 2H), 1.68 (m, 1H), 1.56 (m, 2H), 1.49 (m, 2H), 1.36-1.22 (m, 4H), 1.19 (d, J=6.1 Hz, 3H).

### Example 204

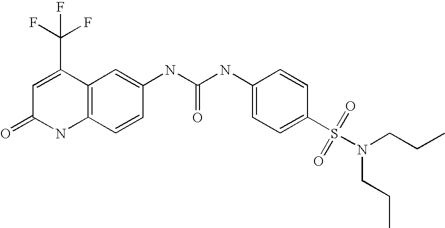

### 4-[3-(2-Oxo-4-trifluoromethyl-1,2-dihydro-quinolin-6-yl)-ureido]-N,N-dipropyl-benzenesulfonamide

### (Compound 110, Structure 6 of Scheme I)

¹H NMR (500 MHz, DMSO) δ 9.17 (s, 1H), 9.11 (s, 1H), 8.11 (m, 1H), 7.70 (d, J=9.0 Hz, 2H), 7.65 (d, J=9.0 Hz, 2H), 7.63 (dd, J=8.8, 2.2 Hz, 1H), 7.40 (d, J=8.8 Hz, 1H), 6.99 (s, 1H), 2.99 (t, J=7.4 Hz, 4H), 1.47 (sext, J=7.4 Hz, 4H), 0.81 (t, J=7.4 Hz, 6H).

### Example 205

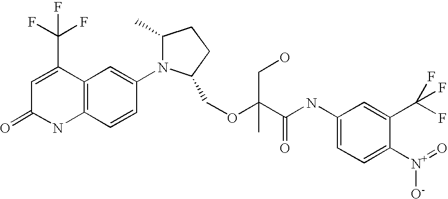

### 3-Hydroxy-2-methyl-2-[(2R,5R)-5-methyl-1-(2-oxo-4-trifluoromethyl-1,2-dihydro-quinolin-6-yl)-pyrrolidin-2-ylmethoxy]-N-(4-nitro-3-trifluoromethyl-phenyl)-propionamide

### (Compound 159, Structure 14 of Scheme IV)

¹H NMR (500 MHz, DMSO) δ 11.99 (s, 1H), 10.45 (s, 1H), 8.49 (d, J=2.1 Hz, 1H), 8.17 (dd, J=8.9, 2.1 Hz, 1H), 8.06 (d, J=8.9 Hz, 1H), 7.22 (d, J=9.2 Hz, 1H), 7.16 (dd, J=9.2, 2.3 Hz, 1H), 6.84 (s, 1H), 6.62 (m, 1H), 3.73 (m, 2H), 3.62 (m, 1H), 3.54 (m, 2H), 3.47 (m, 1H), 2.05 (m, 1H), 1.81-1.75 (m, 2H), 1.64 (m, 1H), 1.31 (s, 3H), 1.15 (d, J=5.9 Hz, 3H).

### Example 206

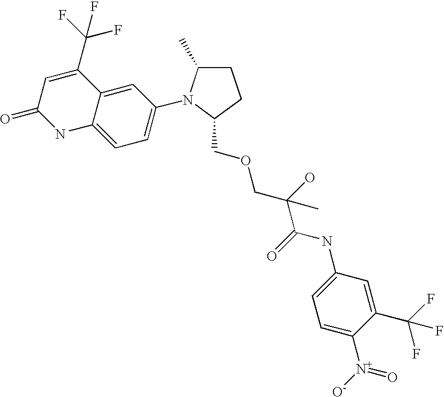

### 2-Hydroxy-2-methyl-3-[(2R,5R)-5-methyl-1-(2-oxo-4-trifluoromethyl-1,2-dihydro-quinolin-6-yl)-pyrrolidin-2-ylmethoxy]-N-(4-nitro-3-trifluoromethyl-phenyl)-propionamide

### (Compound 158, Structure 14 of Scheme IV)

¹H NMR (500 MHz, DMSO) δ 12.02 (s, 1H), 10.50 (s, 1H), 8.52 (d, J=2.2 Hz, 1H), 8.27 (dd, J=9.0, 2.2 Hz, 1H), 8.13 (d, J=9.0 Hz, 1H), 7.26 (d, J=9.0 Hz, 1H), 7.11 (dd, J=9.0, 2.6 Hz, 1H), 6.86 (s, 1H), 6.66 (m, 1H), 6.01 (br s, 1H), 3.75 (m, 1H), 3.72 (d, J=10.1 Hz, 1H), 3.67-3.58 (m, 2H), 3.54 (d, J=10.1 Hz, 1H), 3.40 (m, 1H), 2.03 (m, 1H), 1.86-1.75 (m, 2H), 1.63 (m, 1H), 1.11 (d, J=5.9 Hz, 3H).

### Example 207

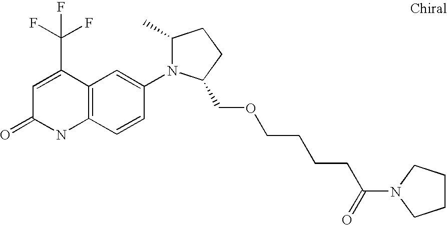

### 6-[(2R,5R)-2-Methyl-5-(5-oxo-5-pyrrolidin-1-yl-pentyloxymethyl)-pyrrolidin-1-yl]-4-trifluoromethyl-1H-quinolin-2-one

### (Compound 156, Structure 14 of Scheme IV)

¹H NMR (500 MHz, DMSO) δ 12.05 (s, 1H), 8.19 (s, 1H), 7.32 (d, J=9.1 Hz, 1H), 7.17 (dd, J=9.1, 2.6 Hz, 1H), 6.90 (s, 1H), 6.79 (m, 1H), 3.78 (m, 1H), 3.72 (m, 1H), 3.48 (dd, J=9.6, 4.6 Hz, 1H), 3.46-3.41 (m, 3H), 3.38-3.31 (m, 4H), 3.24 (t, J=6.8 Hz, 2H), 2.21 (m, 2H), 2.11 (m, 1H), 1.96-1.79 (m, 4H), 1.76-1.67 (m, 3H), 1.53 (m, 4H), 1.21 (d, J=5.9 Hz, 3H).

### Example 208

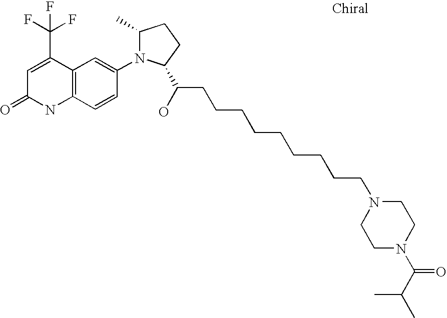

### 6-{(2R,5R)-2-[1-Hydroxy-10-(4-isobutyryl-piperazin-1-yl)-decyl]-5-methyl-pyrrolidin-1-yl}-4-trifluoromethyl-1H-quinolin-2-one

### (Compound 147, Structure 14 of Scheme IV)

¹H NMR (500 MHz, DMSO) δ 12.04 (s, 1H), 7.31 (d, J=9.2 Hz, 1H), 7.09 (dd, J=9.2, 2.3 Hz, 1H), 6.89 (s, 1H), 6.69 (m, 1H), 4.59 (m, 1H), 3.73 (m, 1H), 3.65 (m, 1H), 3.57 (m, 1H), 3.48-3.40 (m, 4H), 2.83 (sept, J=6.7 Hz, 1H), 2.38-2.24 (m, 6H), 2.11 (m, 1H), 1.98 (m, 1H), 1.77-1.64 (m, 2H), 1.51-1.18 (m, 19H), 0.97 (d, J=6.7 Hz, 6H).

### Example 209

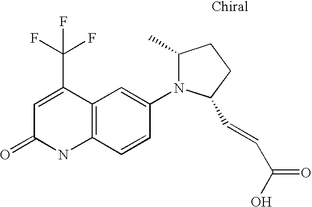

### (E)-3-[(2R,5R)-5-Methyl-1-(2-oxo-4-trifluoromethyl-1,2-dihydro-quinolin-6-yl)-pyrrolidin-2-yl]-acrylic acid

### (Compound 103, Structure 14 of Scheme IV)

¹H NMR (500 MHz, DMSO) δ 12.08 (s, 1H), 7.33 (d, J=9.2 Hz, 1H), 7.06 (dd, J=9.2, 2.5 Hz, 1H), 6.90 (s, 1H), 6.81 (dd, J=15.3, 5.1 Hz, 1H), 6.70 (m, 1H), 5.86 (dd, J=15.3, 1.1 Hz, 1H), 4.27 (m, 1H), 3.87 (m, 1H), 2.18 (m, 1H), 2.10 (m, 1H), 1.87 (m, 1H), 1.64 (m, 1H), 1.26 (d, J=6.1 Hz, 3H).

### Example 210

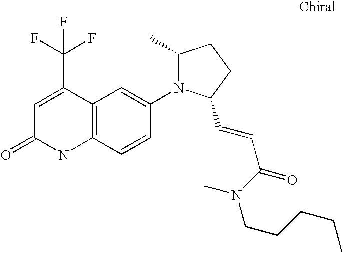

### (E)-N-Methyl-3-[(2R,5R)-5-methyl-1-(2-oxo-4-trifluoromethyl-1,2-dihydro-quinolin-6-yl)-pyrrolidin-2-yl]-N-pentyl-acrylamide

### (Compound 138, Structure 14 of Scheme IV)

¹H NMR (500 MHz, CD₃CN) δ 9.91 (br s, 1H), 7.25 (d, J=9.0 Hz, 1H), 7.04 (dd, J=9.0, 2.5 Hz, 1H), 6.90 (s, 1H), 6.84 (m, 1H), 6.80 (dd, J=15.0, 4.8 Hz, 1H), 6.43 (dd, J=15.0, 1.5 Hz, 1H), 4.32 (m, 1H), 3.93 (m, 1H), 3.25 (m, 1H), 3.17 (m, 1H), 2.89 (s, 3H), 1.91 (m, 2H), 1.71 (m, 2H), 1.50 (m, 2H), 1.34 (d, J=6.2 Hz, 3H), 1.13-1.00 (m, 4H), 0.73 (t, J=7.1 Hz, 3H)

### Example 211

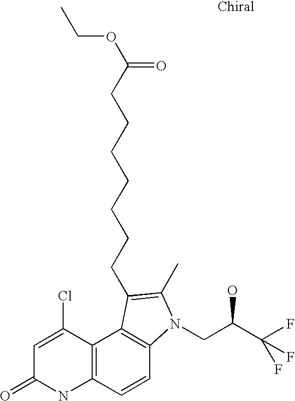

### 8-[9-Chloro-2-methyl-7-oxo-3-((R)-3,3,3-trifluoro-2-hydroxy-propyl)-6,7-dihydro-3H-pyrrolo[3,2-f]quinolin-1-yl]-octanoic acid ethyl ester

### (Compound 182, Structure 27 of Scheme VIII)

¹H NMR (500 MHz, Acetone-d₆) δ 7.75 (d, J=8.8 Hz, 1H), 7.23 (d, J=8.8 Hz, 1H), 6.65 (s, 1H), 4.60 (m, 1H), 4.54-4.47 (m, 2H), 4.07 (q, J=7.1 Hz, 1H), 3.10 (m, 2H), 2.54 (s, 3H), 2.25 (t, J=7.4 Hz, 2H), 1.59-1.52 (m, 4H), 1.35-1.28 (m, 6H), 1.20 (t, J=7.1 Hz, 3H).

### Example 212

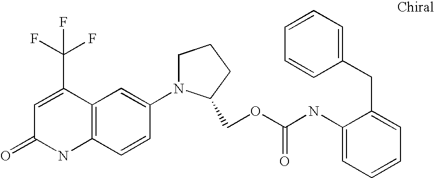

### (2-Benzyl-phenyl)-carbamic acid (R)-1-(2-oxo-4-trifluoromethyl-1,2-dihydro-quinolin-6-yl)-pyrrolidin-2-ylmethyl ester

### (Compound 139, Structure 14 of Scheme IV)

¹H NMR (500 MHz, Acetone-d₆) δ 7.85 (s, 1H), 7.58 (m, 1H), 7.42 (d, J=9.1 Hz, 1H), 7.33 (dd, J=9.1, 2.6 Hz, 1H), 7.29-7.22 (m, 3H), 7.20-7.15 (m, 3H), 7.12 (td, J=7.4, 1.0 Hz, 1H), 6.88 (s, 1H), 6.87 (m, 1H), 4.31 (dd, J=11.0, 3.8 Hz, 1H), 4.05 (s, 2H), 4.02 (m, 1H), 3.88 (dd, J=11.0, 8.2 Hz, 1H), 3.49 (m, 1H), 3.16 (m, 1H), 2.14 (m, 1H), 2.09-1.99 (m, 3H).

### Example 213

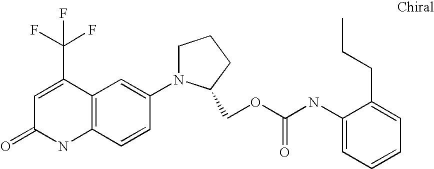

### (2-Propyl-phenyl)-carbamic acid (R)-1-(2-oxo-4-trifluoromethyl-1,2-dihydro-quinolin-6-yl)-pyrrolidin-2-ylmethyl ester

### (Compound 140, Structure 14 of Scheme IV)

¹H NMR (500 MHz, Acetone-d₆) δ 7.98 (s, 1H), 7.55 (m, 1H), 7.43 (d, J=9.1 Hz, 1H), 7.37 (dd, J=9.1, 2.6 Hz, 1H), 7.22 (dd, J=7.5, 1.4 Hz, 1H), 7.19 (td, J=7.5, 1.5 Hz, 1H), 7.11 (t, J=7.5 Hz, 1H), 6.90 (s, 1H), 6.89 (m, 1H), 4.37 (dd, J=11.1, 3.9 Hz, 1H), 4.09 (m, 1H), 3.92 (dd, J=11.1, 8.3 Hz, 1H), 3.52 (m, 1H), 3.18 (m, 1H), 2.64 (t, J=7.5 Hz, 2H), 2.17 (m, 1H), 2.09 (m, 3H), 1.60 (sext, J=7.5 Hz, 2H), 0.92 (t, J=7.5 Hz, 3H).

### Example 214

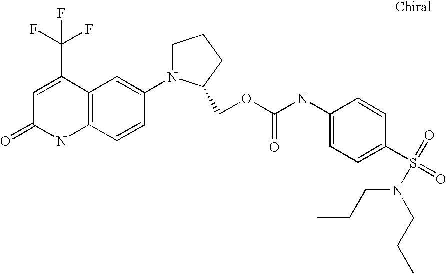

### (4-Dipropylsulfamoyl-phenyl)-carbamic acid (R)-1-(2-oxo-4-trifluoromethyl-1,2-dihydro-quinolin-6-yl)-pyrrolidin-2-ylmethyl ester

### (Compound 166, Structure 14 of Scheme IV)

¹H NMR (500 MHz, Acetone-d₆) δ 9.24 (s, 1H), 7.78 (s, 4H), 7.45 (d, J=9.1 Hz, 1H), 7.36 (dd, J=9.1, 2.7 Hz, 1H), 6.91 (m, 1H), 6.89 (s, 1H), 4.41 (dd, J=11.0, 3.7 Hz, 1H), 4.11 (m, 1H), 3.99 (dd, J=11.0, 8.3 Hz, 1H), 3.55 (m, 1H), 3.19 (m, 1H), 3.08 (m, 4H), 2.21 (m, 1H), 2.13-2.08 (m, 3H), 1.56 (sext, J=7.5 Hz, 4H), 0.87 (t, J=7.4 Hz, 6H).

### Example 215

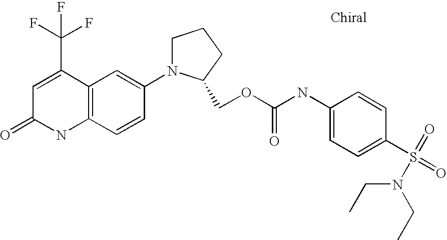

### (4-Diethylsulfamoyl-phenyl)-carbamic acid (R)-1-(2-oxo-4-trifluoromethyl-1,2-dihydro-quinolin-6-yl)-pyrrolidin-2-ylmethyl ester

### (Compound 167, Structure 14 of Scheme IV)

¹H NMR (500 MHz, Acetone-d₆) δ 9.22 (s, 1H), 7.78 (s, 4H), 7.45 (d, J=9.1 Hz, 1H), 7.35 (dd, J=9.1, 2.7 Hz, 1H), 6.91 (m, 1H), 6.89 (s, 1H), 4.40 (dd, J=11.0, 3.7 Hz, 1H), 4.11 (m, 1H), 3.99 (dd, J=11.0, 8.3 Hz, 1H), 3.55 (m, 1H), 3.22 (q, J=7.2 Hz, 4H), 3.19 (m, 1H), 2.20 (m, 1H), 2.13-2.07 (m, 3H), 1.11 (t, J=7.2 Hz, 6H).

### Example 216

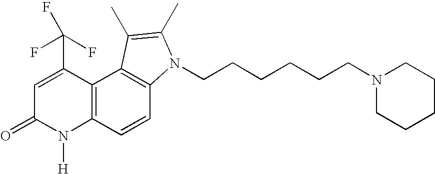

### 1,2-Dimethyl-3-[6-(1-piperidinyl)hexyl]-9-trifluoromethyl-3,6-dihydro-pyrrolo[3,2-f]-quinolin-7-one

(Compound 241, Structure 27 of Scheme VIII, where R¹=Trifluoromethyl, R³=Hydrogen, R⁴³=Methyl, R⁴⁵=Methyl)

¹H NMR (500 MHz, CDCl₃) δ 7.53 (d, J=8.8 Hz, 1H), 7.12 (s, 1H), 7.08 (d, J=8.8 Hz, 1H), 4.12 (t, J=7.4 Hz, 2H), 2.40 (s, 3H), 2.40 (m, 2H), 2.32 (q, J=2.0 Hz, 3H), 2.31 (m, 2H), 1.74 (m, 2H), 1.70-1.60 (m, 6H), 1.55-1.29 (m, 8H).

### Example 217

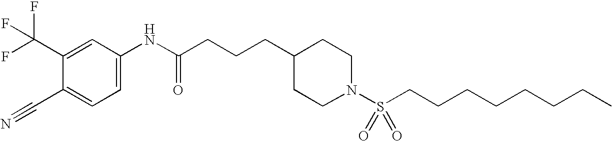

### 4-(1-Octylsulfonylpiperidinyl-4-)butanoic acid (4-nitro-3-trifluoromethyl-phenyl)-amide

(Compound 129, Structure 9 of Scheme II, where R¹¹=Trifluoromethyl, R¹⁰=Cyano, R¹⁴=Hydrogen, G=(C═O))

¹H NMR (500 MHz, CDCl₃) δ 8.00 (d, J=2.1 Hz, 1H), 7.95 (dd, J=8.5, 2.1 Hz, 1H), 7.82 (s, 1H), 7.77 (d, J=8.5 Hz, 1H), 3.80 (m, 2H), 2.89 (m, 2H), 2.73 (td, J=12.1, 2.3 Hz, 2H), 2.42 (t, J=7.4 Hz, 2H), 1.84-1.72 (m, 7H), 1.45-1.21 (m, 14H), 0.88 (t, J=7.1 Hz, 3H).

### Example 218

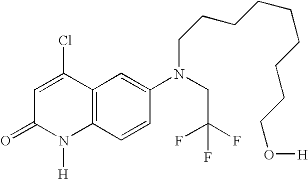

### 6-(N-(9-Hydroxynonanyl)-N-(2,2,2-trifluoroethyl)amino)-4-chloro-2-oxo-1,2-dihydro-quinoline

(Compound 101, Structure 5 of Scheme I, where R¹=Chloro, R²=Hydrogen, R³=Hydrogen, R⁴=R⁵=R⁷=R⁸=Hydrogen, R⁶=Trifluoromethyl, R⁹=8-Hydroxyoctane)

¹H NMR (300 MHz, CD₃OD) δ 7.28 (d, J=9.1 Hz, 1H), 7.24 (dd, J=9.1, 2.2 Hz, 1H), 7.19 (d, J=2.2 Hz, 1H), 6.74 (s, 1H), 4.57 (m, 2H), 4.06 (q, J=9.1 Hz, 2H), 3.44 (m, 2H), 1.60 (m, 2H), 1.46 (m, 2H), 1.38-1.22 (m, 10H).

### Example 219

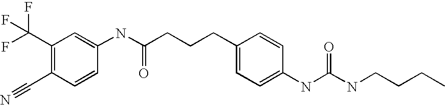

### 4-(4-Butylaminocarbonylaminophenyl)butanoic acid (4-cyano-3-trifluoromethyl-phenyl)-amide

(Compound 128, Structure 9 of Scheme II, where R¹¹=Trifluoromethyl, R¹⁰=Cyano, R¹⁴=Hydrogen, G=(C═O))

¹H NMR (500 MHz, CDCl₃) δ 7.98 (d, J=2.2 Hz, 1H), 7.89 (dd, J=8.5, 2.2 Hz, 1H), 7.77 (d, J=8.5 Hz, 1H), 7.53 (s, 1H), 7.34 (d, J=9.0 Hz, 2H), 7.32 (d, J=9.0 Hz, 2H), 3.76 (t, J=7.4 Hz, 2H), 2.78 (t, J=7.4 Hz, 2H), 2.41 (t, J=7.4 Hz, 2H), 2.10 (qn, J=7.4 Hz, 2H), 1.72 (qn, J=7.4 Hz, 2H), 1.41 (sext, J=7.4 Hz, 2H), 0.98 (t, J=7.4 Hz, 3H).

### Example 220

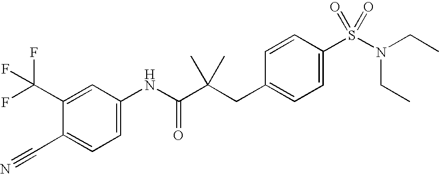

### 2,2-Dimethyl-3-(4-N,N-diethylaminosulfonyl)phenylpropanoic acid (4-cyano-3-trifluoromethyl-phenyl)-amide

(Compound 130, Structure 9 of Scheme II, where R¹¹=Trifluoromethyl, R¹⁰=Cyano, R¹⁴=Hydrogen, G=(C═O))

¹H NMR (500 MHz, CDCl₃) δ 7.98 (d, J=2.1 Hz, 1H), 7.81 (dd, J=8.5, 2.1 Hz, 1H), 7.77 (d, J=8.5 Hz, 1H), 7.68 (d, J=8.2 Hz, 2H), 7.40 (s, 1H), 7.25 (d, J=8.2 Hz, 2H), 3.21 (q, J=7.2 Hz, 4H), 3.01 (s, 2H), 1.33 (s, 6H), 1.10 (t, J=7.2 Hz, 6H).

### Example 221

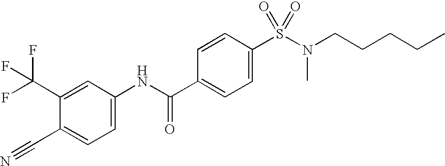

### 4-(N-methyl-N-pentylaminosulfonyl)phenylacetic acid (4-cyano-3-trifluoromethyl-phenyl)-amide

(Compound 131, Structure 9 of Scheme II, where R¹¹=Trifluoromethyl, R¹⁰=Cyano, R¹⁴=Hydrogen, G=(C═O))

¹H NMR (500 MHz, CDCl₃) δ 8.83 (s, 1H), 8.20 (d, J=2.2 Hz, 1H), 8.17 (dd, J=8.5, 2.2 Hz, 1H), 7.94 (d, J=8.5 Hz, 2H), 7.86 (d, J=8.5 Hz, 1H), 7.71 (d, J=8.5 Hz, 2H), 3.01 (t, J=7.3 Hz, 2H), 2.75 (s, 3H), 1.54 (qn, J=7.3 Hz, 2H), 1.37-1.25 (m, 4H), 0.89 (t, J=7.0 Hz, 3H).

### Example 222

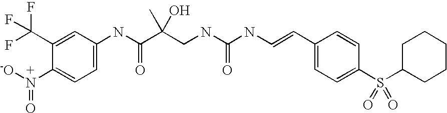

### (±)-2-Hydroxy-2-methyl-3-(4-cyclohexylsulfonylphenyl-(E)-ethylamino-carbonyl-amino)propanoic acid (4-nitro-3-trifluoromethyl-phenyl)-amide

(Compound 132, Structure 9 of Scheme II, where R¹¹=Trifluoromethyl, R¹⁰=Nitro, R¹⁴=Hydrogen, G=(C═O))

¹H NMR (500 MHz, DMSO) δ 10.60 (s, 1H), 9.16 (d, J=10.7 Hz, 1H), 8.55 (d, J=2.2 Hz, 1H), 8.32 (dd, J=9.0, 2.2 Hz, 1H), 8.20 (d, J=9.0 Hz, 1H), 7.62 (d, J=8.5 Hz, 2H), 7.52 (dd, J=14.5, 10.7 Hz, 1H), 7.49 (d, J=8.5 Hz, 2H), 6.58 (t, J=6.0 Hz, 1H), 6.27 (br s, 1H), 5.89 (d, J=14.5 Hz, 1H), 3.45 (dd, J=13.7, 6.0 Hz, 1H), 3.41 (dd, J=13.7, 6.0 Hz, 1H), 3.09 (m, 1H), 1.90-1.85 (m, 2H), 1.76-1.71 (m, 2H), 1.58 (m, 1H), 1.35 (s, 3H), 1.25-1.18 (m, 5H).

### Example 223

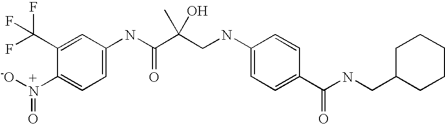

### (±)-2-Hydroxy-2-methyl-3-(4-cyclohexylmethylaminocarbonylphenylamino)propanoic acid (4-nitro-3-trifluoromethyl-phenyl)-amide

(Compound 133, Structure 9 of Scheme II, where R¹¹=Trifluoromethyl, R¹⁰=Nitro, R¹⁴=Hydrogen, G=(C═O))

¹H NMR (300 MHz, CDCl₃) δ 9.46 (s, 1H), 8.12 (d, J=1.0 Hz, 1H), 8.00 (d, J=9.1 Hz, 1H), 7.96 (dd, J=9.1, 1.0 Hz, 1H), 7.47 (d, J=8.8 Hz, 2H), 6.48 (d, J=8.8 Hz, 2H), 6.10 (t, J=6.1 Hz, 1H), 4.36 (br s, 1H), 3.63 (br d, J=12.7 Hz, 1H), 3.25 (t, J=6.1 Hz, 2H), 3.08 (d, J=12.7 Hz, 1H), 1.80-1.58 (m, 6H), 1.56 (s, 3H), 1.32-1.10 (m, 3H), 1.04-0.90 (m, 2H).

### Example 224

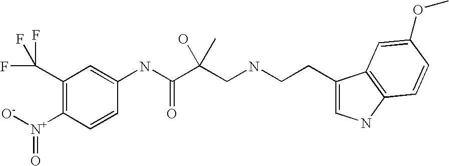

### (±)-2-Hydroxy-2-methyl-3-(N-2-(5-methoxyindolylethyl-3-)aminopropanoic acid (4-nitro-3-trifluoromethyl-phenyl)-amide

(Compound 134, Structure 9 of Scheme II, where R¹¹=Trifluoromethyl, R¹⁰=Nitro, R¹⁴=Hydrogen, G=(C═O))

¹H NMR (500 MHz, CDCl₃) δ9.51 (s, 1H), 8.03 (d, J=2.1 Hz, 1H), 7.93 (m, 1H), 7.92 (d, J=8.8 Hz, 1H), 7.76 (dd, J=8.8, 2.3 Hz, 1H), 7.24 (d, J=8.8 Hz, 1H), 6.99 (d, J=2.3 Hz, 1H), 6.96 (d, J=2.3 Hz, 1H), 6.83 (dd, J=8.8, 2.3 Hz, 1H), 3.80 (s, 3H), 3.39 (d, J=12.6 Hz, 1H), 3.00-2.84 (m, 4H), 2.56 (d, J=12.6 Hz, 1H), 1.38 (s, 3H).

### Example 225

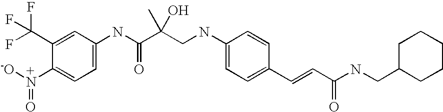

### (±)-2-Hydroxy-2-methyl-3-(4-cyclohexylmethylaminocarbonyl-(E)-ethenylphenylamino)propanoic acid (4-nitro-3-trifluoromethyl-phenyl)-amide

(Compound 135, Structure 9 of Scheme II, where R¹¹=Trifluoromethyl, R¹⁰=Nitro, R¹⁴=Hydrogen, G=(C═O))

¹H NMR (500 MHz, CDCl₃) δ 9.31 (s, 1H), 8.09 (d, J=1.8 Hz, 1H), 7.98 (dd, J=8.9, 1.8 Hz, 1H), 7.96 (d, J=8.9 Hz, 1H), 7.40 (d, J=15.5 Hz, 1H), 7.19 (d, J=8.8 Hz, 2H), 6.61 (d, J=8.8 Hz, 2H), 6.15 (d, J=15.5 Hz, 1H), 5.62 (t, J=6.3 Hz, 1H), 4.27 (s, 1H), 4.13 (s, 1H), 3.82 (dd, J=13.2, 3.2 Hz, 1H), 3.33 (d, J=13.2 Hz, 1H), 3.22 (t, J=6.3 Hz, 2H), 1.78-1.71 (m, 4H), 1.67 (m, 1H), 1.59 (s, 3H), 1.50 (m, 1H), 1.28-1.14 (m, 3H), 1.01-0.91 (m, 2H).

### Example 226

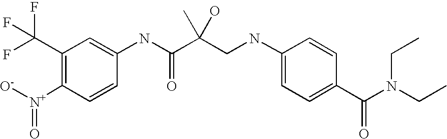

### (±)-2-Hydroxy-2-methyl-3-(4-N,N-dimethylaminocarbonylphenylamino)propanoic acid (4-nitro-3-trifluoromethyl-phenyl)-amide

(Compound 136, Structure 9 of Scheme II, where R¹¹=Trifluoromethyl, R¹⁰=Nitro, R¹⁴=Hydrogen, G=(C═O))

¹H NMR (500 MHz, CDCl₃) δ 9.48 (s, 1H), 8.12 (m, 1H), 7.96-7.94 (m, 2H), 7.15 (d, J=8.5 Hz, 2H), 6.81 (br s, 1H), 6.42 (d, J=8.5 Hz, 2H), 3.91 (br s, 1H), 3.51 (m, 2H), 3.42 (d, J=11.6 Hz, 1H), 3.31 (m, 2H), 2.22 (d, J=11.6 Hz, 1H), 1.59 (s, 3H), 1.23 (m, 3H), 1.13 (m, 3H).

### Example 227

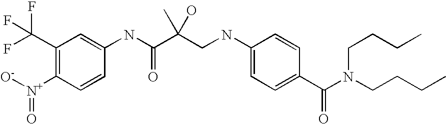

### (±)-2-Hydroxy-2-methyl-3-(4-N,N-dibutylaminocarboylphenylamino)propanoic acid (4-nitro-3-trifluoromethyl-phenyl)-amide

(Compound 137, Structure 9 of Scheme II, where R¹¹=Trifluoromethyl, R¹⁰=Nitro, R¹⁴=Hydrogen, G=(C═O))

¹H NMR (500 MHz, CDCl₃) δ 9.49 (s, 1H), 8.12 (m, 1H), 7.95 (m, 2H), 7.12 (d, J=8.5 Hz, 2H), 6.80 (br s, 1H), 6.41 (d, J=8.5 Hz, 2H), 3.92 (br s, 1H), 3.46 (m, 2H0, 3.42 (d, J=11.6 Hz, 1H), 3.25 (m, 2H), 2.22 (d, J=11.6 Hz, 1H), 1.62 (m, 2H), 1.48 (m, 2H), 1.44 (s, 3H), 1.38 (m, 2H), 1.14 (m, 2H), 0.97 (t, J=6.4 Hz, 3H), 0.82 (t, J=6.4 Hz, 3H).

1. A compound of Formulae I or Ia: 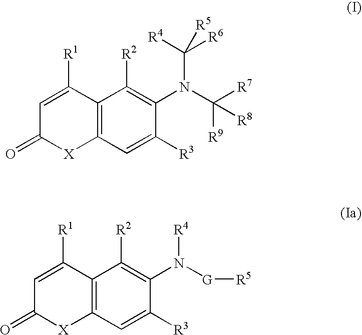
wherein:
R¹, R² and R³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R⁴, R⁵, R⁶, R⁷, R⁸ and R⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
m is selected from 0, 1 and 2;
G is selected from —CO—, —CS—, —SO₂— and a bond;
V is selected from O, S and NR^{E};
X is selected from O, S and NR^{A};
provided that at least one of R^{A}, R¹, R², R³, R⁴, R⁵, R⁶, R⁷, R⁸ and R⁹ is QT,
Q is selected from optionally substituted C₂-C₁₂ alkyl, optionally substituted C₂-C₁₂ haloalkyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl and optionally substituted C₂-C₁₂ heteroarylheteroalkyl; and
T is selected from NO₂, OH, CN, CO₂R¹, JR^{A}(O)R^{C}R^{D}, R^{C}S(O)₂NR^{C}R^{D}, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, SO₂NR^{C}R^{D}, optionally substituted C₇-C₁₀ haloalkyl and optionally substituted C₇-C₁₀ heteroalkyl,
provided that if R⁴, R⁵, R⁶, R⁷, R⁸, or R⁹ is QT, then T is not S(O)ₘR^{C};
or a pharmaceutically acceptable salt or prodrug thereof.

2. The compound of claim 1, wherein:
Q is selected from C₂-C₁₂ alkyl, C₂-C₁₂ haloalkyl, C₂-C₁₂ heteroalkyl, C₂-C₁₂ arylalkyl, C₂-C₁₂ heteroarylalkyl, C₂-C₁₂ arylhaloalkyl, C₂-C₁₂ heteroarylhaloalkyl, C₂-C₁₂ arylheteroalkyl and C₂-C₁₂ heteroarylheteroalkyl; and
T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}O₂R^{C}, S(O)ₘR^{C}, NR^{C}SO₂R^{D}, SO₂NR^{C}, C₇-C₁₀ haloalkyl and C₇-C₁₀ heteroalkyl.

3. A compound of Formulae II, IIa or IIb: 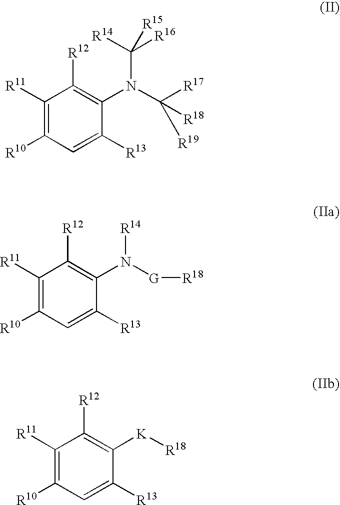
wherein:
R¹⁰ and R¹¹ are independently selected from NO₂, CN, halogen, C₁-C₄ alkyl, C₁-C₄ haloalkyl, C₁-C₄ heteroalkyl and C₁-C₄ heterohaloalkyl;
R¹² and R¹³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R¹⁴, R¹⁵, R¹⁶, R¹⁷, R¹⁸ and R¹⁹ are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, optionally substituted C₁-C₆ alkyl, optionally substituted C₁-C₆ haloalkyl, optionally substituted C₁-C₆ heteroalkyl, optionally substituted C₁-C₆ heterohaloalkyl, optionally substituted aryl, optionally substituted C₂-C₆ alkenyl and optionally substituted C₂-C₆ heteroalkenyl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
m is selected from 0, 1 and 2;
G is selected from —CO—, —CS—, —SO₂— and a bond;
J is selected from optionally substituted aryl, optionally substituted heteroaryl, and optionally substituted non-aromatic heterocycle;
V is selected from O, S and NR^{E};
K is selected from —OP(S)OR^{A}O—, —OP(O)OR^{A}O—, —NR^{B}P(S)OR^{A}O— and —NR^{B}P(O)OR^{A}O—;
provided that at least one of the R^{A}, R¹², R¹³, R¹⁴, R¹⁵, R¹⁶, R¹⁷, R¹⁸ and R¹⁹ is QT,
Q is selected from optionally substituted C₂-C₁₂ alkyl, optionally substituted C₂-C₁₂ haloalkyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₆ alkenyl and optionally substituted C₂-C₆ heteroalkenyl; and
T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, JS(O)ₘR^{C}R^{D}, JNC(V)NR^{C}R^{D}, JS(O)ₘR^{C}R^{D}, RCS(O)ₘR^{D}, NJC(O)NR^{C}R^{D}, R^{C}J, JR^{C}R^{D}, JC(V)NR^{C}R^{D}, SO₂NR^{C}R^{D}, optionally substituted C₇-C₁₀ haloalkyl and optionally substituted C₇-C₁₀ heteroalkyl;
provided that, if Q is selected from C₂-C₁₂ alkyl, C₂-C₁₂ haloalkyl, C₂-C₁₂ alkoxy, C₂-C₁₂ arylalkyl, C₂-C₁₂ arylhaloalkyl and C₂-C₁₂ heteroarylhaloalkyl, then T is not NO₂, CN, COR^{C}, S(O)₂R^{C}; and if Q is selected from noncyclic C₂-C₈ alkyl and noncyclic C₂-C₈ haloalkyl, then T is not NR^{A}COR^{B}, NR^{A}CO₂R^{B}, or NR^{C}SO₂R^{D};
or a pharmaceutically acceptable salt or prodrug thereof.

4. The compound of claim 3, wherein:
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and
T is selected from NO₂, CN, C(V)R^{C}, C(V)OR^{C}, OC(V)R^{C}, C(V)NR^{C}R^{D}, OC(O)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

5. A compound of Formula III: 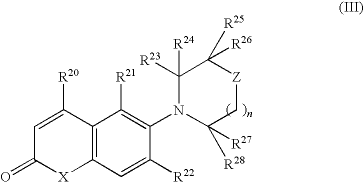
wherein:
R²⁰, R²¹ and R²² are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R²³, R²⁴, R²⁵, R²⁶, R²⁷, R²⁸, R²⁹ and R^{29a} are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl, C₁-C₆ heterohaloalkyl and aryl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
J is selected from optionally substituted aryl, optionally substituted heteroaryl, and optionally substituted non-aromatic heterocycle;
V is selected from O, S and NR^{E};
X is selected from O, S and NR^{A};
Z is selected from O, S, NR²⁹ and CR²⁹R^{29a};
m is selected from 0, 1 and 2;
n is selected from 0, 1, 2 and 3;
provided that at least one of the R^{A}, R²⁰, R²¹, R²², R²³, R²⁴, R²⁵, R²⁶, R²⁷, R²⁸, R²⁹ and R^{29a} is QT;
Q is selected from optionally substituted C₂-C₁₂ alkyl, optionally substituted C₂-C₁₂ haloalkyl, optionally substituted C₂-C₁₂ heteroalkyl, optionally substituted C₂-C₁₂, C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroaryl-heteroalkyl, optionally substituted C₂-C₆ alkenyl and optionally substituted C₂-C₆ heteroalkenyl; and
T is selected from NO₂, CN, CO₂R¹, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, SO₂NR^{C}R^{D}, R^{C}OR^{A}R^{D}, R^{C}S(O)ₘR^{D}R^{E}, NC(O)JNS(O)ₘR^{C}, NR^{C}CR^{D}, VR^{A}(O)J, R^{A}(O)J, R^{A}(O)NR^{C}, C(O)NR^{C}, R^{C}R^{D}, JSO₂R^{D}, VR^{A}(O)NR^{C}, R^{C}S(O)ₘNR^{D}R^{E} and VR^{A}(O)NR^{C}S(O)ₘNR^{C}R^{D}, optionally substituted C₇-C₁₀ haloalkyl and optionally substituted C₇-C₁₀ heteroalkyl;
provided that, if R²³, R²⁴, R²⁵, R²⁶, R²⁷, R²⁸, R²⁹, or R^{29a} is QT, then T is not C₇-C₈ haloalkyl or C₇-C₈ heteroalkyl;
or a pharmaceutically acceptable salt or prodrug thereof.

6. The compound of claim 5, wherein:
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and
T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

7. A compound of Formula IV: 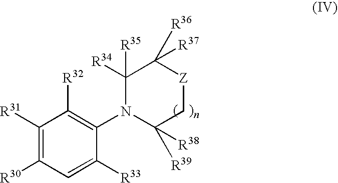
wherein:
R³⁰ and R³¹ are independently selected from NO₂, CN, halogen, C₁-C₄ alkyl, C₁-C₄ haloalkyl, C₁-C₄ heteroalkyl and C₁-C₄ heterohaloalkyl;
R³² and R³³ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R³⁴, R³⁵, R³⁶, R³⁷, R³⁸, R³⁹, R^{39a} and R^{39b} are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
V is selected from O, S and NR^{E};
Z is selected from O, S, NR^{39a} and CR^{39a}R^{39b};
m is selected from 0, 1 and 2;
n is selected from 0, 1, 2 and 3;
provided that at least one of the R^{A}, R³², R³³, R³⁴, R³⁵, R³⁶, R³⁷, R³⁸, R³⁹, R^{39a} and R^{39b} is QT,
Q is selected from optionally substituted C₂-C₁₂ alkyl, optionally substituted C₂-C₁₂ haloalkyl, optionally substituted C₂-C₁₂ heteroalkyl, optionally substituted C₂-C₁₂, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₁₂ alkenyl and optionally substituted C₂-C₁₂ heteroalkenylalkenyl; and
T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, S(O)ₘNR^{C}R^{D}, NR^{C}SO₂R^{D}, SO₂NR^{C}, C₇-C₁₀ haloalkyl and C₇-C₁₀ heteroalkyl;
provided that if R³⁴, R³⁵, R³⁶, R³⁷, R³⁸, R³⁹, R^{39a}, or R^{39b} is QT, then T is not C₃-C₄ haloalkyl or C₃-C₄ heteroalkyl;
or a pharmaceutically acceptable salt or prodrug thereof.

8. The compound of claim 7, wherein:
Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and
T is selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

9. A compound of Formula V: 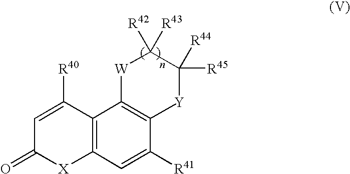
wherein:
R⁴⁰ and R⁴¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R⁴², R⁴³, R⁴⁴, R⁴⁶ and R⁴⁷ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT; or
R⁴² and R⁴⁴ and/or R⁴² and R⁴⁷ and/or R⁴⁴ and R⁴⁶ form a bond;
R⁴⁴ and R⁴⁷ can optionally form a bond when n is 0;
R⁴⁵ is selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl, optionally substituted aryl, R^{C}OR^{D}VJ and QT;
R^{A} and R^{B} are independently selected from hydrogen, C₁-C₆ alkyl, C₁-C₆ haloalkyl, C₁-C₆ heteroalkyl, C₁-C₆ heterohaloalkyl, heteroaryl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl, C₁-C₆ heterohaloalkyl and aryl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
J is selected from optionally substituted aryl, optionally substituted heteroaryl, and optionally substituted non-aromatic heterocycle;
V is selected from O, S and NR^{E};
X is selected from O, S and NR^{A};
Y is selected from O, S, NR⁴⁶ and a bond;
W is selected from O, S, NR⁴⁷ and a bond;
m is selected from 0, 1 and 2;
n is selected from 0, 1, 2 and 3;
provided that at least one of R^{A}, R⁴⁰, R⁴¹, R⁴², R⁴³, R⁴⁴, R⁴⁵, R⁴⁶ and R⁴⁷ is QT,
Q is selected from optionally substituted C₂-C₁₂ alkyl, optionally substituted C₂-C₁₂ haloalkyl, optionally substituted C₂-C₁₂ heteroalkyl, optionally substituted C₂-C₁₂ arylalkyl, optionally substituted C₂-C₁₂ heteroarylalkyl, optionally substituted C₂-C₁₂ arylhaloalkyl, optionally substituted C₂-C₁₂ heteroarylhaloalkyl, optionally substituted C₂-C₁₂ arylheteroalkyl, optionally substituted C₂-C₁₂ heteroarylheteroalkyl, optionally substituted C₂-C₆ alkenyl and optionally substituted C₂-C₆ heteroalkenyl; and
T is a selected from NO₂, CN, OH, C═C, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, R^{C}NR^{D}, NR^{C}R^{D}, R^{C}R^{D}S(O)ₘJV, VR^{C}S(O)ₘVR^{D}R^{E}, VJ, JR^{C}, R^{C}J, JR^{C}J, JCR^{C}R^{D}, JS(O)ₘJ, JR^{C}R^{E}, R^{C}OR^{D}J, JR^{C}JR^{D}R^{E}, JC(O)ₘR^{C}, JR^{C}OR^{D}, optionally substituted C₇-C₁₀ alkyl, optionally substituted C₇-C₁₀ heteroaryl, aryl, optionally substituted C₇-C₁₀ haloalkyl and optionally substituted C₇-C₁₀ heteroalkyl;
or a pharmaceutically acceptable salt or prodrug thereof.

10. The compound of claim 9, wherein:
R⁴², R⁴³, R⁴⁴, R⁴⁵, R⁴⁶ and R⁴⁷ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and
T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl.

11. A compound of Formula VI: 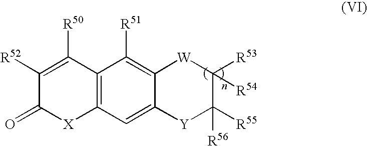
wherein:
R⁵⁰ and R⁵¹ are independently selected from hydrogen, halogen, OR^{A}, SR^{A}, NR^{A}R^{B}, optionally substituted C₁-C₄ alkyl, optionally substituted C₁-C₄ haloalkyl, optionally substituted C₁-C₄ heteroalkyl, optionally substituted C₁-C₄ heterohaloalkyl and QT;
R⁵² is selected from hydrogen, F, Cl, Br, CH₃ and CF₃;
R⁵³, R⁵⁴, R⁵⁵, R⁵⁶, R⁵⁷ and R⁵⁸ are independently selected from hydrogen, OR^{A}, optionally substituted C₁-C₆ alkyl, optionally substituted C₁-C₆ haloalkyl, optionally substituted C₁-C₆ heteroalkyl, optionally substituted C₁-C₆ heterohaloalkyl and QT;
R⁵³ and R⁵⁵, R⁵³ and R⁵⁷, or R⁵⁵ and R⁵⁸ can optionally form a bond;
R⁵⁵ and R⁵⁷ can optionally form a bond when n is 0;
R^{A} and R^{B} are independently selected from hydrogen, C₁-C₆ alkyl, C₁-C₆ haloalkyl, C₁-C₆ heteroalkyl, C₁-C₆ heterohaloalkyl and QT; or R^{A} and R^{B} are linked to form non-aromatic ring;
R^{C} and R^{D} are independently selected from hydrogen, C₁-C₆ alkyl, C₁-C₆ haloalkyl, C₁-C₆ heteroalkyl and C₁-C₆ heterohaloalkyl; or R^{C} and R^{D} are linked to form a non-aromatic ring;
R^{E} is selected from hydrogen, OR^{A}, NR^{A}, COR^{A}, C₁-C₆ alkyl, C₁-C₆ haloalkyl, C₁-C₆ heteroalkyl and C₁-C₆ heterohaloalkyl;
V is selected from O, S and NR^{E};
X is selected from O, S and NR^{A};
Y is selected from O, S, NR⁵⁸ and a bond;
W is selected from O, S, NR⁵⁷ and a bond;
m is selected from 0, 1 and 2;
n is selected from 0, 1, 2 and 3;
provided that at least one of the R^{A}, R⁵⁰, R⁵¹, R⁵³, R⁵⁴, R⁵⁵, R⁵⁶, R⁵⁷ and R⁵⁸ is QT,
Q is selected from C₂-C₁₂ alkyl, C₂-C₁₂ haloalkyl, C₂-C₁₂ heteroalkyl, C₂-C₁₂, C₂-C₁₂ arylalkyl, C₂-C₁₂ heteroarylalkyl, C₂-C₁₂ arylhaloalkyl; C₂-C₁₂ heteroarylhaloalkyl, C₂-C₁₂ arylheteroalkyl and C₂-C₁₂ heteroarylheteroalkyl; and
T is a selected from NO₂, CN, C(V)R^{C}, C(V)OR^{C}, OC(V)R^{C}, C(V)NR^{C}R^{D}, OC(V)NR^{C}R^{D}, NR^{A}C(V)R^{C}, NR^{C}C(V)NR^{C}R^{D}, NR^{C}CO₂R^{C}, S(O)ₘR^{C}, NR^{C}SO₂R^{D}, SO₂NR^{C}, C₇-C₁₀ haloalkyl and C₇-C₁₀ heteroalkyl,
provided that, if W is a bond, and Y is NH, and n is 3, and each of R⁵¹, R⁵², R⁵⁵, and R⁵⁶ is hydrogen, and X is either O or NR^{A}, and either the R⁵³ or the R⁵⁴ bound to the carbon that is closest to Y is QT, then T is not NO₂, CN, C₇-C₈ cycloheteroalkyl, NHCOR^{C}, COR^{C}, O₂CR^{C}, CO₂R^{C}, S(O)ₘR^{C}, CONR^{C}R^{D}, NHCO₂R^{C}, OC(O)NR^{C}R^{D}, NR^{C}C(O)NR^{C}R^{D}, and NR^{C}SO₂R^{D}; and
if W is a bond, and Y is NH, and n is 3, and each of R⁵¹, R⁵², R⁵⁵, and R⁵⁶ is hydrogen, and X is NR^{A} where R^{A} is QT, and where Q is saturated noncyclic alkyl, then T is not CO₂H;
or a pharmaceutically acceptable salt or prodrug thereof.

12. A compound selected from among:
6-(N-(9-Hydroxynonanyl)-N-(2,2,2-trifluoroethyl)amino)-4-chloro-2-oxo-1,2-dihydro-quinoline (Compound 101);
Hexanoic acid 9-[(4-chloro-2-oxo-1,2-dihydro-quinolin-6-yl)-(2,2,2-trifluoro-ethyl)-amino]-nonyl ester (Compound 102,
(E)-3-[(2R,5R)-5-Methyl-1-(2-oxo-4-trifluoromethyl-1,2-dihydro-quinolin-6-yl)-pyrrolidin-2-yl]-acrylic acid (Compound 103);
9-(2-Oxo-4-trifluoromethyl-1,2-dihydro-quinolin-6-ylcarbamoyl)-nonanoic acid methyl ester (Compound 105);
9-(2-Oxo-4-trifluoromethyl-1,2-dihydro-quinolin-6-ylcarbamoyl)-nonanoic acid (Compound 106);
9-[(2-Oxo-4-trifluoromethyl-1,2-dihydro-quinolin-6-yl)-(2,2,2-trifluoro-ethyl)-carbamoyl]-nonanoic acid methyl ester (Compound 107);
Decanedioic acid methyl-pentyl-amide(2-oxo-4-trifluoromethyl-1,2-dihydro-quinolin-6-yl)-amide (Compound 108);
9-[(2-Oxo-4-trifluoromethyl-1,2-dihydro-quinolin-6-yl)-(2,2,2-trifluoro-ethyl)-carbamoyl]-nonanoic acid (Compound 109);
4-[3-(2-Oxo-4-trifluoromethyl-1,2-dihydro-quinolin-6-yl)-ureido]-N,N-dipropyl-benzenesulfonamide (Compound 110);
N-Cyclohexylmethyl-4-[3-(2-oxo-4-trifluoromethyl-1,2-dihydro-quinolin-6-yl)-ureido]-benzenesulfonamide (Compound 111);
10-(4-Nitro-3-trifluoromethyl-phenylamino)-decanoic acid N-methyl-N-pentyl-amide (Compound 114);
10-(4-Nitro-3-trifluoromethyl-phenylamino)-1-piperidin-1-yl-decan-1-one (Compound 115);
10-[(4-Nitro-3-trifluoromethyl-phenyl)-(2,2,2-trifluoro-ethyl)-amino]-decanoic acid N-methyl-N-pentyl-amide (Compound 116);
10-[(4-Nitro-3-trifluoromethyl-phenyl)-(N-2,2,2-trifluoroethyl)amino]-1-piperidin-1-yl-decan-1-one (Compound 117);
10-(4-Nitro-3-trifluoromethyl-phenylamino)-decanoic acid methyl ester (Compound 120);
10-(4-Nitro-3-trifluoromethyl-phenylamino)-decanoic acid (Compound 121);
10-(N-Methyl-N-pentylamino)-decanoic acid (4-nitro-3-trifluoromethyl-phenyl)-amide (Compound 122);
10-(2-Diethylamino-ethylamino)-decanoic acid (4-nitro-3-trifluoromethyl-phenyl)-amide (Compound 123);
12-(N-Methyl-N-pentylamino)-dodecanoic acid (4-nitro-3-trifluoromethyl-phenyl)-amide (Compound 124);
12-(2-Diethylamino-ethylamino)-dodecanoic acid (4-nitro-3-trifluoromethyl-phenyl)-amide (Compound 125);
10-(4-Ethanesulfonyl-piperazin-1-yl)-decanoic acid (4-nitro-3-trifluoromethyl-phenyl)-amide (Compound 126);
12-(4-Ethanesulfonyl-piperazin-1-yl)-dodecanoic acid (4-nitro-3-trifluoromethyl-phenyl)-amide (Compound 127);
4-(4-Butylaminocarbonylaminophenyl)butanoic acid (4-cyano-3-trifluoromethyl-phenyl)-amide (Compound 128);
4-(1-Octylsulfonylpiperidinyl-4-)butanoic acid (4-nitro-3-trifluoromethyl-phenyl)-amide (Compound 129);
2,2-Dimethyl-3-(4-N,N-diethylaminosulfonyl)phenylpropanoic acid (4-cyano-3-trifluoromethyl-phenyl)-amide (Compound 130);
4-(N-methyl-N-pentylaminosulfonyl)phenylacetic acid (4-cyano-3-trifluoromethyl-phenyl)-amide (Compound 131);
(±)-2-Hydroxy-2-methyl-3-(4-cyclohexylsulfonylphenyl-(E)-ethylamino-carbonyl-amino)propanoic acid (4-nitro-3-trifluoromethyl-phenyl)-amide (Compound 132);
(±)-2-Hydroxy-2-methyl-3-(4 cyclohexylmethylaminocarbonylphenylamino)-propanoic acid (4-nitro-3-trifluoromethyl-phenyl)-amide (Compound 133);
(±)-2-Hydroxy-2-methyl-3-(N-2-(5-methoxyindolylethyl-3-)aminopropanoic acid (4-nitro-3-trifluoromethyl-phenyl)-amide (Compound 134);
(±)-2-Hydroxy-2-methyl-3-(4-cyclohexylmethylaminocarbonyl-(E)-ethenylphenylamino)propanoic acid (4-nitro-3-trifluoromethyl-phenyl)-amide (Compound 135);
(±)-2-Hydroxy-2-methyl-3-(4-N,N-dimethylaminocarbonylphenylamino)propanoic acid (4-nitro-3-trifluoromethyl-phenyl)-amide (Compound 136);
(±)-2-Hydroxy-2-methyl-3-(4-N,N-dibutylaminocarbonylphenylamino)-propanoic acid (4-nitro-3-trifluoromethyl-phenyl)-amide (Compound 137);
(E)-N-Methyl-3-[(2R,5R)-5-methyl-1-(2-oxo-4-trifluoromethyl-1,2-dihydro-quinolin-6-yl)-pyrrolidin-2-yl]-N-pentyl-acrylamide (Compound 138);
(2-Benzyl-phenyl)-carbamic acid (R)-1-(2-oxo-4-trifluoromethyl-1,2-dihydro-quinolin-6-yl)-pyrrolidin-2-ylmethyl ester (Compound 139);
(2-Propyl-phenyl)-carbamic acid (R)-1-(2-oxo-4-trifluoromethyl-1,2-dihydro-quinolin-6-yl)-pyrrolidin-2-ylmethyl ester (Compound 140);
4-Dipropylsulfamoyl-N-{2-[(2R,5R)-5-methyl-1-(2-oxo-4-trifluoromethyl-1,2-dihydro-quinolin-6-yl)-pyrrolidin-2-yl]-ethyl}-benzamide (Compound 141);
N-{2-[(2R,5R)-5-Methyl-1-(2-oxo-4-trifluoromethyl-1,2-dihydro-quinolin-6-yl)-pyrrolidin-2-yl]-ethyl}-4-(methyl-pentyl-sulfamoyl)-benzamide (Compound 142);
4-[1-(Butane-1-sulfonyl)-piperidin-4-yl]-N-{2-[(2R,5R)-5-methyl-1-(2-oxo-4-trifluoro-methyl-1,2-dihydro-quinolin-6-yl)-pyrrolidin-2-yl]-ethyl}-butyramide (Compound 143);
4-(3-{2-[(2R,5R)-5-Methyl-1-(2-oxo-4-trifluoromethyl-1,2-dihydro-quinolin-6-yl)-pyrrolidin-2-yl]-ethyl}-ureido)-N,N-dipropyl-benzenesulfonamide (Compound 144);
N-Methyl-4-(3-{2-[(2R,5R)-5-methyl-1-(2-oxo-4-trifluoromethyl-1,2-dihydro-quinolin-6-yl)-pyrrolidin-2-yl]-ethyl}-ureido)-N-pentyl-benzenesulfonamide (Compound 145);
1-{3-[1-(Butane-1-sulfonyl)-piperidin-4-yl]-propyl}-3-{2-[(2R,5R)-5-methyl-1-(2-oxo-4-trifluoromethyl-1,2-dihydro-quinolin-6-yl)-pyrrolidin-2-yl]-ethyl}-urea (Compound 146);
6-{(2R,5R)-2-[1-Hydroxy-10-(4-isobutyryl-piperazin-1-yl)-decyl]-5-methyl-pyrrolidin-1-yl}-4-trifluoromethyl-1H-quinolin-2-one (Compound 147);
3-Dipropylsulfamoyl-N-{2-[(2R,5R)-5-methyl-1-(2-oxo-4-trifluoromethyl-1,2-dihydro-quinolin-6-yl)-pyrrolidin-2-yl]-ethyl}-benzamide (Compound 148);
3-(3-{2-[(2R,5R)-5-Methyl-1-(2-oxo-4-trifluoromethyl-1,2-dihydro-quinolin-6-yl)-pyrrolidin-2-yl]-ethyl}-ureido)-N,N-dipropyl-benzenesulfonamide (Compound 149);
(E)-N-{2-[(2R,5R)-5-Methyl-1-(2-oxo-4-trifluoromethyl-1,2-dihydro-quinolin-6-yl)-pyrrolidin-2-yl]-ethyl}-3-[4-(2-methyl-propane-1-sulfonyl)-phenyl]-acrylamide (Compound 150);
(E)-3-(4-Cyclohexylmethanesulfonyl-phenyl)-N-{2-[(2R,5R)-5-methyl-1-(2-oxo-4-trifluoromethyl-1,2-dihydro-quinolin-6-yl)-pyrrolidin-2-yl]-ethyl}-acrylamide (Compound 151);
(E)-3-(4-Cyclohexanesulfonyl-phenyl)-N-{2-[(2R,5R)-5-methyl-1-(2-oxo-4-trifluoro-methyl-1,2-dihydro-quinolin-6-yl)-pyrrolidin-2-yl]-ethyl}-acrylamide (Compound 152);
(E)-3-[4-(2-Cyclohexyl-ethanesulfonyl)-phenyl]-N-{2-[(2R,5R)-5-methyl-1-(2-oxo-4-trifluoromethyl-1,2-dihydro-quinolin-6-yl)-pyrrolidin-2-yl]-ethyl}-acrylamide (Compound 153);
(E)-N-{2-[(2R,5R)-5-Methyl-1-(2-oxo-4-trifluoromethyl-1,2-dihydro-quinolin-6-yl)-pyrrolidin-2-yl]-ethyl}-3-(4-p-tolylmethanesulfonyl-phenyl)-acrylamide (Compound 154);
(E)-N-{2-[(2R,5R)-5-Methyl-1-(2-oxo-4-trifluoromethyl-1,2-dihydro-quinolin-6-yl)-pyrrolidin-2-yl]-ethyl}-3-[4-(4-trifluoromethyl-phenylmethanesulfonyl)-phenyl]-acrylamide (Compound 155);
6-[(2R,5R)-2-Methyl-5-(5-oxo-5-pyrrolidin-1-yl-pentyloxymethyl)-pyrrolidin-1-yl]-4-trifluoromethyl-1H-quinolin-2-one (Compound 156);
5-(Propane-1-sulfonylamino)-benzo[b]thiophene-2-carboxylic acid {2-[(2R,5R)-5-methyl-1-(2-oxo-4-trifluoromethyl-1,2-dihydro-quinolin-6-yl)-pyrrolidin-2-yl]-ethyl}-amide (Compound 157);
2-Hydroxy-2-methyl-3-[(2R,5R)-5-methyl-1-(2-oxo-4-trifluoromethyl-1,2-dihydro-quinolin-6-yl)-pyrrolidin-2-ylmethoxy]-N-(4-nitro-3-trifluoromethyl-phenyl)-propionamide (Compound 158);
3-Hydroxy-2-methyl-2-[(2R,5R)-5-methyl-1-(2-oxo-4-trifluoromethyl-1,2-dihydro-quinolin-6-yl)-pyrrolidin-2-ylmethoxy]-N-(4-nitro-3-trifluoromethyl-phenyl)-propionamide (Compound 159);
2-{6-[(2R,5R)-5-Methyl-1-(2-oxo-4-trifluoromethyl-1,2-dihydro-quinolin-6-yl)-pyrrolidin-2-ylmethoxy]-hexyl}-isoindole-1,3-dione (Compound 161);
2-Oxo-imidazolidine-1-carboxylic acid 2-[(2R,5R)-5-methyl-1-(2-oxo-4-trifluoromethyl-1,2-dihydro-quinolin-6-yl)-pyrrolidin-2-yl]-ethyl ester (Compound 162);
(E)-3-(4-Benzyloxy-phenyl)-N-{2-[(2R,5R)-5-methyl-1-(2-oxo-4-trifluoromethyl-1,2-dihydro-quinolin-6-yl)-pyrrolidin-2-yl]-ethyl}-acrylamide (Compound 163);
[4-(Cyclohexylmethyl-sulfamoyl)-phenyl]-carbamic acid 2-[(2R,5R)-5-methyl-1-(2-oxo-4-trifluoromethyl-1,2-dihydro-quinolin-6-yl)-pyrrolidin-2-yl]-ethyl ester (Compound 164);
(4-Benzylsulfamoyl-phenyl)-carbamic acid 2-[(2R,5R)-5-methyl-1-(2-oxo-4-trifluoromethyl-1,2-dihydro-quinolin-6-yl)-pyrrolidin-2-yl]-ethyl ester (Compound 165);
(4-Dipropylsulfamoyl-phenyl)-carbamic acid (R)-1-(2-oxo-4-trifluoromethyl-1,2-dihydro-quinolin-6-yl)-pyrrolidin-2-ylmethyl ester (Compound 166);
(4-Diethylsulfamoyl-phenyl)-carbamic acid (R)-1-(2-oxo-4-trifluoromethyl-1,2-dihydro-quinolin-6-yl)-pyrrolidin-2-ylmethyl ester (Compound 167);
8-(7-Oxo-2-phenyl-9-trifluoromethyl-6,7-dihydro-3H-pyrrolo[3,2-f]quinolin-1-yl)-octanoic acid ethyl ester (Compound 170);
8-(7-Oxo-2-phenyl-9-trifluoromethyl-6,7-dihydro-3H-pyrrolo[3,2-f]quinolin-1-yl)-octanoic acid (Compound 171);
8-(7-Oxo-2-phenyl-9-trifluoromethyl-6,7-dihydro-3H-pyrrolo[3,2-f]quinolin-1-yl)-octanoic acid 4,4,5,5,5-pentafluoro-pentyl ester (Compound 172);
8-(7-Oxo-2-phenyl-9-trifluoromethyl-6,7-dihydro-3H-pyrrolo[3,2-f]quinolin-1-yl)-octanoic acid methyl-pentyl-amide (Compound 173);
6-(2-Methyl-7-oxo-9-trifluoromethyl-6,7-dihydro-3H-pyrrolo[3,2-f]quinolin-1-yl)-hexanoic acid methyl-pentyl-amide (Compound 174);
1-[8-(4-Ethanesulfonyl-piperazin-1-yl)-8-oxo-octyl]-2-phenyl-9-trifluoromethyl-3,6-dihydro-pyrrolo[3,2-f]quinolin-7-one (Compound 175);
1-{8-Oxo-8-[4-(piperidine-1-sulfonyl)-piperazin-1-yl]-octyl}-2-phenyl-9-trifluoromethyl-3,6-dihydro-pyrrolo[3,2-f]quinolin-7-one (Compound 176);
8-(7-Oxo-2-phenyl-9-trifluoromethyl-6,7-dihydro-3H-pyrrolo[3,2-f]quinolin-1-yl)-octanoic acid butyl-methyl-amide (Compound 177);
1-(8-Oxo-8-piperidin-1-yl-octyl)-2-phenyl-9-trifluoromethyl-3,6-dihydro-pyrrolo[3,2-f]-quinolin-7-one (Compound 178);
8-(2-Methyl-7-oxo-9-trifluoromethyl-6,7-dihydro-3H-pyrrolo[3,2-f]quinolin-1-yl)-octanoic acid methyl-pentyl-amide (Compound 179);
8-(2-Methyl-7-oxo-9-trifluoromethyl-6,7-dihydro-3H-pyrrolo[3,2-f]quinolin-1-yl)-octanoic acid diisopropylamide (Compound 180);
8-[9-Chloro-7-oxo-2-phenyl-3-((R)-3,3,3-trifluoro-2-hydroxy-propyl)-6,7-dihydro-3H-pyrrolo[3,2-f]quinolin-1-yl]-octanoic acid ethyl ester (Compound 181);
8-[9-Chloro-2-methyl-7-oxo-3-((R)-3,3,3-trifluoro-2-hydroxy-propyl)-6,7-dihydro-3H-pyrrolo[3,2-f]quinolin-1-yl]-octanoic acid ethyl ester (Compound 182);
8-[9-Chloro-7-oxo-2-phenyl-3-((S)-3,3,3-trifluoro-2-hydroxy-propyl)-6,7-dihydro-3H-pyrrolo[3,2-f]quinolin-1-yl]-octanoic acid (Compound 183);
8-[9-Chloro-7-oxo-2-phenyl-3-((S)-3,3,3-trifluoro-2-hydroxy-propyl)-6,7-dihydro-3H-pyrrolo[3,2-f]quinolin-1-yl]-octanoic acid butyl-methyl-amide (Compound 184);
9-Chloro-1-[8-(4-ethanesulfonyl-piperazin-1-yl)-8-oxo-octyl]-2-phenyl-3-((R)-3,3,3-trifluoro-2-hydroxy-propyl)-3,6-dihydro-pyrrolo[3,2-f]quinolin-7-one (Compound 185);
5-[9-Chloro-2-methyl-7-oxo-3-(2,2,3,3,3-pentafluoro-propyl)-6,7-dihydro-3H-pyrrolo-[3,2-f]-quinolin-1-yl]-pentanoic acid (Compound 186);
9-Chloro-1-[3-(4-ethanesulfonyl-piperazin-1-yl)-propyl]-2-methyl-3-(2,2,3,3,3-pentafluoro-propyl)-3,6-dihydro-pyrrolo[3,2-f]quinolin-7-one (Compound 187);
9-Chloro-2-methyl-1-{3-[methyl-(4-morpholin-4-yl-benzyl)-amino]-propyl}-3-(2,2,3,3,3-pentafluoro-propyl)-3,6-dihydro-pyrrolo[3,2-f]quinolin-7-one (Compound 188);
9-Chloro-1-[3-(4-{3-[1-(2-hydroxy-ethyl)-piperidin-4-yl]-propyl}-piperidin-1-yl)-propyl]-2-methyl-3-(2,2,3,3,3-pentafluoro-propyl)-3,6-dihydro-pyrrolo[3,2-f]quinolin-7-one (Compound 189);
9-Chloro-1-{3-[ethyl-(4-hydroxy-butyl)-amino]-propyl}-2-methyl-3-(2,2,3,3,3-pentafluoro-propyl)-3,6-dihydro-pyrrolo[3,2-f]quinolin-7-one (Compound 190);
9-Chloro-2-methyl-1-(3-{methyl-[4-(2-morpholin-4-yl-ethoxy)-benzyl]-amino}-propyl)-3-(2,2,3,3,3-pentafluoro-propyl)-3,6-dihydro-pyrrolo[3,2-f]quinolin-7-one (Compound 191);
9-Chloro-1-(3-{4-[2-(2-hydroxy-ethoxy)-ethyl]-piperazin-1-yl}-propyl)-2-methyl-3-(2,2,3,3,3-pentafluoro-propyl)-3,6-dihydro-pyrrolo[3,2-f]quinolin-7-one (Compound 192);
1-{3-[Bis-(2-hydroxy-propyl)-amino]-propyl}-9-chloro-2-methyl-3-(2,2,3,3,3-pentafluoro-propyl)-3,6-dihydro-pyrrolo[3,2-f]quinolin-7-one (Compound 193);
2-[3-(9-Chloro-2-methyl-7-oxo-6,7-dihydro-3H-pyrrolo[3,2-f]quinolin-1-yl)-propyl]-isoindole-1,3-dione (Compound 194);
9-Chloro-1-methyl-2-{4-[9-(tetrahydro-pyran-2-yloxy)-nonyloxy]-phenyl}-3,6-dihydro-pyrrolo[3,2-f]quinolin-7-one (Compound 195);
(±)-1,2-Dimethyl-3-[9-(tetrahydro-pyran-2-yloxy)-nonyl]-9-trifluoromethyl-3,6-dihydro-pyrrolo[3,2-f]quinolin-7-one (Compound 200);
3-(9-Chloro-nonyl)-1,2-dimethyl-9-trifluoromethyl-3,6-dihydro-pyrrolo[3,2-f]-quinolin-7-one (Compound 201);
3-(9-Iodo-nonyl)-1,2-dimethyl-9-trifluoromethyl-3,6-dihydro-pyrrolo[3,2-f]-quinolin-7-one (Compound 202);
1,2-Dimethyl-3-[9-(2-piperidin-1-yl-ethoxy)-nonyl]-9-trifluoromethyl-3,6-dihydro-pyrrolo[3,2-f]quinolin-7-one (Compound 203);
1,2-Dimethyl-3-[9-(methyl-pentyl-amino)-nonyl]-9-trifluoromethyl-3,6-dihydro-pyrrolo[3,2-f]quinolin-7-one (Compound 204);
3-(9-Butylsulfanyl-nonyl)-1,2-dimethyl-9-trifluoromethyl-3,6-dihydro-pyrrolo[3,2-f]-quinolin-7-one (Compound 205);
(±)-1,2-Dimethyl-3-[6-(tetrahydro-pyran-2-yloxy)-hexyl]-9-trifluoromethyl-3,6-dihydro-pyrrolo[3,2-f]quinolin-7-one (Compound 206);
3-(6-Hydroxy-hexyl)-1,2-dimethyl-9-trifluoromethyl-3,6-dihydro-pyrrolo[3,2-f]-quinolin-7-one (Compound 207);
3-(6-Chloro-hexyl)-1,2-dimethyl-9-trifluoromethyl-3,6-dihydro-pyrrolo[3,2-f]-quinolin-7-one (Compound 208);
3-(6-Butylsulfanyl-hexyl)-1,2-dimethyl-9-trifluoromethyl-3,6-dihydro-pyrrolo[3,2-f]-quinolin-7-one (Compound 209);
1,2-Dimethyl-3-[6-(1-methyl-1H-tetrazol-5-ylsulfanyl)-hexyl]-9-trifluoromethyl-3,6-dihydro-pyrrolo[3,2-f]quinolin-7-one (Compound 210);
3-(6-Heptylsulfanyl-hexyl)-1,2-dimethyl-9-trifluoromethyl-3,6-dihydro-pyrrolo[3,2-f]-quinolin-7-one (Compound 212);
3-(6-Iodo-hexyl)-1,2-dimethyl-9-trifluoromethyl-3,6-dihydro-pyrrolo[3,2-f]-quinolin-7-one (Compound 213);
1,2-Dimethyl-3-[6-(4,4,5,5,5-pentafluoro-pentylsulfanyl)-hexyl]-9-trifluoromethyl-3,6-dihydro-pyrrolo[3,2-f]quinolin-7-one (Compound 215);
(±)-1,2-Dimethyl-3-[8-(tetrahydro-pyran-2-yloxy)-octyl]-9-trifluoromethyl-3,6-dihydro-pyrrolo[3,2-f]quinolin-7-one (Compound 216);
1,2-Dimethyl-3-[6-(4,4,5,5,5-pentafluoro-pentane-1-sulfonyl)-hexyl]-9-trifluoromethyl-3,6-dihydro-pyrrolo[3,2-f]quinolin-7-one (Compound 217);
3-(8-Chloro-octyl)-1,2-dimethyl-9-trifluoromethyl-3,6-dihydro-pyrrolo[3,2-f]-quinolin-7-one (Compound 218);
3-(8-Hydroxy-octyl)-1,2-dimethyl-9-trifluoromethyl-3,6-dihydro-pyrrolo[3,2-f]-quinolin-7-one (Compound 219);
3-(8-Iodo-octyl)-1,2-dimethyl-9-trifluoromethyl-3,6-dihydro-pyrrolo[3,2-f]-quinolin-7-one (Compound 220);
2-Ethyl-1-methyl-3-[10-(tetrahydro-pyran-2-yloxy)-decyl]-9-trifluoromethyl-3,6-dihydro-pyrrolo[3,2-f]quinolin-7-one (Compound 221);
2-Ethyl-3-(10-hydroxy-decyl)-1-methyl-9-trifluoromethyl-3,6-dihydro-pyrrolo[3,2-f]-quinolin-7-one (Compound 223);
2-Ethyl-1-methyl-3-[9-(tetrahydro-pyran-2-yloxy)-nonyl]-9-trifluoromethyl-3,6-dihydro-pyrrolo[3,2-f]quinolin-7-one (Compound 224);
2-Ethyl-3-(9-hydroxy-nonyl)-1-methyl-9-trifluoromethyl-3,6-dihydro-pyrrolo[3,2-f]-quinolin-7-one (Compound 225);
3-(10-Chloro-decyl)-2-ethyl-1-methyl-9-trifluoromethyl-3,6-dihydro-pyrrolo[3,2-f]-quinolin-7-one (Compound 226);
3-(9-Chloro-nonyl)-2-ethyl-1-methyl-9-trifluoromethyl-3,6-dihydro-pyrrolo[3,2-f]-quinolin-7-one (Compound 227);
2-Ethyl-3-(10-iodo-decyl)-1-methyl-9-trifluoromethyl-3,6-dihydro-pyrrolo[3,2-f]-quinolin-7-one (Compound 228);
2-Ethyl-3-(9-iodo-nonyl)-1-methyl-9-trifluoromethyl-3,6-dihydro-pyrrolo[3,2-f]-quinolin-7-one (Compound 229);
3-[9-(Butyl-methyl-amino)-nonyl]-2-ethyl-1-methyl-9-trifluoromethyl-3,6-dihydro-pyrrolo[3,2-f]quinolin-7-one (Compound 230);
3-[10-(Butyl-methyl-amino)-decyl]-2-ethyl-1-methyl-9-trifluoromethyl-3,6-dihydro-pyrrolo[3,2-f]quinolin-7-one (Compound 231);
N-{3-[9-(2-Ethyl-1-methyl-7-oxo-9-trifluoromethyl-6,7-dihydro-pyrrolo[3,2-f]-quinolin-3-yl)-nonyloxy]-phenyl}-acetamide (Compound 232);
2-Ethyl-1-methyl-3-(9-phenoxy-nonyl)-9-trifluoromethyl-3,6-dihydro-pyrrolo[3,2-f]-quinolin-7-one (Compound 233);
2-Ethyl-1-methyl-3-(9-piperidin-1-yl-nonyl)-9-trifluoromethyl-3,6-dihydropyrrolo-[3,2-f]quinolin-7-one (Compound 234);
3-[9-(4-Ethanesulfonyl-piperazin-1-yl)-nonyl]-2-ethyl-1-methyl-9-trifluoromethyl-3,6-dihydro-pyrrolo[3,2-f]quinolin-7-one (Compound 235);
2-Ethyl-1-methyl-3-(9-phenylsulfanyl-nonyl)-9-trifluoromethyl-3,6-dihydropyrrolo-3,2-f]quinolin-7-one (Compound 236);
2-Ethyl-1-methyl-3-[9-(methyl-phenyl-amino)-nonyl]-9-trifluoromethyl-3,6-dihydro-pyrrolo[3,2-f]quinolin-7-one (Compound 237);
10-(2-Ethyl-1-methyl-7-oxo-9-trifluoromethyl-6,7-dihydro-pyrrolo[3,2-f]-quinolin-3-yl)-decanenitrile (Compound 238);
2-Ethyl-1-methyl-3-{9-[4-(piperidine-1-sulfonyl)-piperazin-1-yl]-nonyl}-9-trifluoromethyl-3,6-dihydro-pyrrolo[3,2-f]quinolin-7-one (Compound 239);
3-(9-Benzenesulfonyl-nonyl)-2-ethyl-1-methyl-9-trifluoromethyl-3,6-dihydro-pyrrolo[3,2-f]quinolin-7-one (Compound 240);
1,2-Dimethyl-3-[6-(1-piperidinyl)hexyl]-9-trifluoromethyl-3,6-dihydro-pyrrolo[3,2-f]-quinolin-7-one (Compound 241);
2-Ethyl-1-methyl-3-(10-piperidin-1-yl-decyl)-9-trifluoromethyl-3,6-dihydropyrrolo-3,2-f]-quinolin-7-one (Compound 242);
1,2-Dimethyl-3-(8-piperidin-1-yl-octyl)-9-trifluoromethyl-3,6-dihydro-pyrrolo[3,2-f]-quinolin-7-one (Compound 243);
2-Ethyl-1-methyl-3-(9-morpholin-4-yl-nonyl)-9-trifluoromethyl-3,6-dihydropyrrolo-3,2-f]quinolin-7-one (Compound 244);
2-Ethyl-1-methyl-3-(9-thiomorpholin-4-yl-nonyl)-9-trifluoromethyl-3,6-dihydro-pyrrolo3,2-f]quinolin-7-one (Compound 245);
1,2-Dimethyl-3-(6-pyrrolidin-1-yl-hexyl)-9-trifluoromethyl-3,6-dihydro-pyrrolo[3,2-f]-quinolin-7-one (Compound 246);
2-Ethyl-1-methyl-3-(9-pyrrolidin-1-yl-nonyl)-9-trifluoromethyl-3,6-dihydro-pyrrolo[3,2-f]-quinolin-7-one (Compound 247);
2-Ethyl-1-methyl-3-{9-[methyl-(4-pyridin-4-yl-benzyl)-amino]-nonyl}-9-trifluoromethyl-3,6-dihydro-pyrrolo[3,2-f]quinolin-7-one (Compound 248);
2-Ethyl-1-methyl-3-{9-[methyl-(4-morpholin-4-yl-benzyl)-amino]-nonyl}-9-trifluoromethyl-3,6-dihydro-pyrrolo[3,2-f]quinolin-7-one (Compound 249);
2-Ethyl-1-methyl-3-(9-{methyl-[4-(2-morpholin-4-yl-ethoxy)-benzyl]-amino}-nonyl)-9-trifluoromethyl-3,6-dihydro-pyrrolo[3,2-f]quinolin-7-one (Compound 250);
1,2-Dimethyl-3-{6-[methyl-(4-pyridin-4-yl-benzyl)-amino]-hexyl}-9-trifluoromethyl-3,6-dihydro-pyrrolo[3,2-f]quinolin-7-one (Compound 251);
1,2-Dimethyl-3-(6-{methyl-[4-(2-morpholin-4-yl-ethoxy)-benzyl]-amino}-hexyl)-9-trifluoromethyl-3,6-dihydro-pyrrolo[3,2-f]quinolin-7-one (Compound 252);
1,2-Dimethyl-3-{6-[methyl-(4-morpholin-4-yl-benzyl)-amino]-hexyl}-9-trifluoromethyl-3,6-dihydro-pyrrolo[3,2-f]quinolin-7-one (Compound 253);
2-Ethyl-1-methyl-3-[9-(4-pyrimidin-2-yl-piperazin-1-yl)-nonyl]-9-trifluoromethyl-3,6-dihydro-pyrrolo[3,2-f]quinolin-7-one (Compound 254);
2-Ethyl-3-{9-[4-(furan-2-carbonyl)-piperazin-1-yl]-nonyl}-1-methyl-9-trifluoromethyl-3,6-dihydro-pyrrolo[3,2-f]quinolin-7-one (Compound 255);
3-(9-{4-[Bis-(4-fluoro-phenyl)-methyl]-piperazin-1-yl}-nonyl)-2-ethyl-1-methyl-9-trifluoromethyl-3,6-dihydro-pyrrolo[3,2-f]quinolin-7-one (Compound 256);
3-[9-(4-Benzoyl-piperazin-1-yl)-nonyl]-2-ethyl-1-methyl-9-trifluoromethyl-3,6-dihydro-pyrrolo[3,2-f]quinolin-7-one (Compound 257);
3-{9-[4-(2-Ethoxy-ethyl)-piperazin-1-yl]-nonyl}-2-ethyl-1-methyl-9-trifluoromethyl-3,6-dihydro-pyrrolo[3,2-f]quinolin-7-one (Compound 258);
2-Ethyl-1-methyl-3-[9-(2-thiophen-2-yl-ethylamino)-nonyl]-9-trifluoromethyl-3,6-dihydro-pyrrolo[3,2-f]quinolin-7-one (Compound 259);
1,2-Dimethyl-3-{6-[methyl-(3-methylamino-propyl)-amino]-hexyl}-9-trifluoromethyl-3,6-dihydro-pyrrolo[3,2-f]quinolin-7-one (Compound 260);
9-(Difluoro-hydroxy-methyl)-2-ethyl-1-methyl-3-[9-(2-piperidin-1-yl-ethoxy)-nonyl]-3,6-dihydro-pyrrolo[3,2-f]quinolin-7-one (Compound 261);
2-Ethyl-1-methyl-3-(9-piperazin-1-yl-nonyl)-9-trifluoromethyl-3,6-dihydro-pyrrolo[3,2-f]-quinolin-7-one (Compound 262);
3-{4-[9-(2-Ethyl-1-methyl-7-oxo-9-trifluoromethyl-6,7-dihydro-pyrrolo[3,2-f]-quinolin-3-yl)-nonyl]-piperazin-1-yl}-propionitrile (Compound 263);
2-Ethyl-1-methyl-3-[9-([1,2,4]triazol-4-ylamino)-nonyl]-9-trifluoromethyl-3,6-dihydro-pyrrolo[3,2-f]quinolin-7-one (Compound 264);
2-Ethyl-1-methyl-3-non-8-enyl-9-trifluoromethyl-3,6-dihydro-pyrrolo[3,2-f]-quinolin-7-one (Compound 265);
2-Ethyl-1-methyl-3-[9-(2-morpholin-4-yl-ethoxy)-nonyl]-9-trifluoromethyl-3,6-dihydro-pyrrolo[3,2-f]quinolin-7-one (Compound 266);
3-[9-(Benzooxazol-2-ylsulfanyl)-nonyl]-2-ethyl-1-methyl-9-trifluoromethyl-3,6-dihydro-pyrrolo[3,2-f]quinolin-7-one (Compound 267);
2-Ethyl-1-methyl-3-{9-[methyl-((2R,3R,4R,5R)-2,3,4,5,6-pentahydroxy-hexyl)-amino]-nonyl}-9-trifluoromethyl-3,6-dihydro-pyrrolo[3,2-f]quinolin-7-one (Compound 268);
1,2-Dimethyl-3-{6-[methyl-((2S,3S,4S,5S)-2,3,4,5,6-pentahydroxy-hexyl)-amino]-hexyl}-9-trifluoromethyl-3,6-dihydro-pyrrolo[3,2-f]quinolin-7-one (Compound 269);
(±)-3-[9-(2,3-Dihydroxy-propylamino)-nonyl]-2-ethyl-1-methyl-9-trifluoromethyl-3,6-dihydro-pyrrolo[3,2-f]quinolin-7-one (Compound 270);
(±)-3-{9-[(2,3-Dihydroxy-propyl)-methyl-amino]-nonyl}-2-ethyl-1-methyl-9-trifluoromethyl-3,6-dihydro-pyrrolo[3,2-f]quinolin-7-one (Compound 271);
4-[9-(2-Ethyl-1-methyl-7-oxo-9-trifluoromethyl-6,7-dihydro-pyrrolo[3,2-f]-quinolin-3-yl)-nonyloxy]-N,N-dipropyl-benzenesulfonamide (Compound 272);
4-{2-[9-(2-Ethyl-1-methyl-7-oxo-9-trifluoromethyl-6,7-dihydro-pyrrolo[3,2-f]-quinolin-3-yl)-nonylamino]-ethyl}-N,N-dipropyl-benzenesulfonamide (Compound 273);
(±)-2-Ethyl-1-methyl-3-[3-(tetrahydro-pyran-2-yloxy)-propyl]-9-trifluoromethyl-3,6-dihydro-pyrrolo[3,2-f]quinolin-7-one (Compound 274);
3-{6-[Bis-(2-hydroxy-ethyl)-amino]-hexyl}-1,2-dimethyl-9-trifluoromethyl-3,6-dihydro-pyrrolo[3,2-f]quinolin-7-one (Compound 275);
3-{8-[4-(2-Dimethylamino-ethyl)-piperazin-1-yl]-octyl}-1,2-dimethyl-9-trifluoromethyl-3,6-dihydro-pyrrolo[3,2-f]quinolin-7-one (Compound 276);
2-Ethyl-3-[9-(4-{3-[1-(2-hydroxy-ethyl)-piperidin-4-yl]-propyl}-piperidin-1-yl)-nonyl]-1-methyl-9-trifluoromethyl-3,6-dihydro-pyrrolo[3,2-f]quinolin-7-one (Compound 277);
2-Ethyl-1-methyl-3-[9-(3-piperidin-1-ylmethyl-benzylamino)-nonyl]-9-trifluoromethyl-3,6-dihydro-pyrrolo[3,2-f]quinolin-7-one (Compound 278);
3-[6-(4-Hydroxy-butylamino)-hexyl]-1,2-dimethyl-9-trifluoromethyl-3,6-dihydro-pyrrolo[3,2-f]quinolin-7-one (Compound 279);
2-Ethyl-1-methyl-3-{9-[4-(2-phenoxy-ethyl)-piperazin-1-yl]-nonyl}-9-trifluoromethyl-3,6-dihydro-pyrrolo[3,2-f]quinolin-7-one (Compound 280);
2-Ethyl-1-methyl-3-{9-[4-(2-methyl-thiazol-4-yl)-phenylamino]-nonyl}-9-trifluoromethyl-3,6-dihydro-pyrrolo[3,2-f]quinolin-7-one (Compound 281);
2-Ethyl-1-methyl-3-[9-(4-morpholin-4-ylmethyl-benzylamino)-nonyl]-9-trifluoromethyl-3,6-dihydro-pyrrolo[3,2-f]quinolin-7-one (Compound 282);
2-Ethyl-1-methyl-3-[9-(3-morpholin-4-yl-propylamino)-nonyl]-9-trifluoromethyl-3,6-dihydro-pyrrolo[3,2-f]quinolin-7-one (Compound 283);
2-Ethyl-3-(9-{4-[2-(2-hydroxy-ethoxy)-ethyl]-piperazin-1-yl}-nonyl)-1-methyl-9-trifluoromethyl-3,6-dihydro-pyrrolo[3,2-f]quinolin-7-one (Compound 284);
2-Ethyl-1-methyl-3-{9-[4-(4-phenoxy-butyl)-piperazin-1-yl]-nonyl}-9-trifluoromethyl-3,6-dihydro-pyrrolo[3,2-f]quinolin-7-one (Compound 285);
2-Ethyl-3-{9-[4-(2-hydroxy-ethyl)-piperidin-1-yl]-nonyl}-1-methyl-9-trifluoromethyl-3,6-dihydro-pyrrolo[3,2-f]quinolin-7-one (Compound 286);
3-{(2-Cyanoethyl)-[9-(2-ethyl-1-methyl-7-oxo-9-trifluoromethyl-6,7-dihydro-pyrrolo[3,2-f]quinolin-3-yl)-nonyl]-amino}-propionitrile (Compound 287);
2-Ethyl-1-methyl-3-[9-(3-pyrrolidin-1-yl-propylamino)-nonyl]-9-trifluoromethyl-3,6-dihydro-pyrrolo[3,2-f]quinolin-7-one (Compound 288);
2-Ethyl-1-methyl-3-{9-[4-(3-morpholin-4-yl-propyl)-piperazin-1-yl]-nonyl}-9-trifluoromethyl-3,6-dihydro-pyrrolo[3,2-f]quinolin-7-on (Compound 289);
2-Ethyl-3-{9-[(5-furan-2-yl-isoxazol-3-yl-methyl)-amino]-nonyl}-1-methyl-9-trifluoromethyl-3,6-dihydro-pyrrolo[3,2-f]quinolin-7-one (Compound 290);
2-Ethyl-3-[9-(2-hydroxy-1-hydroxymethyl-ethylamino)-nonyl]-1-methyl-9-trifluoromethyl-3,6-dihydro-pyrrolo[3,2-f]quinolin-7-one (Compound 291);
2-Ethyl-3-[9-(2-hydroxy-1-methyl-2-phenyl-ethylamino)-nonyl]-1-methyl-9-trifluoromethyl-3,6-dihydro-pyrrolo[3,2-f]quinolin-7-one (Compound 292);
2-Ethyl-3-{9-[ethyl-(4-hydroxy-butyl)-amino]-nonyl}-1-methyl-9-trifluoromethyl-3,6-dihydro-pyrrolo[3,2-f]quinolin-7-one (Compound 293);
3-{Ethyl-[9-(2-ethyl-1-methyl-7-oxo-9-trifluoromethyl-6,7-dihydro-pyrrolo[3,2-f]-quinolin-3-yl)-nonyl]-amino}-propionitrile (Compound 294);
2-Ethyl-3-{9-[4-(2-hydroxy-ethyl)-piperazin-1-yl]-nonyl}-1-methyl-9-trifluoromethyl-3,6-dihydro-pyrrolo[3,2-f]quinolin-7-one (Compound 295);
2-Ethyl-3-{9-[4-(3-hydroxy-propyl)-piperazin-1-yl]-nonyl}-1-methyl-9-trifluoromethyl-3,6-dihydro-pyrrolo[3,2-f]quinolin-7-one (Compound 296);
4-[9-(2-Ethyl-1-methyl-7-oxo-9-trifluoromethyl-6,7-dihydro-pyrrolo[3,2-f]-quinolin-3-yl)-nonyl]-piperazine-1-carboxylic acid ethyl ester (Compound 297);
(±)-3-{9-[Bis-(2-hydroxy-propyl)-amino]-nonyl}-2-ethyl-1-methyl-9-trifluoromethyl-3,6-dihydro-pyrrolo[3,2-f]quinolin-7-one (Compound 298);
2-Ethyl-3-[9-((1S,2S)-2-hydroxy-1-methyl-2-phenyl-ethylamino)-nonyl]-1-methyl-9-trifluoromethyl-3,6-dihydro-pyrrolo[3,2-f]quinolin-7-one (Compound 299);
(±)-9-Chloro-3-{9-[(2,3-dihydroxy-propyl)-methyl-amino]-nonyl}-1,2-dimethyl-3,6-dihydro-pyrrolo[3,2-f]quinolin-7-one (Compound 300);
9-Chloro-3-{9-[ethyl-(4-hydroxy-butyl)-amino]-nonyl}-1,2-dimethyl-3,6-dihydro-pyrrolo[3,2-f]quinolin-7-one (Compound 301);
9-Chloro-1,2-dimethyl-3-(9-{methyl-[4-(2-morpholin-4-yl-ethoxy)-benzyl]-amino}-nonyl)-3,6-dihydro-pyrrolo[3,2-f]quinolin-7-one (Compound 302);
9-Chloro-3-[9-(4-ethanesulfonyl-piperazin-1-yl)-nonyl]-1,2-dimethyl-3,6-dihydro-pyrrolo[3,2-f]quinolin-7-one (Compound 303);
2-Ethyl-3-[9-(4-hydroxy-butylamino)-nonyl]-1-methyl-9-trifluoromethyl-3,6-dihydro-pyrrolo[3,2-f]quinolin-7-one (Compound 304);
3-[9-(2,3-Dihydroxy-propylamino)-nonyl]-2-ethyl-1-methyl-9-trifluoromethyl-3,6-dihydro-pyrrolo[3,2-f]quinolin-7-one (Compound 305);
2-Ethyl-1-methyl-3-[9-(2-morpholin-4-yl-ethylamino)-nonyl]-9-trifluoromethyl-3,6-dihydro-pyrrolo[3,2-f]quinolin-7-one (Compound 306);
2-Ethyl-3-[9-(1-hydroxymethyl-cyclopentylamino)-nonyl]-1-methyl-9-trifluoromethyl-3,6-dihydro-pyrrolo[3,2-f]quinolin-7-one (Compound 307);
2-Ethyl-3-{9-[ethyl-(2-hydroxy-ethyl)-amino]-nonyl}-1-methyl-9-trifluoromethyl-3,6-dihydro-pyrrolo[3,2-f]quinolin-7-one (Compound 308);
2-Ethyl-3-[9-(2-hydroxy-1-phenyl-ethylamino)-nonyl]-1-methyl-9-trifluoromethyl-3,6-dihydro-pyrrolo[3,2-f]quinolin-7-one (Compound 309);
2-Ethyl-3-[9-(1-hydroxymethyl-pentylamino)-nonyl]-1-methyl-9-trifluoromethyl-3,6-dihydro-pyrrolo[3,2-f]quinolin-7-one (Compound 310);
3-[9-((1S,2R)-2,3-Dihydroxy-1-phenyl-propylamino)-nonyl]-2-ethyl-1-methyl-9-trifluoromethyl-3,6-dihydro-pyrrolo[3,2-f]quinolin-7-one (Compound 311);
3-{3-[Ethyl-(4-hydroxy-butyl)-amino]-propyl}-1,2-dimethyl-9-trifluoromethyl-3,6-dihydro-pyrrolo[3,2-f]quinolin-7-one (Compound 312);
2-[9-(2-Ethyl-1-methyl-7-oxo-9-trifluoromethyl-6,7-dihydro-pyrrolo[3,2-f]-quinolin-3-yl)-nonyl]-isoindole-1,3-dione (Compound 313);
Formic acid 9-(2-ethyl-1-methyl-7-oxo-9-trifluoromethyl-6,7-dihydro-pyrrolo[3,2-f]-quinolin-3-yl)-nonyl ester (Compound 314);
3-(9-Amino-nonyl)-2-ethyl-1-methyl-9-trifluoromethyl-3,6-dihydro-pyrrolo[3,2-f]-quinolin-7-one (Compound 315);
1-Ethyl-3-[9-(2-ethyl-1-methyl-7-oxo-9-trifluoromethyl-6,7-dihydro-pyrrolo[3,2-f]-quinolin-3-yl)-nonyl]-urea (Compound 316);
C-(4-Aminophenyl)-N-[9-(2-ethyl-1-methyl-7-oxo-9-trifluoromethyl-6,7-dihydro-pyrrolo[3,2-f]quinolin-3-yl)-nonanyl]-N-methyl-methanesulfonamide (Compound 317);
1-[9-(2-Ethyl-1-methyl-7-oxo-9-trifluoromethyl-6,7-dihydro-pyrrolo[3,2-f]-quinolin-3-yl)-nonyl]-3-phenyl-urea (Compound 318);
4-Amino-N-[9-(2-ethyl-1-methyl-7-oxo-9-trifluoromethyl-6,7-dihydro-pyrrolo[3,2-f]-quinolin-3-yl)-nonyl]-N-(2-hydroxy-ethyl)-benzenesulfonamide (Compound 319);
2-Ethyl-3-[9-(4-hydroxy-butylsulfanyl)-nonyl]-1-methyl-9-trifluoromethyl-3,6-dihydro-pyrrolo[3,2-f]quinolin-7-one (Compound 320);
2-Ethyl-3-[9-(4-hydroxy-butylsulfanyl)-nonyl]-1-methyl-9-trifluoromethyl-3,6-dihydro-pyrrolo[3,2-f]quinolin-7-one (Compound 321);
2-Ethyl-3-[9-(2-hydroxy-ethylsulfanyl)-nonyl]-1-methyl-9-trifluoromethyl-3,6-dihydro-pyrrolo[3,2-f]quinolin-7-one (Compound 322);
2-Ethyl-3-[9-(6-hydroxy-hexane-1-sulfinyl)-nonyl]-1-methyl-9-trifluoromethyl-3,6-dihydro-pyrrolo[3,2-f]quinolin-7-one (Compound 323);
2-Ethyl-3-[9-(2-mercapto-ethylsulfanyl)-nonyl]-1-methyl-9-trifluoromethyl-3,6-dihydro-pyrrolo[3,2-f]quinolin-7-one (Compound 324);
2-Ethyl-3-[9-(4-hydroxy-butane-1-sulfonyl)-nonyl]-1-methyl-9-trifluoromethyl-3,6-dihydro-pyrrolo[3,2-f]quinolin-7-one (Compound 325);
2-Ethyl-3-{9-[2-(2-mercapto-ethoxy)-ethylsulfanyl]-nonyl}-1-methyl-9-trifluoromethyl-3,6-dihydro-pyrrolo[3,2-f]quinolin-7-one (Compound 326);
2-Ethyl-1-methyl-3-[9-(1-phenyl-1H-tetrazol-5-ylsulfanyl)-nonyl]-9-trifluoromethyl-3,6-dihydro-pyrrolo[3,2-f]quinolin-7-one (Compound 327);
2-Ethyl-3-[9-(4-mercaptomethyl-benzylsulfanyl)-nonyl]-1-methyl-9-trifluoromethyl-3,6-dihydro-pyrrolo[3,2-f]quinolin-7-one (Compound 328);
N,N-1,4-Bis-(2-Ethyl-1-methyl-9-trifluoromethyl-3,6-dihydro-pyrrolo[3,2-f]-quinolin-7-one-3-(9-nonanyl))piperazine (Compound 329);
2-Ethyl-3-[6-(11-hydroxy-undecylsulfanyl)-hexyl]-1-methyl-9-trifluoromethyl-3,6-dihydro-pyrrolo[3,2-f]quinolin-7-one (Compound 330);
2-Ethyl-3-[9-(11-hydroxy-undecylsulfanyl)-nonyl]-1-methyl-9-trifluoromethyl-3,6-dihydro-pyrrolo[3,2-f]quinolin-7-one (Compound 331);
3-[9-(2-Ethyl-1-methyl-7-oxo-9-trifluoromethyl-6,7-dihydro-pyrrolo[3,2-f]-quinolin-3-yl)-nonylsulfanyl]-propionic acid (Compound 332);
3-(9-Allylamino-nonyl)-2-ethyl-1-methyl-9-trifluoromethyl-3,6-dihydro-pyrrolo[3,2-f]-quinolin-7-one (Compound 333);
2-Ethyl-1-methyl-3,6-bis-(2-{2-[2-(tetrahydro-pyran-2-yloxy)-ethoxy]-ethoxy}-ethyl)-9-trifluoromethyl-3,6-dihydro-pyrrolo[3,2-f]quinolin-7-one (Compound 334);
2-Ethyl-1-methyl-3-(2-{2-[2-(tetrahydro-pyran-2-yloxy)-ethoxy]-ethoxy}-ethyl)-9-trifluoromethyl-3,6-dihydro-pyrrolo[3,2-f]quinolin-7-one (Compound 335);
2-Ethyl-3-{2-[2-(2-hydroxy-ethoxy)-ethoxy]-ethyl}-1-methyl-9-trifluoromethyl-3,6-dihydro-pyrrolo[3,2-f]quinolin-7-one (Compound 336);
3-[9-(2,3-Dihydroxy-propylsulfanyl)-nonyl]-2-ethyl-1-methyl-9-trifluoromethyl-3,6-dihydro-pyrrolo[3,2-f]quinolin-7-one (Compound 337);
2-Ethyl-1-methyl-3-[9-(prop-2-ene-1-sulfinyl)-nonyl]-9-trifluoromethyl-3,6-dihydro-pyrrolo[3,2-f]quinolin-7-one (Compound 338);
2-Ethyl-3-[9-(2-hydroxy-ethanesulfinyl)-nonyl]-1-methyl-9-trifluoromethyl-3,6-dihydro-pyrrolo[3,2-f]quinolin-7-one (Compound 339);
2-Ethyl-1-methyl-3-[9-(prop-2-ene-1-sulfinyl)-nonyl]-9-trifluoromethyl-3,6-dihydro-pyrrolo[3,2-f]quinolin-7-one (Compound 340);
3-(2-{2-[2-(4-Ethanesulfonyl-piperazin-1-yl)-ethoxy]-ethoxy}-ethyl)-2-ethyl-1-methyl-9-trifluoromethyl-3,6-dihydro-pyrrolo[3,2-f]quinolin-7-one (Compound 341); and
(±)-3-[2-(2-{2-[(N-2,3-Dihydroxy-propyl)-N-methyl-amino]-ethoxy}-ethoxy)-ethyl]-2-ethyl-1-methyl-9-trifluoromethyl-3,6-dihydro-pyrrolo[3,2-f]quinolin-7-one (Compound 342).

13. The compound according of claim 1, wherein the compound is a selective androgen receptor modulator.

14. The selective androgen modulator of claim 13, wherein the selective androgen receptor modulator is a selective androgen receptor agonist.

15. The selective androgen modulator of claim 13, wherein the selective androgen receptor modulator is a selective androgen receptor antagonist.

16. The selective androgen modulator of claim 13, wherein the selective androgen receptor modulator is a selective androgen receptor partial agonist.

17. The selective androgen receptor modulator of claim 13, wherein the compound is a tissue-specific modulator

18. The compound of claim 1, wherein the compound is a selective androgen receptor binding compound.

19. A method for modulating an activity of an androgen receptor, comprising contacting an androgen receptor with a compound of claim 1, and thereby modulating its activity.

20. The method of claim 19, wherein the androgen receptor is in a cell.

21. A method for identifying a compound that modulates an activity of an androgen receptor, comprising:
contacting a cell expressing an androgen receptor with a compound of claim 1; and
monitoring an effect of the compound upon the cell.

22. A method for treating a patient having a condition susceptible to treatment with an androgen receptor modulator, comprising administering to the patient a pharmaceutical agent comprising a compound of claim 1.

23. The method of claim 22, wherein the condition is selected from maintenance of muscle strength and function; reversal or prevention of frailty or age-related functional decline in the elderly; treatment of catabolic side effects of glucocorticoids; treatment of reduced bone mass, density or growth; treatment of chronic fatigue syndrome; chronic myalgia; treatment of acute fatigue syndrome and muscle loss; accelerating of wound healing; accelerating bone fracture repair; accelerating healing of complicated fractures; in joint replacement; prevention of post-surgical adhesion formation; acceleration of tooth repair or growth; maintenance of sensory function; treatment of periodontal disease; treatment of wasting secondary to fractures and treatment of wasting in connection with chronic obstructive pulmonary disease, treatment of wasting in connection with chronic liver disease, treatment of wasting in connection with AIDS, cancer cachexia, burn and trauma recovery, chronic catabolic state, eating disorders and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia; treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem; improvement of cognitive function; treatment of catabolism in connection with pulmonary dysfunction and ventilator dependency; treatment of cardiac dysfunction; lowering blood pressure; protection against ventricular dysfunction or prevention of reperfusion events; treatment of adults in chronic dialysis; reversal or slowing of the catabolic state of aging; attenuation or reversal of protein catabolic responses following trauma; reducing cachexia and protein loss due to chronic illness; treatment of hyperinsulinemia; treatment of immunosuppressed patients; treatment of wasting in connection with multiple sclerosis or other neurodegenerative disorders; promotion of myelin repair; maintenance of skin thickness; treatment of metabolic homeostasis and renal homeostasis; stimulation of osteoblasts, bone remodeling and cartilage growth; regulation of food intake; treatment of insulin resistance; treatment of insulin resistance in the heart; treatment of hypothermia; treatment of congestive heart failure; treatment of lipodystrophy; treatment of muscular atrophy; treatment of musculoskeletal impairment; improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate and malignant tumor cells including the androgen receptor; osteosarcoma; hypercalcemia of malignancy; metastatic bone disease; treatment of spermatogenesis, endometriosis and polycystic ovary syndrome; counteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction, male and female contraception, hair loss, Reaven's Syndrome and the enhancement of bone and muscle strength.

24. The method of claim 22, wherein the patient has a condition selected from among acne, male-pattern baldness, wasting diseases, hirsutism, hypogonadism, osteoporoses, infertility, impotence and cancer.

25. A method for stimulating hematopoiesis in a patient, comprising administering to the patient a pharmaceutical agent comprising a compound of claim 1.

26. A method of contraception, comprising administering to patient a pharmaceutical agent comprising a compound of claim 1.

27. A method of improving athletic performance in an athlete, comprising administering to the athlete a pharmaceutical agent comprising a compound of claim 1.

28. A pharmaceutical composition, comprising a compound of claim 1 and a pharmaceutically acceptable carrier.

29.-31. (canceled)

32. An article of manufacture, comprising packaging material, a compound of claim 1 that is effective for modulating the activity of androgen receptor, or for treatment, prevention or amelioration of one or more symptoms of androgen receptor mediated diseases or disorders, or diseases or disorders in which androgen receptor activity is implicated, within the packaging material, and a label that indicates that the compound or composition, or pharmaceutically acceptable derivative thereof, is used for modulating the activity of androgen receptor or for treatment, prevention or amelioration of one or more symptoms of androgen receptor mediated diseases or disorders, or diseases or disorders in which androgen receptor activity is implicated.
